(19) United States
(12) Patent Application Publication
(10) Pub. No.: US 2024/0181030 A1
(43) Pub. Date: Jun. 6, 2024

(54) PERTUSSIS VACCINE

(71) Applicant: ModernaTX, Inc., Cambridge, MA (US)

(72) Inventors: Sunny Himansu, Winchester, MA (US); Elisabeth Narayanan, Cambridge, MA (US); Andrea Carfi, Cambridge, MA (US)

(73) Assignee: ModernaTX, Inc., Cambridge, MA (US)

(21) Appl. No.: 18/552,346

(22) PCT Filed: Mar. 25, 2022
(86) PCT No.: PCT/US2022/021908

§ 371 Date: Sep. 25, 2023

Related U.S. Application Data
(60) Provisional application No. 63/166,838, filed on Mar. 26, 2021.

(51) Int. Cl. A61K 39/02; A61K 9/51; A61K 39/00; A61K 39/116; A61K 47/60; A61P 37/04

(52) U.S. Cl. CPC A61K 39/099; A61K 9/5123; A61K 39/0016; A61K 39/116; A61K 47/60; A61P 37/04; A61K 2039/53; A61K 2039/545; A61K 2039/575; A61K 2039/70

(57) ABSTRACT

The disclosure relates to pertussis nucleic acid vaccines, diphtheria nucleic acid vaccines, tetanus nucleic acid vaccines, and combination vaccines, as well as methods of using the vaccines and compositions comprising the vaccines.

### RELATED APPLICATION

This application claims the benefit under 35 U.S.C. § 119(e) of U.S. provisional application No. 63/166,838, filed Mar. 26, 2021, which is incorporated by reference herein in its entirety.

### SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Mar. 23, 2022, is named M137870114WO00-SEQ-JXV and is 418,946 bytes in size.

### BACKGROUND

Pertussis, a respiratory disease also called whooping cough, is caused by Bordetella pertussis, a highly contagious, non-spore forming Gram-negative coccobacillus. The disease is transmitted primarily via aerosolized droplets, for example from the sneeze, cough, or breath of an infected person. Infected people are the most contagious for about two weeks after the cough begins, approximately five to 10 days after exposure. Early symptoms of pertussis are essentially identical to the symptoms of the common cold: running nose, low-grade fever, and a mild, occasional cough. After a week or two of disease progression, traditional pertussis symptoms, including paroxysm, vomiting, and exhaustion, occur. Worldwide, it is estimated that there are 20 million infections annually, resulting in approximately 200,000 deaths.

### SUMMARY

Provided herein are ribonucleic acid (RNA) vaccines that build on the knowledge that RNA (e.g., messenger RNA (mRNA)) can safely direct the body's cellular machinery to produce nearly any protein of interest, from native proteins to antibodies and other entirely novel protein constructs that can have therapeutic activity inside and outside of cells. The RNA (e.g., mRNA) vaccines of the present disclosure may be used to induce a balanced immune response against Bordetella pertussis (e.g., pertussis, whooping cough), diphtheria, and/or tetanus comprising both cellular and humoral immunity, without risking the possibility of insertional mutagenesis, for example.

The vaccines disclosed herein may be utilized in various settings depending on the prevalence of the infection or the degree or level of unmet medical need. The RNA (e.g. mRNA) vaccines may be utilized to treat and/or prevent pertussis, diphtheria, and/or tetanus of various genotypes, strains, and isolates.

The disclosure provides, in some aspects, a composition comprising at least one messenger ribonucleic acid (mRNA) polynucleotide having at least one open reading frame (ORF) encoding a Bordetella pertussis antigenic polypeptide selected from the group consisting of pertussis toxin antigenic polypeptides, autotransporter subtilisin-like protease (SPHB1) antigenic polypeptides, tracheal colonization factor A (TCFA) antigenic polypeptides, filamentous hemagglutinin (FHA) antigenic polypeptides, pertactin (PRN) antigenic polypeptides, fimbriae (FIM) antigenic polypeptides, adenylate cyclase antigenic polypeptides, Bordetella resistance to killing (Brk) antigenic polypeptides, and virulence-associated gene 8 (Vag8) antigenic polypeptides.

In some embodiments, the composition comprises at least two messenger ribonucleic acid (mRNA) polynucleotides, each having at least one ORF and each encoding a Bordetella pertussis antigenic polypeptide selected from the group consisting of pertussis toxin antigenic polypeptides, autotransporter subtilisin-like protease (SPHB1) antigenic polypeptides, tracheal colonization factor A (TCFA) antigenic polypeptides, filamentous hemagglutinin (FHA) antigenic polypeptides, pertactin (PRN) antigenic polypeptides, fimbriae (FIM) antigenic polypeptides, adenylate cyclase antigenic polypeptides, Bordetella resistance to killing (Brk) antigenic polypeptides, and virulence-associated gene 8 (Vag8) antigenic polypeptides.

In some embodiments, the composition comprises at least three messenger ribonucleic acid (mRNA) polynucleotides, each having at least one ORF and each encoding a Bordetella pertussis antigenic polypeptide selected from the group consisting of pertussis toxin antigenic polypeptides, autotransporter subtilisin-like protease (SPHB1) antigenic polypeptides, tracheal colonization factor A (TCFA) antigenic polypeptides, filamentous hemagglutinin (FHA) antigenic polypeptides, pertactin (PRN) antigenic polypeptides, fimbriae (FIM) antigenic polypeptides, adenylate cyclase antigenic polypeptides, Bordetella resistance to killing (Brk) antigenic polypeptides, and virulence-associated gene 8 (Vag8) antigenic polypeptides.

In some embodiments, the composition comprises at least four messenger ribonucleic acid (mRNA) polynucleotides, each having at least one ORF and each encoding a Bordetella pertussis antigenic polypeptide selected from the group consisting of pertussis toxin antigenic polypeptides, autotransporter subtilisin-like protease (SPHB1) antigenic polypeptides, tracheal colonization factor A (TCFA) antigenic polypeptides, filamentous hemagglutinin (FHA) antigenic polypeptides, pertactin (PRN) antigenic polypeptides, fimbriae (FIM) antigenic polypeptides, adenylate cyclase antigenic polypeptides, Bordetella resistance to killing (Brk) antigenic polypeptides, and virulence-associated gene 8 (Vag8) antigenic polypeptides.

In some embodiments, the composition comprises at least five messenger ribonucleic acid (mRNA) polynucleotides, each having at least one ORF and each encoding a Bordetella pertussis antigenic polypeptide selected from the group consisting of pertussis toxin antigenic polypeptides, autotransporter subtilisin-like protease (SPHB1) antigenic polypeptides, tracheal colonization factor A (TCFA) antigenic polypeptides, filamentous hemagglutinin (FHA) antigenic polypeptides, pertactin (PRN) antigenic polypeptides, fimbriae (FIM) antigenic polypeptides, adenylate cyclase antigenic polypeptides, Bordetella resistance to killing (Brk) antigenic polypeptides, and virulence-associated gene 8 (Vag8) antigenic polypeptides.

In some embodiments, the composition comprises at least six messenger ribonucleic acid (mRNA) polynucleotides, each having at least one ORF and each encoding a Bordetella pertussis antigenic polypeptide selected from the group consisting of pertussis toxin antigenic polypeptides, autotransporter subtilisin-like protease (SPHB1) antigenic polypeptides, tracheal colonization factor A (TCFA) antigenic polypeptides, filamentous hemagglutinin (FHA) antigenic polypeptides, pertactin (PRN) antigenic polypeptides, fimbriae (FIM) antigenic polypeptides, adenylate cyclase antigenic polypeptides, Bordetella resistance to killing (Brk) antigenic polypeptides, and virulence-associated gene 8 (Vag8) antigenic polypeptides.

In some embodiments, the composition comprises at least seven messenger ribonucleic acid (mRNA) polynucleotides, each having at least one ORF and each encoding a Bordetella pertussis antigenic polypeptide selected from the group consisting of pertussis toxin antigenic polypeptides, autotransporter subtilisin-like protease (SPHB1) antigenic polypeptides, tracheal colonization factor A (TCFA) antigenic polypeptides, filamentous hemagglutinin (FHA) antigenic polypeptides, pertactin (PRN) antigenic polypeptides, fimbriae (FIM) antigenic polypeptides, adenylate cyclase antigenic polypeptides, Bordetella resistance to killing (Brk) antigenic polypeptides, and virulence-associated gene 8 (Vag8) antigenic polypeptides.

In some embodiments, the composition comprises at least eight messenger ribonucleic acid (mRNA) polynucleotides, each having at least one ORF and each encoding a Bordetella pertussis antigenic polypeptide selected from the group consisting of pertussis toxin antigenic polypeptides, autotransporter subtilisin-like protease (SPHB1) antigenic polypeptides, tracheal colonization factor A (TCFA) antigenic polypeptides, filamentous hemagglutinin (FHA) antigenic polypeptides, pertactin (PRN) antigenic polypeptides, fimbriae (FIM) antigenic polypeptides, adenylate cyclase antigenic polypeptides, Bordetella resistance to killing (Brk) antigenic polypeptides, and virulence-associated gene 8 (Vag8) antigenic polypeptides.

In some embodiments, the composition comprises at least nine messenger ribonucleic acid (mRNA) polynucleotides, each having at least one ORF and each encoding a Bordetella pertussis antigenic polypeptide selected from the group consisting of pertussis toxin antigenic polypeptides, autotransporter subtilisin-like protease (SPHB1) antigenic polypeptides, tracheal colonization factor A (TCFA) antigenic polypeptides, filamentous hemagglutinin (FHA) antigenic polypeptides, pertactin (PRN) antigenic polypeptides, fimbriae (FIM) antigenic polypeptides, adenylate cyclase antigenic polypeptides, Bordetella resistance to killing (Brk) antigenic polypeptides, and virulence-associated gene 8 (Vag8) antigenic polypeptides.

In some embodiments, the pertussis toxin antigen polypeptide is selected from the group consisting of: S1 subunit, S2 subunit, S3 subunit, S4 subunit, S5 subunit, or a variant thereof. In some embodiments, the S1 subunit comprises an amino acid sequence that is at least 80%, 85%, 90%, 95%, or 98% identical to the sequence identified by SEQ ID NO: 8. In some embodiments, the S1 subunit comprises an amino acid sequence that is identical to the sequence identified by SEQ ID NO: 8. In some embodiments, the mRNA encoding the S1 subunit comprises a nucleotide sequence that is at least 95% or 98% identical to the sequence identified by SEQ ID NO: 7. In some embodiments, the mRNA encoding the S1 subunit comprises a nucleotide sequence that is identical to the sequence identified by SEQ ID NO: 7. In some embodiments, the mRNA encoding the S1 subunit comprises a nucleotide sequence that is at least 95% or 98% identical to the sequence identified by SEQ ID NO: 6. In some embodiments, the mRNA encoding the S1 subunit comprises a nucleotide sequence that is identical to the sequence identified by SEQ ID NO: 6. In some embodiments, the S1 subunit variant comprises an amino acid sequence that is at least 80%, 85%, 90%, 95%, or 98% identical to the sequence identified by SEQ ID NO: 5 or 11. In some embodiments, the S1 subunit variant comprises an amino acid sequence that is identical to the sequence identified by SEQ ID NO: 5 or 11. In some embodiments, the mRNA encoding the S1 subunit variant comprises a nucleotide sequence that is at least 95% or 98% identical to the sequence identified by SEQ ID NO: 3 or 10. In some embodiments, the mRNA encoding the S1 subunit variant comprises a nucleotide sequence that is identical to the sequence identified by SEQ ID NO: 3 or 10. In some embodiments, the mRNA encoding the S1 subunit variant comprises a nucleotide sequence that is at least 95% or 98% identical to the sequence identified by SEQ ID NO: 1 or 9. In some embodiments, the mRNA encoding the S1 subunit variant comprises a nucleotide sequence that is identical to the sequence identified by SEQ ID NO: 1 or 9. In some embodiments, the S2 subunit variant comprises an amino acid sequence that is at least 80%, 85%, 90%, 95%, or 98% identical to the sequence identified by SEQ ID NO: 14. In some embodiments, the S2 subunit variant comprises an amino acid sequence that is identical to the sequence identified by SEQ ID NO: 14. In some embodiments, the mRNA encoding the S2 subunit variant comprises a nucleotide sequence that is at least 95% or 98% identical to the sequence identified by SEQ ID NO: 13. In some embodiments, the mRNA encoding the S2 subunit variant comprises a nucleotide sequence that is identical to the sequence identified by SEQ ID NO: 13. In some embodiments, the mRNA encoding the S2 subunit variant comprises a nucleotide sequence that is at least 95% or 98% identical to the sequence identified by SEQ ID NO: 12. In some embodiments, the mRNA encoding the S2 subunit variant comprises a nucleotide sequence that is identical to the sequence identified by SEQ ID NO: 12. In some embodiments, the S3 subunit variant comprises an amino acid sequence that is at least 80%, 85%, 90%, 95%, or 98% identical to the sequence identified by SEQ ID NO: 17. In some embodiments, the S3 subunit variant comprises an amino acid sequence that is identical to the sequence identified by SEQ ID NO: 17. In some embodiments, the mRNA encoding the S3 subunit variant comprises a nucleotide sequence that is at least 95% or 98% identical to the sequence identified by SEQ ID NO: 16. In some embodiments, the mRNA encoding the S3 subunit variant comprises a nucleotide sequence that is identical to the sequence identified by SEQ ID NO: 16. In some embodiments, the mRNA encoding the S3 subunit variant comprises a nucleotide sequence that is at least 95% or 98% identical to the sequence identified by SEQ ID NO: 15. In some embodiments, the mRNA encoding the S3 subunit variant comprises a nucleotide sequence that is identical to the sequence identified by SEQ ID NO: 15. In some embodiments, the S4 subunit comprises an amino acid sequence that is at least 80%, 85%, 90%, 95%, or 98% identical to the sequence identified by SEQ ID NO: 20. In some embodiments, the S4 subunit variant comprises an amino acid sequence that is identical to the sequence identified by SEQ ID NO: 20. In some embodiments, the mRNA encoding the S4 subunit comprises a nucleotide sequence that is at least 95% or 98% identical to the sequence identified by SEQ ID NO: 19. In some embodiments, the mRNA encoding the S4 subunit comprises a nucleotide sequence that is identical to the sequence identified by SEQ ID NO: 19. In some embodiments, the mRNA encoding the S4 subunit comprises a nucleotide sequence that is at least 95% or 98% identical to the sequence identified by SEQ ID NO: 18. In some embodiments, the mRNA encoding the S4 subunit comprises a nucleotide sequence that is identical to the sequence identified by SEQ ID NO: 18. In some embodiments, the S5 subunit variant comprises an amino acid sequence that is at least 80%, 85%, 90%, 95%, or 98% identical to the sequence identified by SEQ ID NO: 23. In some embodiments, the S5 subunit variant comprises an amino acid sequence that is identical to the sequence identified by SEQ ID NO: 23. In some embodiments, the mRNA encoding the S5 subunit variant comprises a nucleotide sequence that is at least 95% or 98% identical to the sequence identified by SEQ ID NO: 22. In some embodiments, the mRNA encoding the S5 subunit variant comprises a nucleotide sequence that is identical to the sequence identified by SEQ ID NO: 22. In some embodiments, the mRNA encoding the S5 subunit variant comprises a nucleotide sequence that is at least 95% or 98% identical to the sequence identified by SEQ ID NO: 21. In some embodiments, the mRNA encoding the S5 subunit variant comprises a nucleotide sequence that is identical to the sequence identified by SEQ ID NO: 21.

In some embodiments, the SPHB1 antigenic polypeptide comprises an amino acid sequence that is at least 80%, 85%, 90%, 95%, or 98% identical to the sequence identified by SEQ ID NO: 26. In some embodiments, the SPHB1 antigenic polypeptide comprises an amino acid sequence that comprises the sequence identified by SEQ ID NO: 26. In some embodiments, the mRNA encoding the SPHB1 antigenic polypeptide comprises a nucleotide sequence that is at least 95% or 98% identical to the sequence identified by SEQ ID NO: 25. In some embodiments, the mRNA encoding the SPHB1 antigenic polypeptide comprises a nucleotide sequence that is identical to the sequence identified by SEQ ID NO: 25. In some embodiments, the mRNA encoding the SPHB1 antigenic polypeptide comprises a nucleotide sequence that is at least 95% or 98% identical to the sequence identified by SEQ ID NO: 24. In some embodiments, the mRNA encoding the SPHB1 antigenic polypeptide comprises a nucleotide sequence that is identical to the sequence identified by SEQ ID NO: 24.

In some embodiments, the TCFA antigenic polypeptide comprises an amino acid sequence that is at least 80%, 85%, 90%, 95%, or 98% identical to the sequence identified by SEQ ID NO: 29. In some embodiments, the TCFA antigenic polypeptide comprises an amino acid sequence that comprises the sequence identified by SEQ ID NO: 29. In some embodiments, the mRNA encoding the TCFA antigenic polypeptide comprises a nucleotide sequence that is at least 95% or 98% identical to the sequence identified by SEQ ID NO: 28. In some embodiments, the mRNA encoding the TCFA antigenic polypeptide comprises a nucleotide sequence that is identical to the sequence identified by SEQ ID NO: 28. In some embodiments, the mRNA encoding the TCFA antigenic polypeptide comprises a nucleotide sequence that is at least 95% or 98% identical to the sequence identified by SEQ ID NO: 27. In some embodiments, the mRNA encoding the TCFA antigenic polypeptide comprises a nucleotide sequence that is identical to the sequence identified by SEQ ID NO: 27.

In some embodiments, the filamentous hemagglutinin antigenic polypeptide comprises FHA1, FHA2, or FHA3. In some embodiments, the FHA3 antigenic polypeptide comprises an amino acid sequence that is at least 80%, 85%, 90%, 95%, or 98% identical to the sequence identified by SEQ ID NO: 35. In some embodiments, the FHA3 antigenic polypeptide comprises the sequence identified by SEQ ID NO: 35. In some embodiments, the mRNA encoding the FHA3 antigenic polypeptide comprises a nucleotide sequence that is at least 95% or 98% identical to the sequence identified by SEQ ID NO: 34. In some embodiments, the mRNA encoding the FHA3 antigenic polypeptide comprises a nucleotide sequence that is identical to the sequence identified by SEQ ID NO: 34. In some embodiments, the mRNA encoding the FHA3 antigenic polypeptide comprises a nucleotide sequence that is at least 95% or 98% identical to the sequence identified by SEQ ID NO: 33. In some embodiments, the mRNA encoding the FHA3 antigenic polypeptide comprises a nucleotide sequence that is identical to the sequence identified by SEQ ID NO: 33.

In some embodiments, the PRN antigenic polypeptide comprises an amino acid sequence that is at least 80%, 85%, 90%, 95%, or 98% identical to the sequence identified by SEQ ID NO: 32. In some embodiments, the PRN antigenic polypeptide comprises an amino acid sequence that comprises the sequence identified by SEQ ID NO: 32. In some embodiments, the mRNA encoding the PRN antigenic polypeptide comprises a nucleotide sequence that is at least 95% or 98% identical to the sequence identified by SEQ ID NO: 31. In some embodiments, the mRNA encoding the PRN antigenic polypeptide comprises a nucleotide sequence that is identical to the sequence identified by SEQ ID NO: 31. In some embodiments, the mRNA encoding the PRN antigenic polypeptide comprises a nucleotide sequence that is at least 95% or 98% identical to the sequence identified by SEQ ID NO: 30. In some embodiments, the mRNA encoding the PRN antigenic polypeptide comprises a nucleotide sequence that is identical to the sequence identified by SEQ ID NO: 30.

In some embodiments, the FIM antigenic polypeptides are selected from the group consisting of: FIM1, FIM2, FIM 3, and domain-swapped constructs thereof. In some embodiments, the FIM antigenic polypeptide comprises an amino acid sequence that is at least 80%, 85%, 90%, 95%, or 98% identical to the sequence identified by SEQ ID NO: 38. In some embodiments, the FIM antigenic polypeptide comprises an amino acid sequence that comprises the sequence identified by SEQ ID NO: 38. In some embodiments, the mRNA encoding the FIM antigenic polypeptide comprises a nucleotide sequence that is at least 95% or 98% identical to the sequence identified by SEQ ID NO: 37. In some embodiments, the mRNA encoding the FIM antigenic polypeptide comprises a nucleotide sequence that is identical to the sequence identified by SEQ ID NO: 37. In some embodiments, the mRNA encoding the FIM antigenic polypeptide comprises a nucleotide sequence that is at least 95% or 98% identical to the sequence identified by SEQ ID NO: 36. In some embodiments, the mRNA encoding the FIM antigenic polypeptide comprises a nucleotide sequence that is identical to the sequence identified by SEQ ID NO: 36.

In some embodiments, the adenylate cyclase antigenic polypeptides are selected from the group consisting of: ACT_{188LQ}, ACT_{H63A_K65A_S66G}, and the repeats-in-toxin (RTX) domain. In some embodiments, the RTX antigenic polypeptide comprises an amino acid sequence that is at least 80%, 85%, 90%, 95%, or 98% identical to the sequence identified by SEQ ID NO: 41. In some embodiments, the RTX antigenic polypeptide comprises an amino acid sequence that comprises the sequence identified by SEQ ID NO: 41. In some embodiments, the mRNA encoding the RTX antigenic polypeptide comprises a nucleotide sequence that is at least 95% or 98% identical to the sequence identified by SEQ ID NO: 40. In some embodiments, the mRNA encoding the RTX antigenic polypeptide comprises a nucleotide sequence that is identical to the sequence identified by SEQ ID NO: 40. In some embodiments, the mRNA encoding the RTX antigenic polypeptide comprises a nucleotide sequence that is at least 95% or 98% identical to the sequence identified by SEQ ID NO: 39. In some embodiments, the mRNA encoding the RTX antigenic polypeptide comprises a nucleotide sequence that is identical to the sequence identified by SEQ ID NO: 39.

In some embodiments, the Brk antigenic polypeptide comprises an amino acid sequence that is at least 80%, 85%, 90%, 95%, or 98% identical to the sequence identified by SEQ ID NO: 44. In some embodiments, the Brk antigenic polypeptide comprises an amino acid sequence that comprises the sequence identified by SEQ ID NO: 44. In some embodiments, the mRNA encoding the Brk antigenic polypeptide comprises a nucleotide sequence that is at least 95% or 98% identical to the sequence identified by SEQ ID NO: 43. In some embodiments, the mRNA encoding the Brk antigenic polypeptide comprises a nucleotide sequence that is identical to the sequence identified by SEQ ID NO: 43. In some embodiments, the mRNA encoding the Brk antigenic polypeptide comprises a nucleotide sequence that is at least 95% or 98% identical to the sequence identified by SEQ ID NO: 42. In some embodiments, the mRNA encoding the Brk antigenic polypeptide comprises a nucleotide sequence that is identical to the sequence identified by SEQ ID NO: 42.

In some embodiments, the Vag8 antigenic polypeptide comprises an amino acid sequence that is at least 80%, 85%, 90%, 95%, or 98% identical to the sequence identified by SEQ ID NO: 47. In some embodiments, the Vag8 antigenic polypeptide comprises an amino acid sequence that comprises the sequence identified by SEQ ID NO: 47. In some embodiments, the mRNA encoding the Vag8 antigenic polypeptide comprises a nucleotide sequence that is at least 95% or 98% identical to the sequence identified by SEQ ID NO: 46. In some embodiments, the mRNA encoding the Vag8 antigenic polypeptide comprises a nucleotide sequence that is identical to the sequence identified by SEQ ID NO: 46. In some embodiments, the mRNA encoding the Vag8 antigenic polypeptide comprises a nucleotide sequence that is at least 95% or 98% identical to the sequence identified by SEQ ID NO: 45. In some embodiments, the mRNA encoding the Vag8 antigenic polypeptide comprises a nucleotide sequence that is identical to the sequence identified by SEQ ID NO: 45.

In some embodiments, any of the compositions provided herein further comprise at least one mRNA polynucleotide having at least one ORF encoding a diphtheria antigenic polypeptide. In some embodiments, the diphtheria antigenic polypeptide comprises an amino acid sequence that is at least 80%, 85%, 90%, 95%, or 98% identical to the sequence identified by SEQ ID NO: 50. In some embodiments, the diphtheria antigenic polypeptide comprises an amino acid sequence that comprises the sequence identified by SEQ ID NO: 50. In some embodiments, the mRNA encoding the diphtheria antigenic polypeptide comprises a nucleotide sequence that is at least 95% or 98% identical to the sequence identified by SEQ ID NO: 49. In some embodiments, the mRNA encoding the diphtheria antigenic polypeptide comprises a nucleotide sequence that is identical to the sequence identified by SEQ ID NO: 49. In some embodiments, the mRNA encoding the diphtheria antigenic polypeptide comprises a nucleotide sequence that is at least 95% or 98% identical to the sequence identified by SEQ ID NO: 48. In some embodiments, the mRNA encoding the diphtheria antigenic polypeptide comprises a nucleotide sequence that is identical to the sequence identified by SEQ ID NO: 48.

In some embodiments, any one of the compositions provided herein further comprise at least one mRNA polynucleotide having at least one ORF encoding a tetanus antigenic polypeptide. In some embodiments, the tetanus antigenic polypeptide comprises an amino acid sequence that is at least 80%, 85%, 90%, 95%, or 98% identical to the sequence identified by SEQ ID NO: 53. In some embodiments, the tetanus antigenic polypeptide comprises an amino acid sequence that comprises the sequence identified by SEQ ID NO: 53. In some embodiments, the mRNA encoding the tetanus antigenic polypeptide comprises a nucleotide sequence that is at least 95% or 98% identical to the sequence identified by SEQ ID NO: 52. In some embodiments, the mRNA encoding the tetanus antigenic polypeptide comprises a nucleotide sequence that is identical to the sequence identified by SEQ ID NO: 52. In some embodiments, the mRNA encoding the tetanus antigenic polypeptide comprises a nucleotide sequence that is at least 95% or 98% identical to the sequence identified by SEQ ID NO: 51. In some embodiments, the mRNA encoding the tetanus antigenic polypeptide comprises a nucleotide sequence that is identical to the sequence identified by SEQ ID NO: 51.

In some aspects, a composition comprising at least one messenger ribonucleic acid (mRNA) polynucleotide having at least one open reading frame (ORF) encoding a diphtheria antigenic polypeptide, is provided. In some embodiments, the diphtheria antigenic polypeptide comprises an amino acid sequence that is at least 80%, 85%, 90%, 95%, or 98% identical to the sequence identified by SEQ ID NO: 50. In some embodiments, the diphtheria antigenic polypeptide comprises an amino acid sequence that comprises the sequence identified by SEQ ID NO: 50. In some embodiments, the mRNA encoding the diphtheria antigenic polypeptide comprises a nucleotide sequence that is at least 95% or 98% identical to the sequence identified by SEQ ID NO: 49. In some embodiments, the mRNA encoding the diphtheria antigenic polypeptide comprises a nucleotide sequence that is identical to the sequence identified by SEQ ID NO: 49. In some embodiments, the mRNA encoding the diphtheria antigenic polypeptide comprises a nucleotide sequence that is at least 95% or 98% identical to the sequence identified by SEQ ID NO: 48. In some embodiments, the mRNA encoding the diphtheria antigenic polypeptide comprises a nucleotide sequence that is identical to the sequence identified by SEQ ID NO: 48.

In some aspects, a composition comprising at least one messenger ribonucleic acid (mRNA) polynucleotide having at least one open reading frame (ORF) encoding a tetanus antigenic polypeptide, is provided. In some embodiments, the tetanus antigenic polypeptide comprises an amino acid sequence that is at least 80%, 85%, 90%, 95%, or 98% identical to the sequence identified by SEQ ID NO: 53. In some embodiments, the tetanus antigenic polypeptide comprises an amino acid sequence that comprises the sequence identified by SEQ ID NO: 53. In some embodiments, the mRNA encoding the tetanus antigenic polypeptide comprises a nucleotide sequence that is at least 95% or 98% identical to the sequence identified by SEQ ID NO: 52. In some embodiments, the mRNA encoding the tetanus antigenic polypeptide comprises a nucleotide sequence that is identical to the sequence identified by SEQ ID NO: 52. In some embodiments, the mRNA encoding the tetanus antigenic polypeptide comprises a nucleotide sequence that is at least 95% or 98% identical to the sequence identified by SEQ ID NO: 51. In some embodiments, the mRNA encoding the tetanus antigenic polypeptide comprises a nucleotide sequence that is identical to the sequence identified by SEQ ID NO: 51.

In some embodiments, any one of the compositions provided herein further comprise an mRNA encoding an antigenic fusion polypeptide selected from the group consisting of: pertussis antigenic fusion polypeptides, tetanus antigenic fusion polypeptides, diphtheria antigenic fusion polypeptides, or a combination thereof. In some embodiments, the antigenic fusion polypeptide comprises an amino acid sequence that is at least 80%, 85%, 90%, 95%, or 98% identical to a sequence selected from SEQ ID NOs: 56, 59, 62, 65, 68, 71, 74, 77, 80, 83, 86, 89, 92, 95, and 98. In some embodiments, the antigenic fusion polypeptide comprises an amino acid sequence that comprises a sequence selected from SEQ ID NOs: 56, 59, 62, 65, 68, 71, 74, 77, 80, 83, 86, 89, 92, 95, and 98. In some embodiments, the mRNA encoding the antigenic fusion polypeptide comprises a nucleotide sequence that is at least 95% or 98% identical to a sequence selected from SEQ ID NOs: 55, 58, 61, 64, 67, 70, 73, 76, 79, 82, 85, 88, 91, 94, and 97. In some embodiments, the mRNA encoding the antigenic fusion polypeptide comprises a nucleotide sequence that is identical to a sequence selected from SEQ ID NOs: 55, 58, 61, 64, 67, 70, 73, 76, 79, 82, 85, 88, 91, 94, and 97. In some embodiments, the mRNA encoding the antigenic fusion polypeptide comprises a nucleotide sequence that is at least 95% or 98% identical to a sequence selected from SEQ ID NOs: 54, 57, 60, 63, 66, 69, 72, 75, 78, 81, 84, 87, 90, 93, and 96. In some embodiments, the mRNA encoding the antigenic fusion polypeptide comprises a nucleotide sequence that is identical to a sequence selected from SEQ ID NOs: 54, 57, 60, 63, 66, 69, 72, 75, 78, 81, 84, 87, 90, 93, and 96.

In some embodiments, the mRNA comprises a 5′ untranslated region (UTR) comprising the nucleotide sequence of SEQ ID NO: 2. In some embodiments, the mRNA comprises a 3′ UTR comprising the nucleotide sequence of SEQ ID NO: 4. In some embodiments, the mRNA further comprises a chemical modification. In some embodiments, the chemical modification is 1-methylpseudouridine.

In some embodiments, any one of the compositions described herein further comprises a lipid nanoparticle. In some embodiments, the lipid nanoparticle comprises a PEG-modified lipid, a non-cationic lipid, a sterol, an ionizable amino lipid, or any combination thereof. In some embodiments, the lipid nanoparticle comprises 0.5-15 mol % PEG-modified lipid; 5-25 mol % non-cationic lipid; 25-55 mol % sterol; and 20-60 mol % ionizable amino lipid.

In some embodiments, the PEG-modified lipid is 1,2 dimyristoyl-sn-glycerol, methoxypolyethyleneglycol (PEG2000 DMG), the non-cationic lipid is 1,2 distearoyl-sn-glycero-3-phosphocholine (DSPC), the sterol is cholesterol, and the ionizable amino lipid has the structure of Compound 1:

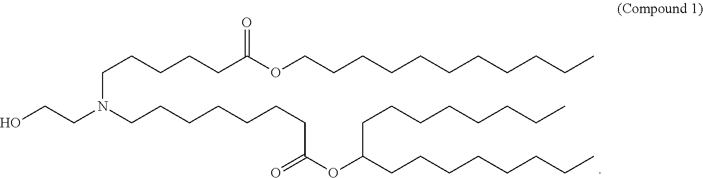 [SMILES: CCCCCCCCCCCOC(=O)CCCCCN(CCO)CCCCCCCC(=O)OC(CCCCCCCC)CCCCCCCC]

In some aspects, the disclosure provides a method comprising administering to a subject the composition of any one of the preceding claims in an amount effective to induce a neutralizing antibody response against Bordetella pertussis in the subject.

In some embodiments, the composition is administered in an amount effective to induce a Th1 immune response, a Th17 immune response, a Th2 response, or a combination thereof in the subject. In some embodiments, the composition is administered in an amount effective to reduce or eliminate symptoms of pertussis in the subject. In some embodiments, the composition is administered in an amount effective to reduce or eliminate colonization of the subject's respiratory tract. In some embodiments, the composition is administered in an amount effective to reduce or eliminate transmissibility of B. pertussis.

In some embodiments, the composition is further administered a second time, as a boost. In some embodiments, the boost dose is administered 28 days after a first dose.

The details of various embodiments of the disclosure are set forth in the description below. Other features, objects, and advantages of the disclosure will be apparent from the description and from the claims.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a graph depicting the percent weight change (weight loss) in mice following injection with the indicated mRNA constructs and then exposure to Bordetella pertussis, as described in Example 1.

FIG. 2 is a graph showing the blood cellularity of mice following injection with the indicated mRNA constructs and then exposure to Bordetella pertussis, as described in Example 1. White blood cell, neutrophil, and lymphocyte counts are illustrated.

FIG. 3 is a graph showing the concentration of IL-6 in mice following injection with the indicated mRNA constructs and then exposure to Bordetella pertussis, as described in Example 1.

FIG. 4 is a graph showing the bacterial load (as measured by CFU) in the trachea and lung of mice following injection with the indicated mRNA constructs and then exposure to Bordetella pertussis, as described in Example 1.

FIG. 5 is a graph showing the bacterial load (as measured by CFU) in the nasal lavage of mice following injection with the indicated mRNA constructs and then exposure to Bordetella pertussis, as described in Example 1.

FIGS. 6A-6B show antibody titers to toxin. FIG. 6A shows antibody titers (anti-PT) one week after the booster dose was administered (Example 1) and FIG. 6B shows anti-RTX antibody titers three days after challenge (Example 1).

FIGS. 7A-7B show additional antibody titer characterization. FIG. 7A shows antibody titers using extracellular pertussis protein as an antigen (100 proteins) and FIG. 7B shows the results from a live Bordetella pertussis binding assay (whole bug titers).

FIG. 8 is a graph showing serum IgG antibody titers to pertussis toxin by ELISA three days after challenge (Example 2). Data are presented as mean±SEM, n=5 per treatment group at each time point. ** significantly different from Mock Vaccinated (p<0.005), *** significantly different from Mock Vaccinated (p<0.0005). Black dotted line indicates lowest limit of detection. Kruskal-Wallis non-parametric test with Dunnet's post-hoc test was used for statistical analysis.

FIG. 9 is a graph showing serum IgG antibody titers to whole bug (UT25) by ELISA three days after challenge (Example 2). Data are presented as mean±SEM, n=5 per treatment group at each time point. *significantly different from Mock Vaccinated (p<0.05), ** significantly different from Mock Vaccinated (p<0.005), *** significantly different from Mock Vaccinated (p<0.0005). Black dotted line indicates lowest limit of detection. Kruskal-Wallis non-parametric test with Dunnet's post-hoc test was used for statistical analysis.

FIGS. 10A-10B are graphs showing the bacterial burden measured three days after challenge in subjects lung and trachea (FIG. 10A) and nasal lavage (FIG. 10B) (Example 2).

FIGS. 11A-11B are graphs showing anti-diphtheria toxin titers (FIG. 11A) and anti-tetanus toxin titers (FIG. 11B) in mice before boost administration and after boost administration of different doses of vaccine (Example 3).

FIG. 12 is a summary graph showing the IC₅₀ resulting from three different antigens administered at 10 μg doses (Example 3).

FIG. 13 shows the colony forming units (CFUs) in lung and trachea samples three days post B. pertussis challenge. Data is presented as mean±SEM (standard error of mean), n=5 per treatment group, NS=No significance, * significantly different from Mock Vaccinated (p<0.0231), **** significantly different from Mock Vaccinated (p<0.0001). The black line indicates lowest limit of detection. One-way ANOVA with Tukeys' post-hoc test was used for statistical analysis.

FIG. 14 shows the CFUs in nasal lavage samples from three days post B. pertussis challenge. Data is presented as mean±SEM, n=5 per treatment group. NS=No significance. Black line indicates lowest limit of detection. One-way ANOVA with Tukeys' post-hoc test was used for statistical analysis.

FIG. 15 shows the CFUs present in nasal-associated lymphoid tissue (NALT) from three days post B. pertussis challenge. Data is presented as mean±SEM, n=5 per treatment group. Black line indicates lowest limit of detection. * significantly different from Mock Vaccinated (p<0.0326), One-way ANOVA with Tukeys' post-hoc test was used for statistical analysis.

FIG. 16 shows the percent change in mouse weight between the day of the challenge and three days post-challenge. Weights were taken day of challenge and then 3 days post-challenge. Data is presented as mean percent weight change±SEM, n=5 per treatment group.

FIG. 17 shows the complete white blood cell counts between different immunization groups three days after B. pertussis challenge. Hemavet was used to determine neutrophil counts after challenge. Data is presented as mean±SEM, n=5 per treatment group. NS=No significance. One-way ANOVA with Tukeys' post-hoc test was used for statistical analysis.

FIG. 18 shows neutrophil counts between different immunization groups three days after B. pertussis challenge. Hemavet was used to determine neutrophil counts after challenge. Data is presented as mean±SEM, n=5 per treatment group at each time point. NS=No significance. One-way ANOVA with Tukeys' post-hoc test was used for statistical analysis.

FIG. 19 shows lymphocyte counts between different immunization groups three days after B. pertussis challenge. Hemavet was used to determine Lymphocyte counts after challenge. Data is presented as mean±SEM, n=5 per treatment group at each time point. NS=No significance. One-way ANOVA with Tukeys' post-hoc test was used for statistical analysis.

FIG. 20 shows monocyte counts between different immunization groups three days after B. pertussis challenge. Hemavet was used to determine Monocytes counts after challenge. Data is presented as mean±SEM, n=5 per treatment group at each time point. NS=No significance. One-way ANOVA with Tukeys' post-hoc test was used for statistical analysis.

FIG. 21 shows eosinophil counts between different immunization groups three days after B. pertussis challenge. Hemavet was used to determine Eosinophils counts after challenge. Data is presented as mean±SEM, n=5 per treatment group at each time point. NS=No significance. One-way ANOVA with Tukeys' post-hoc test was used for statistical analysis.

FIG. 22 shows serum antibody (IgG) titers to whole bug (B. pertussis strain UT25). Data is presented as mean±SEM, n=5 per treatment group. * significantly different from Mock Vaccinated (p<0.0372), ** significantly different from Mock Vaccinated (p<0.0091), *** significantly different from Mock Vaccinated (p<0.0007). Black line indicates lowest limit of detection. Kruskal-Wallis non-parametric test with Dunnet's post-hoc test was used for statistical analysis.

FIG. 23 shows serum antibody (IgG) titers to pertussis toxin (PT), at three days post-challenge. Data is presented as mean±SEM, n=5 per treatment group. * significantly different from Mock Vaccinated (p<0.0313), **** significantly different from Mock Vaccinated (p<0.0001). Black line indicates lowest limit of detection. Kruskal-Wallis non-parametric test with Dunnet's post-hoc test was used for statistical analysis.

FIG. 24 shows serum antibody (IgG) titers to diphtheria toxin three days post-challenge. Data is presented as mean±SEM, n=5 per treatment group. * significantly different from Mock Vaccinated (p<0.0182), *** significantly different from Mock Vaccinated (p<0.0009). Black line indicates lowest limit of detection. Kruskal-Wallis non-parametric test with Dunnet's post-hoc test was used for statistical analysis.

FIG. 25 shows serum antibody (IgG) titers to tetanus toxin three days post-challenge. Data is presented as mean±SEM, n=5 per treatment group at each time point. ** significantly different from Mock Vaccinated (p<0.0040), *** significantly different from Mock Vaccinated (p<0.0009). Black line indicates lowest limit of detection. Kruskal-Wallis non-parametric test with Dunnet's post-hoc test was used for statistical analysis.

FIG. 26 shows IL-6 levels in lung supernatant samples. Data is presented as mean±SEM, n=5 per treatment group at each time point. **** significantly different from NVNC (p<0.0001). All groups were significantly different from Mock Vaccinated (p<0.0001). Gray-filled circles indicate measurement is outside of detection range. One-way ANOVA with Tukeys' post-hoc test was used for statistical analysis.

FIG. 27 shows IL-6 levels in sera samples. Data is presented as mean±SEM, n=5 per treatment group at each time point. **** significantly different from NVNC (p<0.0005), all groups were significantly different from Mock Vaccinated (p<0.0001). Gray-filled circles indicate measurement is outside of detection range. One-way ANOVA with Tukeys' post-hoc test was used for statistical analysis.

FIG. 28 shows anti-B. pertussis antibody titers in mice two weeks after the priming vaccination dose.

FIGS. 29A-29C show the colony-forming units (CFUs) in lung and trachea samples from mice challenged with B. pertussis strain UT25 or D420 on Day 1 (FIG. 29A), Day 3 (FIG. 29B), and Day 7 (FIG. 29C) post-challenge.

FIGS. 30A-30B show a time course of the CFUs in lung and trachea samples from mice challenged with B. pertussis strain UT25 (FIG. 30A) or D420 (FIG. 30B).

### DETAILED DESCRIPTION

Bordetella pertussis is a highly-contagious, aerobic, non-spore forming, Gram-negative coccobacillus. The bacteria attach to the cilia of respiratory epithelial cells and produce toxins, which have a variety of effects on immune cells. For example, pertussis toxin (PT) inhibits the protective anti-bacterial function of resident airway macrophages and inhibits the influx of neutrophils to the airways. As the bacteria multiply, inflammation and mucus hypersecretion occur. Pertussis antigens permit the bacteria to evade host defenses, as lymphocytosis is promoted while chemotaxis is impaired. The virulence factors produced by B. pertussis include those associated with adhesions and playing a role in attachment/colonization, persistence, and carriage, which include: filamentous hemagglutinin (FHA), agglutinogens, pertactin, and fimbriae (FIM). Other virulence factors are toxins, which play roles in pathology and persistence. The B. pertussis toxins include: pertussis toxin (PT), adenylate cyclase toxin (ACT), tracheal cytotoxin, dermo-necrotic toxin, heat-liable toxin, and LPS (lipopolysaccharide; endotoxin).

Two main approaches to pertussis vaccines have been used. In the whole cell vaccine (wP), the vaccine comprises heat-killed or formalin-inactivated B. pertussis culture, including a large number of antigens. There are a number side effects and the vaccines are contraindicated for some individuals. Similar to natural infection, the wP vaccines trigger Th1 and Th17 immune responses. Protection lasts for 3-5 years. The wP vaccines are reactogenic in children and produced in a variety of different ways, resulting in heterogenous vaccines. In contrast, acellular vaccines (aP), which comprise recombinant/purified bacterial antigens (e.g., PT, FHA, PRN and FIM2/3) detoxified by chemical methods. The aP vaccine (DTaP, for children; Tdap, for adults) requires three doses to achieve approximately 85% efficacy and the duration of protection is unknown; however, there is a decline in efficacy after two years. The aP vaccines trigger a Th2-biased immune response. The current aP vaccines do not include certain virulence factors, such as ACT. In addition, aP vaccines prevent symptomatic disease by neutralization; however, they do not prevent colonization or transmission (e.g., vaccinated individuals may be asymptomatic carriers). Children primed with an aP vaccine have been found to have a 2- to 5-fold greater risk of acute infection compared to those primed with a wP vaccine. Furthermore, children are susceptible, as adults who have taken the aP vaccine may be a reservoir of infection.

Provided herein, are nucleic acid vaccines encoding B. pertussis antigens, and optionally, diphtheria and/or tetanus antigens, that provide durable protection, including prevention of colonization and transmission. Combinations of the vaccine antigens delivered as mRNA vaccines are particularly effective. The vaccines described herein also elicit Th1, Th2, and/or Th17 immune responses.

### Antigens

The compositions of the invention, e.g., vaccine compositions, feature nucleic acids, in particular, mRNAs, designed to encode an antigen of interest, e.g., an antigen derived from a Bordetella pertussis, diphtheria, or tetanus protein. The compositions of the invention, e.g., vaccine compositions, do not comprise antigens per se, but rather comprise nucleic acids, in particular, mRNA(s) that encode antigens or antigenic sequences once delivered to a cell, tissue or subject. Delivery of nucleic acids, in particular mRNA(s) is achieved by formulating said nucleic acids in appropriate carriers or delivery vehicles (e.g., lipid nanoparticles) such that upon administration to cells, tissues or subjects, nucleic acid is taken up by cells which, in turn, express protein(s) encoded by the nucleic acids, e.g., mRNAs.

Antigens, as used herein, are proteins capable of inducing an immune response (e.g., causing an immune system to produce antibodies against the antigens). The vaccines of the present disclosure provide a unique advantage over traditional protein-based vaccination approaches in which protein antigens are purified or produced in vitro, e.g., recombinant protein production technologies. The vaccines of the present disclosure feature mRNA encoding the desired antigens, which when introduced into the body, i.e., administered to a mammalian subject (for example a human) in vivo, cause the cells of the body to express the desired antigens.

Bacterial antigens, such as those disclosed herein, are not typically produced in mammalian cells. Therefore, in order to generate mRNA vaccines wherein bacterial antigens are produced by the subject (e.g., by mammalian cells), several modifications are made. First, bacterial comprise autotransporter domains on their outer membrane proteins that form beta barrels, allowing bacteria to export virulence factors (e.g., toxins). This process aids with proper folding of the protein. Mammalian cells do not have autotransporters or beta barrels and, when mRNA encoding such structures is produced in mammalian cells, the resulting structures do not fit properly in the mammalian membrane. To address this, the antigens disclosed herein comprise a secretion signal, such that they are secreted from the mammalian cells in a process analogous to the autotransporter export in bacteria. Second, bacterial systems glycosylate proteins; however, glycosylation does occur somewhat frequently in mammalian cells. To prevent glycosylation, the mRNA described herein encodes antigens wherein residues prone to N-linked glycosylation (e.g., asparagine) have been removed, modified, or substituted in order to prevent glycosylation.

In order to facilitate delivery of the mRNAs of the present disclosure to the cells of the body, the mRNAs are encapsulated in lipid nanoparticles (LNPs). Upon delivery and uptake by cells of the body, the mRNAs are translated in the cytosol and protein antigens are generated by the host cell machinery. The protein antigens are presented and elicit an adaptive humoral and cellular immune response. Neutralizing antibodies are directed against the expressed protein antigens and hence the protein antigens are considered relevant target antigens for vaccine development. Herein, use of the term “antigen” encompasses immunogenic proteins and immunogenic fragments (an immunogenic fragment that induces (or is capable of inducing) an immune response to B. pertussis, and optionally, diphtheria and/or tetanus), unless otherwise stated. It should be understood that the term “protein” encompasses peptides and the term “antigen” encompasses antigenic fragments.

Many proteins have a quaternary or three-dimensional structure, which consists of more than one polypeptide or several polypeptide chains that associate into an oligomeric molecule. As used herein the term “subunit” refers to a single protein molecule, for example, a polypeptide or polypeptide chain resulting from processing of a nascent protein molecule, which subunit assembles (or “coassembles”) with other protein molecules (e.g., subunits or chains) to form a protein complex. Proteins can have a relatively small number of subunits and therefore be described as “oligomeric” or can consist of a large number of subunits and therefore be described as “multimeric”. The subunits of an oligomeric or multimeric protein may be identical, homologous or totally dissimilar and dedicated to disparate tasks.

Proteins or protein subunits can further comprise domains. As used herein, the term “domain” refers to a distinct functional and/or structural unit within a protein. Typically, a “domain” is responsible for a particular function or interaction, contributing to the overall role of a protein. Domains can exist in a variety of biological contexts. Similar domains (i.e., domains sharing structural, functional and/or sequence homology) can exist within a single protein or can exist within distinct proteins having similar or different functions. A protein domain is often a conserved part of a given protein tertiary structure or sequence that can function and exist independently of the rest of the protein or subunit thereof.

As used herein, the term antigen is distinct from the term “epitope” which is a substructure of an antigen, e.g., a polypeptide, such as 7-10 amino acids, or carbohydrate structure, which may be recognized by an antigen binding site but is insufficient to induce an immune response. The art describes protein antigens that are delivered to subjects or immune cells in isolated form, e.g., isolated protein, polypeptide or peptide antigens, however, the design, testing, validation, and production of protein antigens can be costly and time-consuming, especially when producing proteins at large scale. By contrast, mRNA technology is amenable to rapid design and testing of mRNA constructs encoding a variety of antigens. Moreover, rapid production of mRNA coupled with formulation in appropriate delivery vehicles (e.g., lipid nanoparticles), can proceed quickly and can rapidly produce mRNA vaccines at large scale. Potential benefit also arises from the fact that antigens encoded by the mRNAs of the invention are expressed by the cells of the subject, e.g., are expressed by the human body, and thus the subject, e.g., the human body, serves as the “factory” to produce the antigens which, in turn, elicits the desired immune response.

The compositions, as provided herein, may include an RNA or multiple RNAs encoding two or more antigens of the same or different pertussis strains. Also provided herein are combination vaccines that include RNA encoding one or more pertussis antigens and one or more antigen(s) of a different organism. Thus, the vaccines of the present disclosure may be combination vaccines that target one or more antigens of the same strain/species, or one or more antigens of different strains/species, e.g., antigens which induce immunity to organisms which are found in the same geographic areas where the risk of pertussis infection is high or organisms to which an individual is likely to be exposed to when exposed to B. pertussis.

### Encoded Pertussis, Diphtheria, and Tetanus Antigens

The compositions provided herein comprise mRNA encoding at least one pertussis, diphtheria or tetanus antigen, as described below. Each antigen encoded by the mRNA is chemically detoxified, e.g., with mutations.

Pertussis toxin comprises five subunits. The A subunit (S1) is the toxin. It binds to, and inactivates, G proteins, as well as suppresses cytokine signaling. The B pentamer (S2-S5) binds to membrane receptors in the host. As described herein, in some embodiments, the vaccine comprises mRNA encoding soluble S1 (e.g., the S1 subunit is solubilized by removing its C-terminal helix that inserts into the B pentamer). In some embodiments, the soluble S1 is detoxified with at least one mutation (e.g., 9K_129G).

The autotransporter antigens of pertussis include pertactin, SphB1, TcfA, BrkA, and Vag8. Each antigen contains an N-terminal passenger domain and a C-terminal beta barrel exporter (channel) domain. Many passenger domains are cleaved at the surface with a conserved protease site. In some embodiments, the vaccines described herein comprises mRNAs encoding at least one autotransporter antigen (e.g., pertactin, SphB1, TcfA, BrkA, and Vag8). In some embodiments, the autotransporter antigen is truncated at the protease cleavage site (e.g., between the asparagine and alanine residues).

Filamentous hemagglutinin adhesin (FHA) is a large, filamentous protein that functions as a dominant attachment factor for adherence to host ciliated epithelial cells of the respiratory tract (e.g., respiratory epithelium). The protein is a virulence factor for B. pertussis and it is associated with biofilm formation. The protein comprises at least three binding domains which can bind to different cell receptors on the epithelial cell surface, including a heparin-binding domain (HEP), carbohydrate binding domain (FragA), and Mal85 short fragment. In some embodiments, the FHA antigen comprises an FHA truncated protein and is selected from the group consisting of: FHA1_HEP₄₃₀₋₈₇₃, FHA2_FragA₁₁₄₁₋₁₂₇₃, FHA3_MAL85₁₆₅₅₋₂₁₁₁, and FHA4_Long₄₃₀₋₁₂₇₉. The FHA4_Long₄₃₀₋₁₂₇₉ construct comprises both the heparin and carbohydrate binding domains.

In some embodiments, the composition comprises mRNA encoding an adenylate cyclase toxin construct. Adenylate cyclase toxin is a 1706 amino acid residue long protein comprising three domains: an adenylate-cyclase domain, a hydrophobic domain, and calcium binding repeats. The toxin is secreted by the Type I secretion system, which permits the toxin to be secreted from the cytoplasm straight outside the cell. Most of the toxin remains associated with FHA inside the cell, but is inactive. Aggregation also inactivates the toxin, and its quick inactivation highlights the necessity of close contact between secreting bacterium and target cell. Adenylate cyclase toxin binds to target cells, typically phagocytes (e.g., neutrophils) by the complement receptor 3 (CD11b/CD18, or Mac-1). The hemolysin portion of the toxin then binds to the target membrane and inserts itself into the bilayer, resulting in the translocation of the adenylate cyclase (AC) domain into the cytoplasmic membrane of the cytoplasm. The AC domain then binds calmodulin and catalyzes unregulated production of cAMP from ATP. The overproduction of cAMP affects many cellular processes, including bactericidal functions of phagocytes. Repeat-in-toxins (RTX) are also part of the family and comprise repeating aspartate- and glycine-rich nonapeptides. RTX proteins form pore in cell membranes. In some embodiments, the composition (e.g., vaccine) comprises an mRNA encoding at least one adenylate cyclase toxin (ACT) construct selected from the group consisting of: ACT_{188LQ}, ACT_{H63A_K65A_S66G}, and RTX_{1006_1600}. The catalytic domain of ACT may be detoxified by, for example, an LQ188 insertion, or mutation of each catalytic residue to either alanine or glycine (H63A/K65A/S66G).

In some embodiments, the composition (e.g., vaccine) comprises an mRNA encoding at least one fimbriae (FIM) antigen. FIM proteins are long, thin, hair-like polymers comprising thousands of pilin subunits. FIM biogenesis requires a system of bacterial proteins including: an outer membrane B-barrel usher protein to translocate subunits, a tip-adhesive protein at the terminal end of polymer that binds oligosaccharides, and a periplasmic chaperone protein to stabilize the pilus subunits until assembled. The major pilins in B. pertussis are Fim2 and Fim3, which attach to respiratory epithelial cells during infection. In some embodiments, the composition (e.g., vaccine) comprises an mRNA encoding at least one FIM protein, such as a fimbriae domain swapped construct. Examples include FimD_Fim2 and Fim2_Fim2_Fim2.

In some embodiments, the composition (e.g., vaccine) comprises an mRNA encoding at least one diphtheria antigen (e.g., diphtheria toxin). Diphtheria is an infection caused by the bacterium Corynebacterium diphtheriae and causes sore throat and fever but can result in the formation of a gray or white patch in the throat which can block the airway and create a barking cough. In severe case, diphtheria is fatal. Diphtheria toxin is a single, 60-kDa-molecular weight protein comprising two peptide chains, fragment A and fragment B which are linked by a disulfide bond. Fragment B is a recognition subunit that gains the toxin entry into the host cell and Fragment A inhibits the synthesis of new proteins in the affected cell by catalyzing ADP-ribosylation of elongation factor EF-2.

In some embodiments, the composition (e.g., vaccine) comprises an mRNA encoding at least one tetanus antigen (e.g., tetanus toxin). Tetanus, is a bacterial infection characterized by muscle spasms that can be fatal. Tetanus is caused by an infection with the bacterium Clostridium tetani. The tetanus toxin, tetanospasmin, comprises a heavy chain and a light chain. There are three domains, each of which contributes to the pathophysiology of the toxin. The heavy chain comprises two of the domains: the N-terminal side of the heavy chain helps with membrane translocation, and the C-terminal side helps the toxin locate the specific receptor site on the correct neuron. The light chain domain cleaves vesicle associated membrane protein (VAMP), which is necessary for membrane fusion of small synaptic vesicles (SSVs). SSVs carry neurotransmitter to the membrane for release, and inhibition of this process blocks neurotransmitter release, resulting in the characteristic spasms.

In some embodiments, the composition (e.g., vaccine) comprises a first mRNA encoding a first pertussis antigenic polypeptide selected from the group consisting of: pertussis toxin antigenic polypeptides, autotransporter subtilisin-like protease (SPHB1) antigenic polypeptides, tracheal colonization factor A (TCFA) antigenic polypeptides, filamentous hemagglutinin (FHA) antigenic polypeptides, pertactin (PRN) antigenic polypeptides, fimbriae (FIM) antigenic polypeptides, adenylate cyclase antigenic polypeptides, and Bordetella resistance to killing (Brk) antigenic polypeptides.

In some embodiments, the composition (e.g., vaccine) further comprises a second mRNA encoding a second pertussis antigenic polypeptide selected from the group consisting of: pertussis toxin antigenic polypeptides, autotransporter subtilisin-like protease (SPHB1) antigenic polypeptides, tracheal colonization factor A (TCFA) antigenic polypeptides, filamentous hemagglutinin (FHA) antigenic polypeptides, pertactin (PRN) antigenic polypeptides, fimbriae (FIM) antigenic polypeptides, adenylate cyclase antigenic polypeptides, and Bordetella resistance to killing (Brk) antigenic polypeptides.

In some embodiments, the composition (e.g., vaccine) further comprises a third mRNA encoding a third pertussis antigenic polypeptide selected from the group consisting of: pertussis toxin antigenic polypeptides, autotransporter subtilisin-like protease (SPHB1) antigenic polypeptides, tracheal colonization factor A (TCFA) antigenic polypeptides, filamentous hemagglutinin (FHA) antigenic polypeptides, pertactin (PRN) antigenic polypeptides, fimbriae (FIM) antigenic polypeptides, adenylate cyclase antigenic polypeptides, and Bordetella resistance to killing (Brk) antigenic polypeptides.

In some embodiments, the composition (e.g., vaccine) further comprises a fourth mRNA encoding a fourth pertussis antigenic polypeptide selected from the group consisting of: pertussis toxin antigenic polypeptides, autotransporter subtilisin-like protease (SPHB1) antigenic polypeptides, tracheal colonization factor A (TCFA) antigenic polypeptides, filamentous hemagglutinin (FHA) antigenic polypeptides, pertactin (PRN) antigenic polypeptides, fimbriae (FIM) antigenic polypeptides, adenylate cyclase antigenic polypeptides, and Bordetella resistance to killing (Brk) antigenic polypeptides.

In some embodiments, the composition (e.g., vaccine) further comprises a fifth mRNA encoding a fifth pertussis antigenic polypeptide selected from the group consisting of: pertussis toxin antigenic polypeptides, autotransporter subtilisin-like protease (SPHB1) antigenic polypeptides, tracheal colonization factor A (TCFA) antigenic polypeptides, filamentous hemagglutinin (FHA) antigenic polypeptides, pertactin (PRN) antigenic polypeptides, fimbriae (FIM) antigenic polypeptides, adenylate cyclase antigenic polypeptides, and Bordetella resistance to killing (Brk) antigenic polypeptides.

In some embodiments, the composition (e.g., vaccine) further comprises a sixth mRNA encoding a sixth pertussis antigenic polypeptide selected from the group consisting of: pertussis toxin antigenic polypeptides, autotransporter subtilisin-like protease (SPHB1) antigenic polypeptides, tracheal colonization factor A (TCFA) antigenic polypeptides, filamentous hemagglutinin (FHA) antigenic polypeptides, pertactin (PRN) antigenic polypeptides, fimbriae (FIM) antigenic polypeptides, adenylate cyclase antigenic polypeptides, and Bordetella resistance to killing (Brk) antigenic polypeptides.

In some embodiments, the composition (e.g., vaccine) comprises a seventh mRNA encoding a seventh pertussis antigenic polypeptide selected from the group consisting of: pertussis toxin antigenic polypeptides, autotransporter subtilisin-like protease (SPHB1) antigenic polypeptides, tracheal colonization factor A (TCFA) antigenic polypeptides, filamentous hemagglutinin (FHA) antigenic polypeptides, pertactin (PRN) antigenic polypeptides, fimbriae (FIM) antigenic polypeptides, adenylate cyclase antigenic polypeptides, and Bordetella resistance to killing (Brk) antigenic polypeptides.

In some embodiments, the composition (e.g., vaccine) comprises an eighth mRNA encoding an eighth pertussis antigenic polypeptide selected from the group consisting of: pertussis toxin antigenic polypeptides, autotransporter subtilisin-like protease (SPHB1) antigenic polypeptides, tracheal colonization factor A (TCFA) antigenic polypeptides, filamentous hemagglutinin (FHA) antigenic polypeptides, pertactin (PRN) antigenic polypeptides, fimbriae (FIM) antigenic polypeptides, adenylate cyclase antigenic polypeptides, and Bordetella resistance to killing (Brk) antigenic polypeptides.

In some embodiments, the composition (e.g., vaccine) comprises at least one mRNA polynucleotide (e.g., 1, 2, 3, 4, 5, 6, 7, or 8 mRNA polynucleotides) having at least one ORF encoding at least one of each pertussis antigenic polypeptide selected from the group consisting of: pertussis toxin antigenic polypeptides, autotransporter subtilisin-like protease (SPHB1) antigenic polypeptides, tracheal colonization factor A (TCFA) antigenic polypeptides, filamentous hemagglutinin (FHA) antigenic polypeptides, pertactin (PRN) antigenic polypeptides, fimbriae (FIM) antigenic polypeptides, adenylate cyclase antigenic polypeptides, and Bordetella resistance to killing (Brk) antigenic polypeptides.

In some embodiments, the composition (e.g., vaccine) further comprises at least one mRNA polynucleotide having at least one ORF encoding a diphtheria antigenic polypeptide. In some embodiments, the composition (e.g., vaccine) further comprises at least one mRNA polynucleotide having at least one ORF encoding a tetanus antigenic polypeptide. In some embodiments, the composition (e.g., vaccine) further comprises at least one mRNA polynucleotide having at least one ORF encoding a diphtheria antigenic polypeptide and at least one mRNA polynucleotide having at least one ORF encoding a tetanus antigenic polypeptide.

In some embodiments, the composition (e.g., vaccine) comprises at least one mRNA polynucleotide having at least one ORF encoding a diphtheria antigenic polypeptide. In some embodiments, the composition (e.g., vaccine) comprises at least one mRNA polynucleotide having at least one ORF encoding a tetanus antigenic polypeptide.

In each embodiment or aspect of the invention, it is understood that the featured vaccines include the mRNAs encapsulated within lipid nanoparticles (LNPs). While it is possible to encapsulate each unique mRNA in its own LNP, the mRNA vaccine technology enjoys the significant technological advantage of being able to encapsulate several mRNAs in a single LNP product. In other embodiments the vaccines are separate vaccines that are not co-formulated, but may be admixed separately before administration or simply administered separately.

Exemplary sequences of the pertussis, diphtheria, and tetanus antigens encoded by the mRNA of the present disclosure are provided in Table 1. In some embodiments, the mRNA vaccines encode a polypeptide that is 80%, 81%, 82%, 83%, 84%, 85%, 86%, 87%, 88%, 89%, 90%, 91%, 92%, 93%, 94%, 95%, 96%, 97%, 98%, 99% or identical to a sequence selected from SEQ ID NOs: 5, 8, 11, 14, 17, 20, 23, 26, 29, 32, 35, 38, 41, 44, 47, 50, and 53.

### Nucleic Acids

The compositions of the present disclosure comprise a (at least one) messenger RNA (mRNA) having an open reading frame (ORF) encoding a pertussis antigen, diphtheria antigen, or tetanus antigen. In some embodiments, the mRNA further comprises a 5′ UTR, 3′ UTR, a poly(A) tail and/or a 5′ cap analog.

It should also be understood that the compositions (e.g., vaccines) of the present disclosure may include any 5′ untranslated region (UTR) and/or any 3′ UTR. Exemplary UTR sequences include SEQ ID NOs: 11-14; however, other UTR sequences may be used or exchanged for any of the UTR sequences described herein. In some embodiments, a 5′ UTR of the present disclosure comprises a sequence selected from SEQ ID NO: 99 (GGGAAAUA AGAGAGAAAAGAAGAGUAAGAAGAAAUAUAAGAGCCACC) and SEQ ID NO: 2 (GGGAAAUAAGAGAGAAAAGAAGAGUAAGAAGAAAUAUAAGACCCCGGCGCCGCC ACC). In some embodiments, a 3′ UTR of the present disclosure comprises a sequence selected from SEQ ID NO: 100 (UGAUAAUAGGCUGGAGCCUCGGUGGCCAUGCUU CUUGCCCCUUGGGCCUCCCCCCAGCCCCUCCUCCCCUUCCUGCACCCGUACCCCCG UGGUCUUUGAAUAAAGUCUGAGUGGGCGGC) and SEQ ID NO: 4 (UGAUAA UAGGCUGGAGCCUCGGUGGCCUAGCUUCUUGCCCCUUGGGCCUCCCCCCAGCCCC UCCUCCCCUUCCUGCACCCGUACCCCCGUGGUCUUUGAAUAAAGUCUGAGUGGGC GGC). UTRs may also be omitted from the RNA polynucleotides provided herein.

Nucleic acids comprise a polymer of nucleotides (nucleotide monomers). Thus, nucleic acids are also referred to as polynucleotides. Nucleic acids may be or may include, for example, deoxyribonucleic acids (DNAs), ribonucleic acids (RNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA) and/or chimeras and/or combinations thereof.

Messenger RNA (mRNA) is any RNA that encodes a (at least one) protein (a naturally-occurring, non-naturally-occurring, or modified polymer of amino acids) and can be translated to produce the encoded protein in vitro, in vivo, in situ, or ex vivo. The skilled artisan will appreciate that, except where otherwise noted, nucleic acid sequences set forth in the instant application may recite “T”s in a representative DNA sequence but where the sequence represents mRNA, the “T”s would be substituted for “U”s. Thus, any of the DNAs disclosed and identified by a particular sequence identification number herein also disclose the corresponding mRNA sequence complementary to the DNA, where each “T” of the DNA sequence is substituted with “U.”

An open reading frame (ORF) is a continuous stretch of DNA or RNA beginning with a start codon (e.g., methionine (ATG or AUG)) and ending with a stop codon (e.g., TAA, TAG or TGA, or UAA, UAG or UGA). An ORF typically encodes a protein. It will be understood that the sequences disclosed herein may further comprise additional elements, e.g., 5′ and 3′ UTRs, but that those elements, unlike the ORF, need not necessarily be present in an RNA polynucleotide of the present disclosure.

Exemplary sequences of mRNA that encode pertussis, diphtheria, and tetanus antigens of the present disclosure are provided in Table 1. In some embodiments, the mRNA comprises an open reading frame (ORF) that is 80%, 81%, 82%, 83%, 84%, 85%, 86%, 87%, 88%, 89%, 90%, 91%, 92%, 93%, 94%, 95%, 96%, 97%, 98%, 99% or identical to a sequence selected from SEQ ID NOs: 3, 7, 10, 13, 16, 19, 22, 25, 28, 31, 34, 37, 40, 43, 46, 49, and 52. In some embodiments, the mRNA comprises a nucleotide sequence that is 80%, 81%, 82%, 83%, 84%, 85%, 86%, 87%, 88%, 89%, 90%, 91%, 92%, 93%, 94%, 95%, 96%, 97%, 98%, 99% or identical to a sequence selected from SEQ ID NOs: 1, 6, 9, 12, 15, 18, 21, 24, 27, 30, 33, 36, 39, 42, 45, 48, and 51.

### Variants

In some embodiments, the compositions of the present disclosure include RNA that encodes at least one pertussis antigen variant, diphtheria antigen variant, or tetanus antigen variant. Antigen variants or other polypeptide variants refers to molecules that differ in their amino acid sequence from a wild-type, native, or reference sequence. The antigen/polypeptide variants may possess substitutions, deletions, and/or insertions at certain positions within the amino acid sequence, as compared to a native or reference sequence. Ordinarily, variants possess at least 50% identity to a wild-type, native or reference sequence. In some embodiments, variants share at least 80%, or at least 90% identity with a wild-type, native, or reference sequence.

Variant antigens/polypeptides encoded by nucleic acids of the disclosure may contain amino acid changes that confer any of a number of desirable properties, e.g., that enhance their immunogenicity, enhance their expression, and/or improve their stability or PK/PD properties in a subject. Variant antigens/polypeptides can be made using routine mutagenesis techniques and assayed as appropriate to determine whether they possess the desired property. Assays to determine expression levels and immunogenicity are well known in the art. Similarly, PK/PD properties of a protein variant can be measured using art recognized techniques, e.g., by determining expression of antigens in a vaccinated subject over time and/or by looking at the durability of the induced immune response. The stability of protein(s) encoded by a variant nucleic acid may be measured by assaying thermal stability or stability upon urea denaturation or may be measured using in silico prediction. Methods for such experiments and in silico determinations are known in the art.

In some embodiments, a composition comprises an RNA or an RNA ORF that comprises a nucleotide sequence of any one of the sequences provided herein, or comprises a nucleotide sequence at least 90%, at least 95%, at least 96%, at least 97%, at least 98%, or at least 99% identical to a nucleotide sequence of any one of the sequences provided herein.

The term “identity” refers to a relationship between the sequences of two or more polypeptides (e.g. antigens) or polynucleotides (nucleic acids), as determined by comparing the sequences. Identity also refers to the degree of sequence relatedness between or among sequences as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related antigens or nucleic acids can be readily calculated by known methods. “Percent (%) identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Methods and computer programs for the alignment are well known in the art. It is understood that identity depends on a calculation of percent identity but may differ in value due to gaps and penalties introduced in the calculation. Generally, variants of a particular polynucleotide or polypeptide (e.g., antigen) have at least 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Stephen F. Altschul, et al (1997), “Gapped BLAST and PSI-BLAST: a new generation of protein database search programs”, Nucleic Acids Res. 25:3389-3402). Another popular local alignment technique is based on the Smith-Waterman algorithm (Smith, T. F. & Waterman, M. S. (1981) “Identification of common molecular subsequences.” J. Mol. Biol. 147:195-197). A general global alignment technique based on dynamic programming is the Needleman-Wunsch algorithm (Needleman, S. B. & Wunsch, C. D. (1970) “A general method applicable to the search for similarities in the amino acid sequences of two proteins.” J. Mol. Biol. 48:443-453). More recently a Fast Optimal Global Sequence Alignment Algorithm (FOGSAA) has been developed that purportedly produces global alignment of nucleotide and protein sequences faster than other optimal global alignment methods, including the Needleman-Wunsch algorithm.

As such, polynucleotides encoding peptides or polypeptides containing substitutions, insertions and/or additions, deletions, and covalent modifications with respect to reference sequences, in particular the polypeptide (e.g., antigen) sequences disclosed herein, are included within the scope of this disclosure. For example, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble or linked to a solid support. In some embodiments, sequences for (or encoding) signal sequences, termination sequences, transmembrane domains, linkers, multimerization domains (such as, e.g., foldon regions) and the like may be substituted with alternative sequences that achieve the same or a similar function. In some embodiments, cavities in the core of proteins can be filled to improve stability, e.g., by introducing larger amino acids. In other embodiments, buried hydrogen bond networks may be replaced with hydrophobic resides to improve stability. In yet other embodiments, glycosylation sites may be removed and replaced with appropriate residues. Such sequences are readily identifiable to one of skill in the art. It should also be understood that some of the sequences provided herein contain sequence tags or terminal peptide sequences (e.g., at the N-terminal or C-terminal ends) that may be deleted, for example, prior to use in the preparation of an mRNA vaccine.

As recognized by those skilled in the art, protein fragments, functional protein domains, and homologous proteins are also considered to be within the scope of pertussis antigens, diphtheria antigens, and/or tetanus antigens of interest. For example, provided herein is any protein fragment (meaning a polypeptide sequence at least one amino acid residue shorter than a reference antigen sequence but otherwise identical) of a reference protein, provided that the fragment is immunogenic and confers a protective immune response to pertussis, diphtheria, or tetanus. In addition to variants that are identical to the reference protein but are truncated, in some embodiments, an antigen includes 2, 3, 4, 5, 6, 7, 8, 9, 10, or more mutations, as shown in any of the sequences provided or referenced herein. Antigens/antigenic polypeptides can range in length from about 4, 6, or 8 amino acids to full length proteins.

### Stabilizing Elements

Naturally-occurring eukaryotic mRNA molecules can contain stabilizing elements, including, but not limited to untranslated regions (UTR) at their 5′-end (5′ UTR) and/or at their 3′-end (3′ UTR), in addition to other structural features, such as a 5′-cap structure or a 3′-poly(A) tail. Both the 5′ UTR and the 3′ UTR are typically transcribed from the genomic DNA and are elements of the premature mRNA. Characteristic structural features of mature mRNA, such as the 5′-cap and the 3′-poly(A) tail are usually added to the transcribed (premature) mRNA during mRNA processing.

In some embodiments, a composition includes an RNA polynucleotide having an open reading frame encoding at least one antigenic polypeptide having at least one modification, at least one 5′ terminal cap, and is formulated within a lipid nanoparticle. 5′-capping of polynucleotides may be completed concomitantly during the in vitro-transcription reaction using the following chemical RNA cap analogs to generate the 5′-guanosine cap structure according to manufacturer protocols: 3′-O-Me-m7G(5′)ppp(5′) G [the ARCA cap];G(5′)ppp(5′)A; G(5′)ppp(5′)G; m7G(5′)ppp(5′)A; m7G(5′)ppp(5′)G (New England BioLabs, Ipswich, MA). 5′-capping of modified RNA may be completed post-transcriptionally using a Vaccinia Virus Capping Enzyme to generate the “Cap 0” structure: m7G(5′)ppp(5′)G (New England BioLabs, Ipswich, MA). Cap 1 structure may be generated using both Vaccinia Virus Capping Enzyme and a 2′-O methyl-transferase to generate: m7G(5′)ppp(5′)G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase. Enzymes may be derived from a recombinant source.

The 3′-poly(A) tail is typically a stretch of adenine nucleotides added to the 3′-end of the transcribed mRNA. It can, in some instances, comprise up to about 400 adenine nucleotides. In some embodiments, the length of the 3′-poly(A) tail may be an essential element with respect to the stability of the individual mRNA.

In some embodiments, a composition includes a stabilizing element. Stabilizing elements may include for instance a histone stem-loop. A stem-loop binding protein (SLBP), a 32 kDa protein has been identified. It is associated with the histone stem-loop at the 3′-end of the histone messages in both the nucleus and the cytoplasm. Its expression level is regulated by the cell cycle; it peaks during the S-phase, when histone mRNA levels are also elevated. The protein has been shown to be essential for efficient 3′-end processing of histone pre-mRNA by the U7 snRNP. SLBP continues to be associated with the stem-loop after processing, and then stimulates the translation of mature histone mRNAs into histone proteins in the cytoplasm. The RNA binding domain of SLBP is conserved through metazoa and protozoa; its binding to the histone stem-loop depends on the structure of the loop. The minimum binding site includes at least three nucleotides 5′ and two nucleotides 3′ relative to the stem-loop.

In some embodiments, an mRNA includes a coding region, at least one histone stem-loop, and optionally, a poly(A) sequence or polyadenylation signal. The poly(A) sequence or polyadenylation signal generally should enhance the expression level of the encoded protein. The encoded protein, in some embodiments, is not a histone protein, a reporter protein (e.g. Luciferase, GFP, EGFP, β-Galactosidase, EGFP), or a marker or selection protein (e.g. alpha-Globin, Galactokinase and Xanthine:guanine phosphoribosyl transferase (GPT)).

In some embodiments, an mRNA includes the combination of a poly(A) sequence or polyadenylation signal and at least one histone stem-loop, even though both represent alternative mechanisms in nature, acts synergistically to increase the protein expression beyond the level observed with either of the individual elements. The synergistic effect of the combination of poly(A) and at least one histone stem-loop does not depend on the order of the elements or the length of the poly(A) sequence.

In some embodiments, an mRNA does not include a histone downstream element (HDE). “Histone downstream element” (HDE) includes a purine-rich polynucleotide stretch of approximately 15 to 20 nucleotides 3′ of naturally occurring stem-loops, representing the binding site for the U7 snRNA, which is involved in processing of histone pre-mRNA into mature histone mRNA. In some embodiments, the nucleic acid does not include an intron.

An mRNA may or may not contain an enhancer and/or promoter sequence, which may be modified or unmodified or which may be activated or inactivated. In some embodiments, the histone stem-loop is generally derived from histone genes and includes an intramolecular base pairing of two neighbored partially or entirely reverse complementary sequences separated by a spacer, consisting of a short sequence, which forms the loop of the structure. The unpaired loop region is typically unable to base pair with either of the stem loop elements. It occurs more often in RNA, as is a key component of many RNA secondary structures but may be present in single-stranded DNA as well. Stability of the stem-loop structure generally depends on the length, number of mismatches or bulges, and base composition of the paired region. In some embodiments, wobble base pairing (non-Watson-Crick base pairing) may result. In some embodiments, the at least one histone stem-loop sequence comprises a length of 15 to 45 nucleotides.

In some embodiments, an mRNA has one or more AU-rich sequences removed. These sequences, sometimes referred to as AURES are destabilizing sequences found in the 3′UTR. The AURES may be removed from the RNA vaccines. Alternatively, the AURES may remain in the RNA vaccine.

### Signal Peptides

In some embodiments, a composition comprises an mRNA having an ORF that encodes a signal peptide fused to the antigen. Signal peptides, comprising the N-terminal 15-60 amino acids of proteins, are typically needed for the translocation across the membrane on the secretory pathway and, thus, universally control the entry of most proteins both in eukaryotes and prokaryotes to the secretory pathway. In eukaryotes, the signal peptide of a nascent precursor protein (pre-protein) directs the ribosome to the rough endoplasmic reticulum (ER) membrane and initiates the transport of the growing peptide chain across it for processing. ER processing produces mature proteins, wherein the signal peptide is cleaved from precursor proteins, typically by an ER-resident signal peptidase of the host cell, or they remain uncleaved and function as a membrane anchor. A signal peptide may also facilitate the targeting of the protein to the cell membrane.

A signal peptide may have a length of 15-60 amino acids. For example, a signal peptide may have a length of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 amino acids. In some embodiments, a signal peptide has a length of 20-60, 25-60, 30-60, 35-60, 40-60, 45-60, 50-60, 55-60, 15-55, 20-55, 25-55, 30-55, 35-55, 40-55, 45-55, 50-55, 15-50, 20-50, 25-50, 30-50, 35-50, 40-50, 45-50, 15-45, 20-45, 25-45, 30-45, 35-45, 40-45, 15-40, 20-40, 25-40, 30-40, 35-40, 15-35, 20-35, 25-35, 30-35, 15-30, 20-30, 25-30, 15-25, 20-25, or 15-20 amino acids.

Signal peptides from heterologous genes (which regulate expression of genes other than pertussis, diphtheria, and tetanus antigens in nature) are known in the art and can be tested for desired properties and then incorporated into a nucleic acid of the disclosure.

### Fusion Proteins

In some embodiments, a composition of the present disclosure includes an mRNA encoding an antigenic fusion protein. Thus, the encoded antigen or antigens may include two or more proteins (e.g., protein and/or protein fragment) joined together with or without a linker. Alternatively, the protein to which a protein antigen is fused does not promote a strong immune response to itself, but rather to the pertussis antigen, diphtheria antigen, or tetanus antigen. Antigenic fusion proteins, in some embodiments, retain the functional property from each original protein.

In some embodiments, the antigenic fusion proteins comprise 2, 3, 4, 5, 6, 7, 8, 9, or 10 of the following antigenic polypeptides: pertussis toxin antigenic polypeptides, autotransporter subtilisin-like protease (SPHB1) antigenic polypeptides, tracheal colonization factor A (TCFA) antigenic polypeptides, filamentous hemagglutinin (FHA) antigenic polypeptides, pertactin (PRN) antigenic polypeptides, fimbriae (FIM) antigenic polypeptides, adenylate cyclase antigenic polypeptides, Bordetella resistance to killing (Brk) antigenic polypeptides, diphtheria antigenic polypeptides, and tetanus antigenic polypeptides. Exemplary fusion proteins of the disclosure are provided in Table 1. For example, in some embodiments, the mRNA vaccines of the disclosure encode a polypeptide that is 80%, 81%, 82%, 83%, 84%, 85%, 86%, 87%, 88%, 89%, 90%, 91%, 92%, 93%, 94%, 95%, 96%, 97%, 98%, 99% or identical to a sequence selected from SEQ ID NOs: 56, 59, 62, 65, 68, 71, 74, 77, 80, 83, 86, 89, 92, 95, and 98. In some embodiments, the mRNA vaccines of the disclosure comprise an ORF that is 80%, 81%, 82%, 83%, 84%, 85%, 86%, 87%, 88%, 89%, 90%, 91%, 92%, 93%, 94%, 95%, 96%, 97%, 98%, 99% or identical to a sequence selected from SEQ ID NOs: 55, 58, 61, 64, 67, 70, 73, 76, 79, 82, 85, 88, 91, 94, and 97. In some embodiments, the mRNA vaccines of the disclosure comprise a nucleotide sequence that is 80%, 81%, 82%, 83%, 84%, 85%, 86%, 87%, 88%, 89%, 90%, 91%, 92%, 93%, 94%, 95%, 96%, 97%, 98%, 99% or identical to a sequence selected from SEQ ID NOs: 54, 57, 60, 63, 66, 69, 72, 75, 78, 81, 84, 87, 90, 93, and 96.

### Linkers and Cleavable Peptides

In some embodiments, the mRNAs of the disclosure encode more than one polypeptide, referred to herein as fusion proteins. In some embodiments, the mRNA further encodes a linker located between at least one or each domain of the fusion protein. The linker can be, for example, a cleavable linker or protease-sensitive linker. In some embodiments, the linker is selected from the group consisting of F2A linker, P2A linker, T2A linker, E2A linker, and combinations thereof. This family of self-cleaving peptide linkers, referred to as 2A peptides, has been described in the art (see for example, Kim, J. H. et al. (2011) PLoS ONE 6:e18556). In some embodiments, the linker is an F2A linker.

In some embodiments, the linker is a GS linker. GS linkers are polypeptide linkers that include glycine and serine amino acids repeats. They comprise flexible and hydrophilic residues and can be used to perform fusion of protein subunits without interfering in the folding and function of the protein domains, and without formation of secondary structures. In some embodiments, an mRNA encodes a fusion protein that comprises a GS linker that is 3 to 20 amino acids long. For example, the GS linker may have a length of (or have a length of at least) 3, 4, 5, 6, 7, 8, 9, 10, 11, 12, 13, 14, 15, 16, 17, 18, 19, or 20 amino acids. In some embodiments, a GS linker is (or is at least) 15 amino acids long (e.g., GGSGGSGGSGGSGGG (SEQ ID NO: 101)). In some embodiments, a GS linker is (or is at least) 8 amino acids long (e.g., GGGSGGGS (SEQ ID NO: 102)). In some embodiments, a GS linker is (or is at least) 7 amino acids long (e.g., GGGSGGG (SEQ ID NO: 103)). In some embodiments, a GS linker is (or is at least) 4 amino acid long (e.g., GGGS (SEQ ID NO: 104)). In some embodiments, the GS linker comprises (GGGS)n (SEQ ID NO: 105), where n is any integer from 1-5. In some embodiments, a GS linker is (or is at least) 4 amino acid long (e.g., GSGG (SEQ ID NO: 106)). In some embodiments, the GS linker comprises (GSGG)n (SEQ ID NO: 107), where n is any integer from 1-5.

In some embodiments, a linker is a glycine linker, for example having a length of (or a length of at least) 3 amino acids (e.g., GGG).

In some embodiments, a protein encoded by an mRNA vaccine includes more than one linker, which may be the same or different from each other (e.g., GGGSGGG (SEQ ID NO: 103) and GGGS (SEQ ID NO: 104) in the same S protein construct).

Cleavable linkers known in the art may be used in connection with the disclosure. Exemplary such linkers include: F2A linkers, T2A linkers, P2A linkers, and E2A linkers (See, e.g., WO2017127750). The skilled artisan will appreciate that other art-recognized linkers may be suitable for use in the constructs of the disclosure (e.g., encoded by the nucleic acids of the disclosure). The skilled artisan will likewise appreciate that other polycistronic constructs (mRNA encoding more than one antigen/polypeptide separately within the same molecule) may be suitable for use as provided herein.

### Sequence Optimization

In some embodiments, an ORF encoding an antigen of the disclosure is codon optimized. Codon optimization methods are known in the art. For example, an ORF of any one or more of the sequences provided herein may be codon optimized. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or delete restriction sites; modify ribosome binding sites and mRNA degradation sites; adjust translational rates to allow the various domains of the protein to fold properly; or reduce or eliminate problem secondary structures within the polynucleotide. Codon optimization tools, algorithms and services are known in the art—non-limiting examples include services from GeneArt (Life Technologies), DNA2.0 (Menlo Park CA) and/or proprietary methods. In some embodiments, the open reading frame (ORF) sequence is optimized using optimization algorithms.

In some embodiments, a codon optimized sequence shares less than 95% sequence identity to a naturally-occurring or wild-type sequence ORF (e.g., a naturally-occurring or wild-type mRNA sequence encoding a pertussis antigen, diphtheria antigen, or tetanus antigen). In some embodiments, a codon optimized sequence shares less than 90% sequence identity to a naturally-occurring or wild-type sequence (e.g., a naturally-occurring or wild-type mRNA sequence encoding a pertussis antigen, diphtheria antigen, or tetanus antigen). In some embodiments, a codon optimized sequence shares less than 85% sequence identity to a naturally-occurring or wild-type sequence (e.g., a naturally-occurring or wild-type mRNA sequence encoding a pertussis antigen, diphtheria antigen, or tetanus antigen). In some embodiments, a codon optimized sequence shares less than 80% sequence identity to a naturally-occurring or wild-type sequence (e.g., a naturally-occurring or wild-type mRNA sequence encoding a pertussis antigen, diphtheria antigen, or tetanus antigen). In some embodiments, a codon optimized sequence shares less than 75% sequence identity to a naturally-occurring or wild-type sequence (e.g., a naturally-occurring or wild-type mRNA sequence encoding a pertussis antigen, diphtheria antigen, or tetanus antigen).

In some embodiments, a codon optimized sequence shares between 65% and 85% (e.g., between about 67% and about 85% or between about 67% and about 80%) sequence identity to a naturally-occurring or wild-type sequence (e.g., a naturally-occurring or wild-type mRNA sequence encoding a pertussis antigen, diphtheria antigen, or tetanus antigen). In some embodiments, a codon optimized sequence shares between 65% and 75% or about 80% sequence identity to a naturally-occurring or wild-type sequence (e.g., a naturally-occurring or wild-type mRNA sequence encoding a pertussis antigen, diphtheria antigen, or tetanus antigen).

In some embodiments, a codon-optimized sequence encodes an antigen that is as immunogenic as, or more immunogenic than (e.g., at least 10%, at least 20%, at least 30%, at least 40%, at least 50%, at least 100%, or at least 200% more), than a pertussis antigen, diphtheria antigen, or tetanus antigen encoded by a non-codon-optimized sequence.

When transfected into mammalian host cells, the modified mRNAs have a stability of between 12-18 hours, or greater than 18 hours, e.g., 24, 36, 48, 60, 72, or greater than 72 hours and are capable of being expressed by the mammalian host cells.

In some embodiments, a codon optimized RNA may be one in which the levels of G/C are enhanced. The G/C-content of nucleic acid molecules (e.g., mRNA) may influence the stability of the RNA. RNA having an increased amount of guanine (G) and/or cytosine (C) residues may be functionally more stable than RNA containing a large amount of adenine (A) and thymine (T) or uracil (U) nucleotides. As an example, WO02/098443 discloses a pharmaceutical composition containing an mRNA stabilized by sequence modifications in the translated region. Due to the degeneracy of the genetic code, the modifications work by substituting existing codons for those that promote greater RNA stability without changing the resulting amino acid. The approach is limited to coding regions of the RNA.

### Chemically Unmodified Nucleotides

In some embodiments, an mRNA is not chemically modified and comprises the standard ribonucleotides consisting of adenosine, guanosine, cytosine and uridine. In some embodiments, nucleotides and nucleosides of the present disclosure comprise standard nucleoside residues such as those present in transcribed RNA (e.g. A, G, C, or U). In some embodiments, nucleotides and nucleosides of the present disclosure comprise standard deoxyribonucleosides such as those present in DNA (e.g. dA, dG, dC, or dT).

### Chemical Modifications

The compositions of the present disclosure comprise, in some embodiments, an RNA having an open reading frame encoding a pertussis antigen, diphtheria antigen, or tetanus antigen, wherein the nucleic acid comprises nucleotides and/or nucleosides that can be standard (unmodified) or modified as is known in the art. In some embodiments, nucleotides and nucleosides of the present disclosure comprise modified nucleotides or nucleosides. Such modified nucleotides and nucleosides can be naturally-occurring modified nucleotides and nucleosides or non-naturally occurring modified nucleotides and nucleosides. Such modifications can include those at the sugar, backbone, or nucleobase portion of the nucleotide and/or nucleoside as are recognized in the art.

In some embodiments, a naturally-occurring modified nucleotide or nucleotide of the disclosure is one as is generally known or recognized in the art. Non-limiting examples of such naturally occurring modified nucleotides and nucleotides can be found, inter alia, in the widely recognized MODOMICS database.

In some embodiments, a non-naturally occurring modified nucleotide or nucleoside of the disclosure is one as is generally known or recognized in the art. Non-limiting examples of such non-naturally occurring modified nucleotides and nucleosides can be found, inter alia, in published US application Nos. PCT/US2012/058519; PCT/US2013/075177; PCT/US2014/058897; PCT/US2014/058891; PCT/US2014/070413; PCT/US2015/36773; PCT/US2015/36759; PCT/US2015/36771; or PCT/IB2017/051367 all of which are incorporated by reference herein.

Hence, nucleic acids of the disclosure (e.g., DNA nucleic acids and RNA nucleic acids, such as mRNA nucleic acids) can comprise standard nucleotides and nucleosides, naturally-occurring nucleotides and nucleosides, non-naturally-occurring nucleotides and nucleosides, or any combination thereof.

Nucleic acids of the disclosure (e.g., DNA nucleic acids and RNA nucleic acids, such as mRNA nucleic acids), in some embodiments, comprise various (more than one) different types of standard and/or modified nucleotides and nucleosides. In some embodiments, a particular region of a nucleic acid contains one, two or more (optionally different) types of standard and/or modified nucleotides and nucleosides.

In some embodiments, a modified RNA nucleic acid (e.g., a modified mRNA nucleic acid), introduced to a cell or organism, exhibits reduced degradation in the cell or organism, respectively, relative to an unmodified nucleic acid comprising standard nucleotides and nucleosides.

In some embodiments, a modified RNA nucleic acid (e.g., a modified mRNA nucleic acid), introduced into a cell or organism, may exhibit reduced immunogenicity in the cell or organism, respectively (e.g., a reduced innate response) relative to an unmodified nucleic acid comprising standard nucleotides and nucleosides.

Nucleic acids (e.g., RNA nucleic acids, such as mRNA nucleic acids), in some embodiments, comprise non-natural modified nucleotides that are introduced during synthesis or post-synthesis of the nucleic acids to achieve desired functions or properties. The modifications may be present on internucleotide linkages, purine or pyrimidine bases, or sugars. The modification may be introduced with chemical synthesis or with a polymerase enzyme at the terminal of a chain or anywhere else in the chain. Any of the regions of a nucleic acid may be chemically modified.

The present disclosure provides for modified nucleosides and nucleotides of a nucleic acid (e.g., RNA nucleic acids, such as mRNA nucleic acids). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Nucleic acids can comprise a region or regions of linked nucleosides. Such regions may have variable backbone linkages. The linkages can be standard phosphodiester linkages, in which case the nucleic acids would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures, such as, for example, in those nucleic acids having at least one chemical modification. One example of such non-standard base pairing is the base pairing between the modified nucleotide inosine and adenine, cytosine or uracil. Any combination of base/sugar or linker may be incorporated into nucleic acids of the present disclosure.

In some embodiments, modified nucleobases in nucleic acids (e.g., RNA nucleic acids, such as mRNA nucleic acids) comprise 1-methyl-pseudouridine (m1ψ), 1-ethyl-pseudouridine (e1ψ), 5-methoxy-uridine (mo5U), 5-methyl-cytidine (m5C), and/or pseudouridine (ψ). In some embodiments, modified nucleobases in nucleic acids (e.g., RNA nucleic acids, such as mRNA nucleic acids) comprise 5-methoxymethyl uridine, 5-methylthio uridine, 1-methoxymethyl pseudouridine, 5-methyl cytidine, and/or 5-methoxy cytidine. In some embodiments, the polyribonucleotide includes a combination of at least two (e.g., 2, 3, 4 or more) of any of the aforementioned modified nucleobases, including but not limited to chemical modifications.

In some embodiments, a mRNA of the disclosure comprises 1-methyl-pseudouridine (m1ψ) substitutions at one or more or all uridine positions of the nucleic acid.

In some embodiments, a mRNA of the disclosure comprises 1-methyl-pseudouridine (m1ψ) substitutions at one or more or all uridine positions of the nucleic acid and 5-methyl cytidine substitutions at one or more or all cytidine positions of the nucleic acid.

In some embodiments, a mRNA of the disclosure comprises pseudouridine (ψ) substitutions at one or more or all uridine positions of the nucleic acid.

In some embodiments, a mRNA of the disclosure comprises pseudouridine (ψ) substitutions at one or more or all uridine positions of the nucleic acid and 5-methyl cytidine substitutions at one or more or all cytidine positions of the nucleic acid.

In some embodiments, a mRNA of the disclosure comprises uridine at one or more or all uridine positions of the nucleic acid.

In some embodiments, mRNAs are uniformly modified (e.g., fully modified, modified throughout the entire sequence) for a particular modification. For example, a nucleic acid can be uniformly modified with 1-methyl-pseudouridine, meaning that all uridine residues in the mRNA sequence are replaced with 1-methyl-pseudouridine. Similarly, a nucleic acid can be uniformly modified for any type of nucleoside residue present in the sequence by replacement with a modified residue such as those set forth above.

The nucleic acids of the present disclosure may be partially or fully modified along the entire length of the molecule. For example, one or more or all or a given type of nucleotide (e.g., purine or pyrimidine, or any one or more or all of A, G, U, C) may be uniformly modified in a nucleic acid of the disclosure, or in a predetermined sequence region thereof (e.g., in the mRNA including or excluding the poly(A) tail). In some embodiments, all nucleotides X in a nucleic acid of the present disclosure (or in a sequence region thereof) are modified nucleotides, wherein X may be any one of nucleotides A, G, U, C, or any one of the combinations A+G, A+U, A+C, G+U, G+C, U+C, A+G+U, A+G+C, G+U+C or A+G+C.

The nucleic acid may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The mRNAs may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the nucleic acids may contain a modified pyrimidine such as a modified uracil or cytosine. In some embodiments, at least 5%, at least 10%, at least 25%, at least 50%, at least 80%, at least 90% or 100% of the uracil in the nucleic acid is replaced with a modified uracil (e.g., a 5-substituted uracil). The modified uracil can be replaced by a compound having a single unique structure or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the nucleic acid is replaced with a modified cytosine (e.g., a 5-substituted cytosine). The modified cytosine can be replaced by a compound having a single unique structure or can be replaced by a plurality of compounds having different structures (e.g., 2, 3, 4 or more unique structures).

### Untranslated Regions (UTRs)

The mRNAs of the present disclosure may comprise one or more regions or parts which act or function as an untranslated region. Where mRNAs are designed to encode at least one antigen of interest, the nucleic may comprise one or more of these untranslated regions (UTRs). Wild-type untranslated regions of a nucleic acid are transcribed but not translated. In mRNA, the 5′ UTR starts at the transcription start site and continues to the start codon but does not include the start codon; whereas, the 3′ UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into the polynucleotides of the present disclosure to, among other things, enhance the stability of the molecule. The specific features can also be incorporated to ensure controlled down-regulation of the transcript in case they are misdirected to undesired organs sites. A variety of 5′UTR and 3′UTR sequences are known and available in the art.

A 5′ UTR is region of an mRNA that is directly upstream (5′) from the start codon (the first codon of an mRNA transcript translated by a ribosome). A 5′ UTR does not encode a protein (is non-coding). Natural 5′UTRs have features that play roles in translation initiation. They harbor signatures like Kozak sequences which are commonly known to be involved in the process by which the ribosome initiates translation of many genes. Kozak sequences have the consensus CCR(A/G)CCAUGG, where R is a purine (adenine or guanine) three bases upstream of the start codon (AUG), which is followed by another ‘G’.5′UTR also have been known to form secondary structures which are involved in elongation factor binding.

In some embodiments of the disclosure, a 5′ UTR is a heterologous UTR, i.e., is a UTR found in nature associated with a different ORF. In another embodiment, a 5′ UTR is a synthetic UTR, i.e., does not occur in nature. Synthetic UTRs include UTRs that have been mutated to improve their properties, e.g., which increase gene expression as well as those which are completely synthetic. Exemplary 5′ UTRs include Xenopus or human derived α-globin or b-globin (U.S. Pat. Nos. 8,278,063; 9,012,219), human cytochrome b-245 a polypeptide, and hydroxysteroid (17b) dehydrogenase, and Tobacco etch virus (U.S. Pat. Nos. 8,278,063, 9,012,219). CMV immediate-early 1 (IE1) gene (US20140206753, WO2013/185069), the sequence GGGAUCCUACC (SEQ ID NO: 108) (WO2014144196) may also be used. In another embodiment, 5′ UTR of a TOP gene is a 5′ UTR of a TOP gene lacking the 5′ TOP motif (the oligopyrimidine tract) (e.g., WO/2015101414, WO2015101415, WO/2015/062738, WO2015024667, WO2015024667; 5′ UTR element derived from ribosomal protein Large 32 (L32) gene (WO/2015101414, WO2015101415, WO/2015/062738), 5′ UTR element derived from the 5′UTR of an hydroxysteroid (17-0) dehydrogenase 4 gene (HSD17B4) (WO2015024667), or a 5′ UTR element derived from the 5′ UTR of ATP5A1 (WO2015024667) can be used. In some embodiments, an internal ribosome entry site (IRES) is used instead of a 5′ UTR.

In some embodiments, a 5′ UTR of the present disclosure comprises a sequence selected from SEQ ID NO: 2 and SEQ ID NO: 99.

A 3′ UTR is region of an mRNA that is directly downstream (3′) from the stop codon (the codon of an mRNA transcript that signals a termination of translation). A 3′ UTR does not encode a protein (is non-coding). Natural or wild type 3′ UTRs are known to have stretches of adenosines and uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA(U/A)(U/A) nonamers. Molecules containing this type of AREs include GM-CSF and TNF-α. Class III ARES are less well defined. These U rich regions do not contain an AUUUA motif. c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of nucleic acids (e.g., RNA) of the disclosure. When engineering specific nucleic acids, one or more copies of an ARE can be introduced to make nucleic acids of the disclosure less stable and thereby curtail translation and decrease production of the resultant protein. Likewise, AREs can be identified and removed or mutated to increase the intracellular stability and thus increase translation and production of the resultant protein. Transfection experiments can be conducted in relevant cell lines, using nucleic acids of the disclosure and protein production can be assayed at various time points post-transfection. For example, cells can be transfected with different ARE-engineering molecules and by using an ELISA kit to the relevant protein and assaying protein produced at 6 hour, 12 hour, 24 hour, 48 hour, and 7 days post-transfection.

Those of ordinary skill in the art will understand that 5′UTRs that are heterologous or synthetic may be used with any desired 3′ UTR sequence. For example, a heterologous 5′UTR may be used with a synthetic 3′UTR with a heterologous 3′ UTR.

Non-UTR sequences may also be used as regions or subregions within a nucleic acid. For example, introns or portions of introns sequences may be incorporated into regions of nucleic acid of the disclosure. Incorporation of intronic sequences may increase protein production as well as nucleic acid levels.

Combinations of features may be included in flanking regions and may be contained within other features. For example, the ORF may be flanked by a 5′ UTR which may contain a strong Kozak translational initiation signal and/or a 3′ UTR which may include an oligo(dT) sequence for templated addition of a poly-A tail. 5′ UTR may comprise a first polynucleotide fragment and a second polynucleotide fragment from the same and/or different genes such as the 5′ UTRs described in US Patent Application Publication No. 20100293625 and PCT/US2014/069155, herein incorporated by reference in its entirety.

It should be understood that any UTR from any gene may be incorporated into the regions of a nucleic acid. Furthermore, multiple wild-type UTRs of any known gene may be utilized. It is also within the scope of the present disclosure to provide artificial UTRs which are not variants of wild type regions. These UTRs or portions thereof may be placed in the same orientation as in the transcript from which they were selected or may be altered in orientation or location. Hence a 5′ or 3′ UTR may be inverted, shortened, lengthened, made with one or more other 5′ UTRs or 3′ UTRs. As used herein, the term “altered” as it relates to a UTR sequence, means that the UTR has been changed in some way in relation to a reference sequence. For example, a 3′ UTR or 5′ UTR may be altered relative to a wild-type or native UTR by the change in orientation or location as taught above or may be altered by the inclusion of additional nucleotides, deletion of nucleotides, swapping or transposition of nucleotides. Any of these changes producing an “altered” UTR (whether 3′ or 5′) comprise a variant UTR.

In some embodiments, a double, triple or quadruple UTR such as a 5′ UTR or 3′ UTR may be used. As used herein, a “double” UTR is one in which two copies of the same UTR are encoded either in series or substantially in series. For example, a double beta-globin 3′ UTR may be used as described in US Patent publication 20100129877, the contents of which are incorporated herein by reference in its entirety.

It is also within the scope of the present disclosure to have patterned UTRs. As used herein “patterned UTRs” are those UTRs which reflect a repeating or alternating pattern, such as ABABAB or AABBAABBAABB or ABCABCABC or variants thereof repeated once, twice, or more than 3 times. In these patterns, each letter, A, B, or C represent a different UTR at the nucleotide level.

In some embodiments, flanking regions are selected from a family of transcripts whose proteins share a common function, structure, feature, or property. For example, polypeptides of interest may belong to a family of proteins which are expressed in a particular cell, tissue or at some time during development. The UTRs from any of these genes may be swapped for any other UTR of the same or different family of proteins to create a new polynucleotide. As used herein, a “family of proteins” is used in the broadest sense to refer to a group of two or more polypeptides of interest which share at least one function, structure, feature, localization, origin, or expression pattern.

The untranslated region may also include translation enhancer elements (TEE). As a non-limiting example, the TEE may include those described in US Application No. 20090226470, herein incorporated by reference in its entirety, and those known in the art.

### In Vitro Transcription of RNA

cDNA encoding the polynucleotides described herein may be transcribed using an in vitro transcription (IVT) system. In vitro transcription of RNA is known in the art and is described in International Publication WO 2014/152027, which is incorporated by reference herein in its entirety. In some embodiments, the RNA of the present disclosure is prepared in accordance with any one or more of the methods described in WO 2018/053209 and WO 2019/036682, each of which is incorporated by reference herein.

In some embodiments, the RNA transcript is generated using a non-amplified, linearized DNA template in an in vitro transcription reaction to generate the RNA transcript. In some embodiments, the template DNA is isolated DNA. In some embodiments, the template DNA is cDNA. In some embodiments, the cDNA is formed by reverse transcription of an RNA polynucleotide, for example, but not limited to pertussis mRNA. In some embodiments, cells, e.g., bacterial cells, e.g., E. coli, e.g., DH-1 cells are transfected with the plasmid DNA template. In some embodiments, the transfected cells are cultured to replicate the plasmid DNA which is then isolated and purified. In some embodiments, the DNA template includes an RNA polymerase promoter, e.g., a T7 promoter located 5′ to and operably linked to the gene of interest.

In some embodiments, an in vitro transcription template encodes a 5′ untranslated (UTR) region, contains an open reading frame, and encodes a 3′ UTR and a poly(A) tail. The particular nucleic acid sequence composition and length of an in vitro transcription template will depend on the mRNA encoded by the template.

A “5′ untranslated region” (UTR) refers to a region of an mRNA that is directly upstream (i.e., 5′) from the start codon (i.e., the first codon of an mRNA transcript translated by a ribosome) that does not encode a polypeptide. When RNA transcripts are being generated, the 5′ UTR may comprise a promoter sequence. Such promoter sequences are known in the art. It should be understood that such promoter sequences will not be present in a vaccine of the disclosure.

A “3′ untranslated region” (UTR) refers to a region of an mRNA that is directly downstream (i.e., 3′) from the stop codon (i.e., the codon of an mRNA transcript that signals a termination of translation) that does not encode a polypeptide.

An “open reading frame” is a continuous stretch of DNA beginning with a start codon (e.g., methionine (ATG)), and ending with a stop codon (e.g., TAA, TAG or TGA) and encodes a polypeptide.

A “poly(A) tail” is a region of mRNA that is downstream, e.g., directly downstream (i.e., 3′), from the 3′ UTR that contains multiple, consecutive adenosine monophosphates. A poly(A) tail may contain 10 to 300 adenosine monophosphates. For example, a poly(A) tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some embodiments, a poly(A) tail contains 50 to 250 adenosine monophosphates. In a relevant biological setting (e.g., in cells, in vivo) the poly(A) tail functions to protect mRNA from enzymatic degradation, e.g., in the cytoplasm, and aids in transcription termination, and/or export of the mRNA from the nucleus and translation.

In some embodiments, a nucleic acid includes 200 to 3,000 nucleotides. For example, a nucleic acid may include 200 to 500, 200 to 1000, 200 to 1500, 200 to 3000, 500 to 1000, 500 to 1500, 500 to 2000, 500 to 3000, 1000 to 1500, 1000 to 2000, 1000 to 3000, 1500 to 3000, or 2000 to 3000 nucleotides).

An in vitro transcription system typically comprises a transcription buffer, nucleotide triphosphates (NTPs), an RNase inhibitor and a polymerase.

The NTPs may be manufactured in house, may be selected from a supplier, or may be synthesized as described herein. The NTPs may be selected from, but are not limited to, those described herein including natural and unnatural (modified) NTPs.

Any number of RNA polymerases or variants may be used in the method of the present disclosure. The polymerase may be selected from, but is not limited to, a phage RNA polymerase, e.g., a T7 RNA polymerase, a T3 RNA polymerase, a SP6 RNA polymerase, and/or mutant polymerases such as, but not limited to, polymerases able to incorporate modified nucleic acids and/or modified nucleotides, including chemically modified nucleic acids and/or nucleotides. Some embodiments exclude the use of DNase.

In some embodiments, the RNA transcript is capped via enzymatic capping. In some embodiments, the RNA comprises 5′ terminal cap, for example, 7mG(5′)ppp(5′)NlmpNp.

### Chemical Synthesis

Solid-phase chemical synthesis. Nucleic acids the present disclosure may be manufactured in whole or in part using solid phase techniques. Solid-phase chemical synthesis of nucleic acids is an automated method wherein molecules are immobilized on a solid support and synthesized step by step in a reactant solution. Solid-phase synthesis is useful in site-specific introduction of chemical modifications in the nucleic acid sequences.

Liquid Phase Chemical Synthesis. The synthesis of nucleic acids of the present disclosure by the sequential addition of monomer building blocks may be carried out in a liquid phase.

Combination of Synthetic Methods. The synthetic methods discussed above each has its own advantages and limitations. Attempts have been conducted to combine these methods to overcome the limitations. Such combinations of methods are within the scope of the present disclosure. The use of solid-phase or liquid-phase chemical synthesis in combination with enzymatic ligation provides an efficient way to generate long chain nucleic acids that cannot be obtained by chemical synthesis alone.

### Ligation of Nucleic Acid Regions or Subregions

Assembling nucleic acids by a ligase may also be used. DNA or RNA ligases promote intermolecular ligation of the 5′ and 3′ ends of polynucleotide chains through the formation of a phosphodiester bond. Nucleic acids such as chimeric polynucleotides and/or circular nucleic acids may be prepared by ligation of one or more regions or subregions. DNA fragments can be joined by a ligase catalyzed reaction to create recombinant DNA with different functions. Two oligodeoxynucleotides, one with a 5′ phosphoryl group and another with a free 3′ hydroxyl group, serve as substrates for a DNA ligase.

### Purification

Purification of the nucleic acids described herein may include, but is not limited to, nucleic acid clean-up, quality assurance and quality control. Clean-up may be performed by methods known in the arts such as, but not limited to, AGENCOURT® beads (Beckman Coulter Genomics, Danvers, MA), poly-T beads, LNATM oligo-T capture probes (EXIQON® Inc, Vedbaek, Denmark) or HPLC based purification methods such as, but not limited to, strong anion exchange HPLC, weak anion exchange HPLC, reverse phase HPLC (RP-HPLC), and hydrophobic interaction HPLC (HIC-HPLC). The term “purified” when used in relation to a nucleic acid such as a “purified nucleic acid” refers to one that is separated from at least one contaminant. A “contaminant” is any substance that makes another unfit, impure or inferior. Thus, a purified nucleic acid (e.g., DNA and RNA) is present in a form or setting different from that in which it is found in nature, or a form or setting different from that which existed prior to subjecting it to a treatment or purification method.

A quality assurance and/or quality control check may be conducted using methods such as, but not limited to, gel electrophoresis, UV absorbance, or analytical HPLC.

In some embodiments, the nucleic acids may be sequenced by methods including, but not limited to reverse-transcriptase-PCR.

### Quantification

In some embodiments, the nucleic acids of the present disclosure may be quantified in exosomes or when derived from one or more bodily fluid. Bodily fluids include peripheral blood, serum, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, sweat, fecal matter, hair, tears, cyst fluid, pleural and peritoneal fluid, pericardial fluid, lymph, chyme, chyle, bile, interstitial fluid, menses, pus, sebum, vomit, vaginal secretions, mucosal secretion, stool water, pancreatic juice, lavage fluids from sinus cavities, bronchopulmonary aspirates, blastocyl cavity fluid, and umbilical cord blood. Alternatively, exosomes may be retrieved from an organ selected from the group consisting of lung, heart, pancreas, stomach, intestine, bladder, kidney, ovary, testis, skin, colon, breast, prostate, brain, esophagus, liver, and placenta.

Assays may be performed using construct specific probes, cytometry, qRT-PCR, real-time PCR, PCR, flow cytometry, electrophoresis, mass spectrometry, or combinations thereof while the exosomes may be isolated using immunohistochemical methods such as enzyme linked immunosorbent assay (ELISA) methods. Exosomes may also be isolated by size exclusion chromatography, density gradient centrifugation, differential centrifugation, nanomembrane ultrafiltration, immunoabsorbent capture, affinity purification, microfluidic separation, or combinations thereof.

These methods afford the investigator the ability to monitor, in real time, the level of nucleic acids remaining or delivered. This is possible because the nucleic acids of the present disclosure, in some embodiments, differ from the endogenous forms due to the structural or chemical modifications.

In some embodiments, the nucleic acid may be quantified using methods such as, but not limited to, ultraviolet visible spectroscopy (UV/Vis). A non-limiting example of a UV/Vis spectrometer is a NANODROP® spectrometer (ThermoFisher, Waltham, MA). The quantified nucleic acid may be analyzed in order to determine if the nucleic acid may be of proper size, check that no degradation of the nucleic acid has occurred. Degradation of the nucleic acid may be checked by methods such as, but not limited to, agarose gel electrophoresis, HPLC based purification methods such as, but not limited to, strong anion exchange HPLC, weak anion exchange HPLC, reverse phase HPLC (RP-HPLC), and hydrophobic interaction HPLC (HIC-HPLC), liquid chromatography-mass spectrometry (LCMS), capillary electrophoresis (CE) and capillary gel electrophoresis (CGE).

### Lipid Nanoparticles (LNPs)

In some embodiments, the mRNA of the disclosure is formulated in a lipid nanoparticle (LNP). Lipid nanoparticles typically comprise ionizable amino lipid, non-cationic lipid, sterol and PEG lipid components along with the nucleic acid cargo of interest. The lipid nanoparticles of the disclosure can be generated using components, compositions, and methods as are generally known in the art, see for example PCT/US2016/052352; PCT/US2016/068300; PCT/US2017/037551; PCT/US2015/027400; PCT/US2016/047406; PCT/US2016000129; PCT/US2016/014280; PCT/US2016/014280; PCT/US2017/038426; PCT/US2014/027077; PCT/US2014/055394; PCT/US2016/52117; PCT/US2012/069610; PCT/US2017/027492; PCT/US2016/059575 and PCT/US2016/069491 all of which are incorporated by reference herein in their entirety.

Vaccines of the present disclosure are typically formulated in lipid nanoparticle. In some embodiments, the lipid nanoparticle comprises at least one ionizable amino lipid, at least one non-cationic lipid, at least one sterol, and/or at least one polyethylene glycol (PEG)-modified lipid.

In some embodiments, the lipid nanoparticle comprises 20-60 mol % ionizable amino lipid. In some embodiments, the lipid nanoparticle comprises 20-55 mol % ionizable amino lipid. For example, the lipid nanoparticle may comprise 20-50 mol %, 20-40 mol %, 20-30 mol %, 30-60 mol %, 30-50 mol %, 30-40 mol %, 40-60 mol %, 40-50 mol %, or 50-60 mol % ionizable amino lipid. In some embodiments, the lipid nanoparticle comprises 20 mol %, 30 mol %, 40 mol %, 50 mol %, or 60 mol % ionizable amino lipid. In some embodiments, the lipid nanoparticle comprises 40 mol %, 41 mol %, 42 mol %, 43 mol %, 44 mol % 45 mol %, 46 mol %, 47 mol %, 48 mol %, 49 mol %, 50 mol %, or 60 mol % ionizable amino lipid.

In some embodiments, the lipid nanoparticle comprises 5-25 mol % non-cationic lipid. For example, the lipid nanoparticle may comprise 5-20 mol %, 5-15 mol %, 5-10 mol %, 10-25 mol %, 10-20 mol %, 10-25 mol %, 15-25 mol %, 15-20 mol %, or 20-25 mol % non-cationic lipid. In some embodiments, the lipid nanoparticle comprises 5 mol %, 10 mol %, 15 mol %, 20 mol %, or 25 mol % non-cationic lipid.

In some embodiments, the lipid nanoparticle comprises 25-55 mol % sterol. For example, the lipid nanoparticle may comprise 25-50 mol %, 25-45 mol %, 25-40 mol %, 25-35 mol %, 25-30 mol %, 30-55 mol %, 30-50 mol %, 30-45 mol %, 30-40 mol %, 30-35 mol %, 35-55 mol %, 35-50 mol %, 35-45 mol %, 35-40 mol %, 40-55 mol %, 40-50 mol %, 40-45 mol %, 45-55 mol %, 45-50 mol %, or 50-55 mol % sterol. In some embodiments, the lipid nanoparticle comprises 25 mol %, 30 mol %, 35 mol %, 40 mol %, 45 mol %, 50 mol %, or 55 mol % sterol.

In some embodiments, the lipid nanoparticle comprises 0.5-15 mol % PEG-modified lipid. For example, the lipid nanoparticle may comprise 0.5-10 mol %, 0.5-5 mol %, 1-15 mol %, 1-10 mol %, 1-5 mol %, 2-15 mol %, 2-10 mol %, 2-5 mol %, 5-15 mol %, 5-10 mol %, or 10-15 mol %. In some embodiments, the lipid nanoparticle comprises 0.5 mol %, 1 mol %, 2 mol %, 3 mol %, 4 mol %, 5 mol %, 6 mol %, 7 mol %, 8 mol %, 9 mol %, 10 mol %, 11 mol %, 12 mol %, 13 mol %, 14 mol %, or 15 mol % PEG-modified lipid.

In some embodiments, the lipid nanoparticle comprises 20-60 mol % ionizable amino lipid, 5-25 mol % non-cationic lipid, 25-55 mol % sterol, and 0.5-15 mol % PEG-modified lipid. In some embodiments, the lipid nanoparticle comprises 40-50 mol % ionizable amino lipid, 5-15 mol % neutral lipid, 20-40 mol % cholesterol, and 0.5-3 mol % PEG-modified lipid. In some embodiments, the lipid nanoparticle comprises 45-50 mol % ionizable amino lipid, 9-13 mol % neutral lipid, 35-45 mol % cholesterol, and 2-3 mol % PEG-modified lipid. In some embodiments, the lipid nanoparticle comprises 48 mol % ionizable amino lipid, 11 mol % neutral lipid, 68.5 mol % cholesterol, and 2.5 mol % PEG-modified lipid.

In some embodiments, an ionizable amino lipid of the disclosure comprises a compound of Formula (I):

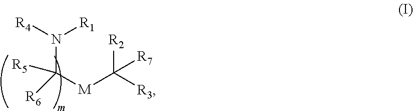

or a salt or isomer thereof, wherein:
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13.

In some embodiments, a subset of compounds of Formula (I) includes those in which when R₄ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

In some embodiments, another subset of compounds of Formula (I) includes those in which R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and a 5- to 14-membered heterocycloalkyl having one or more heteroatoms selected from N, O, and S which is substituted with one or more substituents selected from oxo (═O), OH, amino, mono- or di-alkylamino, and C₁₋₃ alkyl, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heterocycle having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5; and when Q is a 5- to 14-membered heterocycle and (i) R₄ is —(CH₂)ₙQ in which n is 1 or 2, or (ii) R₄ is —(CH₂)ₙCHQR in which n is 1, or (iii) R₄ is —CHQR, and —CQ(R)₂, then Q is either a 5- to 14-membered heteroaryl or 8- to 14-membered heterocycloalkyl;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (I) includes those in which R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)R, —C(O)N(R)OR, and —C(═NR₉)N(R)₂, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₂₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is —(CH₂)ₙQ or —(CH₂)ₙCHQR, where Q is —N(R)₂, and n is selected from 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, another subset of compounds of Formula (I) includes those in which
R₁ is selected from the group consisting of C₅₋₃₀ alkyl, C₅₋₂₀ alkenyl, —R*YR″, —YR″, and —R″M′ R′;
R₂ and R₃ are independently selected from the group consisting of C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of —(CH₂)ₙQ, —(CH₂)ₙCHQR, —CHQR, and —CQ(R)₂, where Q is —N(R)₂, and n is selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or isomers thereof.

In some embodiments, a subset of compounds of Formula (I) includes those of Formula (IA):

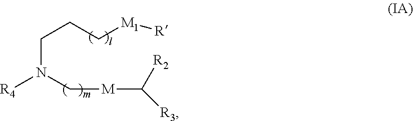

or a salt or isomer thereof, wherein 1 is selected from 1, 2, 3, 4, and 5; m is selected from 5, 6, 7, 8, and 9; M1 is a bond or M′; R₄ is unsubstituted C₁₋₃ alkyl, or —(CH₂)ₙQ, in which Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In some embodiments, a subset of compounds of Formula (I) includes those of Formula (II):

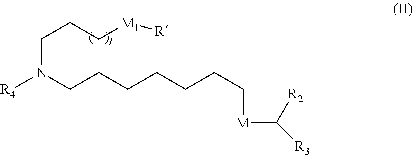

or a salt or isomer thereof, wherein 1 is selected from 1, 2, 3, 4, and 5; M₁ is a bond or M′; R₄ is unsubstituted C₁₋₃ alkyl, or —(CH₂)ₙQ, in which n is 2, 3, or 4, and Q is OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected from —C(O)O—, —OC(O)—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In some embodiments, a subset of compounds of Formula (I) includes those of Formula (IIa), (IIb), (IIc), or (IIe):

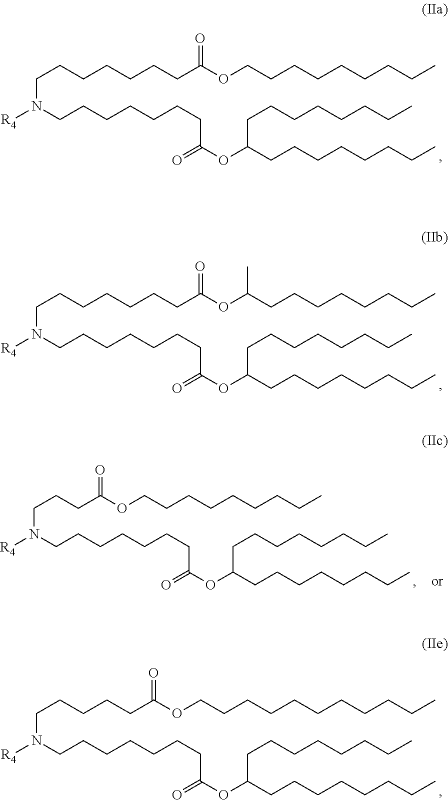

or a salt or isomer thereof, wherein R₄ is as described herein.

In some embodiments, a subset of compounds of Formula (I) includes those of Formula (IId):

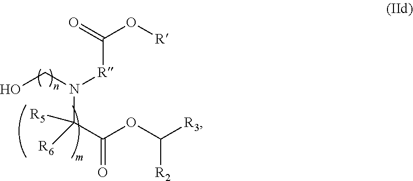

or a salt or isomer thereof, wherein n is 2, 3, or 4; and m, R′, R″, and R₂ through R₆ are as described herein. For example, each of R₂ and R₃ may be independently selected from the group consisting of C₅₋₁₄ alkyl and C₅₋₁₄ alkenyl.

In some embodiments, an ionizable amino lipid of the disclosure comprises a compound having structure:

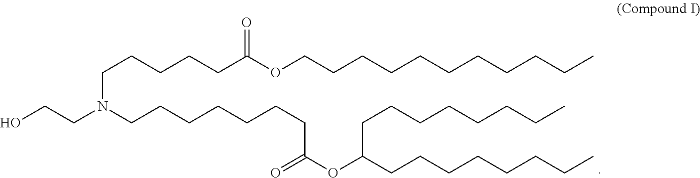 [SMILES: CCCCCCCCCCCOC(=O)CCCCCN(CCO)CCCCCCCC(=O)OC(CCCCCCCC)CCCCCCCC]

In some embodiments, an ionizable amino lipid of the disclosure comprises a compound having structure:

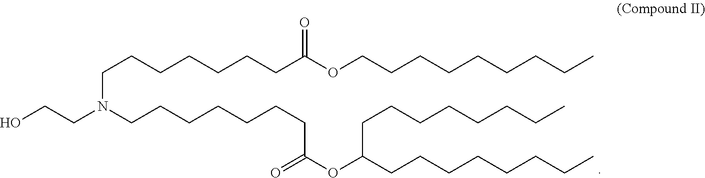 [SMILES: CCCCCCCCCOC(=O)CCCCCCCN(CCO)CCCCCCCC(=O)OC(CCCCCCCC)CCCCCCCC]

In some embodiments, a non-cationic lipid of the disclosure comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine,1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

In some embodiments, a PEG modified lipid of the disclosure comprises a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is DMG-PEG, PEG-c-DOMG (also referred to as PEG-DOMG), PEG-DSG and/or PEG-DPG.

In some embodiments, a sterol of the disclosure comprises cholesterol, fecosterol, sitosterol, ergosterol, campesterol, stigmasterol, brassicasterol, tomatidine, ursolic acid, alpha-tocopherol, and mixtures thereof.

In some embodiments, a LNP of the disclosure comprises an ionizable amino lipid of Compound 1, wherein the non-cationic lipid is DSPC, the structural lipid that is cholesterol, and the PEG lipid is DMG-PEG (e.g., PEG2000-DMG).

In some embodiments, the lipid nanoparticle comprises 45-55 mole percent (mol %) ionizable amino lipid (e.g., Compound 1). For example, lipid nanoparticle may comprise 45-47, 45-48, 45-49, 45-50, 45-52, 46-48, 46-49, 46-50, 46-52, 46-55, 47-48, 47-49, 47-50, 47-52, 47-55, 48-50, 48-52, 48-55, 49-50, 49-52, 49-55, or 50-55 mol % ionizable amino lipid (e.g., Compound 1). For example, lipid nanoparticle may comprise 45, 46, 47, 48, 49, 50, 51, 52, 53, 54, or 55 mol % ionizable amino lipid.

In some embodiments, the lipid nanoparticle comprises 5-15 mol % non-cationic (neutral) lipid (e.g., DSPC). For example, the lipid nanoparticle may comprise 5-6, 5-7, 5-8, 5-9, 5-10, 5-11, 5-12, 5-13, 5-14, 5-15, 6-7, 6-8, 6-9, 6-10, 6-11, 6-12, 6-13, 6-14, 6-15, 7-8, 7-9, 7-10, 7-11, 7-12, 7-13, 7-14, 7-15, 8-9, 8-10, 8-11, 8-12, 8-13, 8-14, 8-15, 9-10, 9-11, 9-12, 9-13, 9-14, 9-15, 10-11, 10-12, 10-13, 10-14, 10-15, 11-12, 11-13, 11-14, 11-15, 12-13, 12-14, 13-14, 13-15, or 14-15 mol % non-cationic (neutral) lipid (e.g., DSPC). For example, the lipid nanoparticle may comprise 5, 6, 7, 8, 9, 10, 11, 12, 13, 14, or 15 mol % DSPC.

In some embodiments, the lipid nanoparticle comprises 35-40 mol % sterol (e.g., cholesterol). For example, the lipid nanoparticle may comprise 35-36, 35-37, 35-38, 35-39, 35-40, 36-37, 36-38, 36-39, 36-40, 37-38, 37-39, 37-40, 38-39, 38-40, or 39-40 mol % cholesterol. For example, the lipid nanoparticle may comprise 35, 35.5, 36, 36.5, 37, 37.5, 38, 38.5, 39, 39.5, or 40 mol % cholesterol.

In some embodiments, the lipid nanoparticle comprises 1-3 mol % DMG-PEG. For example, the lipid nanoparticle may comprise 1-1.5, 1-2, 1-2.5, 1-3, 1.5-2, 1.5-2.5, 1.5-3, 2-2.5, 2-3, or 2.5-3. mol % DMG-PEG. For example, the lipid nanoparticle may comprise 1, 1.5, 2, 2.5, or 3 mol % DMG-PEG.

In some embodiments, the lipid nanoparticle comprises 50 mol % ionizable amino lipid, 10 mol % DSPC, 38.5 mol % cholesterol, and 1.5 mol % DMG-PEG. In some embodiments, the lipid nanoparticle comprises 48 mol % ionizable amino lipid, 11 mol % DSPC, 38.5 mol % cholesterol, and 2.5 mol % PEG2000-DMG.

In some embodiments, an LNP of the disclosure comprises an N:P ratio of from about 2:1 to about 30:1.

In some embodiments, an LNP of the disclosure comprises an N:P ratio of about 6:1.

In some embodiments, an LNP of the disclosure comprises an N:P ratio of about 3:1.

In some embodiments, an LNP of the disclosure comprises a wt/wt ratio of the ionizable amino lipid component to the RNA of from about 10:1 to about 100:1.

In some embodiments, an LNP of the disclosure comprises a wt/wt ratio of the ionizable amino lipid component to the RNA of about 20:1.

In some embodiments, an LNP of the disclosure comprises a wt/wt ratio of the ionizable amino lipid component to the RNA of about 10:1.

In some embodiments, an LNP of the disclosure has a mean diameter from about 50 nm to about 150 nm.

In some embodiments, an LNP of the disclosure has a mean diameter from about 70 nm to about 120 nm.

### Multivalent Vaccines

The compositions, as provided herein, may include RNA or multiple RNAs encoding two or more antigens of the same or different species. In some embodiments, composition includes an RNA or multiple RNAs encoding two or more pertussis antigens, diphtheria antigens, and/or tetanus. In some embodiments, the RNA may encode 1, 2, 3, 4, 5, 6, 7, 8, 9, or 10, or more pertussis antigens, diphtheria antigens, and/or tetanus antigens.

In some embodiments, two or more different mRNA encoding antigens may be formulated in the same lipid nanoparticle. In other embodiments, two or more different RNA encoding antigens may be formulated in separate lipid nanoparticles (each RNA formulated in a single lipid nanoparticle). The lipid nanoparticles may then be combined and administered as a single vaccine composition (e.g., comprising multiple RNA encoding multiple antigens) or may be administered separately.

### Pharmaceutical Formulations

Provided herein are compositions (e.g., pharmaceutical compositions), methods, kits and reagents for prevention or treatment of pertussis in humans and other mammals, for example. The compositions provided herein can be used as therapeutic or prophylactic agents. They may be used in medicine to prevent and/or treat a pertussis infection, diphtheria infection, and/or tetanus infection.

In some embodiments, the vaccine containing RNA as described herein can be administered to a subject (e.g., a mammalian subject, such as a human subject), and the RNA polynucleotides are translated in vivo to produce an antigenic polypeptide (antigen).

An “effective amount” of a composition (e.g., comprising RNA) is based, at least in part, on the target tissue, target cell type, means of administration, physical characteristics of the RNA (e.g., length, nucleotide composition, and/or extent of modified nucleosides), other components of the vaccine, and other determinants, such as age, body weight, height, sex and general health of the subject. Typically, an effective amount of a composition provides an induced or boosted immune response as a function of antigen production in the cells of the subject. In some embodiments, an effective amount of the composition containing RNA polynucleotides having at least one chemical modification are more efficient than a composition containing a corresponding unmodified polynucleotide encoding the same antigen or a peptide antigen.

Increased antigen production may be demonstrated by increased cell transfection (the percentage of cells transfected with the RNA vaccine), increased protein translation and/or expression from the polynucleotide, decreased nucleic acid degradation (as demonstrated, for example, by increased duration of protein translation from a modified polynucleotide), or altered antigen specific immune response of the host cell.

The term “pharmaceutical composition” refers to the combination of an active agent with a carrier, inert or active, making the composition especially suitable for diagnostic or therapeutic use in vivo or ex vivo. A “pharmaceutically acceptable carrier,” after administered to or upon a subject, does not cause undesirable physiological effects. The carrier in the pharmaceutical composition must be “acceptable” also in the sense that it is compatible with the active ingredient and can be capable of stabilizing it. One or more solubilizing agents can be utilized as pharmaceutical carriers for delivery of an active agent. Examples of a pharmaceutically acceptable carrier include, but are not limited to, biocompatible vehicles, adjuvants, additives, and diluents to achieve a composition usable as a dosage form. Examples of other carriers include colloidal silicon oxide, magnesium stearate, cellulose, and sodium lauryl sulfate. Additional suitable pharmaceutical carriers and diluents, as well as pharmaceutical necessities for their use, are described in Remington's Pharmaceutical Sciences.

In some embodiments, the compositions (comprising polynucleotides and their encoded polypeptides) in accordance with the present disclosure may be used for treatment or prevention of a pertussis infection, diphtheria infection, and/or tetanus infection. A composition may be administered prophylactically or therapeutically as part of an active immunization scheme to healthy individuals or early in infection during the incubation phase or during active infection after onset of symptoms. In some embodiments, the amount of RNA provided to a cell, a tissue or a subject may be an amount effective for immune prophylaxis.

A composition may be administered with other prophylactic or therapeutic compounds. As a non-limiting example, a prophylactic or therapeutic compound may be an adjuvant or a booster. As used herein, when referring to a prophylactic composition, such as a vaccine, the term “booster” refers to an extra administration of the prophylactic (vaccine) composition. A booster (or booster vaccine) may be given after an earlier administration of the prophylactic composition. The time of administration between the initial administration of the prophylactic composition and the booster may be, but is not limited to, 12 hours, 1 day, 36 hours, 2 days, 3 days, 4 days, 5 days, 6 days, 1 week, 10 days, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, or more. In exemplary embodiments, the time of administration between the initial administration of the prophylactic composition and the booster may be, but is not limited to, 1 week, 2 weeks, 3 weeks, 1 month, 2 months, 3 months, or 6 months. As is described herein, the booster may comprise the same or different mRNAs as compared to the earlier administration of the prophylactic composition. The booster, in some embodiments is monovalent (e.g., the mRNA encodes a single antigen). In some embodiments, the booster is multivalent (e.g., the mRNA encodes more than one antigen).

In some embodiments, a composition may be administered intramuscularly, intranasally or intradermally, similarly to the administration of inactivated vaccines known in the art.

A composition may be utilized in various settings depending on the prevalence of the infection or the degree or level of unmet medical need. As a non-limiting example, the RNA vaccines may be utilized to treat and/or prevent a variety of bacterial diseases (e.g, pertussis, tetanus, diphtheria). RNA vaccines have superior properties in that they produce much larger antibody titers, better neutralizing immunity, produce more durable immune responses, and/or produce responses earlier than commercially available vaccines.

Provided herein are pharmaceutical compositions including RNA and/or complexes optionally in combination with one or more pharmaceutically acceptable excipients.

The RNA may be formulated or administered alone or in conjunction with one or more other components. For example, an immunizing composition may comprise other components including, but not limited to, adjuvants.

In some embodiments, an immunizing composition does not include an adjuvant (they are adjuvant free).

An RNA may be formulated or administered in combination with one or more pharmaceutically-acceptable excipients. In some embodiments, vaccine compositions comprise at least one additional active substance, such as, for example, a therapeutically-active substance, a prophylactically-active substance, or a combination of both. Vaccine compositions may be sterile, pyrogen-free or both sterile and pyrogen-free. General considerations in the formulation and/or manufacture of pharmaceutical agents, such as vaccine compositions, may be found, for example, in Remington: The Science and Practice of Pharmacy 21st ed., Lippincott Williams & Wilkins, 2005 (incorporated herein by reference in its entirety).

In some embodiments, an immunizing composition is administered to humans, human patients or subjects. For the purposes of the present disclosure, the phrase “active ingredient” generally refers to the RNA vaccines or the polynucleotides contained therein, for example, RNA polynucleotides (e.g., mRNA polynucleotides) encoding antigens.

Formulations of the vaccine compositions described herein may be prepared by any method known or hereafter developed in the art of pharmacology. In general, such preparatory methods include the step of bringing the active ingredient (e.g., mRNA polynucleotide) into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

In some embodiments, an RNA is formulated using one or more excipients to: (1) increase stability; (2) increase cell transfection; (3) permit the sustained or delayed release (e.g., from a depot formulation); (4) alter the biodistribution (e.g., target to specific tissues or cell types); (5) increase the translation of encoded protein in vivo; and/or (6) alter the release profile of encoded protein (antigen) in vivo. In addition to traditional excipients such as any and all solvents, dispersion media, diluents, or other liquid vehicles, dispersion or suspension aids, surface active agents, isotonic agents, thickening or emulsifying agents, preservatives, excipients can include, without limitation, lipidoids, liposomes, lipid nanoparticles, polymers, lipoplexes, core-shell nanoparticles, peptides, proteins, cells transfected with the RNA (e.g., for transplantation into a subject), hyaluronidase, nanoparticle mimics and combinations thereof.

### Dosing/Administration

Provided herein are immunizing compositions (e.g., RNA vaccines), methods, kits and reagents for prevention and/or treatment of a pertussis infection, diphtheria infection, and/or tetanus infection in humans and other mammals. Immunizing compositions can be used as therapeutic or prophylactic agents. In some embodiments, immunizing compositions are used to provide prophylactic protection from a pertussis infection, diphtheria infection, and/or tetanus infection. In some embodiments, immunizing compositions are used to treat a pertussis infection, diphtheria infection, and/or tetanus infection. In some embodiments, embodiments, immunizing compositions are used in the priming of immune effector cells, for example, to activate peripheral blood mononuclear cells (PBMCs) ex vivo, which are then infused (re-infused) into a subject.

A subject may be any mammal, including non-human primate and human subjects. Typically, a subject is a human subject.

In some embodiments, an immunizing composition (e.g., RNA vaccine) is administered to a subject (e.g., a mammalian subject, such as a human subject) in an effective amount to induce an antigen-specific immune response. The RNA encoding the pertussis antigen, tetanus antigen, and/or diphtheria antigen is expressed and translated in vivo to produce the antigen, which then stimulates an immune response in the subject.

Prophylactic protection from a pertussis, diphtheria, and/or tetanus can be achieved following administration of an immunizing composition (e.g., an RNA vaccine) of the present disclosure. Immunizing compositions can be administered once, twice, three times, four times or more but it is likely sufficient to administer the vaccine once (optionally followed by a single booster). It is possible, although less desirable, to administer an immunizing composition to an infected individual to achieve a therapeutic response. Dosing may need to be adjusted accordingly.

A method of eliciting an immune response in a subject against a pertussis antigen, diphtheria antigen, and/or tetanus antigen (or multiple antigens) is provided in aspects of the present disclosure. In some embodiments, a method involves administering to the subject an immunizing composition comprising a mRNA having an open reading frame encoding a pertussis antigen, diphtheria antigen, and/or tetanus antigen (or multiple antigens), thereby inducing in the subject an immune response specific to the pertussis antigen, diphtheria antigen, and/or tetanus antigen (or multiple antigens), wherein anti-antigen antibody titer in the subject is increased following vaccination relative to anti-antigen antibody titer in a subject vaccinated with a prophylactically effective dose of a traditional vaccine against the antigen. An “anti-antigen antibody” is a serum antibody the binds specifically to the antigen.

A prophylactically effective dose is an effective dose that prevents infection with the virus at a clinically acceptable level. In some embodiments, the effective dose is a dose listed in a package insert for the vaccine. A traditional vaccine, as used herein, refers to a vaccine other than the mRNA vaccines of the present disclosure. For instance, a traditional vaccine includes, but is not limited, to live microorganism vaccines, killed microorganism vaccines, subunit vaccines, protein antigen vaccines, DNA vaccines, virus like particle (VLP) vaccines, etc. In exemplary embodiments, a traditional vaccine is a vaccine that has achieved regulatory approval and/or is registered by a national drug regulatory body, for example the Food and Drug Administration (FDA) in the United States or the European Medicines Agency (EMA).

In some embodiments, the anti-antigen antibody titer in the subject is increased 1 log to 10 log following vaccination relative to anti-antigen antibody titer in a subject vaccinated with a prophylactically effective dose of a traditional vaccine against pertussis, tetanus, or diphtheria or an unvaccinated subject. In some embodiments, the anti-antigen antibody titer in the subject is increased 1 log, 2 log, 3 log, 4 log, 5 log, or 10 log following vaccination relative to anti-antigen antibody titer in a subject vaccinated with a prophylactically effective dose of a traditional vaccine against pertussis, tetanus, or diphtheria or an unvaccinated subject.

A method of eliciting an immune response in a subject against pertussis, tetanus, and/or diphtheria is provided in other aspects of the disclosure. The method involves administering to the subject a composition comprising an mRNA comprising an open reading frame encoding a pertussis antigen, tetanus antigen, or diphtheria antigen, thereby inducing in the subject an immune response specific to pertussis, tetanus, or diphtheria, wherein the immune response in the subject is equivalent to an immune response in a subject vaccinated with a traditional vaccine against pertussis, tetanus, or diphtheria at 2 times to 100 times the dosage level relative to the composition.

In some embodiments, the immune response in the subject is equivalent to an immune response in a subject vaccinated with a traditional vaccine at twice the dosage level relative to a composition of the present disclosure. In some embodiments, the immune response in the subject is equivalent to an immune response in a subject vaccinated with a traditional vaccine at three times the dosage level relative to a composition of the present disclosure. In some embodiments, the immune response in the subject is equivalent to an immune response in a subject vaccinated with a traditional vaccine at 4 times, 5 times, 10 times, 50 times, or 100 times the dosage level relative to a composition of the present disclosure. In some embodiments, the immune response in the subject is equivalent to an immune response in a subject vaccinated with a traditional vaccine at 10 times to 1000 times the dosage level relative to a composition of the present disclosure. In some embodiments, the immune response in the subject is equivalent to an immune response in a subject vaccinated with a traditional vaccine at 100 times to 1000 times the dosage level relative to a composition of the present disclosure.

In other embodiments, the immune response is assessed by determining [protein] antibody titer in the subject. In other embodiments, the ability of serum or antibody from an immunized subject is tested for its ability to neutralize viral uptake or reduce pertussis, diphtheria, and/or tetanus transformation of human B lymphocytes. In other embodiments, the ability to promote a robust T cell response(s) is measured using art recognized techniques.

Other aspects the disclosure provide methods of eliciting an immune response in a subject against pertussis, diphtheria, and/or tetanus by administering to the subject composition comprising an mRNA having an open reading frame encoding a pertussis, diphtheria, and/or tetanus antigen, thereby inducing in the subject an immune response specific to the pertussis, diphtheria, and/or tetanus antigen, wherein the immune response in the subject is induced 2 days to 10 weeks earlier relative to an immune response induced in a subject vaccinated with a prophylactically effective dose of a traditional vaccine against pertussis, diphtheria, and/or tetanus. In some embodiments, the immune response in the subject is induced in a subject vaccinated with a prophylactically effective dose of a traditional vaccine at 2 times to 100 times the dosage level relative to a composition of the present disclosure.

In some embodiments, the immune response in the subject is induced 2 days, 3 days, 1 week, 2 weeks, 3 weeks, 5 weeks, or 10 weeks earlier relative to an immune response induced in a subject vaccinated with a prophylactically effective dose of a traditional vaccine.

Also provided herein are methods of eliciting an immune response in a subject against pertussis, tetanus, or diphtheria by administering to the subject an mRNA having an open reading frame encoding at least one pertussis antigen, tetanus antigen, and/or diphtheria antigen, wherein the RNA does not include a stabilization element, and wherein an adjuvant is not co-formulated or co-administered with the vaccine.

A composition may be administered by any route that results in a therapeutically effective outcome. These include, but are not limited, to intradermal, intramuscular, intranasal, and/or subcutaneous administration. The present disclosure provides methods comprising administering RNA vaccines to a subject in need thereof. The exact amount required will vary from subject to subject, depending on the species, age, and general condition of the subject, the severity of the disease, the particular composition, its mode of administration, its mode of activity, and the like. The RNA is typically formulated in dosage unit form for ease of administration and uniformity of dosage. It will be understood, however, that the total daily usage of the RNA may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The effective amount of the RNA (e.g., an effective dose), as provided herein, may be as low as 20 μg, administered for example as a single dose or as two 10 μg doses (e.g., a first effective vaccine dose and a second effective vaccine dose). In some embodiments, the first effective vaccine dose and the second effective vaccine dose are the same amount. In some embodiments, the first effective vaccine dose and the second effective vaccine dose are different amounts. In some embodiments, the effective amount is a total dose of 5 μg-30 μg, 5 μg-25 μg, 5 μg-20 μg, 5 μg-15 μg, 5 μg-10 μg, 10 μg-30 μg, 10 μg-25 μg, 10 μg-20 μg, 10 μg-15 μg, 15 μg-30 μg, 15 μg-25 μg, 15 μg-20 μg, 20 μg-30 μg, 25 μg-30 μg, or 25 μg-300 μg. In some embodiments, the effective dose (e.g., effective amount) is at least 10 μg and less than 25 μg of the composition. In some embodiments, the effective dose (e.g., effective amount) is at least 5 μg and less than 25 μg of the composition. For example, the effective amount may be a total dose of 5 μg, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 110 μg, 120 μg, 130 μg, 140 μg, 150 μg, 160 μg, 170 μg, 180 μg, 190 μg, 200 μg, 250 μg, or 300 μg. In some embodiments, the effective amount (e.g., effective dose) is a total dose of 10 μg. In some embodiments, the effective amount is a total dose of 20 μg (e.g., two 10 μg doses). In some embodiments, the effective amount is a total dose of 25 μg. In some embodiments, the effective amount is a total dose of 30 μg. In some embodiments, the effective amount is a total dose of 50 μg. In some embodiments, the effective amount is a total dose of 60 μg (e.g., two 30 μg doses). In some embodiments, the effective amount is a total dose of 75 μg. In some embodiments, the effective amount is a total dose of 100 g. In some embodiments, the effective amount is a total dose of 150 μg. In some embodiments, the effective amount is a total dose of 200 μg. In some embodiments, the effective amount is a total dose of 250 μg. In some embodiments, the effective amount is a total dose of 300 μg.

The RNA described herein can be formulated into a dosage form described herein, such as an intranasal, intratracheal, or injectable (e.g., intravenous, intraocular, intravitreal, intramuscular, intradermal, intracardiac, intraperitoneal, and subcutaneous).

### Vaccine Efficacy

Some aspects of the present disclosure provide formulations of the compositions (e.g., RNA vaccines), wherein the RNA is formulated in an effective amount to produce an antigen specific immune response in a subject (e.g., production of antibodies specific to a pertussis, tetanus, or diphtheria antigen). “An effective amount” is a dose of the RNA effective to produce an antigen-specific immune response. Also provided herein are methods of inducing an antigen-specific immune response in a subject.

As used herein, an immune response to a vaccine or LNP of the present disclosure is the development in a subject of a humoral and/or a cellular immune response to a (one or more) pertussis, diphtheria, and/or tetanus protein(s) present in the vaccine. For purposes of the present disclosure, a “humoral” immune response refers to an immune response mediated by antibody molecules, including, e.g., secretory (IgA) or IgG molecules, while a “cellular” immune response is one mediated by T-lymphocytes (e.g., CD4+ helper and/or CD8+ T cells (e.g., CTLs) and/or other white blood cells. One important aspect of cellular immunity involves an antigen-specific response by cytolytic T-cells (CTLs). CTLs have specificity for peptide antigens that are presented in association with proteins encoded by the major histocompatibility complex (MHC) and expressed on the surfaces of cells. CTLs help induce and promote the destruction of intracellular microbes or the lysis of cells infected with such microbes. Another aspect of cellular immunity involves and antigen-specific response by helper T-cells. Helper T-cells act to help stimulate the function and focus the activity nonspecific effector cells against cells displaying peptide antigens in association with MHC molecules on their surface. A cellular immune response also leads to the production of cytokines, chemokines, and other such molecules produced by activated T-cells and/or other white blood cells including those derived from CD4+ and CD8+ T-cells. Humoral immune responses may be further divided into Th1 and Th2 responses, resulting the production of Th1-type cytokines and Th2-type cytokines, respectively. Th1-type cytokines tend to produce the proinflammatory responses responsible for killing intracellular parasites and for perpetuating autoimmune responses. The main Th1 cytokine is interferon gamma. Excessive proinflammatory responses (e.g., Th1-based responses), in some embodiments, can lead to uncontrolled tissue damage, and are counteracted by the Th2-type cytokines. The Th2-type cytokines include interleukins 4, 5, and 13, which are associated with the promotion of IgE and eosinophilic responses in atopy, and also interleukin-10, which is anti-inflammatory. In excess, Th2 responses will counteract the Th1 mediated microbicidal action. Accordingly, in some embodiments, the vaccines provided herein elicit a balanced Th1 and Th2 response. In some embodiments, administration of the vaccines provided herein may result in a Th17 response. T helper 17 cells (Th17) are a subset of pro-inflammatory T helper cells defined by their production of interleukin 17. Th17 cells maintain mucosal barriers and contribute to pathogen clearance at the mucosal surfaces. The Th17-type cytokines target innate immune cells and epithelial cells to produce G-CSF and Il-8, leading to neutrophil production and recruitment. In some embodiments, the compositions (e.g., vaccines) of the present disclosure produce a Th1 response. In some embodiments, the compositions (e.g., vaccines) of the present disclosure produce a Th2 response. In some embodiments, the compositions (e.g., vaccines) of the present disclosure produce a Th17 response. In some embodiments, the compositions (e.g., vaccines) of the present disclosure produce Th1 and Th2 responses, Th1 and Th17 responses, Th2 and Th17 responses, or Th1, Th2, and Th17 responses.

In some embodiments, the antigen-specific immune response is characterized by measuring an anti-pertussis antigen antibody titer produced in a subject administered a composition as provided herein. In some embodiments, the antigen-specific immune response is characterized by measuring an anti-diphtheria antigen antibody titer produced in a subject administered a composition as provided herein. In some embodiments, the antigen-specific immune response is characterized by measuring an anti-tetanus antigen antibody titer produced in a subject administered a composition as provided herein. An antibody titer is a measurement of the amount of antibodies within a subject, for example, antibodies that are specific to a particular antigen or epitope of an antigen. Antibody titer is typically expressed as the inverse of the greatest dilution that provides a positive result. Enzyme-linked immunosorbent assay (ELISA) is a common assay for determining antibody titers, for example.

A variety of serological tests can be used to measure antibody against encoded antigen of interest, for example, a pertussis antigen, a diphtheria antigen, or a tetanus antigen. These tests include the hemagglutination-inhibition test, complement fixation test, fluorescent antibody test, enzyme-linked immunosorbent assay (ELISA), and plaque reduction neutralization test (PRNT). Each of these tests measures different antibody activities. In exemplary embodiments, A plaque reduction neutralization test, or PRNT (e.g., PRNT50 or PRNT90) is used as a serological correlate of protection. PRNT measures the biological parameter of in vitro virus neutralization and is the most serologically virus-specific test among certain classes of viruses, correlating well to serum levels of protection from virus infection. The basic design of the PRNT allows for virus-antibody interaction to occur in a test tube or microtiter plate, and then measuring antibody effects on viral infectivity by plating the mixture on virus-susceptible cells, preferably cells of mammalian origin. The cells are overlaid with a semi-solid media that restricts spread of progeny virus. Each virus that initiates a productive infection produces a localized area of infection (a plaque), that can be detected in a variety of ways. Plaques are counted and compared back to the starting concentration of virus to determine the percent reduction in total virus infectivity. In PRNT, the serum sample being tested is usually subjected to serial dilutions prior to mixing with a standardized amount of virus. The concentration of virus is held constant such that, when added to susceptible cells and overlaid with semi-solid media, individual plaques can be discerned and counted. In this way, PRNT end-point titers can be calculated for each serum sample at any selected percent reduction of virus activity.

In functional assays intended to assess vaccinal immunogenicity, the serum sample dilution series for antibody titration should ideally start below the “seroprotective” threshold titer. Regarding pertussis neutralizing antibodies, diphtheria neutralizing antibodies, or tetanus neutralizing antibodies, a seropositivity threshold of 1:10 can be considered a seroprotection threshold in certain embodiments.

PRNT end-point titers are expressed as the reciprocal of the last serum dilution showing the desired percent reduction in plaque counts. The PRNT titer can be calculated based on a 50% or greater reduction in plaque counts (PRNT50). A PRNT50 titer is preferred over titers using higher cut-offs (e.g., PRNT90) for vaccine sera, providing more accurate results from the linear portion of the titration curve.

There are several ways to calculate PRNT titers. The simplest and most widely used way to calculate titers is to count plaques and report the titer as the reciprocal of the last serum dilution to show >50% reduction of the input plaque count as based on the back-titration of input plaques. Use of curve fitting methods from several serum dilutions may permit calculation of a more precise result. There are a variety of computer analysis programs available for this (e.g., SPSS or GraphPad Prism).

In some embodiments, an antibody titer is used to assess whether a subject has had an infection or to determine whether immunizations are required. In some embodiments, an antibody titer is used to determine the strength of an autoimmune response, to determine whether a booster immunization is needed, to determine whether a previous vaccine was effective, and to identify any recent or prior infections. In accordance with the present disclosure, an antibody titer may be used to determine the strength of an immune response induced in a subject by a composition (e.g., RNA vaccine).

In some embodiments, the anti-pertussis antigen antibody titer, anti-diphtheria antigen antibody titer, or anti-tetanus antigen antibody titer produced in a subject is increased by at least 1 log relative to a control. For example, the anti-pertussis antigen antibody titer, anti-diphtheria antigen antibody titer, or anti-tetanus antigen antibody titer produced in a subject may be increased by at least 1.5, at least 2, at least 2.5, or at least 3 log relative to a control. In some embodiments, the anti-pertussis antigen antibody titer, anti-diphtheria antigen antibody titer, or anti-tetanus antigen antibody titer produced in the subject is increased by 1, 1.5, 2, 2.5 or 3 log relative to a control. In some embodiments, the anti-pertussis antigen antibody titer, anti-diphtheria antigen antibody titer, or anti-tetanus antigen antibody titer produced in the subject is increased by 1-3 log relative to a control. For example, t the anti-pertussis antigen antibody titer, anti-diphtheria antigen antibody titer, or anti-tetanus antigen antibody titer produced in a subject may be increased by 1-1.5, 1-2, 1-2.5, 1-3, 1.5-2, 1.5-2.5, 1.5-3, 2-2.5, 2-3, or 2.5-3 log relative to a control.

In some embodiments, the anti-pertussis antigen antibody titer, anti-diphtheria antigen antibody titer, or anti-tetanus antigen antibody titer produced in a subject is increased at least 2 times relative to a control. For example, the anti-pertussis antigen antibody titer, anti-diphtheria antigen antibody titer, or anti-tetanus antigen antibody titer produced in a subject may be increased at least 3 times, at least 4 times, at least 5 times, at least 6 times, at least 7 times, at least 8 times, at least 9 times, or at least 10 times relative to a control. In some embodiments, the anti-pertussis antigen antibody titer, anti-diphtheria antigen antibody titer, or anti-tetanus antigen antibody titer produced in the subject is increased 2, 3, 4, 5, 6, 7, 8, 9, or 10 times relative to a control. In some embodiments, the anti-pertussis antigen antibody titer, anti-diphtheria antigen antibody titer, or anti-tetanus antigen antibody titer produced in a subject is increased 2-10 times relative to a control. For example, the anti-pertussis antigen antibody titer, anti-diphtheria antigen antibody titer, or anti-tetanus antigen antibody titer produced in a subject may be increased 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10 times relative to a control.

In some embodiments, an antigen-specific immune response is measured as a ratio of geometric mean titer (GMT), referred to as a geometric mean ratio (GMR), of serum neutralizing antibody titers to pertussis, diphtheria, and/or tetanus. A geometric mean titer (GMT) is the average antibody titer for a group of subjects calculated by multiplying all values and taking the nth root of the number, where n is the number of subjects with available data.

A control, in some embodiments, is an anti-pertussis antigen antibody titer, anti-diphtheria antigen antibody titer, or anti-tetanus antigen antibody titer produced in a subject who has not been administered a composition (e.g., RNA vaccine). In some embodiments, a control is an anti-pertussis antigen antibody titer, anti-diphtheria antigen antibody titer, or anti-tetanus antigen antibody titer produced in a subject administered a recombinant or purified protein vaccine. Recombinant protein vaccines typically include protein antigens that either have been produced in a heterologous expression system (e.g., bacteria or yeast) or purified from large amounts of the pathogenic organism.

In some embodiments, the ability of a composition (e.g., RNA vaccine) to be effective is measured in a murine model. For example, a composition may be administered to a murine model and the murine model assayed for induction of neutralizing antibody titers. Bacterial challenge studies may also be used to assess the efficacy of a vaccine of the present disclosure. For example, a composition may be administered to a murine model, the murine model challenged with bacterial, and the murine model assayed for survival and/or immune response (e.g., neutralizing antibody response, T cell response (e.g., cytokine response)).

In some embodiments, an effective amount of a composition (e.g., RNA vaccine) is a dose that is reduced compared to the standard of care dose of a recombinant protein vaccine. A “standard of care,” as provided herein, refers to a medical or psychological treatment guideline and can be general or specific. “Standard of care” specifies appropriate treatment based on scientific evidence and collaboration between medical professionals involved in the treatment of a given condition. It is the diagnostic and treatment process that a physician/clinician should follow for a certain type of patient, illness or clinical circumstance. A “standard of care dose,” as provided herein, refers to the dose of a recombinant or purified protein vaccine, or a live attenuated or inactivated vaccine, or a VLP vaccine, that a physician/clinician or other medical professional would administer to a subject to treat or prevent pertussis, diphtheria, and/or tetanus infections or a related condition, while following the standard of care guideline for treating or preventing pertussis, diphtheria, and/or tetanus infection or a related condition.

In some embodiments, the anti-pertussis antigen antibody titer, anti-diphtheria antigen antibody titer, or anti-tetanus antigen antibody titer produced in a subject administered an effective amount of an composition is equivalent to the anti-pertussis antigen antibody titer, anti-diphtheria antigen antibody titer, or anti-tetanus antigen antibody titer produced in a control subject administered a standard of care dose of a recombinant or purified protein vaccine, or a live attenuated or inactivated vaccine, or a VLP vaccine.

Vaccine efficacy may be assessed using standard analyses (see, e.g., Weinberg et al., J Infect Dis. 2010 Jun. 1; 201(11):1607-10). For example, vaccine efficacy may be measured by double-blind, randomized, clinical controlled trials. Vaccine efficacy may be expressed as a proportionate reduction in disease attack rate (AR) between the unvaccinated (ARU) and vaccinated (ARV) study cohorts and can be calculated from the relative risk (RR) of disease among the vaccinated group with use of the following formulas:

Efficacy=(ARU−ARV)/ARU×100; and

Efficacy=(1−RR)×100.

Likewise, vaccine effectiveness may be assessed using standard analyses (see, e.g., Weinberg et al., J Infect Dis. 2010 Jun. 1; 201(11):1607-10). Vaccine effectiveness is an assessment of how a vaccine (which may have already proven to have high vaccine efficacy) reduces disease in a population. This measure can assess the net balance of benefits and adverse effects of a vaccination program, not just the vaccine itself, under natural field conditions rather than in a controlled clinical trial. Vaccine effectiveness is proportional to vaccine efficacy (potency) but is also affected by how well target groups in the population are immunized, as well as by other non-vaccine-related factors that influence the ‘real-world’ outcomes of hospitalizations, ambulatory visits, or costs. For example, a retrospective case control analysis may be used, in which the rates of vaccination among a set of infected cases and appropriate controls are compared. Vaccine effectiveness may be expressed as a rate difference, with use of the odds ratio (OR) for developing infection despite vaccination:

Effectiveness=(1−OR)×100.

In some embodiments, efficacy of the composition (e.g., RNA vaccine) is at least 60% relative to unvaccinated control subjects. For example, efficacy of the composition may be at least 65%, at least 70%, at least 75%, at least 80%, at least 85%, at least 95%, at least 98%, or 100% relative to unvaccinated control subjects.

Sterilizing Immunity. Sterilizing immunity refers to a unique immune status that prevents effective pathogen infection into the host. In some embodiments, the effective amount of a composition of the present disclosure is sufficient to provide sterilizing immunity in the subject for at least 1 year. For example, the effective amount of a composition of the present disclosure is sufficient to provide sterilizing immunity in the subject for at least 2 years, at least 3 years, at least 4 years, at least 5 years, at least 6 years, at least 7 years, at least 8 years, at least 9 years, at least 10 years, or more. In some embodiments, the effective amount of a composition of the present disclosure is sufficient to provide sterilizing immunity in the subject at an at least 5-fold lower dose relative to control. For example, the effective amount may be sufficient to provide sterilizing immunity in the subject at an at least 10-fold lower, 15-fold, or 20-fold lower dose relative to a control.

Detectable Antigen. In some embodiments, the effective amount of a composition of the present disclosure is sufficient to produce detectable levels of pertussis, diphtheria, and/or tetanus antigen as measured in serum of the subject at 1-72 hours post administration.

Titer. An antibody titer is a measurement of the number of antibodies within a subject, for example, antibodies that are specific to a particular antigen (e.g., an anti-pertussis antigen, anti-diphtheria antigen, or anti-tetanus antigen). Antibody titer is typically expressed as the inverse of the greatest dilution that provides a positive result. Enzyme-linked immunosorbent assay (ELISA) is a common assay for determining antibody titers, for example.

In some embodiments, the effective amount of a composition of the present disclosure is sufficient to produce a 1,000-10,000 neutralizing antibody titer produced by neutralizing antibody against the specific antigen as measured in serum of the subject at 1-72 hours post administration. In some embodiments, the effective amount is sufficient to produce a 1,000-5,000 neutralizing antibody titer produced by neutralizing antibody against the specific antigen as measured in serum of the subject at 1-72 hours post administration. In some embodiments, the effective amount is sufficient to produce a 5,000-10,000 neutralizing antibody titer produced by neutralizing antibody against the specific antigen as measured in serum of the subject at 1-72 hours post administration.

In some embodiments, the neutralizing antibody titer is at least 100 NT₅₀. For example, the neutralizing antibody titer may be at least 200, 300, 400, 500, 600, 700, 800, 900 or 1000 NT₅₀. In some embodiments, the neutralizing antibody titer is at least 10,000 NT₅₀.

In some embodiments, the neutralizing antibody titer is at least 100 neutralizing units per milliliter (NU/mL). For example, the neutralizing antibody titer may be at least 200, 300, 400, 500, 600, 700, 800, 900 or 1000 NU/mL. In some embodiments, the neutralizing antibody titer is at least 10,000 NU/mL.

In some embodiments, an anti-pertussis antigen antibody titer, anti-diphtheria antigen antibody titer, or anti-tetanus antigen antibody titer produced in the subject is increased by at least 1 log relative to a control. For example, an anti-pertussis antigen antibody titer, anti-diphtheria antigen antibody titer, or anti-tetanus antigen antibody titer produced in the subject may be increased by at least 2, 3, 4, 5, 6, 7, 8, 9 or 10 log relative to a control.

In some embodiments, an anti-pertussis antigen antibody titer, anti-diphtheria antigen antibody titer, or anti-tetanus antigen antibody titer produced in the subject is increased at least 2 times relative to a control. For example, an anti-pertussis antigen antibody titer, anti-diphtheria antigen antibody titer, or anti-tetanus antigen antibody titer produced in the subject is increased by at least 3, 4, 5, 6, 7, 8, 9 or 10 times relative to a control.

In some embodiments, a geometric mean, which is the nth root of the product of n numbers, is generally used to describe proportional growth. Geometric mean, in some embodiments, is used to characterize antibody titer produced in a subject.

A control may be, for example, an unvaccinated subject, or a subject administered a whole cell bacterial vaccine or an acellular vaccine (e.g., DTaP or Tdap).

### EXAMPLES

### Example 1. In Vivo Study of mRNA Vaccine (Single Antigen)

To study the effects of the mRNA vaccine, mice were injected with a negative control (no vaccine, no Bordetella pertussis), a positive control (no vaccine, Bordetella pertussis-infected), or an mRNA pertussis vaccine (50 μL intramuscular injection) formulated with lipid nanoparticles (LNPs) on day 0. Twenty-eight days later, the different groups received a booster dose. Fifty-six days after the initial vaccination, the mice were challenged with 2×10⁷ CFU Bordetella pertussis. Three days after the challenge (59 days after the initial vaccination), the bacterial burden, serological response, and immune profile of the mice were examined.

In each vaccine, the mRNA is formulated in lipid nanoparticles (LNPs) including 0.5-15% PEG-modified lipid, 5-25% non-cationic lipid, 25-55% sterol, and 20-60% ionizable cationic lipid. The PEG-modified lipid is 1,2 dimyristoyl-sn-glycerol, methoxypolyethyleneglycol (PEG2000 DMG), the non-cationic lipid is 1,2 distearoyl-sn-glycero-3-phosphocholine (DSPC), the sterol is cholesterol, and the ionizable cationic lipid has the structure of Compound 1, for example.

mRNA vaccines expressing individual pertussis toxins (C180, PTX, RTX, and ACT-CAT), different adhesion proteins (FHA1, FHA2, FHA3, FHA4, PRN, and BrkA) were designed and synthesized. Other antigens and controls were also generated, including VagI, TcfA, SphB2, Fim, PT-alum (genetically detoxified, 1.25 μg), wP ( 1/20 human dose), and DTaP ( 1/20 human dose). The toxin endpoints measured included: total white blood cell, neutrophil, and lymphocytes, systemic IL-6 (indicative of inflammation), weight loss, and PT and ACT neutralization. A toxin-binding ELISA was also performed. The adhesion/surface antigen endpoints were as follows: lung and nasal bacterial load, systemic IL-6, toxin-binding ELISA, and live Bordetella pertussis staining (as an OPA surrogate).

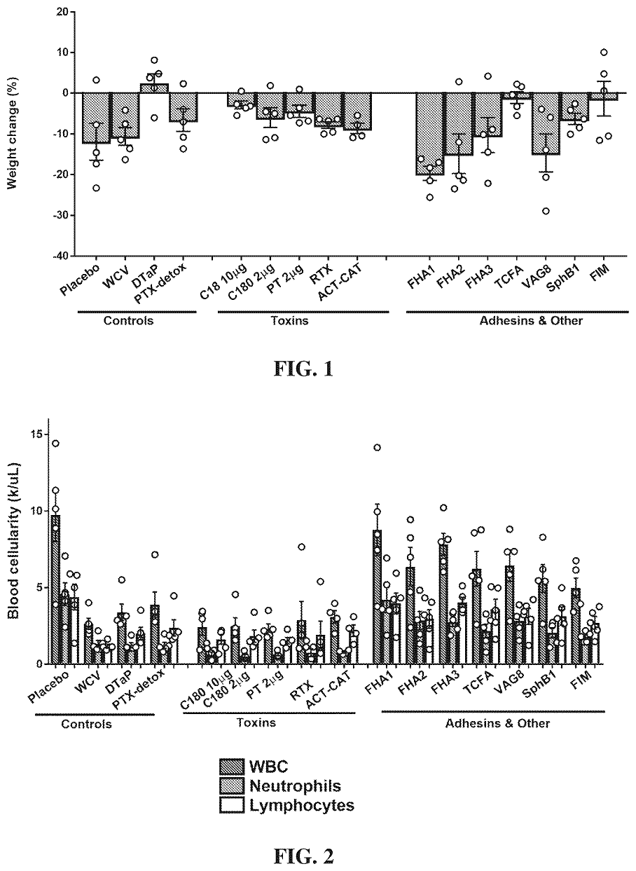

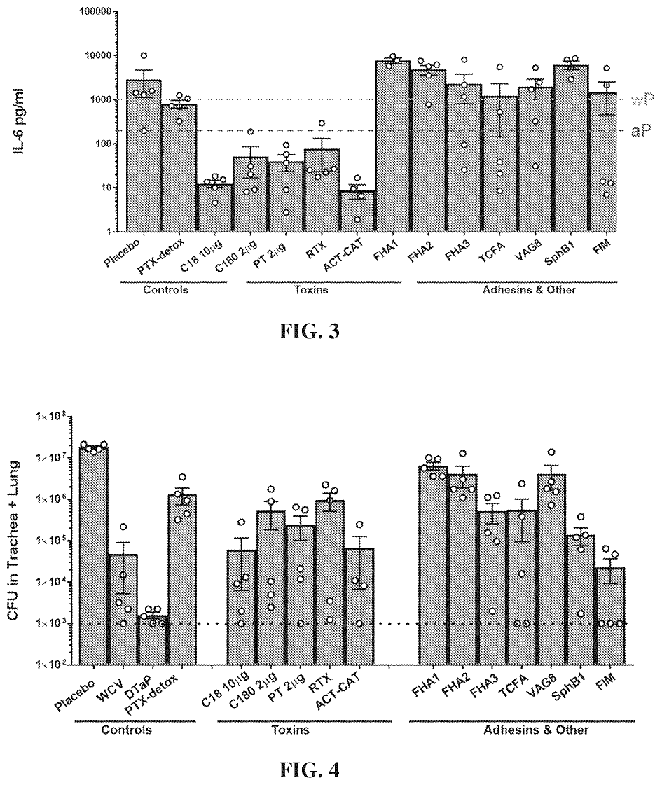

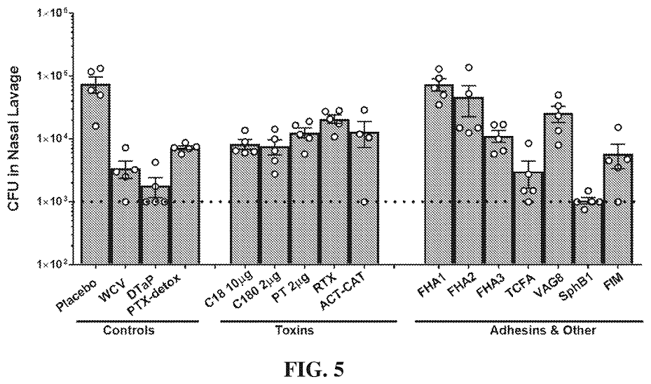

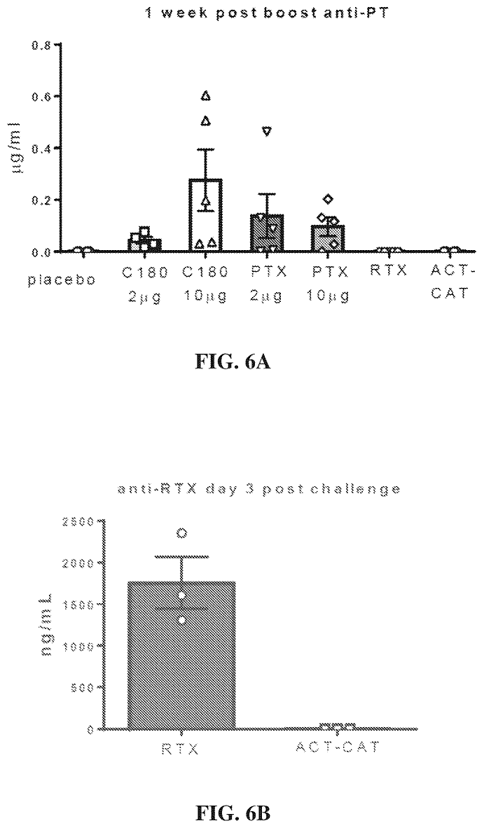

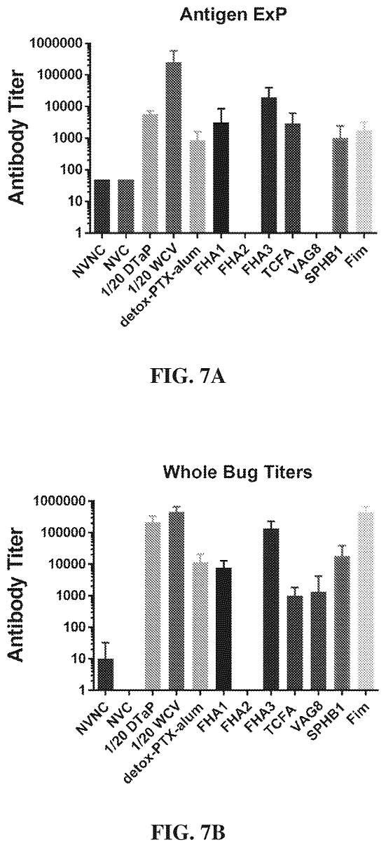

The results are shown in FIGS. 1-7B. FIG. 1 shows the percent weight change (weight loss) among the different groups. All of the toxin mRNA constructs showed average weight losses less than that of the placebo. As shown in FIG. 2, the toxin mRNA constructs also showed reduced cell counts (white blood cells, neutrophils, and lymphocytes) relative to the placebo. FIG. 3, which shows the concentration of IL-6 in samples, demonstrates that the toxin mRNA constructs prevent systemic inflammation comparable to that of the 1/20 DTaP dose. The bacterial load in the trachea and lung and in nasal lavage was determined. As shown in FIGS. 4 and 5, TCFA, SPHB1, FHA3, and FIM3 were found to impact lung/nasal bacterial load. FIGS. 6A-6B show antibody titers following administration of the toxin mRNA constructs booster (FIG. 6A) and then post-challenge (FIG. 6B). C180 and PTX were found to prevent toxin-mediated pathology (FIG. 6A), as did RTX and its catalytic domain constructs. Further, antibody titers to secreted antigens (extracellular pertussis protein, 100 proteins) were examined (FIG. 7A), and it was found that the antibody titer was increased in most of the mRNA constructs relative to the controls. When the live Bordetella pertussis binding assay was performed, antibody titers in the mRNA constructs were also increased relative to the controls (FIG. 7B).

The mouse experiments demonstrated that mRNA expressed pertussis toxin constructs were able to prevent toxin-mediated pathology (see, e.g., C180 and PTX). Further, TCFA, SPHB1, FHA3, and FIM3 were found to impact lung/nasal bacterial load.

### Example 2. In Vivo Study of mRNA Vaccine (Antigen Combinations)

To study the effects of the mRNA vaccine, mice were injected with a negative control (no vaccine, no Bordetella pertussis), a positive control (no vaccine, Bordetella pertussis-infected), or an mRNA pertussis vaccine (50 μL intramuscular injection) with lipid nanoparticles on day 0. Twenty-eight days later, the different groups received a booster dose. Fifty-six days after the initial vaccination, the mice were challenged with 2×10⁷ CFU Bordetella pertussis. Three days after the challenge (59 days after the initial vaccination), the bacterial burden, serological response, and immune profile of the mice were examined.

In each vaccine, the mRNA is formulated in lipid nanoparticles (LNPs) including 0.5-15% PEG-modified lipid, 5-25% non-cationic lipid, 25-55% sterol, and 20-60% ionizable cationic lipid. The PEG-modified lipid is 1,2 dimyristoyl-sn-glycerol, methoxypolyethyleneglycol (PEG2000 DMG), the non-cationic lipid is 1,2 distearoyl-sn-glycero-3-phosphocholine (DSPC), the sterol is cholesterol, and the ionizable cationic lipid has the structure of Compound 1, for example.

mRNA vaccines expressing different pertussis antigens or combinations of different pertussis antigens were designed and synthesized. The groups tested and dose of mRNA administered are shown in the table below:

| Group | Antigen(s) | Dose(ug) |
|---|---|---|
| 1 | Fim2-3 | 2 |
| 2 | Fim2 | 2 |
| 3 | RTX | 2 |
| 4 | SPHB1 | 2 |
| 5 | TCFA | 2 |
| 6 | FHA3 | 2 |
| 7 | C180 | 2 |
| 8 | FHA3 + FIM2 + SPHB1 | 6 |
| 9 | FHA3 + FIM2 + C180 | 6 |
| 10 | FHA3 + FIM2-3 + C180 | 6 |
| 11 | FIM2 + TCFA + C180 | 6 |
| 12 | C180 + RTX | 4 |
| 13 | FHA3 + FIM2 + TCFA + SPHB1 | 8 |
| 14 | C180 + FHA3 + FIM2 + RTX | 8 |
| 15 | FHA3 + FIM2 + SBHB1 + TCFA | 8 |
| 16 | C180 + FHA3 + FIM2 + PRN | 8 |
| 17 | C180 + FHA3 + FIM2 + SPHB1 + RTX | 10 |
| 18 | C180 + FHA3 + FIM2 + SPHB1 | 8 |
| 19 | C180 + FHA3 + FIM2 + SPHB1 + RTX + TCFA | 12 |

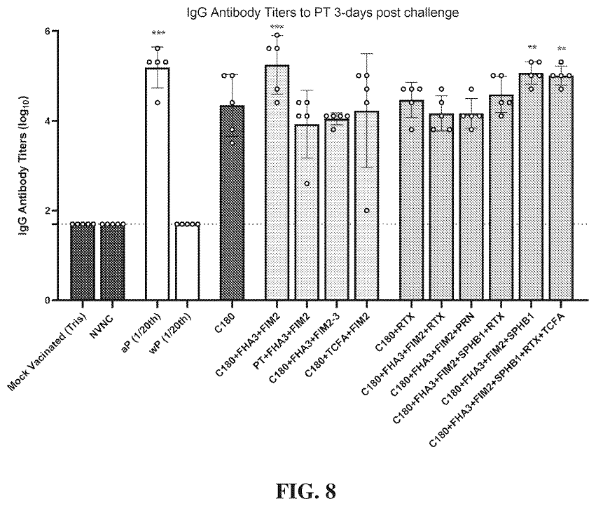

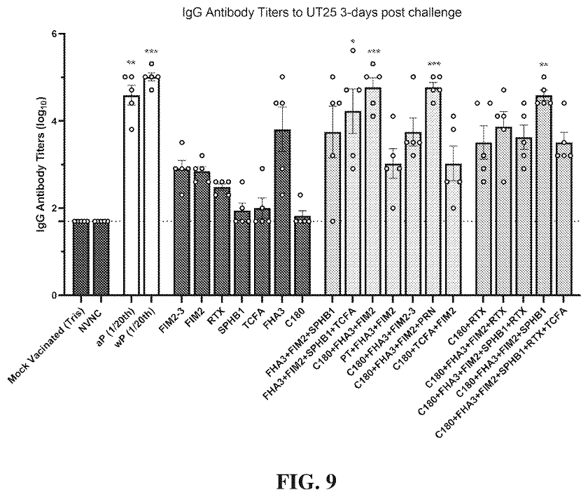

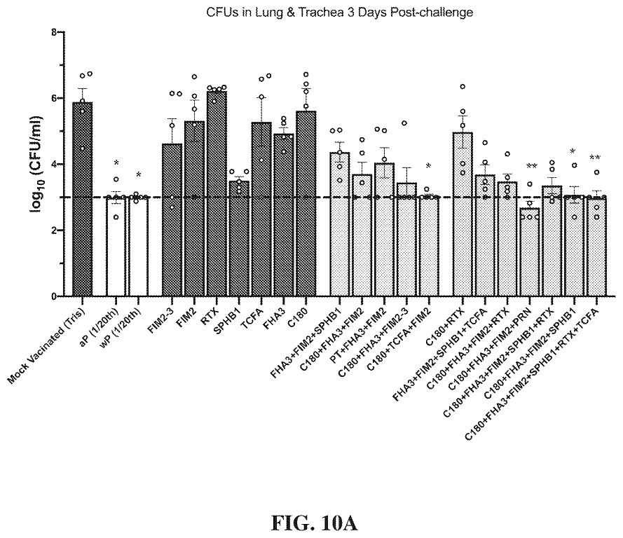

Serum IgG antibody titers to pertussis toxin (PT) were measured by ELISA using plates coated with 0.1 μg/mL PT. The data is shown in FIG. 8 and demonstrates that IgG antibody titers were increased in every vaccine group tested above that of the whole cell vaccine (wP) and controls. The increases in antibody titers were statistically significant for the C180+FHA3+FIM2+SPHB1 (p<0.005) and C180+FHA3+FIM2+SPHB1+RTX+TCFA (p<0.005), and C180+FHA3+FIM2 (p<0.0005) combination vaccines, as well as the acellular vaccine (aP; p<0.0005) compared to the mock vaccinated group. A second ELISA was performed to examine serum IgG antibody titers to UT25 (“whole bug” pertussis). Plates were coated with UT25 at 0.243 OD. The results are shown in FIG. 9 and show that the IgG antibody titers were increased in every group above that of the mock-vaccinated group. In particular, the FHA3+FIM2+SPHB1+TCFA (p<0.05), C180+FHA3+FIM2+SPHB1 (p<0.005), acellular vaccine (aP; p<0.005), whole cell vaccine (wP; p<0.0005), C180+FHA3+FIM2 (p<0.0005), and C180+FHA3+FIM2+PRN (p<0.0005) groups showed statistically significant increases relative to the mock vaccinated group.

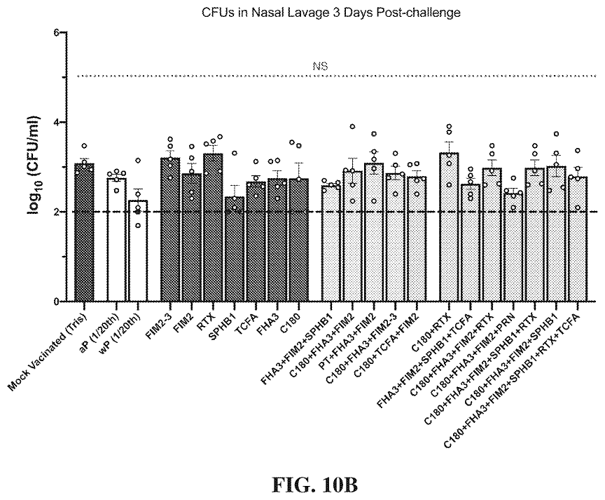

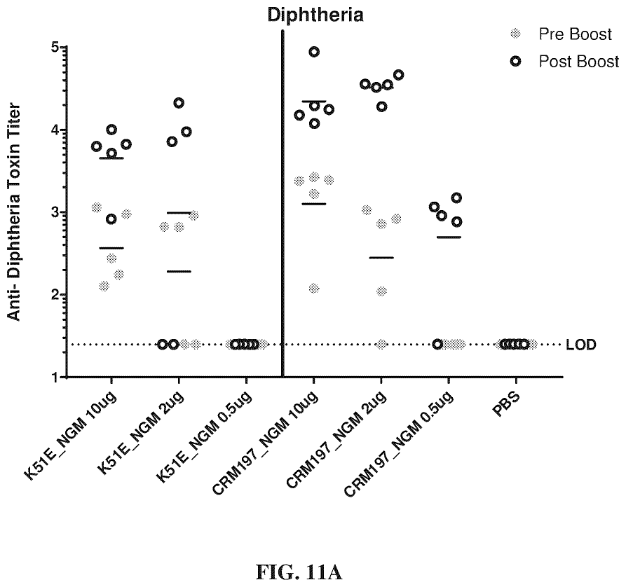

The bacterial burden in the lung, trachea, and nose of test animals was also measured and the results are shown in FIGS. 10A (lung and trachea) and 10B (nose). The challenge dose was too low for meaningful nasal measurements; however, as shown in FIG. 10A, four of the combination vaccines had statistically significant reduced bacterial burden (lung and trachea): C180+TCFA+FIM2, C180+FHA3+FIM2+PRN, C180+FHA3+FIM2+SPHB1, and C180+FHA3+FIM2+SPHB1+RTX+TCFA.

Summaries of the studies in Examples 1 and 2 (comparing the single antigens at two different dosages) are presented below, where “X” represents that the values were less than the wP/aP controls, “XX” represents that the values were equivalent or better than the wP/aP controls, and no mark signifies there was no activity.

STUDY 1
10 μg Dose

| | Reduction in | Reduction in | | |
|---|---|---|---|---|
| | systemic | bacterial | Bactericidal | Anti-toxin |
| Antigen | pathology | load | antibodies | antibodies |
| C180 | XX | X | | XX |
| RTX | XX | | | XX |
| FIM2 | | XX | XX | |
| SPHB1 | | XX | X | |
| TCFA | X | XX | X | |
| FHA3 | X | X | XX | |

STUDY 2
2 μg Dose

| | Systemic | Bacterial | Bug Binding | PT/RTX |
|---|---|---|---|---|
| Antigen | pathology | load | antibodies | antibodies |
| C180 | X | | | XX |
| RTX | X | | X | |
| FIM2 | | X | X | |
| FIM2-3 | | X | X | |
| SPHB1 | | XX | | |
| TCFA | | X | | |
| FHA3 | | X | XX | |

A summary of the antigen combination results is shown in the table below (X” represents that the values were less than the wP/aP controls, “XX” represents that the values were equivalent or better than the wP/aP controls, and no mark signifies there was no activity):

STUDY 2
Antigen Combinations

| | Systemic | Bacterial | Bug Binding | |
|---|---|---|---|---|
| Antigen | pathology | load | antibodies | PT/RTX antibodies |
| aP | XX | XX | X | XX |
| wp | X | XX | XX | |
| C180 + FHA3 + FIM2 + PRN | XX | XX | XX | X |
| C180 + FHA3 + FIM2 + SPHB1 + RTX + | XX | XX | X | XX |
| TCFA | | | | |
| C180 + FHA3 + FIM2 + SPHB1 + RTX | XX | XX | XX | XX |
| C180 + FHA3 + FIM2 + RTX | XX | XX | X | X |
| C180 + RTX | XX | | X | XX |

### Example 3—In Vivo Immunization of Mice (Diphtheria Detoxified Toxins and Tetanus Toxin Fragment C)

To study the effects of the diphtheria and tetanus mRNA vaccines with lipid nanoparticles, C57BL/6 mice (n=5/group) were pre-bled on day −3 and then injected with an mRNA vaccine (50 μL intramuscular injection; 0.5 μg, 2 μg, or 10 μg mRNA concentration) on day 0. On day 27. blood was collected for serum analysis. Twenty-eight days after the initial immunization, the different groups received a booster dose in the same amount as the initial dose. Fifty-six days after the initial vaccination, blood was collected.

In each vaccine, the mRNA is formulated in lipid nanoparticles (LNPs) including 0.5-15% PEG-modified lipid, 5-25% non-cationic lipid, 25-55% sterol, and 20-60% ionizable cationic lipid. The PEG-modified lipid is 1,2 dimyristoyl-sn-glycerol, methoxypolyethyleneglycol (PEG2000 DMG), the non-cationic lipid is 1,2 distearoyl-sn-glycero-3-phosphocholine (DSPC), the sterol is cholesterol, and the ionizable cationic lipid has the structure of Compound 1, for example.

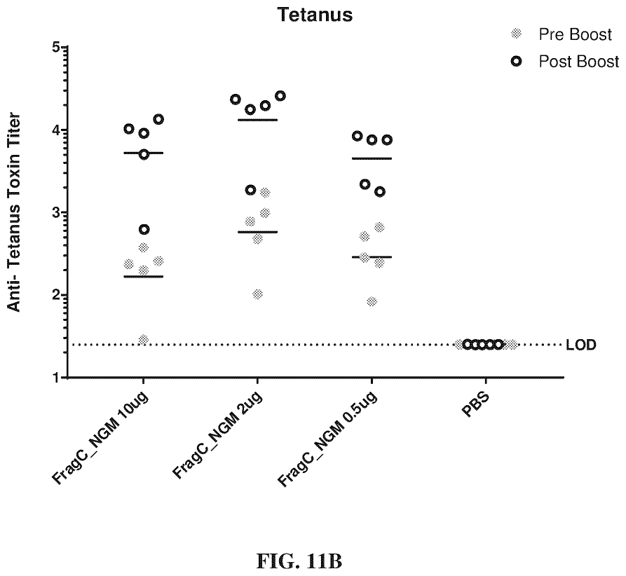

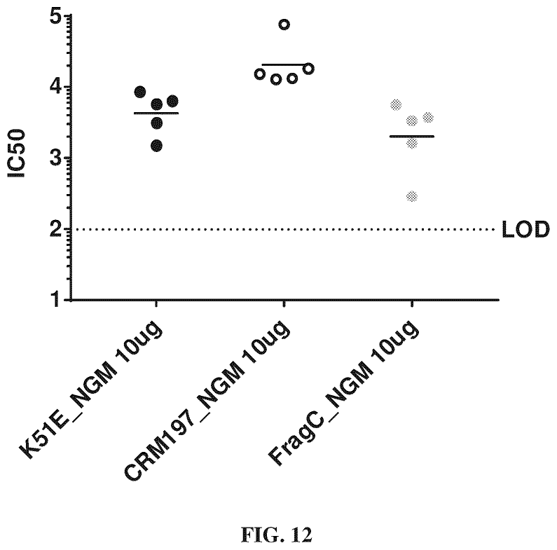

Two different diphtheria detoxified toxins were tested (K51E_NGM and CRM197_NGM), as was tetanus toxin fragment C (FragC_NGM). ELISAs were performed on the collected serum using native diphtheria toxin (mutated G52 E) protein and tetanus toxoid coated at 1ug/ml. As shown in FIG. 11A, the CRM197 detoxified diphtheria toxin elicited higher binding titers than the K5AE detoxified diphtheria toxin across all dose levels. In tetanus, FragC elicited similar titers across all dose levels tested (FIG. 180). The IC₅₀ values for each vaccine at the 10 μg dose were compared (FIG. 12) and illustrates the same trend.

### Example 4—Immunogenicity and Efficacy of mRNA Vaccine Formulations

The immunogenicity and efficacy of mRNA vaccine formulations were tested in a mouse model. Briefly, female BALB/c mice, 4 weeks of age, were immunized with a priming dose of the vaccine on Day 0, and a boost dose on day 28. The groups are shown in the table below. Each dose was administered as a 50 μL dose intramuscularly to the subject's leg.

| Group | Antigens | total dose |
|---|---|---|
| 1 | FHA + FIM + SPHB1 + TCFA + RTX + PRN + C180 | 10 ug |
| 2 | FHA + FIM + SPHB1 + TCFA + RTX + PRN + C180 + TET + DIP | 10 ug |
| 3 | FHA + FIM + SPHB1 + TCFA + RTX + PRN + C180 | 2 ug |
| 4 | FHA + FIM + SPHB1 + TCFA + RTX + PRN + C180 + TET + DIP | 2 ug |
| 5 | C180_TET | 10 ug |
| 6 | PRN_BRKA | 10 ug |
| 7 | FHA_FIM | 10 ug |
| 8 | TCFA_SPHB1 | 10 ug |
| 9 | RTX_DIP | 10 ug |
| 10 | C180_TET | 2 ug |
| 11 | PRN_BRKA | 2 ug |
| 12 | FHA_FIM | 2 ug |
| 13 | TCFA_SPHB1 | 2 ug |
| 14 | RTX_DIP | 2 ug |
| 15 | C180_PRN + FHA_FIM + RTX_DIP + C180_TET + TCFA_SPHB1 | 10 ug |
| 16 | DTap | 1/20th |
| 17 | wP | 1/20th |
| 18 | NVC − PBS control | NA |
| 19 | NVNC | NA |

Blood samples were taken on Days 53, 58 (Groups 1-5, 10, 16-19), and 59 (Groups 6-9, 11-15), and subjects were challenged with Bordetella pertussis (strain UT25) on days 55 (Groups 1-5, 10, 16-19) or 56 (Groups 6-9, 11-15).

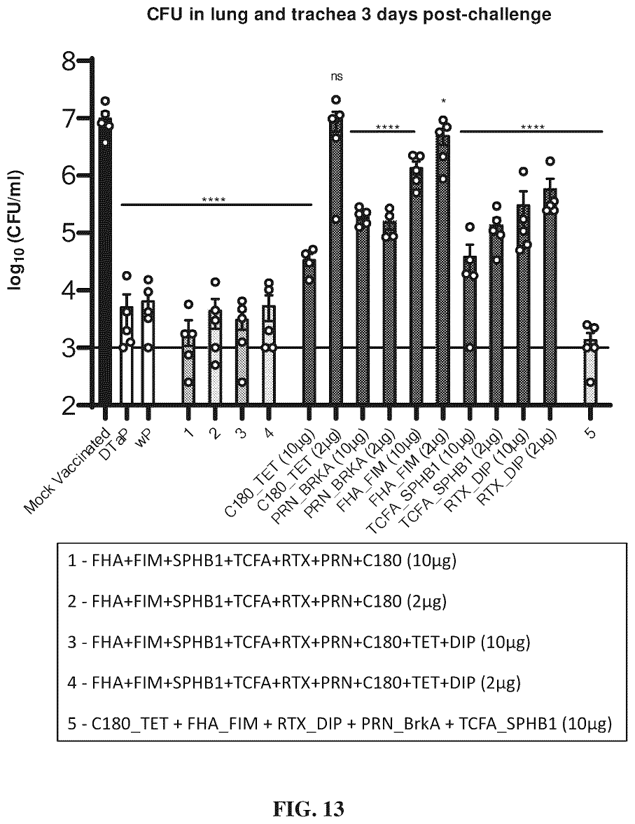

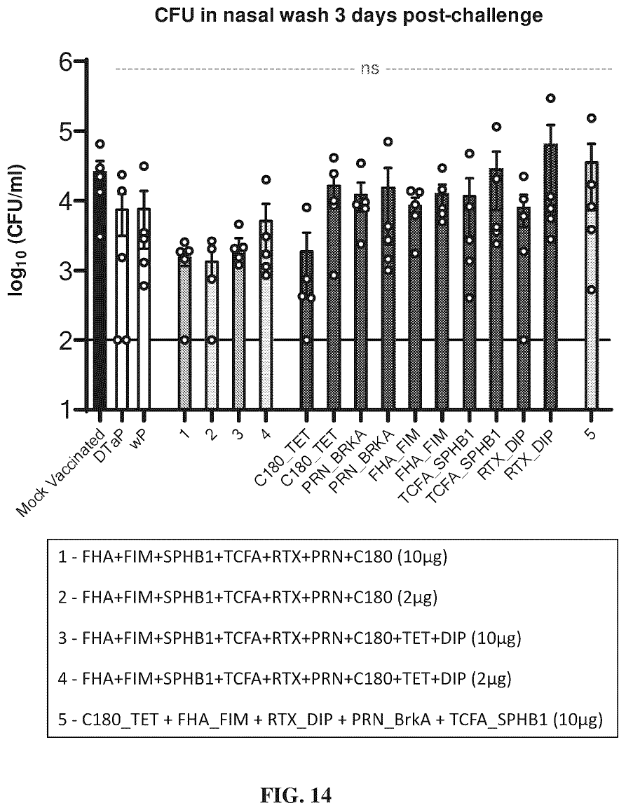

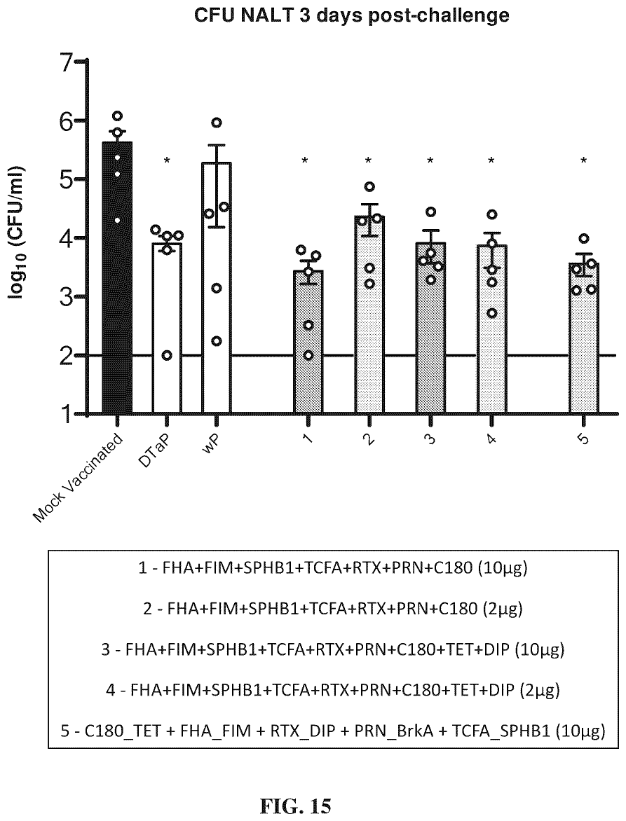

The data is shown in FIGS. 13-27. FIG. 13 shows the colony forming units (CFUs) in lung and trachea samples three days post B. pertussis challenge. The data demonstrates that CFUs were significantly increased in the singular antigen formulation groups, as compared to the combination groups. FIG. 14 shows the nasal lavage data from three days post B. pertussis challenge. There were no significant differences between groups. FIG. 15 shows the CFUs present in nasal-associated lymphoid tissue from three days post B. pertussis challenge. All of the combination vaccines showed significantly less CFU/mL than the mock vaccinated group and the whole cell pertussis vaccine group (wP).

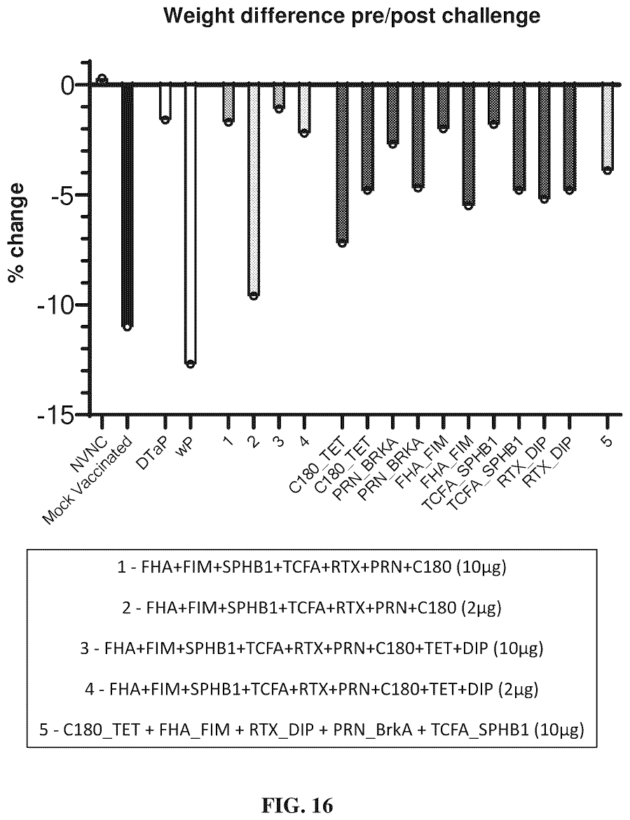

FIG. 16 shows the percent change in mouse weight between the day of the challenge and three days post-challenge. The largest weight loss was observed in the wP group, while several of the combination vaccines and the DTaP had smaller weight losses.

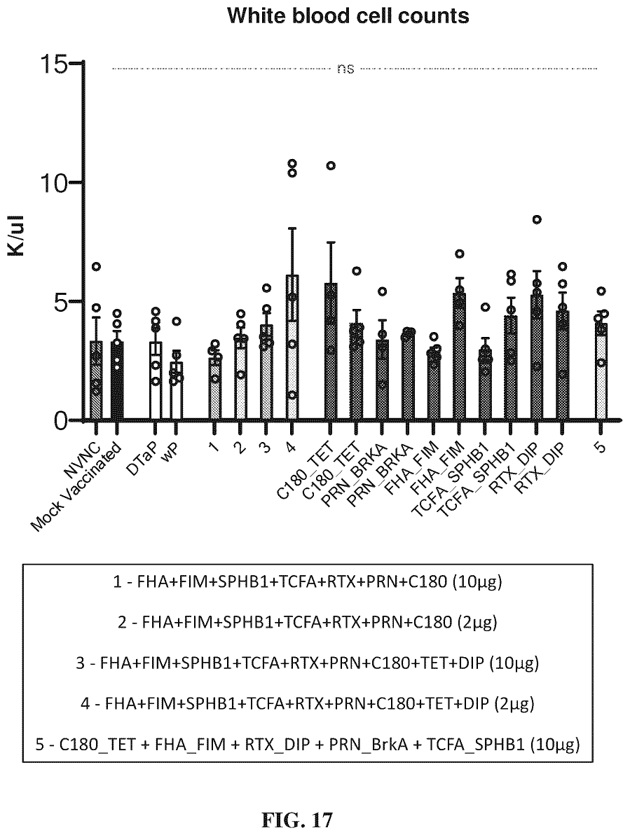

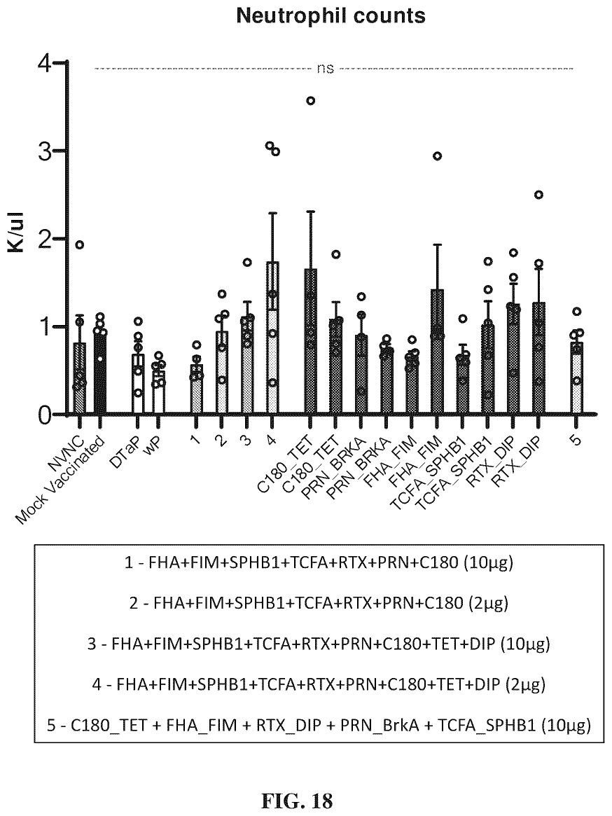

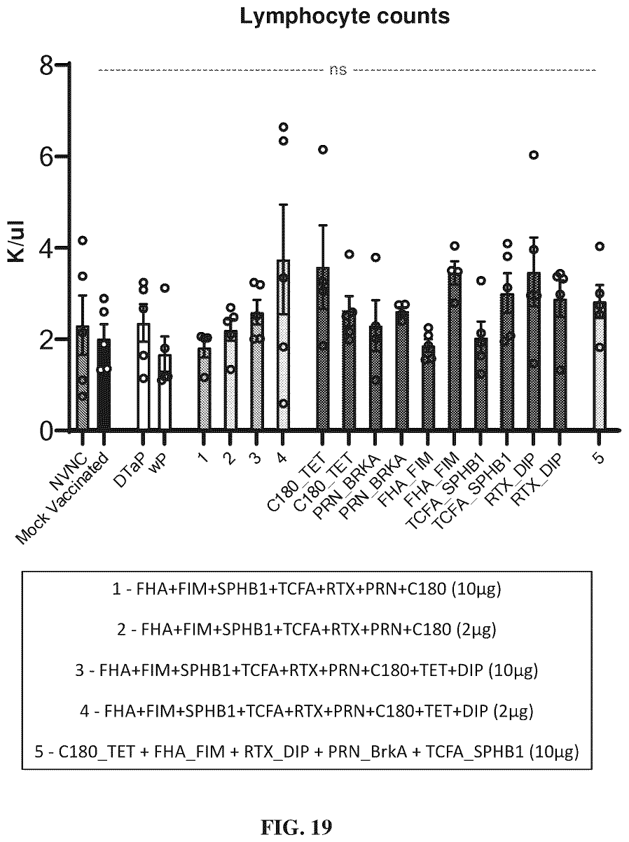

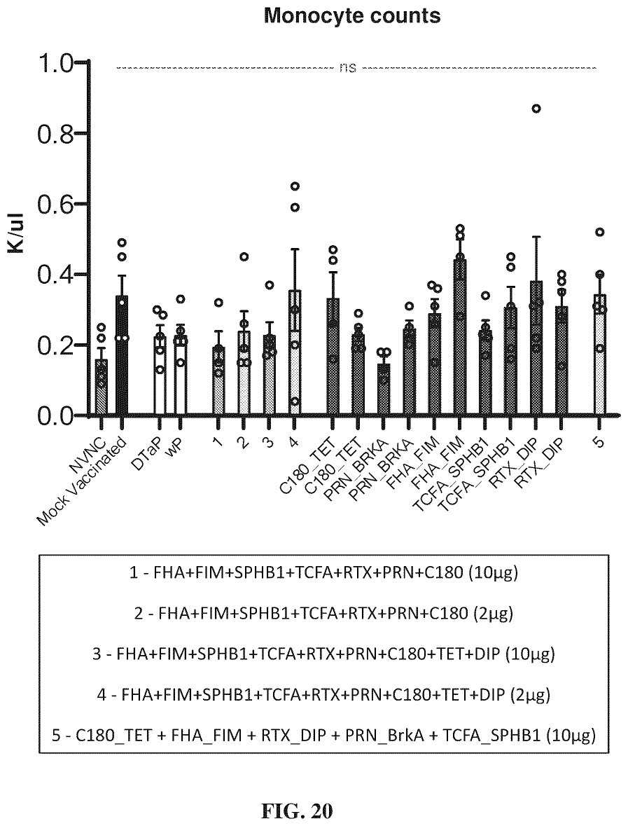

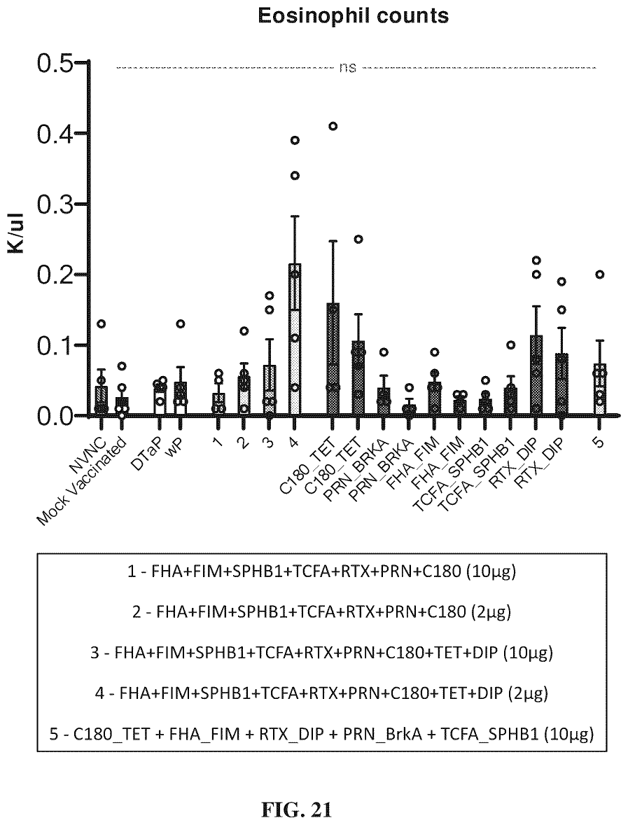

FIG. 17 shows the complete white blood cell counts between different immunization groups three days after B. pertussis challenge. There were no significant differences across groups. FIG. 18 shows neutrophil counts between different immunization groups three days after B. pertussis challenge. Hemavet was used to determine the neutrophil counts. No significant differences were observed between groups. FIG. 19 shows lymphocyte counts between different immunization groups three days after B. pertussis challenge. Hemavet was used to determine the lymphocyte counts. No significant differences were observed between groups. FIG. 20 shows monocyte counts between different immunization groups three days after B. pertussis challenge. Hemavet was used to determine the monocyte counts. No significant differences were observed between groups. FIG. 21 shows eosinophil counts between different immunization groups three days after B. pertussis challenge. Hemavet was used to determine the eosinophil counts. No significant differences were observed between groups.

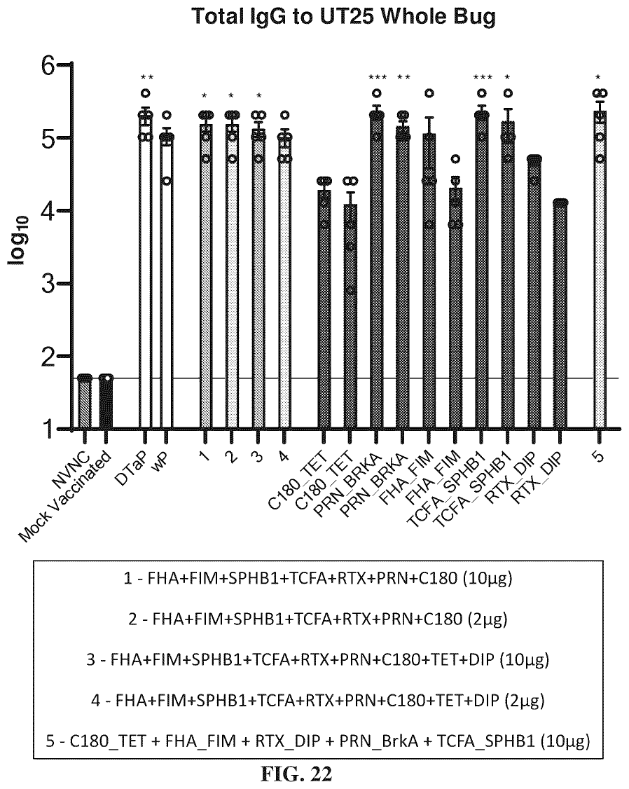

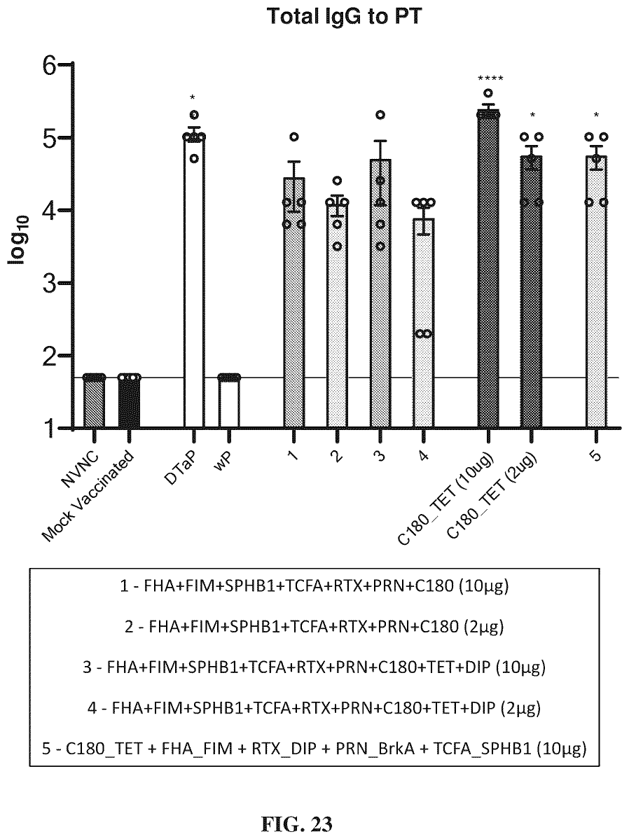

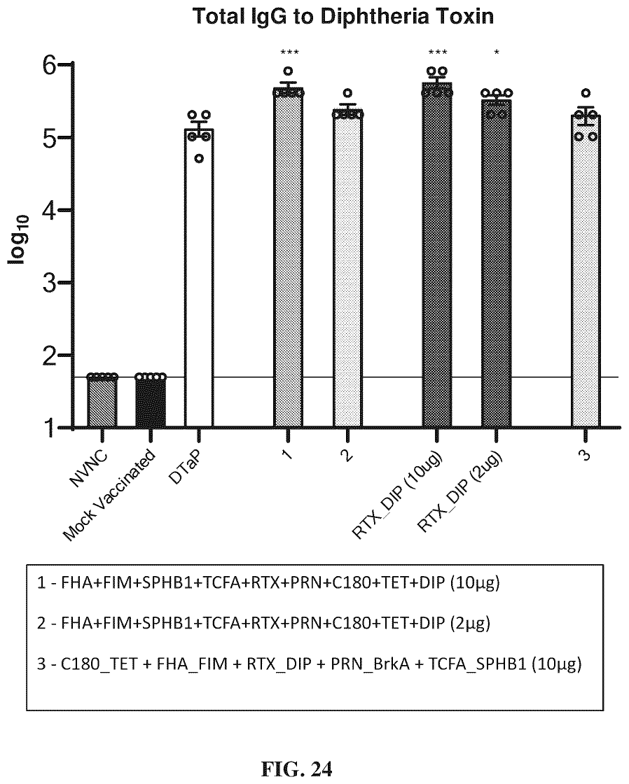

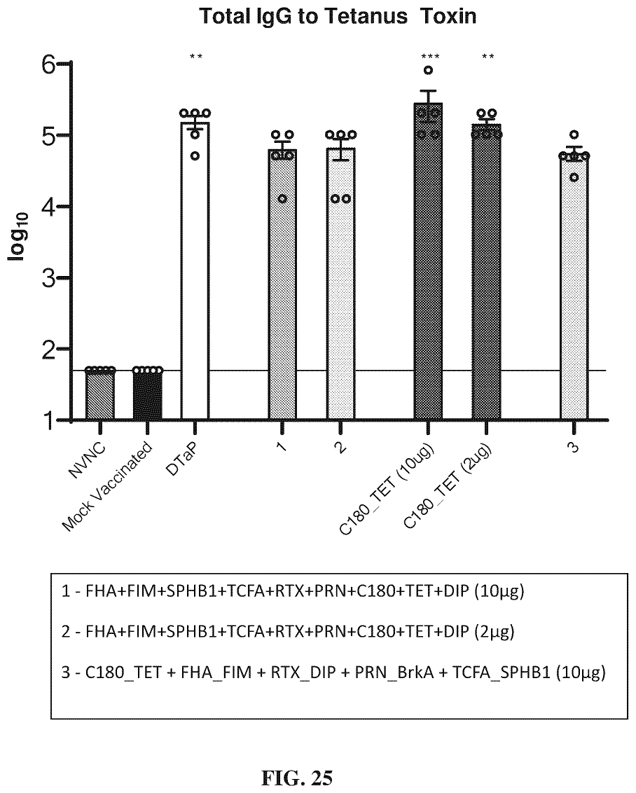

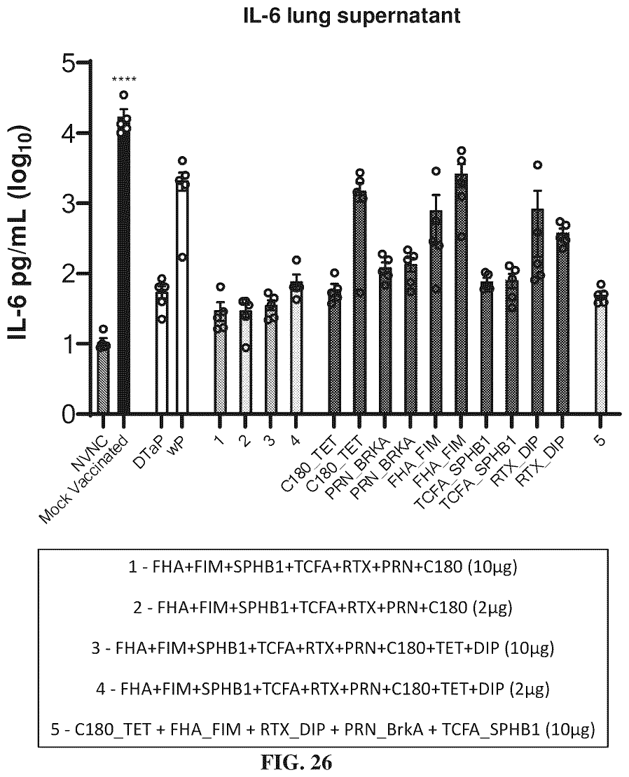

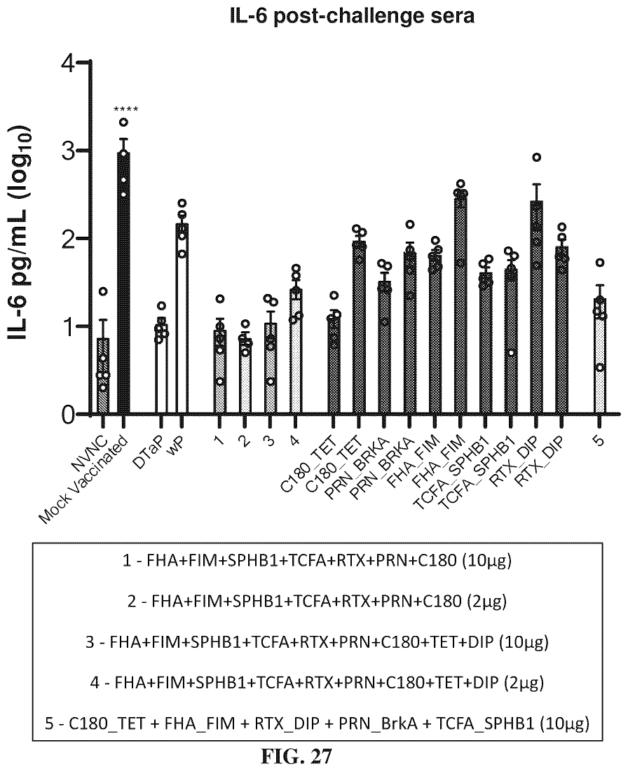

FIG. 22 shows serum antibody (IgG) titers to whole bug (B. pertussis strain UT25). Plates were coated with UT25 at 0.245 OD and antibody titers were determined using ELISA assay. Significant differences were observed relative to the non-vaccinated negative control (NVNC) and the mock vaccinated control. FIG. 23 shows serum antibody (IgG) titers to pertussis toxin (PT), at three days post-challenge. Plates were coated with PT at 0.1 μg/mL (List Biological Laboratories #181) and antibody titers were determined using ELISA assay. Significant differences were observed between the experimental groups and controls, with the exception of the wP vaccination group. FIG. 24 shows serum antibody (IgG) titers to diphtheria toxin three days post-challenge. Plates were coated with Diphtheria toxin (Abcam 188505) at 0.1 μg/mL and antibody titers were determined using ELISA assays. Significant increases were observed for two of the formulations tested. FIG. 25 shows serum antibody (IgG) titers to tetanus toxin three days post-challenge. Plates were coated with tetanus toxin (Enzo ALX630108) at 0.1 μg/mL and antibody titers were determined using ELISA assays. Significant increases were observed for the singular formulation tested, but not for the combination vaccines. IL-6 levels were measured in lung supernatant (FIG. 26) and sera (FIG. 27) three days post-challenge. Single spot 96-well plates were used to determine IL-6 cytokine levels in the samples. Reductions in IL-6 were observed in all of the samples from subjects administered mRNA vaccines.

### Example 5—Immunogenicity of mRNA Vaccine Comprising 10 Antigens against Different Clinical Isolates

The immunogenicity and efficacy of an mRNA vaccine (“mRNA-DTP-10”) comprising ten different antigens (eight pertussis-related antigens, as well as diphtheria and tetanus antigens), each synthesized and individual mRNA molecules and then combined into the same lipid nanoparticle (LNP) formulation. The LNP formulation comprised Compound 1, DSPC, a PEG-modified lipid, and cholesterol. Other vaccine formulations were tested, including the DTaP vaccine, the whole cell vaccine (SI WCV), and positive controls (MCV-UT25 and MCV-D420).

Briefly, female BALB/c mice, approximately 4 weeks of age, were vaccinated on Day 0 and then received a boost dose of the vaccine on Day 30. On Day 44, the mice were challenged with Bordetella pertussis (strain UT25 or D420). Three days later, samples were taken for serology, complete blood count (CBC) analysis, and to count colony-forming units (CFUs).

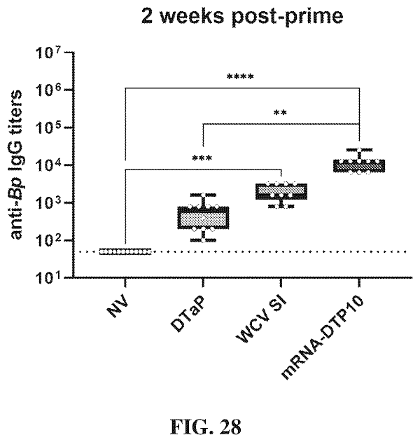

The anti-B. pertussis antibody (IgG) titers two weeks after the initial (priming) vaccination are shown in FIG. 28. Statistically significant differences were observed between all groups and the control (NV), with the mRNA vaccine having the highest antigen-specific antibody titer.

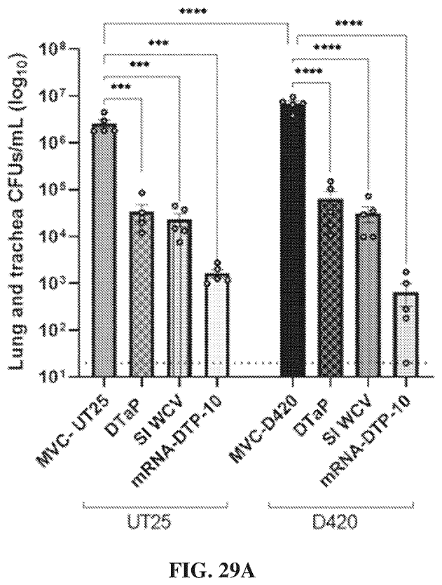

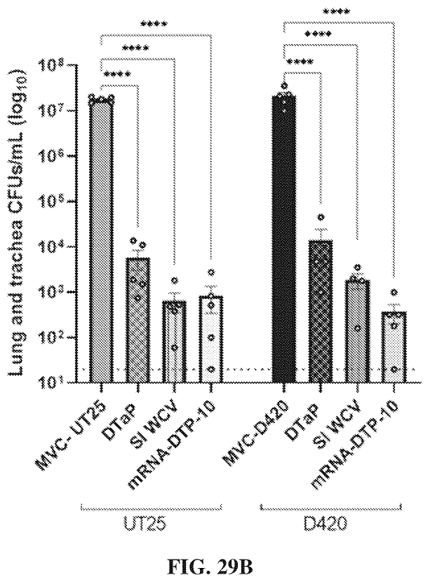

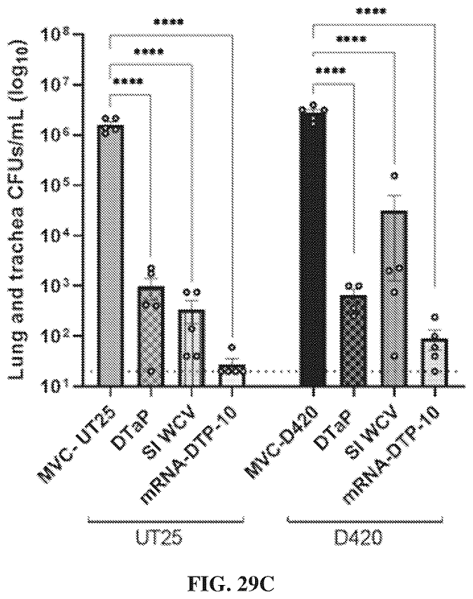

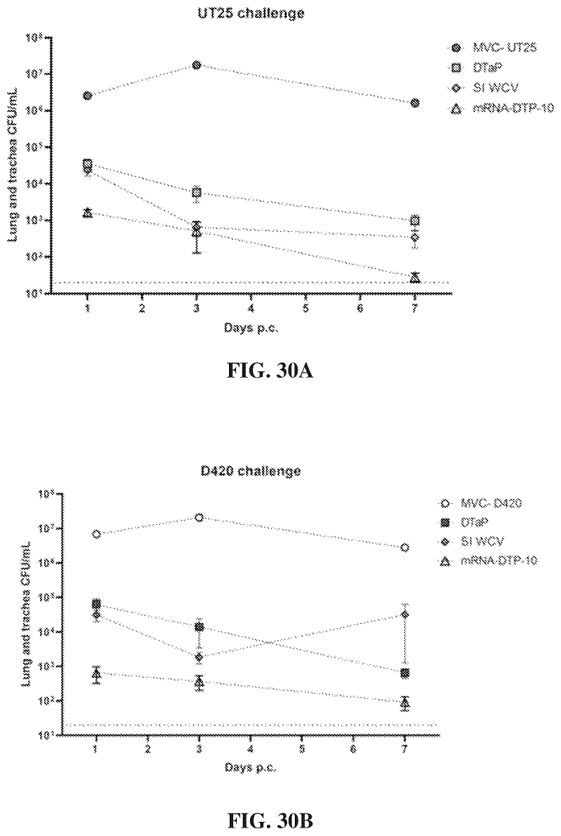

Next, the CFUs in mouse lung and tracheal samples (representing the lower respiratory tract) at 1, 3, and 7 days post-challenge were measured. Similar relative results were noted for each of the strains (UT25 and D420) tested: the mRNA vaccine formulations yielded significantly lower CFU counts at each time point and with each strain tested (FIGS. 29A-29C). Time courses of the results are presented in FIG. 30A (UT25) and FIG. 30B (D420).

### Additional Sequences

It should be understood that any of the mRNA sequences described herein may include a 5′ UTR and/or a 3′ UTR. The UTR sequences may be selected from the following sequences, or other known UTR sequences may be used. It should also be understood that any of the mRNA constructs described herein may further comprise a poly(A) tail and/or cap (e.g., 7mG(5′)ppp(5′)NlmpNp). Further, while many of the mRNAs and encoded antigen sequences described herein include a signal peptide and/or a peptide tag (e.g., C-terminal His tag), it should be understood that the indicated signal peptide and/or peptide tag may be substituted for a different signal peptide and/or peptide tag, or the signal peptide and/or peptide tag may be omitted.

5′ UTR:
(SEQ ID NO: 99)
GGGAAAUAAGAGAGAAAAGAAGAGUAAGAAGAAAUAUAAGAGCCACC
5′ UTR:
(SEQ ID NO: 2)
GGGAAAUAAGAGAGAAAAGAAGAGUAAGAAGAAAUAUAAGACCCCGGCGC
CGCCACC
3′ UTR:
(SEQ ID NO: 100)
UGAUAAUAGGCUGGAGCCUCGGUGGCCAUGCUUCUUGCCCCUUGGGCCUC
CCCCCAGCCCCUCCUCCCCUUCCUGCACCCGUACCCCCGUGGUCUUUGAA
UAAAGUCUGAGUGGGCGGC
3′ UTR:
(SEQ ID NO: 4)
UGAUAAUAGGCUGGAGCCUCGGUGGCCUAGCUUCUUGCCCCUUGGGCCUC
CCCCCAGCCCCUCCUCCCCUUCCUGCACCCGUACCCCCGUGGUCUUUGAA
UAAAGUCUGAGUGGGCGGC

TABLE 1
Sequence Listing

| | |
|---|---|
| Chemistry | 1-methylpseudouridine |
| Cap | C1 |

C180

| SEQ ID NO: 1 consists of from 5′ end to 3′ end: | | 1 |
|---|---|---|
| 5′ UTR SEQ ID NO: 2, mRNA ORF SEQ ID NO: 3, and | | |
| 3′ UTR SEQ ID NO: 4. | | |
| 5′ UTR | GGGAAAUAAGAGAGAAAAGAAGAGUAAGAAGAAAUAUAA | 2 |
| | GACCCCGGCGCCGCCACC | |
| ORF of mRNA | AUGGAAACCCCUGCUCAGCUGCUGUUCCUGCUGCUGCUGU | 3 |
| Construct | GGCUGCCUGAUACCACAGGGGAUGAUCCUCCAGCCACCGU | |
| (excluding the stop | GUAUAAGUACGACAGCAGACCUCCUGAGGACGUGUUCCA | |
| codon) | GAAUGGCUUUACCGCCUGGGGCAACAACGACAACGUGCU | |
| | GGAUCACCUGACCGGCAGAUCCUGUCAAGUGGGCAGCAG | |
| | CAAUAGCGCCUUCGUGUCCACCUCUAGCAGCAGACGGUAC | |
| | ACAGAGGUGUACCUGGAACACCGGAUGCAAGAGGCCGUG | |
| | GAAGCCGAAAGAGCCGGCAGAGGAACAGGCCACUUCAUC | |
| | GGCUACAUCUACGAAGUGCGGGCCGACAACAACUUCUAC | |
| | GGCGCUGCCAGCAGCUACUUCGAGUACGUGGACACCUACG | |
| | GCGACAACGCCGGAAGAAUACUGGCUGGCGCUCUGGCCAC | |
| | AUACCAGUCUGGAUAUCUGGCCCACAGACGGAUCCCUCCA | |
| | GAGAACAUUCGGAGAGUGACCCGGGUGUACCACAACGGC | |
| | AUUACCGGCGAGACAACCACCACCGAGUACAGCAACGCCA | |
| | GAUACGUGUCCCAGCAGACCCGGGCCAAUCCUAAUCCUUA | |
| | CACCAGC | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding amino | METPAQLLFLLLLWLPDTTGDDPPATVYKYDSRPPEDVFQNG | 5 |
| acid sequence | FTAWGNNDNVLDHLTGRSCQVGSSNSAFVSTSSSRRYTEVYL | |
| | EHRMQEAVEAERAGRGTGHFIGYIYEVRADNNFYGAASSYFE | |
| | YVDTYGDNAGRILAGALATYQSGYLAHRRIPPENIRRVTRVY | |
| | HNGITGETTTTEYSNARYVSQQTRANPNPYTS | |
| PolyA tail | 100 nt | |

Pertussis_S1

| SEQ ID NO: 6 consists of from 5′ end to 3′ end: | | 6 |
|---|---|---|
| 5′ UTR SEQ ID NO: 2, mRNA ORF SEQ ID NO: 7, and | | |
| 3′ UTR SEQ ID NO: 4. | | |
| 5′ UTR | GGGAAAUAAGAGAGAAAAGAAGAGUAAGAAGAAAUAUAA | 2 |
| | GACCCCGGCGCCGCCACC | |
| ORF of mRNA | AUGACUGUCAUGUUGCAGAGGGAAAUGCCGGUGAGGGCA | 7 |
| Construct | UAGGUCUGGAAUGUGGCGUCCUCGUAGUCGUAAUACUGC | |
| (excluding the stop | UCUUCCUUGCUGACGUGGACGCGUACGGCAAGGCCGGAC | |
| codon) | AUAUAGAUCAUGUACAGCAGGCGCCGCAGCGCGUCGUAC | |
| | AUGUCUCUGUACCUGCCUUCAUACGGGCUGGCGCAGGCUC | |
| | CGAUGACCGAUUGCCCGUCCCUGACGAAUACCGCGCACAG | |
| | CCUGCUGUUGGUGCUGGCGAGCAGGCGCGUGGCCGUGAC | |
| | CUUGCUGUAGUAGUGAUCCGCAGCCGGUUGCCCGGUCUU | |
| | GUAUAUGGUCGUGAUGCAGAAAGUUUCGCGAUAAAUGAA | |
| | CCCCGCGCCUGGCGGCGCGUCCUUGAUGAUGCCGCCGUAC | |
| | GCCUGGCCCAGGUACGUACCGUCAUAGAGACCGUAUAUG | |
| | GACCAGCCGGGCGCUAUCUGGCGCAAAUACGUCUGCAAU | |
| | UCGGCGUUGCCGCGCAGUUCGGCCACGGUCAAGGCGCGGG | |
| | UUCCGUUCGGGCAGCGUCCAUAGGCGCCGCCCUGUUGGGU | |
| | GAACAGUGCCUUCGGCGGGAUGACGAUGCCUGGCGCAAC | |
| | GGCCUGGGCCGUGCGCAUGCCCAGCAGGGCGAGCACCAGG | |
| | AUGGGCAGAAUGUGAUGAAGCAGCUUCUUGUUGUUGAUC | |
| | AGCAUGUUGCGGUGUUCCCGGAAUGGAACCAGGGCGGAA | |
| | GAUCGUCUCAAGCGGAUUGCGCGGAAACGUCGAAACCUC | |
| | CGGAAGGGUUCAUUCGCAAUAUCCGUUGAGCGGGCAGAU | |
| | CUGCAGUUCGAGCAGAUCGCCGGGAGUGCCCGGAUACGG | |
| | CGAGUCUUCCACCGUCAGCGCGAUCCGGCUCUUGAGCGCA | |
| | UACGCGGAUAUGGCAAAGCCAAGCAUGGUGUCGAACCAC | |
| | GUGUCGUGCUCGUGUCCCGCGUCGGACAGGCACGCCCGGA | |
| | CCAGGCUUCUGCCCGACAUGAAGGCGGUCAGGCAGAACUC | |
| | CUGAUUCUUGCCCUUCAGUUUCAAGGCCAGCUCCUGGACA | |
| | GUGAAGUUCUUGUACAGAUGGGUCGGCAAGCCGGCGACG | |
| | UCAGCCGGGCUGUAUAUGCCGAGCACGGACAACAGGAUG | |
| | GAUGCGAUGGUAUGCAUGGGGUUCGCCUUCAGGGGCAAU | |
| | CCUGCUUGCCGCUGCAUUCGACCAUCCGGAUCAGUUCGAG | |
| | CGCGGGCUUGCCUUCGAAAGUGAGUUGCUUGGGACCCAG | |
| | AAACAUGCGCAGCGGCCGUUGUUGCAUGAAGACGGCGCG | |
| | CAACAUGACUUCCAUGGGACUGCUGCCGGGACGCUUGAG | |
| | AUCCUUGCCGAAGCAGAACGGCACGUGCGCGUCCGGGCUG | |
| | CUGGCCGCGGCGCCCAGUUUGGCGGCGAUGCCGCAGACCA | |
| | GCAUGCGCGUCGGGGUGACUUCAUACGGCUUCACGGCUA | |
| | CGCUGGUGACCACCAUAUUGGUCUUCACCAGCACAUAAG | |
| | GAACGUCGGCCAGGGCGGGGGAAAGAUGCGUCAUCGCGC | |
| | CGGAUGCCAGCAACCACGCCAGGGCGCGCACGCGCGACCG | |
| | CCGGGCGCCGCCCUGUCCCGGGGCGUUGGUUCGAGUGGGG | |
| | AAGCGUCUCAGCAUAAGGAUGAUCCAGGAUUGCAGAUGG | |
| | AGAUGUCGGUAAGGGCGUAAGUCUCGAACGUUGCGUCCU | |
| | CAUAGUCGUAAUACUGUUCUUCCUUGCUGACAUGGACGC | |
| | GUACGGAGAUGCCGGCCACGUAGAUCAGGUAAAGCAUUC | |
| | UCCGCAGCCGGCUGUACAUGCUCCAGUACUUGCCGUCAUA | |
| | CGGGCUGGUGCAGGCGCCAAUGACCGGUUGCCCGCUUCUG | |
| | ACGAAGACCGCGCAUAGCCUGCUGUUGGUGCUGGAGAGC | |
| | AGGCGAGUGGCGGUGACGUUGCUGUAGUAGUGAUCCGUU | |
| | GCGGGUUGACCCGUAUUGCGCGUGGUCAUGAUGCAGAAC | |
| | GUCGUUUUCAGGUCGAAUGCGCCGCCGGGUGUUCCGUCC | |
| | UUGAUCACGCCGCCAUAUUCGCCGCCGAGAUAGGUGCCAU | |
| | CGUAGAGCGCAAAUAUUGACCAGCCGCGCGUCACAUGAC | |
| | GCAGGUACUCCUGCAGAUCGCCGCUGCCGCGCAAUUCCGC | |
| | CACGGUCAGGGCACGGGUCUUGUUCGCGCAGCGUCCAUA | |
| | GGGGCCGCCAUGCUGGGUAAUCUGUUCCUGCGGCGGAAU | |
| | GACGAUGCCUGGCGUGGAGGCCCGCGCCACGUGAGAUCCG | |
| | AGGAGGGCCAACGGCAGAACGGACAGGAGAUGGCAGAGC | |
| | GUCUUGCGGUCGAUCGGCAUGCUGUUCAAUUACCGGAGU | |
| | UGGGCGGGGCUGGGCCUGGUCUAGAACGAAUACGCGAUG | |
| | CUUUCGUAGUACACGAGAACCAUCGCCUCGCCGGCGCGUU | |
| | CGGACCAGGCUGCCAUGGCCUCGGAGCUUUCGGCCUGCCG | |
| | CGCCAUGCAAGCGCCUAUCACCGGCGCCAUGCGCACCAAU | |
| | GUGCCGACGAUCGACGCUACGGACCUUCGCGAUGUGUAG | |
| | GGGUUGGGAUUGGCGCGAGUCUGCUGGCUGACGUAGCGA | |
| | GCGUUGGAAUACUCCGUGGUCGUGGUCUCGCCGGUGAUG | |
| | CCGUUGUGAUAGACCCGCGUUACCCUGCGGAUGUUUUCG | |
| | GGCGGAAUGCGCCGGUGUGCCAGAUAUUCGCUCUGGUAG | |
| | GUGGCCAGCGCGCCGGCGAGGAUACGGCCGGCAUUGUCGC | |
| | CAUAAGUGUCGACGUAUUCGAAGUACGAGCUGGCGGCGC | |
| | CGUAGAAAUUGCUGUCGGCGCGGACUUCGUAGAUGUAGC | |
| | CGAUGAAGUGGCCGGUGCCCCUGCCGGCGCGUUCGGCCUC | |
| | GACCGCUUCCUGCAUGCGAUGUUCGAGAUAGACCUCGGU | |
| | AUAGCGCCGGCUGCUGCUGGUGGAGACGAAAGCGCUGUU | |
| | GCUGCUGCCGACCUGGCAGGAACGUCCGGUCAGAUGGUC | |
| | GAGCACAUUGUCGUUGUUUCCCCACGCCGUGAAUCCGUUC | |
| | UGGAAAACGUCCUCCGGCGGGGGGAGUCAUAGCGGUAU | |
| | ACGGUGGCGGGAGGAUCGUCGGCCCAUGCCGGCGAAGUC | |
| | ACGGGCGCCGUGACGGCAAGAAUCGCCAGCCACGUCAGCC | |
| | AGCCUGUUCUUGCGGUUUGGCGAAUUGCCCGAGUGCAAC | |
| | GCAUCCCGU | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding amino | MRCTRAIRQTARTGWLTWLAILAVTAPVTSPAWADDPPATVY | 8 |
| acid sequence | RYDSRPPEDVFQNGFTAWGNNDNVLDHLTGRSCQVGSSNSAF | |
| | VSTSSSRRYTEVYLEHRMQEAVEAERAGRGTGHFIGYIYEVRA | |
| | DNNFYGAASSYFEYVDTYGDNAGRILAGALATYQSEYLAHRR | |
| | IPPENIRRVTRVYHNGITGETTTTEYSNARYVSQQTRANPNPYT | |
| | SRRSVASIVGTLVRMAPVIGACMARQAESSEAMAAWSERAGE | |
| | AMVLVYYESIAYSF | |
| PolyA tail | 100 nt | |

Pertussis_S1_9K_129G_cons

| SEQ ID NO: 9 consists of from 5′ end to 3′ end: | | 9 |
|---|---|---|
| 5′ UTR SEQ ID NO: 2, mRNA ORF SEQ ID NO: 10, and | | |
| 3′ UTR SEQ ID NO: 4. | | |
| 5′ UTR | GGGAAAUAAGAGAGAAAAGAAGAGUAAGAAGAAAUAUAA | 2 |
| | GACCCCGGCGCCGCCACC | |
| ORF of mRNA | AUGGAAACCCCUGCUCAGCUGCUGUUCCUGCUGCUGCUGU | 10 |
| Construct | GGCUGCCUGAUACCACAGGGGAUGAUCCUCCAGCCACCGU | |
| (excluding the stop | GUAUAAGUACGACAGCAGACCUCCUGAGGACGUGUUCCA | |
| codon) | GAAUGGCUUUACCGCCUGGGGCAACAACGACAACGUGCU | |
| | GGAUCACCUGACCGGCAGAUCCUGUCAAGUGGGCAGCAG | |
| | CAAUAGCGCCUUCGUGUCCACCUCUAGCAGCAGACGGUAC | |
| | ACAGAGGUGUACCUGGAACACCGGAUGCAAGAGGCCGUG | |
| | GAAGCCGAAAGAGCCGGCAGAGGAACAGGCCACUUCAUC | |
| | GGCUACAUCUACGAAGUGCGGGCCGACAACAACUUCUAC | |
| | GGCGCUGCCAGCAGCUACUUCGAGUACGUGGACACCUACG | |
| | GCGACAACGCCGGAAGAAUACUGGCUGGCGCUCUGGCCAC | |
| | AUACCAGUCUGGAUAUCUGGCCCACAGACGGAUCCCUCCA | |
| | GAGAACAUUCGGAGAGUGACCCGGGUGUACCACAACGGC | |
| | AUUACCGGCGAGACAACCACCACCGAGUACAGCAACGCCA | |
| | GAUACGUGUCCCAGCAGACCCGGGCCAAUCCUAAUCCUUA | |
| | CACCAGCAGAAGAAGCGUGGCCAGCAUCGUGGGCACCCUG | |
| | GUGAGAAUGGCCCCUGUGAUCGGCGCCUGCAUGGCCAGA | |
| | CAGGCCGAGAGCAGCGAGGCCAUGGCCGCCUGGAGCGAG | |
| | AGAGCCGGAGAGGCCAUGGUGCUGGUGUACUACGAGAGC | |
| | AUCGCCUACAGCUUC | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding amino | METPAQLLFLLLLWLPDTTGDDPPATVYKYDSRPPEDVFQNG | 11 |
| acid sequence | FTAWGNNDNVLDHLTGRSCQVGSSNSAFVSTSSSRRYTEVYL | |
| | EHRMQEAVEAERAGRGTGHFIGYIYEVRADNNFYGAASSYFE | |
| | YVDTYGDNAGRILAGALATYQSGYLAHRRIPPENIRRVTRVY | |
| | HNGITGETTTTEYSNARYVSQQTRANPNPYTSRRSVASIVGTL | |
| | VRMAPVIGACMARQAESSEAMAAWSERAGEAMVLVYYESIA | |
| | YSF | |
| PolyA tail | 100 nt | |

Pertussis_NGM_S2

| SEQ ID NO: 12 consists of from 5′ end to 3′ end: | | 12 |
|---|---|---|
| 5′ UTR SEQ ID NO: 2, mRNA ORF SEQ ID NO: 13, and | | |
| 3′ UTR SEQ ID NO: 4. | | |
| 5′ UTR | GGGAAAUAAGAGAGAAAAGAAGAGUAAGAAGAAAUAUAA | 2 |
| | GACCCCGGCGCCGCCACC | |
| ORF of mRNA | AUGGAGACCCCUGCCCAGCUGCUGUUCCUGCUGCUGCUGU | 13 |
| Construct | GGCUGCCUGACACCACCGGCGGCAUCGUGAUCCCUCCUCA | |
| (excluding the stop | GGAGCAGAUCACCCAGCACGGCGGCCCUUACGGCAGAUGC | |
| codon) | GCCAACAAGGCCAGAGCCCUGACCGUGGCCGAGCUGAGAG | |
| | GCAGCGGCGACCUGCAGGAGUACCUGAGACACGUGACCA | |
| | GAGGCUGGAGCAUCUUCGCCCUGUACGACGGCACCUACCU | |
| | GGGCGGCGAGUACGGCGGCGUGAUCAAGGACGGCACCCC | |
| | UGGCGGCGCCUUCGACCUGAAGACCACCUUCUGCAUCAUG | |
| | ACCACCAGAAACACCGGCCAGCCUGCCACCGACCACUACU | |
| | ACAGCAAGGUGACCGCCACCAGACUGCUGAGCAGCACCAA | |
| | CAGCAGACUGUGCGCCGUGUUCGUGAGAAGCGGCCAGCC | |
| | UGUGAUCGGCGCCUGCACCAGCCCUUACGACGGCAAGUAC | |
| | UGGAGCAUGUACAGCAGACUGAGAAAGAUGCUGUACCUG | |
| | AUCUACGUGGCCGGCAUCAGCGUGAGAGUGCACGUGAGC | |
| | AAGGAGGAGCAGUACUACGACUACGAGGACGCCACCUUC | |
| | GAGACCUACGCCCUGACCGGCAUCAGCAUCUGCAACCCUG | |
| | GCAGCAGCCUGUGC | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding amino | METPAQLLFLLLLWLPDTTGGIVIPPQEQITQHGGPYGRCANK | 14 |
| acid sequence | ARALTVAELRGSGDLQEYLRHVTRGWSIFALYDGTYLGGEYG | |
| | GVIKDGTPGGAFDLKTTFCIMTTRNTGQPATDHYYSKVTATR | |
| | LLSSTNSRLCAVFVRSGQPVIGACTSPYDGKYWSMYSRLRKM | |
| | LYLIYVAGISVRVHVSKEEQYYDYEDATFETYALTGISICNPGS | |
| | SLC | |
| PolyA tail | 100 nt | |

Pertussis_NGM_S3

| SEQ ID NO: 15 consists of from 5′ end to 3′ end: | | 15 |
|---|---|---|
| 5′ UTR SEQ ID NO: 2, mRNA ORF SEQ ID NO: 16, and | | |
| 3′ UTR SEQ ID NO: 4. | | |
| 5′ UTR | GGGAAAUAAGAGAGAAAAGAAGAGUAAGAAGAAAUAUAA | 2 |
| | GACCCCGGCGCCGCCACC | |
| ORF of mRNA | AUGGAGACCCCUGCCCAGCUGCUGUUCCUGCUGCUGCUGU | 16 |
| Construct | GGCUGCCUGACACCACCGGCGGCAUCGUGAUCCCUCCUAA | |
| (excluding the stop | GGCCCUGUUCACCCAGCAGGGCGGCGCCUACGGCAGAUGC | |
| codon) | CCUAACGGCACCAGAGCCCUGACCGUGGCCGAGCUGAGAG | |
| | GCAACGCCGAGCUGCAGACCUACCUGAGACAGAUCACCCC | |
| | UGGCUGGAGCAUCUACGGCCUGUACGACGGCACCUACCUG | |
| | GGCCAGGCCUACGGCGGCAUCAUCAAGGACGCCCCUCCUG | |
| | GCGCCGGCUUCAUCUACAGAGAGACCUUCUGCAUCACCAC | |
| | CAUCUACAAGACCGGCCAGCCUGCCGCCGACCACUACUAC | |
| | AGCAAGGUGACCGCCACCAGACUGCUGGCCAGCACCAACA | |
| | GCAGACUGUGCGCCGUGUUCGUGAGAGACGGCCAGAGCG | |
| | UGAUCGGCGCCUGCGCCAGCCCUUACGAGGGCAGAUACAG | |
| | AGACAUGUACGACGCCCUGAGAAGACUGCUGUACAUGAU | |
| | CUACAUGAGCGGCCUGGCCGUGAGAGUGCACGUGAGCAA | |
| | GGAGGAGCAGUACUACGACUACGAGGACGCCACCUUCCA | |
| | GACCUACGCCCUGACCGGCAUCAGCCUGUGCAACCCUGCC | |
| | GCCAGCAUCUGC | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding amino | METPAQLLFLLLLWLPDTTGGIVIPPKALFTQQGGAYGRCPNG | 17 |
| acid sequence | TRALTVAELRGNAELQTYLRQITPGWSIYGLYDGTYLGQAYG | |
| | GIIKDAPPGAGFIYRETFCITTIYKTGQPAADHYYSKVTATRLL | |
| | ASTNSRLCAVFVRDGQSVIGACASPYEGRYRDMYDALRRLLY | |
| | MIYMSGLAVRVHVSKEEQYYDYEDATFQTYALTGISLCNPAA | |
| | SIC | |
| PolyA tail | 100 nt | |

Pertussis_S4

| SEQ ID NO: 18 consists of from 5′ end to 3′ end: | | 18 |
|---|---|---|
| 5′ UTR SEQ ID NO: 2, mRNA ORF SEQ ID NO: 19, and | | |
| 3′ UTR SEQ ID NO: 4. | | |
| 5′ UTR | GGGAAAUAAGAGAGAAAAGAAGAGUAAGAAGAAAUAUAA | 2 |
| | GACCCCGGCGCCGCCACC | |
| ORF of mRNA | AUGGAGACCCCUGCCCAGCUGCUGUUCCUGCUGCUGCUGU | 19 |
| Construct | GGCUGCCUGACACCACCGGCGACGUGCCUUACGUGCUGGU | |
| (excluding the stop | GAAGACCAACAUGGUGGUGACCAGCGUGGCCAUGAAGCC | |
| codon) | UUACGAGGUGACCCCUACCAGAAUGCUGGUGUGCGGCAU | |
| | CGCCGCCAAGCUGGGCGCCGCCGCCAGCAGCCCUGACGCC | |
| | CACGUGCCUUUCUGCUUCGGCAAGGACCUGAAGAGACCU | |
| | GGCAGCAGCCCUAUGGAGGUGAUGCUGAGAGCCGUGUUC | |
| | AUGCAGCAGAGACCUCUGAGAAUGUUCCUGGGCCCUAAG | |
| | CAGCUGACCUUCGAGGGCAAGCCUGCCCUGGAGCUGAUCA | |
| | GAAUGGUGGAGUGCAGCGGCAAGCAGGACUGCCCU | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding amino | METPAQLLFLLLLWLPDTTGDVPYVLVKTNMVVTSVAMKPY | 20 |
| acid sequence | EVTPTRMLVCGIAAKLGAAASSPDAHVPFCFGKDLKRPGSSP | |
| | MEVMLRAVFMQQRPLRMFLGPKQLTFEGKPALELIRMVECSG | |
| | KQDCP | |
| PolyA tail | 100 nt | |

Pertussis_NGM_S5

| SEQ ID NO: 21 consists of from 5′ end to 3′ end: | | 21 |
|---|---|---|
| 5′ UTR SEQ ID NO: 2, mRNA ORF SEQ ID NO: 22, and | | |
| 3′ UTR SEQ ID NO: 4. | | |
| 5′ UTR | GGGAAAUAAGAGAGAAAAGAAGAGUAAGAAGAAAUAUAA | 2 |
| | GACCCCGGCGCCGCCACC | |
| ORF of mRNA | AUGGAGACCCCUGCCCAGCUGCUGUUCCUGCUGCUGCUGU | 22 |
| Construct | GGCUGCCUGACACCACCGGCCUGCCUACCCACCUGUACAA | |
| (excluding the stop | GAACUUCGCCGUGCAGGAGCUGGCCCUGAAGCUGAAGGG | |
| codon) | CAAGAACCAGGAGUUCUGCCUGACCGCCUUCAUGAGCGGC | |
| | AGAAGCCUGGUGAGAGCCUGCCUGAGCGACGCCGGCCACG | |
| | AGCACGACACCUGGUUCGACACCAUGCUGGGCUUCGCCAU | |
| | CAGCGCCUACGCCCUGAAGAGCAGAAUCGCCCUGACCGUG | |
| | GAGGACAGCCCUUACCCUGGCACCCCUGGCGACCUGCUGG | |
| | AGCUGCAGAUCUGCCCUCUGAACGGCUACUGCGAG | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding amino | METPAQLLFLLLLWLPDTTGLPTHLYKNFAVQELALKLKGKN | 23 |
| acid sequence | QEFCLTAFMSGRSLVRACLSDAGHEHDTWFDTMLGFAISAYA | |
| | LKSRIALTVEDSPYPGTPGDLLELQICPLNGYCE | |
| PolyA tail | 100 nt | |

SPHB1

| SEQ ID NO: 24 consists of from 5′ end to 3′ end: | | 24 |
|---|---|---|
| 5′ UTR SEQ ID NO: 2, mRNA ORF SEQ ID NO: 25, and | | |
| 3′ UTR SEQ ID NO: 4. | | |
| 5′ UTR | GGGAAAUAAGAGAGAAAAGAAGAGUAAGAAGAAAUAUAA | 2 |
| | GACCCCGGCGCCGCCACC | |
| ORF of mRNA | AUGGAAACCCCUGCACAGCUGCUGUUCCUGCUGCUGCUGU | 25 |
| Construct | GGCUGCCCGACACGACCGGCGGCUCUCCUGGCGGACGAGC | |
| (excluding the stop | UCCUAGCGCACCCCAACCCGCUCCUUCUCCUAGACCUGAG | |
| codon) | CCUGCCCCAGAACCAGCCCCUAAUCCGGCUCCACGGCCUG | |
| | CCCCUCAGCCUCCUGCACCUGCUCCUGGAGCACCAAGACC | |
| | UCCUGCUCCUCCACCUGAGGCUCCUCCUCCCGUUAUGCCU | |
| | CCUCCUGCCGUUCCUCCUCAAUUGCCGGAGGUGCCUGCUG | |
| | CAGAUCUUCCUCGGGUCCGCGCUCCCCUGAGCACUUAUAG | |
| | ACGCCCUCAGCGCACCGAUUUCGUUACCCCUACCGGCGGC | |
| | CCGUUCUUCGCCAAACAGGACAAAGCGCUCAACACCAUUG | |
| | AUCUGAAGAUGGCCCACGAUCUGAAGCUGAGAGGAUACA | |
| | GAGUAAAGGUUGCAGUAGUGGACGAGGGAGUAAGAUCCG | |
| | ACCACCCACUGCUGAACGUGGAGAAGAAGUACGGCGGCG | |
| | ACUACAUGGCCGACGGCACCCGCACCUACCCUGACCCUAA | |
| | GCGACAGGGCAGACACGGGACGUCGGUGGCCCUGGUGCU | |
| | UGCCGGCCAGGACACCGACACCUAUAGAGGCGGAGUGGCC | |
| | CCUAACGCGGACUUGUAUAGCGCCAACAUUGGCACUCGU | |
| | GCCGGCCACGUAAGCGACGAAGCCGCAUUUCACGCCUGGA | |
| | ACGACCUACUCGGCCACGGCAUCAAGAUUUUCAAUAACA | |
| | GCUUCGCCACUGAAGGCCCCGAGGGAGAGCAGAGAGUUA | |
| | AGGAGGACAGAAACGAGUACCAUUCCGCCGCCAAUAAGC | |
| | AGAAUACCUACAUAGGCAGACUGGAUCGCCUCGUUCGGG | |
| | ACGGAGCUCUCCUUAUCUUUGCUGCAGGCAACGGGCGACC | |
| | UAGCGGACGGGCCUACUCAGAGGUGGGCAGCGUAGGAAG | |
| | GACACCUCGCGUCGAGCCACAUCUUCAGCGCGGCCUGAUC | |
| | GUGGUCACUGCAGUCGACGAGAACGGUCGGCUGGAAACC | |
| | UGGGCCAACCGGUGCGGCCAGGCCCAGCAGUGGUGCCUUG | |
| | CUGCUCCUUCAACAGCAUAUCUGCCAGGGCUGGAUAAGG | |
| | ACAACCCUGACAGCAUCCACGUUGAACAGGGUACCGCCCU | |
| | GAGCGCACCGCUCGUGACCGGCGCCGCAGUGCUGGUACAG | |
| | GACAGAUUUCGCUGGAUGGACAACGACAACCUGCGCACC | |
| | ACCCUUCUAACCACCGCUCAGGAUAAGGGGCCUUACGGGG | |
| | UCGAUCCUCAGUACGGCUGGGGCGUGCUGGACGUGGGCC | |
| | GCGCCGUGCAAGGCCCUGCCCAGUUUGCUUUUGGAGACU | |
| | UCGUGGCCAGAGUCACCGAUACUUCCACCUUCGGGAACGA | |
| | UAUUAGCGGCGCUGGCGGCCUUGUGGUAGACGGCCCUGG | |
| | CGCCUUAGUCCUGGCAGGGAGUAAUACAUACGCCGGCAG | |
| | GACCACUAUCAAGAGAGGCACCCUGGACGUGUUCGGAAG | |
| | CGUGACCUCCGCAGUCACCGUGGAACCUGGAGGCACUCUC | |
| | ACCGGCAUAGGGACUGUAGGUACCGUGACCAAUCAGGGC | |
| | ACGGUGGUUAAUAAGGAAGCCGGCCUCCACGUGAAAGGG | |
| | GAUUAUUCCCAGACCGCACAGGGCCUACUGGUGACAGAU | |
| | AUUGGCUCUCUGCUGGACGUCAGCGGCAGAGCUAGCCUC | |
| | GCCGGUAGACUGCACGUUGACGAUAUCAGACCUGGGUAC | |
| | GUAGGCGGCGACGGAAAGUCCGUCCCUGUGAUUAAGGCC | |
| | GGUGCGGUCAGCGGGGUGUUCGCCACCCUGACACGCUCAC | |
| | CAGGCCUGCUCUUGAACGCGAGGCUGGACUACAGACCUCA | |
| | GGCUGUAUAUCUGACUAUGAGACGCGCUGAAAGAGUCCA | |
| | CGCCGCCGCCCAGAGAGGAGCCGACGACGGCAGACGGGCU | |
| | UCCGUGCUUGCCGUCGCCGAGAGACUCGACGCAGCCAUGC | |
| | GAGAGCUGGACGCUUUGCCGGAAAGUCAGAGGGACGCCG | |
| | CAGCCCCUGCCGCAGCGAUUGGACGGAUUCAGCGGGUGCA | |
| | GAGCAGAAAGGUUUUGCAGGACAACCUGUACUCUUUAGC | |
| | UGGCGCAACCUACGCCAACGCUGCUGCCGUGAACACCCUA | |
| | GAACAGAACAGGUGGAUGGAUAGACUGGAGAAC | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding amino | METPAQLLFLLLLWLPDTTGGSPGGRAPSAPQPAPSPRPEPAPE | 26 |
| acid sequence | PAPNPAPRPAPQPPAPAPGAPRPPAPPPEAPPPVMPPPAVPPQL | |
| | PEVPAADLPRVRAPLSTYRRPQRTDFVTPTGGPFFAKQDKALN | |
| | TIDLKMAHDLKLRGYRVKVAVVDEGVRSDHPLLNVEKKYGG | |
| | DYMADGTRTYPDPKRQGRHGTSVALVLAGQDTDTYRGGVAP | |
| | NADLYSANIGTRAGHVSDEAAFHAWNDLLGHGIKIFNNSFAT | |
| | EGPEGEQRVKEDRNEYHSAANKQNTYIGRLDRLVRDGALLIF | |
| | AAGNGRPSGRAYSEVGSVGRTPRVEPHLQRGLIVVTAVDENG | |
| | RLETWANRCGQAQQWCLAAPSTAYLPGLDKDNPDSIHVEQG | |
| | TALSAPLVTGAAVLVQDRFRWMDNDNLRTTLLTTAQDKGPY | |
| | GVDPQYGWGVLDVGRAVQGPAQFAFGDFVARVTDTSTFGND | |
| | ISGAGGLVVDGPGALVLAGSNTYAGRTTIKRGTLDVFGSVTSA | |
| | VTVEPGGTLTGIGTVGTVTNQGTVVNKEAGLHVKGDYSQTA | |
| | QGLLVTDIGSLLDVSGRASLAGRLHVDDIRPGYVGGDGKSVP | |
| | VIKAGAVSGVFATLTRSPGLLLNARLDYRPQAVYLTMRRAER | |
| | VHAAAQRGADDGRRASVLAVAERLDAAMRELDALPESQRDA | |
| | AAPAAAIGRIQRVQSRKVLQDNLYSLAGATYANAAAVNTLEQ | |
| | NRWMDRLEN | |
| PolyA tail | 100 nt | |

TCFA

| SEQ ID NO: 27 consists of from 5′ end to 3′ end: | | 27 |
|---|---|---|
| 5′ UTR SEQ ID NO: 2, mRNA ORF SEQ ID NO: 28, and | | |
| 3′ UTR SEQ ID NO: 4. | | |
| 5′ UTR | GGGAAAUAAGAGAGAAAAGAAGAGUAAGAAGAAAUAUAA | 2 |
| | GACCCCGGCGCCGCCACC | |
| ORF of mRNA | AUGGAAACUCCUGCCCAGCUGCUGUUCCUGCUCCUUCUGU | 28 |
| Construct | GGCUGCCUGACACCACCGGCCUGAAGCUGCCUAGCCUGCU | |
| (excluding the stop | GACCGACGACGAGCUUAAGCUGGUGCUGCCUACCGGCAU | |
| codon) | GAGCCUGGAGGACUUCAAGAGAAGCCUGCAGGAGAGCGC | |
| | CCCUAGCGCCCUGGCCACCCCUCCUAGCAGCAGCCCUCCU | |
| | GUGGCCAAGCCUGGCCCUGGCAGCGUGGCCGAGGCUCCGU | |
| | CAGGCAGCGGCCACAAGGACAACCCUUCACCUCCUGUUGU | |
| | GGGCGUGGGACCAGGCAUGGCCGAGAGCAGCGGCGGACA | |
| | UAACCCUGGCGUAGGAGGCGGCACCCACGAGAACGGCCUG | |
| | CCUGGCAUCGGCAAGGUCGGUGGAAGUGCCCCUGGCCCAG | |
| | AUACCAGCACUGGAAGCGGCCCUGACGCCGGCAUGGCUAG | |
| | CGGCGCCGGCAGCACCAGCCCUGGCGCCUCAGGUAUGCCU | |
| | CCUUCCGAGGGCGAGCGCCCUGAUAGUGGAAUGUCUGAC | |
| | UCAGGAAGAGGCGGAGAGAGCAGUGCCGGCGGCCUAAAC | |
| | CCUGACGGAGCUGGCAAGCCUCCUAGAGAGGAGGGAGAG | |
| | CCGGGCUCCAAGAGCCCUGCCGACGGCGGCCAGGACGGCC | |
| | CUCCUCCGCCUAGGGACGGUGGCGACGCCGACCCUCAGCC | |
| | UCCUCGAGACGACGGCAACGGCGAACAGCAGCCACCUAAG | |
| | GGUGGCGGCGACGAGGGCCAGCGCCCACCUCCGGCGGCCG | |
| | GAAACGGCGGUAACGGAGGCAACGGCAACGCGCAGUUGC | |
| | CAGAGAGAGGCGACGACGCCGGUCCUAAGCCACCAGAGG | |
| | GCGAAGGCGGCGACGAAGGACCACAGCCUCCACAAGGCGG | |
| | AGGUGAACAGGACGCCCCUGAGGUGCCUCCUGUAGCUCCG | |
| | GCUCCACCUGCGGGAAACGGUGUGUACGACCCUGGUACAC | |
| | ACACCCUGACCACUCCAGCCAGCGCCGCCGUAUCUCUUGC | |
| | UUCAUCCUCACACGGUGUCUGGCAGGCCGAGAUGAAC | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding amino | METPAQLLFLLLLWLPDTTGLKLPSLLTDDELKLVLPTGMSLE | 29 |
| acid sequence | DFKRSLQESAPSALATPPSSSPPVAKPGPGSVAEAPSGSGHKD | |
| | NPSPPVVGVGPGMAESSGGHNPGVGGGTHENGLPGIGKVGGS | |
| | APGPDTSTGSGPDAGMASGAGSTSPGASGMPPSEGERPDSGM | |
| | SDSGRGGESSAGGLNPDGAGKPPREEGEPGSKSPADGGQDGP | |
| | PPPRDGGDADPQPPRDDGNGEQQPPKGGGDEGQRPPPAAGNG | |
| | GNGGNGNAQLPERGDDAGPKPPEGEGGDEGPQPPQGGGEQD | |
| | APEVPPVAPAPPAGNGVYDPGTHTLTTPASAAVSLASSSHGV | |
| | WQAEMN | |
| PolyA tail | 100 nt | |

PRN

| SEQ ID NO: 30 consists of from 5′ end to 3′ end: | | 30 |
|---|---|---|
| 5′ UTR SEQ ID NO: 2, mRNA ORF SEQ ID NO: 31, and | | |
| 3′ UTR SEQ ID NO: 4. | | |
| 5′ UTR | GGGAAAUAAGAGAGAAAAGAAGAGUAAGAAGAAAUAUAA | 2 |
| | GACCCCGGCGCCGCCACC | |
| ORF of mRNA | AUGGAGACCCCUGCCCAGCUGCUGUUCCUGCUGCUGCUUU | 31 |
| Construct | GGCUGCCAGACACCACCGGAGACUGGAACAACCAGGCCAU | |
| (excluding the stop | CGUGAAGACCGGCGAGAGACAGCACGGCAUCCACAUCCAA | |
| codon) | GGCAGCGACCCCGGCGGCGUGAGAACCGCCUCCGGCACCA | |
| | CAAUCAAGGUGAGCGGCAGACAGGCCCAGGGCAUCCUGC | |
| | UGGAGAACCCUGCCGCCGAGCUGCAGUUCAGAAACGGCGC | |
| | CGUGACCAGCAGCGGCCAGCUGUCCGACGACGGCAUCAGA | |
| | AGAUUCCUGGGCACCGUGACCGUGAAGGCCGGCAAGCUG | |
| | GUGGCCGACCACGCCACCCUGGCCAACGUGGGUGAUACCU | |
| | GGGACGACGACGGUAUUGCCCUGUACGUGGCCGGCGAGC | |
| | AAGCACAGGCCAGCAUCGCCGACAGCACCCUGCAAGGCGC | |
| | AGGCGGCGUGCAGAUCGAGAGAGGCGCCAACGUGGUGGU | |
| | GCAGAGAAGCGCCAUUGUUGACGGCGGCCUGCACAUCGG | |
| | CGCCCUGCAGAGCCUGCAGCCUGAGGACCUGCCUCCUAGC | |
| | AGAGUGGUGCUGAGAGACACAAACGUCGUCGCCGUGCCA | |
| | GCGUCCGGAGCUCCAGCAGCCGUGAGCGUGCUGGGCGCCA | |
| | GCGAGCUGACCCUCGACGGAGGACACAUCACCGGUGGCAG | |
| | GGCCGCCGGUGUGGCCGCCAUGCAGGGAGCCGUUGUGCA | |
| | UCUACAAAGAGCUACCAUCCGACGGGGCGACGCUCCGGCU | |
| | GGUGGAGCGGUCCCUGGCGGUGCCGUCCCCGGAGGAGCA | |
| | GUGCCAGGCGGUUUCGGCCCGGGUGGAUUCGGCCCUGUG | |
| | CUAGACGGCUGGUACGGCGUGGACGUGAGUGGCUCCAGC | |
| | GUGGAGCUGGCCCAGAGUAUUGUUGAGGCCCCUGAGCUC | |
| | GGCGCUGCCAUCAGAGUGGGCCGUGGUGCCAGAGUGACC | |
| | GUUAGCGGUGGCUCACUUAGCGCCCCUCACGGCAACGUGA | |
| | UCGAAACUGGAGGAGCGAGACGAUUCGCACCUCAGGCCG | |
| | CCCCUCUGAGCAUAACGCUGCAAGCGGGCGCCCACGCGCA | |
| | AGGCAAGGCCCUGCUGUACAGAGUGCUGCCUGAGCCUGU | |
| | GAAGUUAACUCUGACGGGAGGUGCCGACGCACAAGGUGA | |
| | CAUCGUGGCCACGGAACUUCCUAGCAUCCCUGGCACAUCU | |
| | AUCGGCCCUCUGGACGUUGCGUUAGCUUCGCAGGCCCGUU | |
| | GGACCGGCGCCACCAGAGCCGUGGACUCCCUAAGCAUCGA | |
| | CAACGCCGUGUGGGUCAUGACCGACAACUCAAACGUGGG | |
| | CGCGCUGAGAUUGGCCUCAGACGGCAGUGUCGAUUUCCA | |
| | ACAACCUGCCGAAGCAGGAAGAUUCAAGGUGCUGACUGU | |
| | CAAUACACUGGCCGGAAGCGGCCUGUUCAGAAUGAACGU | |
| | GUUCGCCGACCUGGGACUCUCAGAUAAGCUGGUCGUGAU | |
| | GCAGGACGCAAGUGGCCAGCACAGACUGUGGGUCAGAAA | |
| | CUCGGGCUCCGAGCCAGCCAGUGCCAAUACUUUGCUGUUG | |
| | GUGCAAACCCCUCUGGGCAGUGCCGCCACCUUCACUCUCG | |
| | CCAACAAGGACGGCAAGGUGGACAUCGGCACCUACAGAU | |
| | ACAGGCUGGCAGCAAACGGAAACGGCCAGUGGAGCCUAG | |
| | UCGGAGCAAAGGCCCCUCCUGCCCCUAAGCCUGCUCCACA | |
| | GCCUGGCCCACAGCCUCCGCAACCUCCGCAGCCUCAGCCA | |
| | GAGGCCCCAGCCCCACAACCUCCUGCAGGCAGAGAGCUGA | |
| | GUGCCGCUGCUAACGCCGCCGUUAAUACCGGAGGUGUCG | |
| | GCCUCGCGUCUACCUUGUGGUACGCCGAGAGCAACCGGGC | |
| | CAAGAGAAGCGGAAGCGGCGCUCCC | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding amino | METPAQLLFLLLLWLPDTTGDWNNQAIVKTGERQHGIHIQGS | 32 |
| acid sequence | DPGGVRTASGTTIKVSGRQAQGILLENPAAELQFRNGAVTSSG | |
| | QLSDDGIRRFLGTVTVKAGKLVADHATLANVGDTWDDDGIA | |
| | LYVAGEQAQASIADSTLQGAGGVQIERGANVVVQRSAIVDGG | |
| | LHIGALQSLQPEDLPPSRVVLRDTNVVAVPASGAPAAVSVLGA | |
| | SELTLDGGHITGGRAAGVAAMQGAVVHLQRATIRRGDAPAG | |
| | GAVPGGAVPGGAVPGGFGPGGFGPVLDGWYGVDVSGSSVEL | |
| | AQSIVEAPELGAAIRVGRGARVTVSGGSLSAPHGNVIETGGAR | |
| | RFAPQAAPLSITLQAGAHAQGKALLYRVLPEPVKLTLTGGAD | |
| | AQGDIVATELPSIPGTSIGPLDVALASQARWTGATRAVDSLSID | |
| | NAVWVMTDNSNVGALRLASDGSVDFQQPAEAGRFKVLTVNT | |
| | LAGSGLFRMNVFADLGLSDKLVVMQDASGQHRLWVRNSGSE | |
| | PASANTLLLVQTPLGSAATFTLANKDGKVDIGTYRYRLAANG | |
| | NGQWSLVGAKAPPAPKPAPQPGPQPPQPPQPQPEAPAPQPPAG | |
| | RELSAAANAAVNTGGVGLASTLWYAESN | |
| PolyA tail | 100 nt | |

FHA3

| SEQ ID NO: 33 consists of from 5′ end to 3′ end: | | 33 |
|---|---|---|
| 5′ UTR SEQ ID NO: 2, mRNA ORF SEQ ID NO: 34, and | | |
| 3′ UTR SEQ ID NO: 4. | | |
| 5′ UTR | GGGAAAUAAGAGAGAAAAGAAGAGUAAGAAGAAAUAUAA | 2 |
| | GACCCCGGCGCCGCCACC | |
| ORF of mRNA | AUGGAGACCCCUGCCCAGCUGCUGUUCCUGCUGCUGCUUU | 34 |
| Construct | GGCUGCCUGACACCACCGGAAGCCUGUACGCCGAACACGA | |
| (excluding the stop | CGCCACCCUGACCCUGGCCCAAGGCACCCAGAGGGAUCUG | |
| codon) | GUGGUGGACCAGGACCACAUCCUGCCCGUGGCCGAGGGA | |
| | ACCCUGAGAGUCAAAGCCAAGAGCCUCACCACCGAGAUCG | |
| | AGACAGGCAACCCUGGCAGCCUCAUCGCCGAGGUGCAGGA | |
| | GAACAUCGACAACAAGCAGGCCAUCGUCGUGGGCAAAGA | |
| | CCUGACUCUGUCCAGCGCCCACGGCAACGUAGCCAACGAG | |
| | GCCAACGCCCUGCUGUGGGCUGCCGGUGAGCUGACCGUGA | |
| | AGGCCCAGAACAUCACCAACAAGAGAGCCGCCCUGAUUGA | |
| | GGCCGGCGGGAACGCCAGACUGACCGCUGCCGUCGCCCUU | |
| | CUGAACAAGCUGGGCAGAAUCAGAGCCGGCGAGGACAUG | |
| | CACCUGGACGCCCCUAGAAUCGAGAACACCGCCAAGCUGA | |
| | GCGGCGAGGUGCAGAGAAAGGGCGUGCAGGACGUCGGCG | |
| | GAGGCGAGCACGGCAGGUGGAGCGGCAUCGGCUACGUGA | |
| | ACUACUGGCUUCGGGCCGGCAACGGCAAGAAAGCCGGCAC | |
| | CAUCGCCGCACCUUGGUACGGUGGCGAUCUGACCGCAGAG | |
| | CAGAGCCUGAUCGAGGUGGGCAAGGACCUGUACCUGAAC | |
| | GCCGGCGCCAGAAAGGACGAGCACAGACACCUGCUGAACG | |
| | AGGGCGUGAUCCAGGCCGGAGGUCACGGCCACAUCGGCG | |
| | GCGACGUGGACAACAGGGCCGUGGUCCGUACCGUGAGCG | |
| | CCAUGGAGUACUUCAAGACCCCUCUGCCUGUGAGCCUGAC | |
| | CGCCCUGGACAAUAGAGCCGGACUCAGCCCAGCCACCUGG | |
| | AACUUCCAGAGCACCUACGAGCUGCUGGACUACCUGCUGG | |
| | ACCAGAACAGAUACGAGUACAUCUGGGGCCUGUACCCUA | |
| | CCUACACCGAGUGGAGCGUGAACACCCUGAAGAACCUGG | |
| | ACCUGGGCUACCAGGCCAAGCCUGCCCCUACCGCCCCUCC | |
| | UAUGCCUAAGGCCCCUGAGCUGGACCUGAGAGGCCAUACC | |
| | CUGGAGAGCGCCGAGGGCAGAAAGAUCUUCGGCGAGUAC | |
| | AAGAAGCUGCAGGGCGAGUACGAGAAGGCCAAGAUGGCC | |
| | GUGCAAGCCGUGGAGGCCUACGGCGAGGCCACCAGAAGA | |
| | GUGCACGACCAGCUCGGGCAGCGAUACGGCAAGGCCCUGG | |
| | GCGGCAUGGACGCCGAAACCAAGGAGGUGGACGGCAUCA | |
| | UCCAGGAGUUUGCGGCCGAUCUGAGAACCGUGUACGCCA | |
| | AACAGGCCGACCAAGCCACUAUCGACGCCGAGACUGACAA | |
| | GGUGGCCCAGAGAUACAAGAGCCAGAUCGACGCCGUGAG | |
| | ACUCCAACGGGCCAAGAGAAGCGGAAGCGGCGCUCCC | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding amino | METPAQLLFLLLLWLPDTTGSLYAEHDATLTLAQGTQRDLVV | 35 |
| acid sequence | DQDHILPVAEGTLRVKAKSLTTEIETGNPGSLIAEVQENIDNKQ | |
| | AIVVGKDLTLSSAHGNVANEANALLWAAGELTVKAQNITNK | |
| | RAALIEAGGNARLTAAVALLNKLGRIRAGEDMHLDAPRIENT | |
| | AKLSGEVQRKGVQDVGGGEHGRWSGIGYVNYWLRAGNGKK | |
| | AGTIAAPWYGGDLTAEQSLIEVGKDLYLNAGARKDEHRHLLN | |
| | EGVIQAGGHGHIGGDVDNRAVVRTVSAMEYFKTPLPVSLTAL | |
| | DNRAGLSPATWNFQSTYELLDYLLDQNRYEYIWGLYPTYTE | |
| | WSVNTLKNLDLGYQAKPAPTAPPMPKAPELDLRGHTLESAEG | |
| | RKIFGEYKKLQGEYEKAKMAVQAVEAYGEATRRVHDQLGQR | |
| | YGKALGGMDAETKEVDGIIQEFAADLRTVYAKQADQATIDAE | |
| | TDKVAQRYKSQIDAVRLQ | |
| PolyA tail | 100 nt | |

FIM2-3

| SEQ ID NO: 36 consists of from 5′ end to 3′ end: | | 36 |
|---|---|---|
| 5′ UTR SEQ ID NO: 2, mRNA ORF SEQ ID NO: 37, and | | |
| 3′ UTR SEQ ID NO: 4. | | |
| 5′ UTR | GGGAAAUAAGAGAGAAAAGAAGAGUAAGAAGAAAUAUAA | 2 |
| | GACCCCGGCGCCGCCACC | |
| ORF of mRNA | AUGGAGACCCCUGCCCAGCUGCUGUUCCUGCUGCUGCUUU | 37 |
| Construct | GGCUGCCUGACACCACCGGAGUGGAUCCACCUGUGGACUG | |
| (excluding the stop | CGGACGUGCAUUAGGACUGCACUUCUGGUCAAGUGCGAG | |
| codon) | CCUUAUAUCGGAUCAGACCCCUGACGGCACAUUGAUCGGC | |
| | AAGCCGGUGGUCGGACGGUCACUAUUGAGCAAGUCUUGC | |
| | AAGGUGCCUGACGACAUUAAGGAAGACCUGUCAGACAAU | |
| | CACGACGGCGAGCCAGUUGAUAUCGUACUGGAGCUUGGC | |
| | AGCAACUAUAAGAUUAGGCCACAGUCGUACGGUCAUCCU | |
| | GGAAUAGUGGUCGACCUGCCUUCCGGUUCAACAGAGGAG | |
| | ACAGGUAUCGCUAUCUACAUCGACUUCGGCUCUAGCCCAA | |
| | UGCAGAAGGUCGGCGAGAGACAGUGGCUGUACCCACAGA | |
| | AGGGUGAGGUGCUUUUCGACGUGCUGACUAUUAACGGAG | |
| | ACAACGCCGAGGUUCGGUACCAGGCCAUCAAGGUCGGUCC | |
| | ACUGAAGCGCCCAAGAAAGCUGGUCCUUAGUCAGUUCCC | |
| | AAACCUCUUCACUUACAAGUGGGUCUUCAUGAGAGGCAC | |
| | AAGCCAGGAGCGGGUCCUCGCACAGGGCACCAUAGAUAC | |
| | UGACGUGGCCACGAGCACCAUCGACCUGAAGACGUGCAG | |
| | AUACACCAGUCAGACCGUGUCGCUCCCAAUCAUUCAGAGG | |
| | AGUGCCUUGACCGGUGUUGGCACCACACUUGGCAUGACU | |
| | GACUUCCAAAUGCCAUUCUGGUGUUACGGCUGGCCUAAG | |
| | GUGUCAGUUUACAUGUCUGCGACAAAGACUCAAACAGGC | |
| | GUAGACGGCGUAGCACUCCCAGCAACCGGACAGGCCGCCG | |
| | GUAUGGCCUCCGGCGUGGGCGUUCAGUUGAUUAACGGUA | |
| | AGACGCAGCAGCCUGUCAAGCUGGGACUCCAGGGCAAGA | |
| | UUGCCCUGCCUGAGGCACAGCAGACCGAAAGUGCAACAU | |
| | UCUCACUGCCAAUGAAGGCCCAGUACUACCAGACCAGUAC | |
| | CUCUACAUCUGCCGGCAAGCUGUCCGUUACCUACGCCGUC | |
| | ACUCUGAAUUACGACGGCGGAGGCUCCGACGACGGCACA | |
| | AUCGUGAUAACCGGUACAAUUACUGACACAACCUGUGUA | |
| | AUCGAGGAUCCUUCCGGCCCAUCGCAUACUAAGGUUGUU | |
| | CAGCUGCCAAAGAUUUCCAAGAACGCCCUCAAGGCCAACG | |
| | GAGAUCAAGCUGGUAGGACACCAUUCAUAAUUAAGCUAA | |
| | AGGACUGUCCAUCCUCCCUCGGUAACGGCGUGAAGGCCUA | |
| | UUUCGAGCCAGGACCUACGACUGACUACUCUACAGGUGA | |
| | UCUCAGAGCAUAUAAGAUGGUUUACGCAACAAAUCCUCA | |
| | GACACAGCUCAGCAAUAUCGUCGCCGCCACUGAGGCUCAG | |
| | GGAGUCCAGGUCCGGAUCUCCAACUUAGACGACUCAAAG | |
| | AUCACCAUGGGCGCCAACGAGGCAACUCAGCAGGCCGCUG | |
| | GAUUCGACCCAGAGGUACAGACUGGCGGUACGUCCCGCAC | |
| | AGUCACCAUGCGGUACCUGGCAAGUUACGUUAAGAAGAA | |
| | CGGUGACGUGGAGGCGUCCGCCAUAACCACAUACGUGGG | |
| | AUUCUCAGUGGUGUACCCUGGUGGCGGCUCUGGCGGUGG | |
| | CAGUAACGACGGUACUAUUGUAAUUACUGGAAGUAUUUC | |
| | AGACCAAACGUGCGUUAUCGAAGAACCAAGCACAUUAAA | |
| | CCAUAUUAAGGUGGUGCAGUUGCCUAAGAUAUCAAAGAA | |
| | CGCUUUGAGGAACGACGGCGAUACUGCAGGCGCAACACC | |
| | UUUCGAUAUAAAGCUUAAGGAGUGCCCUCAGGCCCUGGG | |
| | CGCCCUGAAGCUCUACUUCGAACCAGGCAUAACAACUAAC | |
| | UACGACACCGGUGAUCUUAUUGCUUACAAGCAGACAUAU | |
| | AACGCAGCCGGCAACGGCCAGCUUUCCACUGUAUCAUCCG | |
| | CCACAAAGGCUAAGGGCGUCGAGUUCAGGCUGGCGAAUC | |
| | UCAACGGACAACACAUUAGAAUGGGUACUGAUAAGACCA | |
| | CCCAAGCUGCACAGACCUUCACAGGUAAGGUCACUAACGG | |
| | CGGUAAGAGUUAUACACUGCGCUACCUGGCUAGCUACGU | |
| | GAAGAAGCCAAAGGAGGACGUCGACGCAGCACAGAUAAC | |
| | AUCUUACGUAGGAUUCUCUGUUGUUUAUCCGGGUGGCGG | |
| | UAGCAACGACGGAACCAUUGUGAUCACUGGCUCAAUUUC | |
| | CGACCAGACAGUCAUU | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding amino | METPAQLLFLLLLWLPDTTGVDPPVDCGRALGLHFWSSASLIS | 38 |
| acid sequence | DQTPDGTLIGKPVVGRSLLSKSCKVPDDIKEDLSDNHDGEPVD | |
| | IVLELGSNYKIRPQSYGHPGIVVDLPSGSTEETGIAIYIDFGSSP | |
| | MQKVGERQWLYPQKGEVLFDVLTINGDNAEVRYQAIKVGPL | |
| | KRPRKLVLSQFPNLFTYKWVFMRGTSQERVLAQGTIDTDVAT | |
| | STIDLKTCRYTSQTVSLPIIQRSALTGVGTTLGMTDFQMPFWC | |
| | YGWPKVSVYMSATKTQTGVDGVALPATGQAAGMASGVGVQ | |
| | LINGKTQQPVKLGLQGKIALPEAQQTESATFSLPMKAQYYQTS | |
| | TSTSAGKLSVTYAVTLNYDGGGSDDGTIVITGTITDTTCVIEDP | |
| | SGPSHTKVVQLPKISKNALKANGDQAGRTPFIIKLKDCPSSLGN | |
| | GVKAYFEPGPTTDYSTGDLRAYKMVYATNPQTQLSNIVAATE | |
| | AQGVQVRISNLDDSKITMGANEATQQAAGFDPEVQTGGTSRT | |
| | VTMRYLASYVKKNGDVEASAITTYVGFSVVYPGGGSGGGSN | |
| | DGTIVITGSISDQTCVIEEPSTLNHIKVVQLPKISKNALRNDGDT | |
| | AGATPFDIKLKECPQALGALKLYFEPGITTNYDTGDLIAYKQT | |
| | YNAAGNGQLSTVSSATKAKGVEFRLANLNGQHIRMGTDKTT | |
| | QAAQTFTGKVTNGGKSYTLRYLASYVKKPKEDVDAAQITSYV | |
| | GFSVVYPGGGSNDGTIVITGSISDQTVI | |
| PolyA tail | 100 nt | |

RTX

| SEQ ID NO: 39 consists of from 5′ end to 3′ end: | | 39 |
|---|---|---|
| 5′ UTR SEQ ID NO: 2, mRNA ORF SEQ ID NO: 40, and | | |
| 3′ UTR SEQ ID NO: 4. | | |
| 5′ UTR | GGGAAAUAAGAGAGAAAAGAAGAGUAAGAAGAAAUAUAA | 2 |
| | GACCCCGGCGCCGCCACC | |
| ORF of mRNA | AUGGAGACCCCUGCCCAGCUGCUGUUCCUGCUGCUGCUUU | 40 |
| Construct | GGCUGCCAGACACCACCGGAACCCUGGAGCACGUGCAGCA | |
| (excluding the stop | CAUCAUCGGCGGCGCCGGCAACGACGCCAUCACUGGCAAC | |
| codon) | GCCCACGACAACUUCCUGGCCGGCGGCAGCGGCGACGACA | |
| | GACUGGACGGCGGAGCAGGUAACGACGUGCUCGUCGGAG | |
| | GCGAGGGGCAGAACACCGUGAUUGGCGGAGCCGGAGACG | |
| | ACGUGUUCCUGCAGGACCUGGGCGUGUGGAGCAACCAGU | |
| | UAGACGGCGGCGCAGGUGUGGAUACUGUUAAGUACAACG | |
| | UGCACCAGCCUAGCGAGGAGAGACUGGAGAGAAUGGGCG | |
| | ACACCGGCAUCCACGCCGACCUGCAGAAGGGAACCGUGGA | |
| | GAAGUGGCCUGCCCUGAACCUGUUCAGCGUGGACCACGU | |
| | GAAGAACAUCGAGAACCUGCACGGCAGUAGACUAAACGA | |
| | CAGAAUUGCAGGAGACGAUCAGGACAACGAACUGUGGGG | |
| | UCACGACGGAAACGACGUGAUACGUGGCAGAGGAGGCGA | |
| | CGACAUCCUGCGGGGCGGCCUGGGCCUGGACACCCUGUAC | |
| | GGCGAGGACGGAAACGACAUCUUCCUCCAAGACGACGAA | |
| | ACCGUGAGCGACGAUAUCGACGGCGGUGCCGGAUUAGAC | |
| | ACCGUCGACUACAGCGCCAUGAUCCACCCUGGCAGAAUCG | |
| | UGGCCCCUCACGAGUACGGCUUCGGCAUCGAGGCUGACCU | |
| | GAGCAGAGAGUGGGUGAGAAAGGCCAGCGCGUUGGGUGU | |
| | AGACUACUACGACAACGUGAGAAACGUCGAGAACGUCAU | |
| | UGGCACCAGCAUGAAAGACGUGCUCAUCGGUGACGCUCA | |
| | GGCCAACACCCUGAUGGGCCAGGGCGGUGACGACACAGU | |
| | GCGAGGCGGAGACGGAGACGACCUGCUGUUCGGUGGCGA | |
| | CGGCAACGAUAUGUUAUACGGCGACGCUGGAAACGACGU | |
| | GUUAUACGGUGGACUGGGUGACGAUACAUUGGAGGGUGG | |
| | CGCGGGCAACGAUUGGUUCGGCCAGACACAAGCCAGAGA | |
| | GCACGACGUUCUACGUGGCGGCGACGGAGUGGACACUGU | |
| | GGACUACUCACAAACCGGCGCCCACGCCGGAAUCGCGGCC | |
| | GGGAGAAUCGGCUUGGGAAUCCUUGCUGACUUGGGUGCA | |
| | GGCCGGGUGGACAAGCUGGGUGAGGCAGGCUCAAGUGCC | |
| | UACGAUACAGUGAGCGGCAUUGAGAACGUAGUCGGCACG | |
| | GAGCUUGCUGAUCGGAUUACCGGAGACGCCCAAGCAAAC | |
| | GUGUUACGGGGUGCUGGAGGCGCUGACGUCCUCGCUGGC | |
| | GGUGAGGGCGACGACGUGCUGUUGGGAGGAGACGGUGAC | |
| | GACCAACUGUCGGGCGACGCGGGAAGGGAUAGACUGUAC | |
| | GGAGAAGCAGGAGACGACUGGUUCUUCCAGGACGCCGCC | |
| | AACGCGGGAAACCUGCUUGACGGUGGUGACGGACGAGAU | |
| | ACCGUAGAUUUCUCUGGUCCAGGCAGAGGCCUCGACGCA | |
| | GGCGCCAAGGGCGUCUUCCUGUCCCUCGGUAAGGGCUUCG | |
| | CCAGCCUUAUGGACGAACCUGAGACUUCUAACGUGCUGA | |
| | GAAAUAUUGAGAACGCCGUCGGCAGCGCCAGGGACGACG | |
| | UCUUAAUCGGCGACGCCGGUGCGAACGUCCUAAACGGCU | |
| | UGGCCGGAAACGACGUUUUAAGCGGAGGAGCUGGCGACG | |
| | ACGUACUGCUUGGCGACGAGGGCAGCGACCUCCUAUCAG | |
| | GUGACGCCGGUAACGACGACCUCUUCGGCGGACAGGGUG | |
| | ACGAUACUUAUUUGUUCGGCGUGGGCUACGGACACGACA | |
| | CUCGGGCCAAGAGAAGCGGAAGCGGCGCUCCC | |
| 3' UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding amino | METPAQLLFLLLLWLPDTTGTLEHVQHIIGGAGNDAITGNAHD | 41 |
| acid sequence | NFLAGGSGDDRLDGGAGNDVLVGGEGQNTVIGGAGDDVFLQ | |
| | DLGVWSNQLDGGAGVDTVKYNVHQPSEERLERMGDTGIHAD | |
| | LQKGTVEKWPALNLFSVDHVKNIENLHGSRLNDRIAGDDQDN | |
| | ELWGHDGNDVIRGRGGDDILRGGLGLDTLYGEDGNDIFLQDD | |
| | ETVSDDIDGGAGLDTVDYSAMIHPGRIVAPHEYGFGIEADLSR | |
| | EWVRKASALGVDYYDNVRNVENVIGTSMKDVLIGDAQANTL | |
| | MGQGGDDTVRGGDGDDLLFGGDGNDMLYGDAGNDVLYGG | |
| | LGDDTLEGGAGNDWFGQTQAREHDVLRGGDGVDTVDYSQT | |
| | GAHAGIAAGRIGLGILADLGAGRVDKLGEAGSSAYDTVSGIEN | |
| | VVGTELADRITGDAQANVLRGAGGADVLAGGEGDDVLLGGD | |
| | GDDQLSGDAGRDRLYGEAGDDWFFQDAANAGNLLDGGDGR | |
| | DTVDFSGPGRGLDAGAKGVFLSLGKGFASLMDEPETSNVLRN | |
| | IENAVGSARDDVLIGDAGANVLNGLAGNDVLSGGAGDDVLL | |
| | GDEGSDLLSGDAGNDDLFGGQGDDTYLFGVGYGHDT | |
| PolyA tail | 100 nt | |

Brk

| SEQ ID NO: 42 consists of from 5′ end to 3′ end: | | 42 |
|---|---|---|
| 5′ UTR SEQ ID NO: 2, mRNA ORF SEQ ID NO: 43, and | | |
| 3′ UTR SEQ ID NO: 4. | | |
| 5′ UTR | GGGAAAUAAGAGAGAAAAGAAGAGUAAGAAGAAAUAUAA | 2 |
| | GACCCCGGCGCCGCCACC | |
| ORF of mRNA | AUGGACAGCAAGGGCAGCAGCCAGAAGGGCUCAAGACUG | 43 |
| Construct | CUCUUGUUGCUCGUGGUGAGCAACCUGCUGCUGCCUCAA | |
| (excluding the stop | GGUGUGGUGGGACAGGCCCCUCAGCCUCCGGUGGCCGGCG | |
| codon) | CCCCACACGCCCAAGACGCAGGGCAGGAGGGCGAGUUCGA | |
| | CCACAGAGACAACACCCUGAUCGCCGUGUUCGACGACGGC | |
| | GUGGGCAUCAACCUGGACGACGACCCUGACGAGCUGGGC | |
| | GAAACAGCCCCUCCUACCCUGAAGGACAUCCACAUCAGCG | |
| | UGGAGCACAAGAACCCUAUGAGCAAGCCUGCCAUCGGCG | |
| | UGAGAGUGAGCGGGGCCGGUAGGGCCCUGACCCUCGCUG | |
| | GCAGCACCAUCGACGCUACCGAGGGCGGCAUCCCUGCCGU | |
| | UGUCAGAAGAGGCGGCACCCUGGAACUGGACGGGGUGAC | |
| | CGUCGCCGGCGGAGAGGGCAUGGAGCCUAUGACCGUGAG | |
| | CGACGCAGGCAGCCGACUCAGUGUGCGGGGAGGUGUGCU | |
| | GGGAGGAGAGGCCCCGGGCGUGGGGUUAGUGCGAGCCGC | |
| | UCAGGGCGGACAGGCUAGCAUAAUCGACGCAACCCUGCA | |
| | GAGCAUCCUGGGACCCGCCCUGAUAGCGGACGGUGGCUCA | |
| | AUCUCAGUGGCUGGAGGCUCCAUUGACAUGGACAUGGGC | |
| | CCAGGCUUCCCUCCUCCACCACCUCCUCUGCCUGGAGCAC | |
| | CUCUGGCCGCCCACCCUCCGCUCGACAGAGUGGCCGCCGU | |
| | GCACGCUGGACAGGACGGCAAGGUGACCCUGAGAGAGGU | |
| | GGCGCUCCGGGCGCACGGACCCCAGGCCACAGGAGUGUAC | |
| | GCCUACAUGCCUGGCAGCGAGAUCACACUGCAGGGAGGU | |
| | ACUGUGUCCGUCCAGGGAGACGACGGUGCCGGAGUCGUC | |
| | GCAGGAGCCGGCCUGCUGGACGCCCUGCCUCCUGGAGGAA | |
| | CCGUGAGACUCGACGGUACCACCGUGUCCACCGACGGCGC | |
| | UAACACCGACGCCGUGCUGGUGAGAGGGGACGCCGCCAG | |
| | AGCCGAGGUGGUGAACACCGUGCUGAGAACCGCCAAGAG | |
| | CUUGGCCGCAGGAGUUUCCGCGCAGCACGGAGGCCGGGU | |
| | AACCCUGCGGCAGACCAGAAUUGAAACGGCUGGUGCCGG | |
| | UGCCGAAGGUAUUUCGGUACUAGGAUUCGAGCCUCAGAG | |
| | CGGCAGCGGUCCAGCUUCGGUAGACAUGCAGGGUGGUAG | |
| | CAUUACCACGACCGGCAACAGGGCCGCCGGGAUCGCGCUG | |
| | ACCCACGGCUCCGCGAGACUGGAGGGAGUCGCCGUGCGAG | |
| | CAGAAGGAUCCGGAUCCAGCGCCGCCCAGCUGGCAAACGG | |
| | AGUUCUUGUGGUAUCCGCAGGCUCACUCGCCUCUGCGCAG | |
| | UCUGGCGCUAUAUCUGUGACUGACACCCCUCUGAAGCUG | |
| | AUGCCAGGAGCUCUCGCUUCCAGCACUGUAAGCGUCAGAC | |
| | UUACUGACGGAGCCACUGCACAAGGAGGUAACGGCGUAU | |
| | UCCUGCAGCAGCAUUCAACUAUACCUGUGGCUGUGGCUC | |
| | UUGAGAGCGGAGCGCUGGCACGCGGCGACAUCGUGGCCG | |
| | ACGGGAACAAGCCCCUCGACGCCGGUAUCAGCUUGUCAGU | |
| | GGCCUCUGGAGCCGCCUGGCACGGGGCUACCCAAGUGCUG | |
| | CAGUCUGCCACGCUGGGCAAGGGUGGCACCUGGGUUGUU | |
| | AACGCCGACAGCAGAGUGCAGGACAUGAGCAUGCGGGGC | |
| | GGAAGGGUUGAGUUCCAGGCUCCGGCCCCUGAGGCCAGC | |
| | UACAAGACCUUAACACUCCAGACCUUAGACGGAAACGGC | |
| | GUGUUCGUGCUGAACACCAACGUCGCAGCGGGACAGAAC | |
| | GACCAGCUGAGAGUUACCGGCAGGGCUGACGGUCAGCAC | |
| | AGAGUCCUGGUCAGGAACGCCGGAGGCGAGGCCGAUAGC | |
| | CGGGGUGCGCGGUUAGGACUGGUUCACACCCAGGGCCAG | |
| | GGCAACGCAGUAUUCAGAUUAGCAAACGUGGGUAAGGCC | |
| | GUGGACCUGGGCACGUGGAGAUACAGUUUGGCCGAGGAC | |
| | CCUAAGACCCACGUGUGGAGUCUGCAAAGAGCCGGUCAG | |
| | GCUCUGUCAGGCGCUGCUAACGCCGCCGUUAACGCUGCGG | |
| | ACCUGAGUAGUAUCGCCCUAGCAGAAAGCAACGCCCACCA | |
| | UCACCAUCAUCAC | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding amino | MDSKGSSQKGSRLLLLLVVSNLLLPQGVVGQAPQPPVAGAPH | 44 |
| acid sequence | AQDAGQEGEFDHRDNTLIAVFDDGVGINLDDDPDELGETAPP | |
| | TLKDIHISVEHKNPMSKPAIGVRVSGAGRALTLAGSTIDATEG | |
| | GIPAVVRRGGTLELDGVTVAGGEGMEPMTVSDAGSRLSVRG | |
| | GVLGGEAPGVGLVRAAQGGQASIIDATLQSILGPALIADGGSIS | |
| | VAGGSIDMDMGPGFPPPPPPLPGAPLAAHPPLDRVAAVHAGQ | |
| | DGKVTLREVALRAHGPQATGVYAYMPGSEITLQGGTVSVQG | |
| | DDGAGVVAGAGLLDALPPGGTVRLDGTTVSTDGANTDAVLV | |
| | RGDAARAEVVNTVLRTAKSLAAGVSAQHGGRVTLRQTRIETA | |
| | GAGAEGISVLGFEPQSGSGPASVDMQGGSITTTGNRAAGIALT | |
| | HGSARLEGVAVRAEGSGSSAAQLANGVLVVSAGSLASAQSG | |
| | AISVTDTPLKLMPGALASSTVSVRLTDGATAQGGNGVFLQQH | |
| | STIPVAVALESGALARGDIVADGNKPLDAGISLSVASGAAWH | |
| | GATQVLQSATLGKGGTWVVNADSRVQDMSMRGGRVEFQAP | |
| | APEASYKTLTLQTLDGNGVFVLNTNVAAGQNDQLRVTGRAD | |
| | GQHRVLVRNAGGEADSRGARLGLVHTQGQGNAVFRLANVG | |
| | KAVDLGTWRYSLAEDPKTHVWSLQRAGQALSGAANAAVNA | |
| | ADLSSIALAESNA | |
| PolyA tail | 100 nt | |

Vag8

| SEQ ID NO: 45 consists of from 5′ end to 3′ end: | | 45 |
|---|---|---|
| 5′ UTR SEQ ID NO: 2, mRNA ORF SEQ ID NO: 46, and | | |
| 3′ UTR SEQ ID NO: 4. | | |
| 5′ UTR | GGGAAAUAAGAGAGAAAAGAAGAGUAAGAAGAAAUAUAA | 2 |
| | GACCCCGGCGCCGCCACC | |
| ORF of mRNA | AUGGAGACUCCAGCCCAGUUGUUAUUUCUUUUGUUGCUU | 46 |
| Construct | UGGCUCCCAGACACCACCGGGCAGAGAAUCGACGGCGGUG | |
| (excluding the stop | CCGCCUUCUUGGGCGACGUGGCCAUCGCCACCACCAAGGC | |
| codon) | CAGCGAGCACGGUAUCAACGUGACCGGCAGAACCGCUGA | |
| | GGUGAGAGUCACGGGAGGCACCAUCCGCACUAGCGGAAA | |
| | CCAGGCCCAGGGCCUGAGAGUAGGCACCGAGAACGCCCCU | |
| | GACAACACCGCCCUGGGCGCUAGCGUGUUCCUGCAGAACU | |
| | UGAUUAUCGAGACGAGCGGCACCGGUGCUCUAGGCGUGA | |
| | GCGUUCACGAACCACAGGGCGGCGGCGGUACGAGACUGA | |
| | GCAUGUCCGGCACCACGGUGAGAACCAGAGGGGAUGACA | |
| | GUUUCGCCCUGCAGCUCAGCGGCCCUGCCAGCGCCACCUU | |
| | GAACGACGUCGCCCUGGAGACUGCCGGCCAGCAGGCCCCU | |
| | GCCGUCGUGCUCUGGCAGGGCGCACAGUUAAACGCUCAG | |
| | GGACUGGUGGUGCAGGUGAACGGAGCCGGUGUCUCAGCG | |
| | AUCCAUGCGCAAGACGCCGGCAGCUUCACACUGAGCGGCA | |
| | GCGACAUCACCGCGCGAGGCCUGGAGGUGGUGGGCAUCU | |
| | ACGUGCAGGAGGGCAUGCAGGGCACCCUCACUGGCACACG | |
| | GGUGACAACCCAGGGCGACACUGCACCUGCACUGCAGGUG | |
| | GAGGAUGCUGGCACCCACGUGAGCAUGAAUGGCGGCGCU | |
| | UUGUCCACCUCAGGCGCCAACAGCCCUGCCGCCUGGCUGC | |
| | UGGCGGGCGGAUCUGCCCAGUUCAGAGACACCGUCCUGA | |
| | GAACCGUAGGCGAAGCUAGCCACGGCGUGGACGUGGCAG | |
| | CCCACAGUGAGGUGGAGCUCGCUCAUGCCCAGGUAAGGG | |
| | CAGACGGCCAGGGAGCCCACGGGCUGGUGGUUACCAGAA | |
| | GCUCUGCUAUGGUGCGAGCUGGAUCUCUGGUUGAGAGUA | |
| | CUGGAGAUGGCGCAGCCGCCCUGCUGGAGAGCGGCCAUCU | |
| | GACAGUGGAUGGAUCAGUGGUCCACGGCCAUGGAGCGGC | |
| | UGGUUUGGAGGUUGACGGGGAGAGCAACGUUAGUCUCCU | |
| | GAACGGCGCUCGAUUGAGCAGCGACCAGCCUACCGCCAUC | |
| | AGACUGAUCGACCCUAGAUCAGUGCUGAACCUGGACAUC | |
| | AAGGACAGAGCCCAACUGCUGGGCGACAUCGCACCUGAG | |
| | GCCCAGCAGCCUGACGGCUCCCCUGAGCAGGCGAGAGUGA | |
| | GAGUGGCCCUGGCUGACGGUGGAACGUGGGCCGGCCGGA | |
| | CGGACGGCGCCGUGCACACCGUGAGACUGCUGGACAGAG | |
| | GAGUGUGGACCGUAACCGGCGACAGCCGAGUGGCCGAGG | |
| | UGAAACUGGAGGGCGGCACUUUGGCCUUCGCCCCUCCUGC | |
| | CCAGCCUAAGGGCGCCUUCAAGACCCUGGUGGCCACCCAG | |
| | GGAAUUUCCGGCACGGGAACCAUUGUUAUGAAUGCCCAC | |
| | CUGCCUAGUGGCACAGCCGACGUGCUUGUGGCCCCUCAGG | |
| | GCUUCGGAGAUAGACAGGUAUUGGUGGUGAAUAACACCG | |
| | ACGACGGCACAGAGUCCGGCGCUACCAAGGUGCCUCUGAU | |
| | CGAGGACGAGCAGGGCCACACCGCCUUUACCCUGGGAAAC | |
| | AUGGGCGGCAGAGUAGAUGCCGGAGCCAGACAGUACGAA | |
| | CUGACCGCCAGUGAGGCACAGGCCGACAAAGCCCGGACCU | |
| | GGCAGCUGACCCCUACCAACGAGCUGAGCACCACCGCCAC | |
| | CGCCGCCGUAAAUGCCAUGGCUAUUGCUGCUAGCCAAAG | |
| | AAUUUGGCAGGCGGAAAUGGAUCAUCACCAUCAUCAUCA | |
| | U | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding amino | METPAQLLFLLLLWLPDTTGQRIDGGAAFLGDVAIATTKASEH | 47 |
| acid sequence | GINVTGRTAEVRVTGGTIRTSGNQAQGLRVGTENAPDNTALG | |
| | ASVFLQNLIIETSGTGALGVSVHEPQGGGGTRLSMSGTTVRTR | |
| | GDDSFALQLSGPASATLNDVALETAGQQAPAVVLWQGAQLN | |
| | AQGLVVQVNGAGVSAIHAQDAGSFTLSGSDITARGLEVVGIY | |
| | VQEGMQGTLTGTRVTTQGDTAPALQVEDAGTHVSMNGGALS | |
| | TSGANSPAAWLLAGGSAQFRDTVLRTVGEASHGVDVAAHSE | |
| | VELAHAQVRADGQGAHGLVVTRSSAMVRAGSLVESTGDGAA | |
| | ALLESGHLTVDGSVVHGHGAAGLEVDGESNVSLLNGARLSSD | |
| | QPTAIRLIDPRSVLNLDIKDRAQLLGDIAPEAQQPDGSPEQARV | |
| | RVALADGGTWAGRTDGAVHTVRLLDRGVWTVTGDSRVAEV | |
| | KLEGGTLAFAPPAQPKGAFKTLVATQGISGTGTIVMNAHLPSG | |
| | TADVLVAPQGFGDRQVLVVNNTDDGTESGATKVPLIEDEQGH | |
| | TAFTLGNMGGRVDAGARQYELTASEAQADKARTWQLTPTNE | |
| | LSTTATAAVNAMAIAASQRIWQAEMDHHHHHH | |
| PolyA tail | 100 nt | |

Diphtheria toxin

| SEQ ID NO: 48 consists of from 5′ end to 3′ end: | | 48 |
|---|---|---|
| 5′ UTR SEQ ID NO: 2, mRNA ORF SEQ ID NO: 49, and | | |
| 3′ UTR SEQ ID NO: 4. | | |
| 5′ UTR | GGGAAAUAAGAGAGAAAAGAAGAGUAAGAAGAAAUAUAA | 2 |
| | GACCCCGGCGCCGCCACC | |
| ORF of mRNA | AUGAGCCGGAAGCUGUUCGCCAGCAUCCUGAUCGGCGCCC | 49 |
| Construct | UGCUGGGCAUUGGUGCCCCACCAAGCGCCCACGCUGGAGC | |
| (excluding the stop | UGACGACGUGGUGGACAGCAGCAAGAGCUUCGUGAUGGA | |
| codon) | GAACUUCGCCAGCUACCACGGCACCAAGCCCGGCUACGUG | |
| | GACAGUAUCCAGAAGGGCAUCCAGAAGCCCAAGAGCGGC | |
| | ACCCAGGGCAACUACGACGACGACUGGGAGGGCUUCUAC | |
| | AGCACCGACAACAAGUACGACGCCGCCGGCUACAGCGUCG | |
| | ACAACGAGAACCCACUGUCCGGCAAAGCCGGAGGCGUGG | |
| | UGAAGGUGACCUACCCCGGCCUGACCAAGGUGCUGGCCCU | |
| | GAAGGUGGACAACGCCGAGACCAUUAAGAAGGAGCUGGG | |
| | CCUGAGCCUGACCGAGCCCCUGAUGGAGCAGGUGGGCACU | |
| | GAGGAGUUCAUCAAGCGUUUCGGGGACGGGGCGAGUAGA | |
| | GUGGUGCUGAGCCUGCCCUUCGCCGAGGGCAGCAGCAGCG | |
| | UGAAGUACAUCAACAACUGGGAGCAGGCCAAGGCCCUGA | |
| | GCGUGGAGCUGGAGAUCAACUUCGAGACCCGGGGCAAAC | |
| | GAGGCCAGGACGCCAUGUACGAGUACAUGGCACAGGCUU | |
| | GCGCCGGCAAUCGGGUGCGGAGAAGCGUGGGCUCUAGCC | |
| | UGAGCUGCAUCAAUCUGGACUGGGACGUGAUCCGGGACA | |
| | AGACGAAGACCAAGAUCGAGAGCCUGAAGGAGCACGGCC | |
| | CCAUCAAGAACAAGAUGAGCGAGAGCCCCAACAAGGCCG | |
| | UGAGCGAGGAGAAGGCCAAGCAGUACCUGGAGGAGUUCC | |
| | ACCAGACCGCCUUGGAACACCCCGAGCUGAGCGAGCUGAA | |
| | GACUGUGACCGGCACCAACCCCGUUUUCGCCGGCGCCAAC | |
| | UACGCCGCCUGGGCCGUGAACGUGGCUCAGGUGAUCGAC | |
| | AGCGAGACCGCCGACAACCUGGAGAAGACCACCGCCGCCC | |
| | UGAGCAUCCUGCCCGGCAUCGGCAGCGUGAUGGGCAUCGC | |
| | UGACGGCGCCGUGCACCACAACACCGAGGAGAUCGUGGCC | |
| | CAGAGCAUCGCCCUCAGCAGCCUGAUGGUGGCCCAGGCCA | |
| | UUCCCCUGGUGGGCGAGCUGGUGGACAUCGGCUUCGCCGC | |
| | CUACAACUUCGUGGAGAGCAUCAUCAACCUGUUCCAGGU | |
| | GGUGCACAACAGCUACAACCGGCCCGCCUAUAGCCCCGGA | |
| | CACAAGACCCAGCCCUUUCUGCACGACGGCUACGCCGUGA | |
| | GCUGGAACACCGUGGAGGACAGCAUCAUCCGGACCGGCU | |
| | UCCAGGGCGAGAGCGGCCACGACAUCAAGAUCACCGCCGA | |
| | GAACACCCCUCUGCCCAUCGCCGGCGUUCUGCUGCCCACC | |
| | AUCCCCGGCAAGCUGGACGUGAACAAGGCCAAGACCCACA | |
| | UCAGCGUGAACGGCCGGAAGAUCCGAAUGCGGUGCCGGG | |
| | CAAUCGACGGCGACGUGACCUUCUGCCGGCCUAAGAGCCC | |
| | CGUGUACGUGGGCAACGGCGUUCACGCCAACCUGCACGUG | |
| | GCCUUCCACCGGAGCAGCUCUGAGAAGAUCCACAGCAACG | |
| | AGAUCAGCAGCGACAGCAUCGGCGUGCUGGGCUACCAGA | |
| | AGACCGUGGACCACACCAAGGUGAACAGCAAACUGAGCC | |
| | UGUUCUUCGAGAUCAAGAGC | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding amino | MSRKLFASILIGALLGIGAPPSAHAGADDVVDSSKSFVMENFA | 50 |
| acid sequence | SYHGTKPGYVDSIQKGIQKPKSGTQGNYDDDWEGFYSTDNKY | |
| | DAAGYSVDNENPLSGKAGGVVKVTYPGLTKVLALKVDNAET | |
| | IKKELGLSLTEPLMEQVGTEEFIKRFGDGASRVVLSLPFAEGSS | |
| | SVKYINNWEQAKALSVELEINFETRGKRGQDAMYEYMAQAC | |
| | AGNRVRRSVGSSLSCINLDWDVIRDKTKTKIESLKEHGPIKNK | |
| | MSESPNKAVSEEKAKQYLEEFHQTALEHPELSELKTVTGTNPV | |
| | FAGANYAAWAVNVAQVIDSETADNLEKTTAALSILPGIGSVM | |
| | GIADGAVHHNTEEIVAQSIALSSLMVAQAIPLVGELVDIGFAA | |
| | YNFVESIINLFQVVHNSYNRPAYSPGHKTQPFLHDGYAVSWN | |
| | TVEDSIIRTGFQGESGHDIKITAENTPLPIAGVLLPTIPGKLDVN | |
| | KAKTHISVNGRKIRMRCRAIDGDVTFCRPKSPVYVGNGVHAN | |
| | LHVAFHRSSSEKIHSNEISSDSIGVLGYQKTVDHTKVNSKLSLF | |
| | FEIKS | |
| PolyA tail | 100 nt | |

Tetanus toxin

| SEQ ID NO: 51 consists of from 5′ end to 3′ end: | | 51 |
|---|---|---|
| 5′ UTR SEQ ID NO: 2, mRNA ORF SEQ ID NO: 52, and | | |
| 3′ UTR SEQ ID NO: 4. | | |
| 5′ UTR | GGGAAAUAAGAGAGAAAAGAAGAGUAAGAAGAAAUAUAA | 2 |
| | GACCCCGGCGCCGCCACC | |
| ORF of mRNA | AUGGAGACACCCGCCCAGCUGCUGUUCCUGCUGCUGCUGU | 52 |
| Construct | GGCUGCCCGACACCACCGGCAAGAACCUGGACUGCUGGGU | |
| (excluding the stop | GGACAACGAGGAGGACAUCGACGUGAUCCUGAAGAAGAG | |
| codon) | CACCAUCCUGAACCUGGACAUCAACAACGACAUCAUCAGC | |
| | GACAUCAGCGGCUUCAACAGCGCCGUGAUCACCUACCCCG | |
| | ACGCCCAACUGGUGCCCGGCAUCAACGGCAAGGCCAUCCA | |
| | CCUGGUGAACAACGAGGCCAGCGAGGUGAUCGUGCACAA | |
| | GGCCAUGGACAUCGAGUACAACGACAUGUUCAACAACUU | |
| | CGCCGUGAGCUUCUGGCUGCGGGUGCCCAAGGUGAGCGCC | |
| | AGCCACCUGGAGCAGUACGGCACCAACGAGUACAGCAUCA | |
| | UCAGCAGCAUGAAGAAGCACAGCCUGAGCAUCGGCAGCG | |
| | GCUGGAGCGUGAGCCUGAAGGGCAACAACCUGAUCUGGA | |
| | CCCUGAAGGAUAGCGCCGGCGAGGUGAGACAGAUCACCU | |
| | UCCGGGACCUGCCCGACAAGUUCAACGCCUACCUGGCCAA | |
| | CAAGUGGGUGUUCAUUACCAUCACCAACGACCGGCUGAG | |
| | CAGCGCCAACCUGUACAUCAACGGCGUGCUGAUGGGCAGC | |
| | GCCGAGAUCACCGGCCUGGGAGCCAUCCGGGAGGACAACA | |
| | ACAUCGCCCUGAAGCUGGACCGGUGCAACAACAAUAACCA | |
| | GUACGUGAGCAUCGACAAGUUCCGGAUCUUCUGCAAGGC | |
| | CCUGAACCCCAAGGAGAUCGAGAAGCUGUACACCAGCUAC | |
| | CUGAGCAUCACCUUCCUGCGGGACUUCUGGGGCAAUCCCC | |
| | UGCGGUACGACACCGAGUACUACCUGAUCCCCGUGGCCUC | |
| | AAGCAGCAAGGACGUGCAGUUGAAGAACAUCGCCGACUA | |
| | CAUGUACCUGACCAACGCCCCGAGCUACACCAACGGCAAG | |
| | CUGAACAUCUACUACCGGCGGCUGUACAACGGCCUGAAG | |
| | UUCAUCAUCAAGCGGUACACCCCUAACAACGAGAUCGACA | |
| | GCUUCGUGAAGAGCGGCGACUUCAUCAAGCUGUACGUGA | |
| | GCUACAACAACAACGAGCACAUCGUGGGCUACCCCAAGGA | |
| | CGGCAACGCCUUCAACAACCUGGACCGGAUCCUGCGGGUG | |
| | GGCUACAACGCCCCAGGCAUUCCCCUGUACAAGAAGAUGG | |
| | AGGCCGUGAAGCUGCGGGACCUGAAGACCUACAGCGUGC | |
| | AGCUGAAACUGUACGACGACAAGAACGCCAGCCUGGGCC | |
| | UGGUGGGCACCCACAACGGCCAGAUCGGCAACGACCCCAA | |
| | CCGGGACAUCCUGAUCGCCAGCAACUGGUACUUUAACCAC | |
| | CUGAAGGACAAGAUCCUGGGCUGCGACUGGUACUUCGUG | |
| | CCCACCGACGAGGGCUGGACCAACGAC | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding amino | METPAQLLFLLLLWLPDTTGKNLDCWVDNEEDIDVILKKSTIL | 53 |
| acid sequence | NLDINNDIISDISGFNSAVITYPDAQLVPGINGKAIHLVNNEASE | |
| | VIVHKAMDIEYNDMFNNFAVSFWLRVPKVSASHLEQYGTNE | |
| | YSIISSMKKHSLSIGSGWSVSLKGNNLIWTLKDSAGEVRQITFR | |
| | DLPDKFNAYLANKWVFITITNDRLSSANLYINGVLMGSAEITG | |
| | LGAIREDNNIALKLDRCNNNNQYVSIDKFRIFCKALNPKEIEKL | |
| | YTSYLSITFLRDFWGNPLRYDTEYYLIPVASSSKDVQLKNIAD | |
| | YMYLTNAPSYTNGKLNIYYRRLYNGLKFIIKRYTPNNEIDSFV | |
| | KSGDFIKLYVSYNNNEHIVGYPKDGNAFNNLDRILRVGYNAP | |
| | GIPLYKKMEAVKLRDLKTYSVQLKLYDDKNASLGLVGTHNG | |
| | QIGNDPNRDILIASNWYFNHLKDKILGCDWYFVPTDEGWTND | |
| PolyA tail | 100 nt | |

Prn_FurinF2A_Brka

| SEQ ID NO: 54 consists of from 5′ end to 3′ end: | | 54 |
|---|---|---|
| 5′ UTR SEQ ID NO: 2, mRNA ORF SEQ ID NO: 55, and | | |
| 3′ UTR SEQ ID NO: 4. | | |
| 5′ UTR | GGGAAAUAAGAGAGAAAAGAAGAGUAAGAAGAAAUAUAA | 2 |
| | GACCCCGGCGCCGCCACC | |
| ORF of mRNA | AUGGAGACCCCUGCCCAGCUGCUGUUCCUGCUGCUGCUUU | 55 |
| Construct | GGCUGCCAGACACCACCGGAGACUGGAACAACCAGGCCAU | |
| (excluding the stop | CGUGAAGACCGGCGAGAGACAGCACGGCAUCCACAUCCAA | |
| codon) | GGCAGCGACCCCGGCGGCGUGAGAACCGCCUCCGGCACCA | |
| | CAAUCAAGGUGAGCGGCAGACAGGCCCAGGGCAUCCUGC | |
| | UGGAGAACCCUGCCGCCGAGCUGCAGUUCAGAAACGGCGC | |
| | CGUGACCAGCAGCGGCCAGCUGUCCGACGACGGCAUCAGA | |
| | AGAUUCCUGGGCACCGUGACCGUGAAGGCCGGCAAGCUG | |
| | GUGGCCGACCACGCCACCCUGGCCAACGUGGGUGAUACCU | |
| | GGGACGACGACGGUAUUGCCCUGUACGUGGCCGGCGAGC | |
| | AAGCACAGGCCAGCAUCGCCGACAGCACCCUGCAAGGCGC | |
| | AGGCGGCGUGCAGAUCGAGAGAGGCGCCAACGUGGUGGU | |
| | GCAGAGAAGCGCCAUUGUUGACGGCGGCCUGCACAUCGG | |
| | CGCCCUGCAGAGCCUGCAGCCUGAGGACCUGCCUCCUAGC | |
| | AGAGUGGUGCUGAGAGACACAAACGUCGUCGCCGUGCCA | |
| | GCGUCCGGAGCUCCAGCAGCCGUGAGCGUGCUGGGCGCCA | |
| | GCGAGCUGACCCUCGACGGAGGACACAUCACCGGUGGCAG | |
| | GGCCGCCGGUGUGGCCGCCAUGCAGGGAGCCGUUGUGCA | |
| | UCUACAAAGAGCUACCAUCCGACGGGGCGACGCUCCGGCU | |
| | GGUGGAGCGGUCCCUGGCGGUGCCGUCCCCGGAGGAGCA | |
| | GUGCCAGGCGGUUUCGGCCCGGGUGGAUUCGGCCCUGUG | |
| | CUAGACGGCUGGUACGGCGUGGACGUGAGUGGCUCCAGC | |
| | GUGGAGCUGGCCCAGAGUAUUGUUGAGGCCCCUGAGCUC | |
| | GGCGCUGCCAUCAGAGUGGGCCGUGGUGCCAGAGUGACC | |
| | GUUAGCGGUGGCUCACUUAGCGCCCCUCACGGCAACGUGA | |
| | UCGAAACUGGAGGAGCGAGACGAUUCGCACCUCAGGCCG | |
| | CCCCUCUGAGCAUAACGCUGCAAGCGGGCGCCCACGCGCA | |
| | AGGCAAGGCCCUGCUGUACAGAGUGCUGCCUGAGCCUGU | |
| | GAAGUUAACUCUGACGGGAGGUGCCGACGCACAAGGUGA | |
| | CAUCGUGGCCACGGAACUUCCUAGCAUCCCUGGCACAUCU | |
| | AUCGGCCCUCUGGACGUUGCGUUAGCUUCGCAGGCCCGUU | |
| | GGACCGGCGCCACCAGAGCCGUGGACUCCCUAAGCAUCGA | |
| | CAACGCCGUGUGGGUCAUGACCGACAACUCAAACGUGGG | |
| | CGCGCUGAGAUUGGCCUCAGACGGCAGUGUCGAUUUCCA | |
| | ACAACCUGCCGAAGCAGGAAGAUUCAAGGUGCUGACUGU | |
| | CAAUACACUGGCCGGAAGCGGCCUGUUCAGAAUGAACGU | |
| | GUUCGCCGACCUGGGACUCUCAGAUAAGCUGGUCGUGAU | |
| | GCAGGACGCAAGUGGCCAGCACAGACUGUGGGUCAGAAA | |
| | CUCGGGCUCCGAGCCAGCCAGUGCCAAUACUUUGCUGUUG | |
| | GUGCAAACCCCUCUGGGCAGUGCCGCCACCUUCACUCUCG | |
| | CCAACAAGGACGGCAAGGUGGACAUCGGCACCUACAGAU | |
| | ACAGGCUGGCAGCAAACGGAAACGGCCAGUGGAGCCUAG | |
| | UCGGAGCAAAGGCCCCUCCUGCCCCUAAGCCUGCUCCACA | |
| | GCCUGGCCCACAGCCUCCGCAACCUCCGCAGCCUCAGCCA | |
| | GAGGCCCCAGCCCCACAACCUCCUGCAGGCAGAGAGCUGA | |
| | GUGCCGCUGCUAACGCCGCCGUUAAUACCGGAGGUGUCG | |
| | GCCUCGCGUCUACCUUGUGGUACGCCGAGAGCAACCGGGC | |
| | CAAGAGAAGCGGAAGCGGCGCUCCCGUGAAGCAGACCCU | |
| | GAACUUCGACCUGCUGAAGCUGGCCGGCGACGUGGAGAG | |
| | CAACCCCGGCCCCAUGGAGACUCCCGCCCAGCUCCUGUUU | |
| | CUGCUGUUGCUGUGGCUGCCCGACACAACCGGCCAGGCCC | |
| | CUCAGCCUCCGGUGGCCGGCGCCCCACACGCCCAAGACGC | |
| | AGGGCAGGAGGGCGAGUUCGACCACAGAGACAACACCCU | |
| | GAUCGCCGUGUUCGACGACGGCGUGGGCAUCAACCUGGA | |
| | CGACGACCCUGACGAGCUGGGCGAAACAGCCCCUCCUACC | |
| | CUGAAGGACAUCCACAUCAGCGUGGAGCACAAGAACCCU | |
| | AUGAGCAAGCCUGCCAUCGGCGUGAGAGUGAGCGGGGCC | |
| | GGUAGGGCCCUGACCCUCGCUGGCAGCACCAUCGACGCUA | |
| | CCGAGGGCGGCAUCCCUGCCGUUGUCAGAAGAGGCGGCAC | |
| | CCUGGAACUGGACGGGGUGACCGUCGCCGGCGGAGAGGG | |
| | CAUGGAGCCUAUGACCGUGAGCGACGCAGGCAGCCGACUC | |
| | AGUGUGCGGGGAGGUGUGCUGGGAGGAGAGGCCCCGGGC | |
| | GUGGGGUUAGUGCGAGCCGCUCAGGGCGGACAGGCUAGC | |
| | AUAAUCGACGCAACCCUGCAGAGCAUCCUGGGACCCGCCC | |
| | UGAUAGCGGACGGUGGCUCAAUCUCAGUGGCUGGAGGCU | |
| | CCAUUGACAUGGACAUGGGCCCAGGCUUCCCUCCUCCACC | |
| | ACCUCCUCUGCCUGGAGCACCUCUGGCCGCCCACCCUCCG | |
| | CUCGACAGAGUGGCCGCCGUGCACGCUGGACAGGACGGU | |
| | AAGGUGACCCUGAGAGAGGUGGCGCUCCGGGCGCACGGA | |
| | CCCCAGGCCACAGGAGUGUACGCCUACAUGCCUGGCAGCG | |
| | AGAUCACACUGCAGGGAGGUACUGUGUCCGUCCAGGGAG | |
| | ACGACGGUGCCGGAGUCGUCGCAGGAGCCGGCCUGCUGG | |
| | ACGCCCUGCCUCCUGGAGGAACCGUGAGACUCGACGGUAC | |
| | CACCGUGUCCACCGACGGCGCUAACACCGACGCCGUGCUG | |
| | GUGAGAGGGGACGCCGCCAGAGCCGAGGUGGUGAACACC | |
| | GUGCUGAGAACCGCCAAGAGCUUGGCCGCAGGAGUUUCC | |
| | GCGCAGCACGGAGGCCGGGUAACCCUGCGGCAGACCAGAA | |
| | UUGAAACGGCUGGUGCCGGUGCCGAAGGUAUUUCGGUAC | |
| | UAGGAUUCGAGCCUCAGAGCGGCAGCGGUCCAGCUUCGG | |
| | UAGACAUGCAGGGUGGUAGCAUUACCACGACCGGCAACA | |
| | GGGCCGCCGGGAUCGCGCUGACCCACGGCUCCGCGAGACU | |
| | GGAGGGAGUCGCCGUGCGAGCAGAAGGAUCCGGAUCCAG | |
| | CGCCGCCCAGCUGGCAAACGGAGUUCUUGUGGUAUCCGCA | |
| | GGCUCACUCGCCUCUGCGCAGUCUGGCGCUAUAUCUGUGA | |
| | CUGACACCCCUCUGAAGCUGAUGCCAGGAGCUCUCGCUUC | |
| | CAGCACUGUAAGCGUCAGACUUACUGACGGAGCCACUGC | |
| | ACAAGGAGGUAACGGCGUAUUCCUGCAGCAGCAUUCAAC | |
| | UAUACCUGUGGCUGUGGCUCUUGAGAGCGGAGCGCUGGC | |
| | ACGCGGCGACAUCGUGGCCGACGGGAACAAGCCCCUCGAC | |
| | GCCGGUAUCAGCUUGUCAGUGGCCUCUGGAGCCGCCUGGC | |
| | ACGGGGCUACCCAAGUGCUGCAGUCUGCCACGCUGGGCAA | |
| | GGGUGGCACCUGGGUUGUUAACGCCGACAGCAGAGUGCA | |
| | GGACAUGAGCAUGCGGGGCGGAAGGGUUGAGUUCCAGGC | |
| | UCCGGCCCCUGAGGCCAGCUACAAGACCUUAACACUCCAG | |
| | ACCUUAGACGGAAACGGCGUGUUCGUGCUGAACACCAAC | |
| | GUCGCAGCGGGACAGAACGACCAGCUGAGAGUUACCGGC | |
| | AGGGCUGACGGUCAGCACAGAGUCCUGGUCAGGAACGCC | |
| | GGAGGCGAGGCCGAUAGCCGGGGUGCGCGGUUAGGACUG | |
| | GUUCACACCCAGGGCCAGGGCAACGCAGUAUUCAGAUUA | |
| | GCAAACGUGGGUAAGGCCGUGGACCUGGGCACGUGGAGA | |
| | UACAGUUUGGCCGAGGACCCUAAGACCCACGUGUGGAGU | |
| | CUGCAAAGAGCCGGUCAGGCUCUGUCAGGCGCUGCCAACG | |
| | CCGCCGUGAACGCUGCGGACCUGAGUAGUAUCGCCCUAGC | |
| | AGAAAGCAACGCC | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding amino | METPAQLLFLLLLWLPDTTGDWNNQAIVKTGERQHGIHIQGS | 56 |
| acid sequence | DPGGVRTASGTTIKVSGRQAQGILLENPAAELQFRNGAVTSSG | |
| | QLSDDGIRRFLGTVTVKAGKLVADHATLANVGDTWDDDGIA | |
| | LYVAGEQAQASIADSTLQGAGGVQIERGANVVVQRSAIVDGG | |
| | LHIGALQSLQPEDLPPSRVVLRDTNVVAVPASGAPAAVSVLGA | |
| | SELTLDGGHITGGRAAGVAAMQGAVVHLQRATIRRGDAPAG | |
| | GAVPGGAVPGGAVPGGFGPGGFGPVLDGWYGVDVSGSSVEL | |
| | AQSIVEAPELGAAIRVGRGARVTVSGGSLSAPHGNVIETGGAR | |
| | RFAPQAAPLSITLQAGAHAQGKALLYRVLPEPVKLTLTGGAD | |
| | AQGDIVATELPSIPGTSIGPLDVALASQARWTGATRAVDSLSID | |
| | NAVWVMTDNSNVGALRLASDGSVDFQQPAEAGRFKVLTVNT | |
| | LAGSGLFRMNVFADLGLSDKLVVMQDASGQHRLWVRNSGSE | |
| | PASANTLLLVQTPLGSAATFTLANKDGKVDIGTYRYRLAANG | |
| | NGQWSLVGAKAPPAPKPAPQPGPQPPQPPQPQPEAPAPQPPAG | |
| | RELSAAANAAVNTGGVGLASTLWYAESNRAKRSGSGAPVKQ | |
| | TLNFDLLKLAGDVESNPGPMETPAQLLFLLLLWLPDTTGQAP | |
| | QPPVAGAPHAQDAGQEGEFDHRDNTLIAVFDDGVGINLDDDP | |
| | DELGETAPPTLKDIHISVEHKNPMSKPAIGVRVSGAGRALTLA | |
| | GSTIDATEGGIPAVVRRGGTLELDGVTVAGGEGMEPMTVSDA | |
| | GSRLSVRGGVLGGEAPGVGLVRAAQGGQASIIDATLQSILGPA | |
| | LIADGGSISVAGGSIDMDMGPGFPPPPPPLPGAPLAAHPPLDRV | |
| | AAVHAGQDGKVTLREVALRAHGPQATGVYAYMPGSEITLQG | |
| | GTVSVQGDDGAGVVAGAGLLDALPPGGTVRLDGTTVSTDGA | |
| | NTDAVLVRGDAARAEVVNTVLRTAKSLAAGVSAQHGGRVTL | |
| | RQTRIETAGAGAEGISVLGFEPQSGSGPASVDMQGGSITTTGN | |
| | RAAGIALTHGSARLEGVAVRAEGSGSSAAQLANGVLVVSAGS | |
| | LASAQSGAISVTDTPLKLMPGALASSTVSVRLTDGATAQGGN | |
| | GVFLQQHSTIPVAVALESGALARGDIVADGNKPLDAGISLSVA | |
| | SGAAWHGATQVLQSATLGKGGTWVVNADSRVQDMSMRGG | |
| | RVEFQAPAPEASYKTLTLQTLDGNGVFVLNTNVAAGQNDQLR | |
| | VTGRADGQHRVLVRNAGGEADSRGARLGLVHTQGQGNAVF | |
| | RLANVGKAVDLGTWRYSLAEDPKTHVWSLQRAGQALSGAA | |
| | NAAVNAADLSSIALAESNA | |
| PolyA tail | 100 nt | |

Prn_20AALinker_Brka

| SEQ ID NO: 57 consists of from 5′ end to 3′ end: | | 57 |
|---|---|---|
| 5′ UTR SEQ ID NO: 2, mRNA ORF SEQ ID NO: 58, and | | |
| 3′ UTR SEQ ID NO: 4. | | |
| 5′ UTR | GGGAAAUAAGAGAGAAAAGAAGAGUAAGAAGAAAUAUAA | 2 |
| | GACCCCGGCGCCGCCACC | |
| ORF of mRNA | AUGGAGACCCCUGCCCAGCUGCUGUUCCUGCUGCUGCUUU | 58 |
| Construct | GGCUGCCAGACACCACCGGAGACUGGAACAACCAGGCCAU | |
| (excluding the stop | CGUGAAGACCGGCGAGAGACAGCACGGCAUCCACAUCCAA | |
| codon) | GGCAGCGACCCCGGCGGCGUGAGAACCGCCUCCGGCACCA | |
| | CAAUCAAGGUGAGCGGCAGACAGGCCCAGGGCAUCCUGC | |
| | UGGAGAACCCUGCCGCCGAGCUGCAGUUCAGAAACGGCGC | |
| | CGUGACCAGCAGCGGCCAGCUGUCCGACGACGGCAUCAGA | |
| | AGAUUCCUGGGCACCGUGACCGUGAAGGCCGGCAAGCUG | |
| | GUGGCCGACCACGCCACCCUGGCCAACGUGGGUGAUACCU | |
| | GGGACGACGACGGUAUUGCCCUGUACGUGGCCGGCGAGC | |
| | AAGCACAGGCCAGCAUCGCCGACAGCACCCUGCAAGGCGC | |
| | AGGCGGCGUGCAGAUCGAGAGAGGCGCCAACGUGGUGGU | |
| | GCAGAGAAGCGCCAUUGUUGACGGCGGCCUGCACAUCGG | |
| | CGCCCUGCAGAGCCUGCAGCCUGAGGACCUGCCUCCUAGC | |
| | AGAGUGGUGCUGAGAGACACAAACGUCGUCGCCGUGCCA | |
| | GCGUCCGGAGCUCCAGCAGCCGUGAGCGUGCUGGGCGCCA | |
| | GCGAGCUGACCCUCGACGGAGGACACAUCACCGGUGGCAG | |
| | GGCCGCCGGUGUGGCCGCCAUGCAGGGAGCCGUUGUGCA | |
| | UCUACAAAGAGCUACCAUCCGACGGGGCGACGCUCCGGCU | |
| | GGUGGAGCGGUCCCUGGCGGUGCCGUCCCCGGAGGAGCA | |
| | GUGCCAGGCGGUUUCGGCCCGGGUGGAUUCGGCCCUGUG | |
| | CUAGACGGCUGGUACGGCGUGGACGUGAGUGGCUCCAGC | |
| | GUGGAGCUGGCCCAGAGUAUUGUUGAGGCCCCUGAGCUC | |
| | GGCGCUGCCAUCAGAGUGGGCCGUGGUGCCAGAGUGACC | |
| | GUUAGCGGUGGCUCACUUAGCGCCCCUCACGGCAACGUGA | |
| | UCGAAACUGGAGGAGCGAGACGAUUCGCACCUCAGGCCG | |
| | CCCCUCUGAGCAUAACGCUGCAAGCGGGCGCCCACGCGCA | |
| | AGGCAAGGCCCUGCUGUACAGAGUGCUGCCUGAGCCUGU | |
| | GAAGUUAACUCUGACGGGAGGUGCCGACGCACAAGGUGA | |
| | CAUCGUGGCCACGGAACUUCCUAGCAUCCCUGGCACAUCU | |
| | AUCGGCCCUCUGGACGUUGCGUUAGCUUCGCAGGCCCGUU | |
| | GGACCGGCGCCACCAGAGCCGUGGACUCCCUAAGCAUCGA | |
| | CAACGCCGUGUGGGUCAUGACCGACAACUCAAACGUGGG | |
| | CGCGCUGAGAUUGGCCUCAGACGGCAGUGUCGAUUUCCA | |
| | ACAACCUGCCGAAGCAGGAAGAUUCAAGGUGCUGACUGU | |
| | CAAUACACUGGCCGGAAGCGGCCUGUUCAGAAUGAACGU | |
| | GUUCGCCGACCUGGGACUCUCAGAUAAGCUGGUCGUGAU | |
| | GCAGGACGCAAGUGGCCAGCACAGACUGUGGGUCAGAAA | |
| | CUCGGGCUCCGAGCCAGCCAGUGCCAAUACUUUGCUGUUG | |
| | GUGCAAACCCCUCUGGGCAGUGCCGCCACCUUCACUCUCG | |
| | CCAACAAGGACGGCAAGGUGGACAUCGGCACCUACAGAU | |
| | ACAGGCUGGCAGCAAACGGAAACGGCCAGUGGAGCCUAG | |
| | UCGGAGCAAAGGCCCCUCCUGCCCCUAAGCCUGCUCCACA | |
| | GCCUGGCCCACAGCCUCCGCAACCUCCGCAGCCUCAGCCA | |
| | GAGGCCCCAGCCCCACAACCUCCUGCAGGCAGAGAGCUGA | |
| | GUGCCGCUGCUAACGCCGCCGUUAAUACCGGAGGUGUCG | |
| | GCCUCGCGUCUACCUUGUGGUACGCCGAGAGCAACGGAG | |
| | GAGGUGGCUCUGGCGGAGGUGGAAGCGGUGGAGGCGGAA | |
| | GUGGUGGCGGAGGCAGCCAGGCCCCUCAGCCUCCGGUGGC | |
| | CGGCGCCCCACACGCCCAAGACGCAGGGCAGGAGGGCGAG | |
| | UUCGACCACAGAGACAACACCCUGAUCGCCGUGUUCGACG | |
| | ACGGCGUGGGCAUCAACCUGGACGACGACCCUGACGAGCU | |
| | GGGCGAAACAGCCCCUCCUACCCUGAAGGACAUCCACAUC | |
| | AGCGUGGAGCACAAGAACCCUAUGAGCAAGCCUGCCAUC | |
| | GGCGUGAGAGUGAGCGGGGCCGGUAGGGCCCUGACCCUC | |
| | GCUGGCAGCACCAUCGACGCUACCGAGGGCGGCAUCCCUG | |
| | CCGUUGUCAGAAGAGGCGGCACCCUGGAACUGGACGGGG | |
| | UGACCGUCGCCGGCGGAGAGGGCAUGGAGCCUAUGACCG | |
| | UGAGCGACGCAGGCAGCCGACUCAGUGUGCGGGGAGGUG | |
| | UGCUGGGAGGAGAGGCCCCGGGCGUGGGGUUAGUGCGAG | |
| | CCGCUCAGGGCGGACAGGCUAGCAUAAUCGACGCAACCCU | |
| | GCAGAGCAUCCUGGGACCCGCCCUGAUAGCGGACGGUGGC | |
| | UCAAUCUCAGUGGCUGGAGGCUCCAUUGACAUGGACAUG | |
| | GGCCCAGGCUUCCCUCCUCCACCACCUCCUCUGCCUGGAG | |
| | CACCUCUGGCCGCCCACCCUCCGCUCGACAGAGUGGCCGC | |
| | CGUGCACGCUGGACAGGACGGUAAGGUGACCCUGAGAGA | |
| | GGUGGCGCUCCGGGCGCACGGACCCCAGGCCACAGGAGUG | |
| | UACGCCUACAUGCCUGGCAGCGAGAUCACACUGCAGGGA | |
| | GGUACUGUGUCCGUCCAGGGAGACGACGGUGCCGGAGUC | |
| | GUCGCAGGAGCCGGCCUGCUGGACGCCCUGCCUCCUGGAG | |
| | GAACCGUGAGACUCGACGGUACCACCGUGUCCACCGACGG | |
| | CGCUAACACCGACGCCGUGCUGGUGAGAGGGGACGCCGCC | |
| | AGAGCCGAGGUGGUGAACACCGUGCUGAGAACCGCCAAG | |
| | AGCUUGGCCGCAGGAGUUUCCGCGCAGCACGGAGGCCGG | |
| | GUAACCCUGCGGCAGACCAGAAUUGAAACGGCUGGUGCC | |
| | GGUGCCGAAGGUAUUUCGGUACUAGGAUUCGAGCCUCAG | |
| | AGCGGCAGCGGUCCAGCUUCGGUAGACAUGCAGGGUGGU | |
| | AGCAUUACCACGACCGGCAACAGGGCCGCCGGGAUCGCGC | |
| | UGACCCACGGCUCCGCGAGACUGGAGGGAGUCGCCGUGCG | |
| | AGCAGAAGGAUCCGGAUCCAGCGCCGCCCAGCUGGCAAAC | |
| | GGAGUUCUUGUGGUAUCCGCAGGCUCACUCGCCUCUGCGC | |
| | AGUCUGGCGCUAUAUCUGUGACUGACACCCCUCUGAAGC | |
| | UGAUGCCAGGAGCUCUCGCUUCCAGCACUGUAAGCGUCA | |
| | GACUUACUGACGGAGCCACUGCACAAGGAGGUAACGGCG | |
| | UAUUCCUGCAGCAGCAUUCAACUAUACCUGUGGCUGUGG | |
| | CUCUUGAGAGCGGAGCGCUGGCACGCGGCGACAUCGUGG | |
| | CCGACGGGAACAAGCCCCUCGACGCCGGUAUCAGCUUGUC | |
| | AGUGGCCUCUGGAGCCGCCUGGCACGGGGCUACCCAAGUG | |
| | CUGCAGUCUGCCACGCUGGGCAAGGGUGGCACCUGGGUU | |
| | GUUAACGCCGACAGCAGAGUGCAGGACAUGAGCAUGCGG | |
| | GGCGGAAGGGUUGAGUUCCAGGCUCCGGCCCCUGAGGCC | |
| | AGCUACAAGACCUUAACACUCCAGACCUUAGACGGAAAC | |
| | GGCGUGUUCGUGCUGAACACCAACGUCGCAGCGGGACAG | |
| | AACGACCAGCUGAGAGUUACCGGCAGGGCUGACGGUCAG | |
| | CACAGAGUCCUGGUCAGGAACGCCGGAGGCGAGGCCGAU | |
| | AGCCGGGGUGCGCGGUUAGGACUGGUUCACACCCAGGGC | |
| | CAGGGCAACGCAGUAUUCAGAUUAGCAAACGUGGGUAAG | |
| | GCCGUGGACCUGGGCACGUGGAGAUACAGUUUGGCCGAG | |
| | GACCCUAAGACCCACGUGUGGAGUCUGCAAAGAGCCGGU | |
| | CAGGCUCUGUCAGGCGCUGCCAACGCCGCCGUGAACGCUG | |
| | CGGACCUGAGUAGUAUCGCCCUAGCAGAAAGCAACGCC | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding amino | METPAQLLFLLLLWLPDTTGDWNNQAIVKTGERQHGIHIQGS | 59 |
| acid sequence | DPGGVRTASGTTIKVSGRQAQGILLENPAAELQFRNGAVTSSG | |
| | QLSDDGIRRFLGTVTVKAGKLVADHATLANVGDTWDDDGIA | |
| | LYVAGEQAQASIADSTLQGAGGVQIERGANVVVQRSAIVDGG | |
| | LHIGALQSLQPEDLPPSRVVLRDTNVVAVPASGAPAAVSVLGA | |
| | SELTLDGGHITGGRAAGVAAMQGAVVHLQRATIRRGDAPAG | |
| | GAVPGGAVPGGAVPGGFGPGGFGPVLDGWYGVDVSGSSVEL | |
| | AQSIVEAPELGAAIRVGRGARVTVSGGSLSAPHGNVIETGGAR | |
| | RFAPQAAPLSITLQAGAHAQGKALLYRVLPEPVKLTLTGGAD | |
| | AQGDIVATELPSIPGTSIGPLDVALASQARWTGATRAVDSLSID | |
| | NAVWVMTDNSNVGALRLASDGSVDFQQPAEAGRFKVLTVNT | |
| | LAGSGLFRMNVFADLGLSDKLVVMQDASGQHRLWVRNSGSE | |
| | PASANTLLLVQTPLGSAATFTLANKDGKVDIGTYRYRLAANG | |
| | NGQWSLVGAKAPPAPKPAPQPGPQPPQPPQPQPEAPAPQPPAG | |
| | RELSAAANAAVNTGGVGLASTLWYAESNGGGGSGGGGSGGG | |
| | GSGGGGSQAPQPPVAGAPHAQDAGQEGEFDHRDNTLIAVFDD | |
| | GVGINLDDDPDELGETAPPTLKDIHISVEHKNPMSKPAIGVRVS | |
| | GAGRALTLAGSTIDATEGGIPAVVRRGGTLELDGVTVAGGEG | |
| | MEPMTVSDAGSRLSVRGGVLGGEAPGVGLVRAAQGGQASIID | |
| | ATLQSILGPALIADGGSISVAGGSIDMDMGPGFPPPPPPLPGAPL | |
| | AAHPPLDRVAAVHAGQDGKVTLREVALRAHGPQATGVYAY | |
| | MPGSEITLQGGTVSVQGDDGAGVVAGAGLLDALPPGGTVRL | |
| | DGTTVSTDGANTDAVLVRGDAARAEVVNTVLRTAKSLAAGV | |
| | SAQHGGRVTLRQTRIETAGAGAEGISVLGFEPQSGSGPASVDM | |
| | QGGSITTTGNRAAGIALTHGSARLEGVAVRAEGSGSSAAQLA | |
| | NGVLVVSAGSLASAQSGAISVTDTPLKLMPGALASSTVSVRLT | |
| | DGATAQGGNGVFLQQHSTIPVAVALESGALARGDIVADGNKP | |
| | LDAGISLSVASGAAWHGATQVLQSATLGKGGTWVVNADSRV | |
| | QDMSMRGGRVEFQAPAPEASYKTLTLQTLDGNGVFVLNTNV | |
| | AAGQNDQLRVTGRADGQHRVLVRNAGGEADSRGARLGLVH | |
| | TQGQGNAVFRLANVGKAVDLGTWRYSLAEDPKTHVWSLQR | |
| | AGQALSGAANAAVNAADLSSIALAESNA | |
| PolyA tail | 100 nt | |

Brka_20AALinker_Prn

| SEQ ID NO: 60 consists of from 5′ end to 3′ end: | | 60 |
|---|---|---|
| 5′ UTR SEQ ID NO: 2, mRNA ORF SEQ ID NO: 61, and | | |
| 3′ UTR SEQ ID NO: 4. | | |
| 5′ UTR | GGGAAAUAAGAGAGAAAAGAAGAGUAAGAAGAAAUAUAA | 2 |
| | GACCCCGGCGCCGCCACC | |
| ORF of mRNA | AUGGAGACCCCUGCCCAGCUGCUGUUCCUGCUGCUGCUUU | 61 |
| Construct | GGCUGCCAGACACCACCGGACAGGCCCCUCAGCCUCCGGU | |
| (excluding the stop | GGCCGGCGCCCCACACGCCCAAGACGCAGGGCAGGAGGGC | |
| codon) | GAGUUCGACCACAGAGACAACACCCUGAUCGCCGUGUUCG | |
| | ACGACGGCGUGGGCAUCAACCUGGACGACGACCCUGACGA | |
| | GCUGGGCGAAACAGCCCCUCCUACCCUGAAGGACAUCCAC | |
| | AUCAGCGUGGAGCACAAGAACCCUAUGAGCAAGCCUGCC | |
| | AUCGGCGUGAGAGUGAGCGGGGCCGGUAGGGCCCUGACC | |
| | CUCGCUGGCAGCACCAUCGACGCUACCGAGGGCGGCAUCC | |
| | CUGCCGUUGUCAGAAGAGGCGGCACCCUGGAACUGGACG | |
| | GGGUGACCGUCGCCGGCGGAGAGGGCAUGGAGCCUAUGA | |
| | CCGUGAGCGACGCAGGCAGCCGACUCAGUGUGCGGGGAG | |
| | GUGUGCUGGGAGGAGAGGCCCCGGGCGUGGGGUUAGUGC | |
| | GAGCCGCUCAGGGCGGACAGGCUAGCAUAAUCGACGCAA | |
| | CCCUGCAGAGCAUCCUGGGACCCGCCCUGAUAGCGGACGG | |
| | UGGCUCAAUCUCAGUGGCUGGAGGCUCCAUUGACAUGGA | |
| | CAUGGGCCCAGGCUUCCCUCCUCCACCACCUCCUCUGCCU | |
| | GGAGCACCUCUGGCCGCCCACCCUCCGCUCGACAGAGUGG | |
| | CCGCCGUGCACGCUGGACAGGACGGUAAGGUGACCCUGA | |
| | GAGAGGUGGCGCUCCGGGCGCACGGACCCCAGGCCACAGG | |
| | AGUGUACGCCUACAUGCCUGGCAGCGAGAUCACACUGCA | |
| | GGGAGGUACUGUGUCCGUCCAGGGAGACGACGGUGCCGG | |
| | AGUCGUCGCAGGAGCCGGCCUGCUGGACGCCCUGCCUCCU | |
| | GGAGGAACCGUGAGACUCGACGGUACCACCGUGUCCACCG | |
| | ACGGCGCUAACACCGACGCCGUGCUGGUGAGAGGGGACG | |
| | CCGCCAGAGCCGAGGUGGUGAACACCGUGCUGAGAACCGC | |
| | CAAGAGCUUGGCCGCAGGAGUUUCCGCGCAGCACGGAGG | |
| | CCGGGUAACCCUGCGGCAGACCAGAAUUGAAACGGCUGG | |
| | UGCCGGUGCCGAAGGUAUUUCGGUACUAGGAUUCGAGCC | |
| | UCAGAGCGGCAGCGGUCCAGCUUCGGUAGACAUGCAGGG | |
| | UGGUAGCAUUACCACGACCGGCAACAGGGCCGCCGGGAUC | |
| | GCGCUGACCCACGGCUCCGCGAGACUGGAGGGAGUCGCCG | |
| | UGCGAGCAGAAGGAUCCGGAUCCAGCGCCGCCCAGCUGGC | |
| | AAACGGAGUUCUUGUGGUAUCCGCAGGCUCACUCGCCUC | |
| | UGCGCAGUCUGGCGCUAUAUCUGUGACUGACACCCCUCUG | |
| | AAGCUGAUGCCAGGAGCUCUCGCUUCCAGCACUGUAAGC | |
| | GUCAGACUUACUGACGGAGCCACUGCACAAGGAGGUAAC | |
| | GGCGUAUUCCUGCAGCAGCAUUCAACUAUACCUGUGGCU | |
| | GUGGCUCUUGAGAGCGGAGCGCUGGCACGCGGCGACAUC | |
| | GUGGCCGACGGGAACAAGCCCCUCGACGCCGGUAUCAGCU | |
| | UGUCAGUGGCCUCUGGAGCCGCCUGGCACGGGGCUACCCA | |
| | AGUGCUGCAGUCUGCCACGCUGGGCAAGGGUGGCACCUG | |
| | GGUUGUUAACGCCGACAGCAGAGUGCAGGACAUGAGCAU | |
| | GCGGGGCGGAAGGGUUGAGUUCCAGGCUCCGGCCCCUGA | |
| | GGCCAGCUACAAGACCUUAACACUCCAGACCUUAGACGGA | |
| | AACGGCGUGUUCGUGCUGAACACCAACGUCGCAGCGGGA | |
| | CAGAACGACCAGCUGAGAGUUACCGGCAGGGCUGACGGU | |
| | CAGCACAGAGUCCUGGUCAGGAACGCCGGAGGCGAGGCC | |
| | GAUAGCCGGGGUGCGCGGUUAGGACUGGUUCACACCCAG | |
| | GGCCAGGGCAACGCAGUAUUCAGAUUAGCAAACGUGGGU | |
| | AAGGCCGUGGACCUGGGCACGUGGAGAUACAGUUUGGCC | |
| | GAGGACCCUAAGACCCACGUGUGGAGUCUGCAAAGAGCC | |
| | GGUCAGGCUCUGUCAGGCGCUGCCAACGCCGCCGUGAACG | |
| | CUGCGGACCUGAGUAGUAUCGCCCUAGCAGAAAGCAACG | |
| | CCGGAGGAGGUGGCUCUGGCGGAGGUGGAAGCGGUGGAG | |
| | GCGGAAGUGGUGGCGGAGGCAGCGACUGGAACAACCAGG | |
| | CCAUCGUGAAGACCGGCGAGAGACAGCACGGCAUCCACAU | |
| | CCAAGGCAGCGACCCCGGCGGCGUGAGAACCGCCUCCGGC | |
| | ACCACAAUCAAGGUGAGCGGCAGACAGGCCCAGGGCAUCC | |
| | UGCUGGAGAACCCUGCCGCCGAGCUGCAGUUCAGAAACG | |
| | GCGCCGUGACCAGCAGCGGCCAGCUGUCCGACGACGGCAU | |
| | CAGAAGAUUCCUGGGCACCGUGACCGUGAAGGCCGGCAA | |
| | GCUGGUGGCCGACCACGCCACCCUGGCCAACGUGGGUGAU | |
| | ACCUGGGACGACGACGGUAUUGCCCUGUACGUGGCCGGC | |
| | GAGCAAGCACAGGCCAGCAUCGCCGACAGCACCCUGCAAG | |
| | GCGCAGGCGGCGUGCAGAUCGAGAGAGGCGCCAACGUGG | |
| | UGGUGCAGAGAAGCGCCAUUGUUGACGGCGGCCUGCACA | |
| | UCGGCGCCCUGCAGAGCCUGCAGCCUGAGGACCUGCCUCC | |
| | UAGCAGAGUGGUGCUGAGAGACACAAACGUCGUCGCCGU | |
| | GCCAGCGUCCGGAGCUCCAGCAGCCGUGAGCGUGCUGGGC | |
| | GCCAGCGAGCUGACCCUCGACGGAGGACACAUCACCGGUG | |
| | GCAGGGCCGCCGGUGUGGCCGCCAUGCAGGGAGCCGUUG | |
| | UGCAUCUACAAAGAGCUACCAUCCGACGGGGCGACGCUCC | |
| | GGCUGGUGGAGCGGUCCCUGGCGGUGCCGUCCCCGGAGG | |
| | AGCAGUGCCAGGCGGUUUCGGCCCGGGUGGAUUCGGCCC | |
| | UGUGCUAGACGGCUGGUACGGCGUGGACGUGAGUGGCUC | |
| | CAGCGUGGAGCUGGCCCAGAGUAUUGUUGAGGCCCCUGA | |
| | GCUCGGCGCUGCCAUCAGAGUGGGCCGUGGUGCCAGAGU | |
| | GACCGUUAGCGGUGGCUCACUUAGCGCCCCUCACGGCAAC | |
| | GUGAUCGAAACUGGAGGAGCGAGACGAUUCGCACCUCAG | |
| | GCCGCCCCUCUGAGCAUAACGCUGCAAGCGGGCGCCCACG | |
| | CGCAAGGCAAGGCCCUGCUGUACAGAGUGCUGCCUGAGCC | |
| | UGUGAAGUUAACUCUGACGGGAGGUGCCGACGCACAAGG | |
| | UGACAUCGUGGCCACGGAACUUCCUAGCAUCCCUGGCACA | |
| | UCUAUCGGCCCUCUGGACGUUGCGUUAGCUUCGCAGGCCC | |
| | GUUGGACCGGCGCCACCAGAGCCGUGGACUCCCUAAGCAU | |
| | CGACAACGCCGUGUGGGUCAUGACCGACAACUCAAACGU | |
| | GGGCGCGCUGAGAUUGGCCUCAGACGGCAGUGUCGAUUU | |
| | CCAACAACCUGCCGAAGCAGGAAGAUUCAAGGUGCUGAC | |
| | UGUCAAUACACUGGCCGGAAGCGGCCUGUUCAGAAUGAA | |
| | CGUGUUCGCCGACCUGGGACUCUCAGAUAAGCUGGUCGU | |
| | GAUGCAGGACGCAAGUGGCCAGCACAGACUGUGGGUCAG | |
| | AAACUCGGGCUCCGAGCCAGCCAGUGCCAAUACUUUGCUG | |
| | UUGGUGCAAACCCCUCUGGGCAGUGCCGCCACCUUCACUC | |
| | UCGCCAACAAGGACGGCAAGGUGGACAUCGGCACCUACA | |
| | GAUACAGGCUGGCAGCAAACGGAAACGGCCAGUGGAGCC | |
| | UAGUCGGAGCAAAGGCCCCUCCUGCCCCUAAGCCUGCUCC | |
| | ACAGCCUGGCCCACAGCCUCCGCAACCUCCGCAGCCUCAG | |
| | CCAGAGGCCCCAGCCCCACAACCUCCUGCAGGCAGAGAGC | |
| | UGAGUGCCGCUGCUAACGCCGCCGUUAAUACCGGAGGUG | |
| | UCGGCCUCGCGUCUACCUUGUGGUACGCCGAGAGCAAC | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding amino | METPAQLLFLLLLWLPDTTGQAPQPPVAGAPHAQDAGQEGEF | 62 |
| acid sequence | DHRDNTLIAVFDDGVGINLDDDPDELGETAPPTLKDIHISVEH | |
| | KNPMSKPAIGVRVSGAGRALTLAGSTIDATEGGIPAVVRRGGT | |
| | LELDGVTVAGGEGMEPMTVSDAGSRLSVRGGVLGGEAPGVG | |
| | LVRAAQGGQASIIDATLQSILGPALIADGGSISVAGGSIDMDMG | |
| | PGFPPPPPPLPGAPLAAHPPLDRVAAVHAGQDGKVTLREVALR | |
| | AHGPQATGVYAYMPGSEITLQGGTVSVQGDDGAGVVAGAGL | |
| | LDALPPGGTVRLDGTTVSTDGANTDAVLVRGDAARAEVVNT | |
| | VLRTAKSLAAGVSAQHGGRVTLRQTRIETAGAGAEGISVLGFE | |
| | PQSGSGPASVDMQGGSITTTGNRAAGIALTHGSARLEGVAVR | |
| | AEGSGSSAAQLANGVLVVSAGSLASAQSGAISVTDTPLKLMP | |
| | GALASSTVSVRLTDGATAQGGNGVFLQQHSTIPVAVALESGA | |
| | LARGDIVADGNKPLDAGISLSVASGAAWHGATQVLQSATLGK | |
| | GGTWVVNADSRVQDMSMRGGRVEFQAPAPEASYKTLTLQTL | |
| | DGNGVFVLNTNVAAGQNDQLRVTGRADGQHRVLVRNAGGE | |
| | ADSRGARLGLVHTQGQGNAVFRLANVGKAVDLGTWRYSLA | |
| | EDPKTHVWSLQRAGQALSGAANAAVNAADLSSIALAESNAG | |
| | GGGSGGGGSGGGGSGGGGSDWNNQAIVKTGERQHGIHIQGS | |
| | DPGGVRTASGTTIKVSGRQAQGILLENPAAELQFRNGAVTSSG | |
| | QLSDDGIRRFLGTVTVKAGKLVADHATLANVGDTWDDDGIA | |
| | LYVAGEQAQASIADSTLQGAGGVQIERGANVVVQRSAIVDGG | |
| | LHIGALQSLQPEDLPPSRVVLRDTNVVAVPASGAPAAVSVLGA | |
| | SELTLDGGHITGGRAAGVAAMQGAVVHLQRATIRRGDAPAG | |
| | GAVPGGAVPGGAVPGGFGPGGFGPVLDGWYGVDVSGSSVEL | |
| | AQSIVEAPELGAAIRVGRGARVTVSGGSLSAPHGNVIETGGAR | |
| | RFAPQAAPLSITLQAGAHAQGKALLYRVLPEPVKLTLTGGAD | |
| | AQGDIVATELPSIPGTSIGPLDVALASQARWTGATRAVDSLSID | |
| | NAVWVMTDNSNVGALRLASDGSVDFQQPAEAGRFKVLTVNT | |
| | LAGSGLFRMNVFADLGLSDKLVVMQDASGQHRLWVRNSGSE | |
| | PASANTLLLVQTPLGSAATFTLANKDGKVDIGTYRYRLAANG | |
| | NGQWSLVGAKAPPAPKPAPQPGPQPPQPPQPQPEAPAPQPPAG | |
| | RELSAAANAAVNTGGVGLASTLWYAESN | |
| PolyA tail | 100 nt | |

C180_FurinF2A_Tetanus

| SEQ ID NO: 63 consists of from 5′ end to 3′ end: | | 63 |
|---|---|---|
| 5′ UTR SEQ ID NO: 2, mRNA ORF SEQ ID NO: 64, and | | |
| 3′ UTR SEQ ID NO: 4. | | |
| 5′ UTR | GGGAAAUAAGAGAGAAAAGAAGAGUAAGAAGAAAUAUAA | 2 |
| | GACCCCGGCGCCGCCACC | |
| ORF of mRNA | AUGGAAACCCCUGCUCAGCUGCUGUUCCUGCUGCUGCUGU | 64 |
| Construct | GGCUGCCUGAUACCACAGGGGACGAUCCUCCAGCCACCGU | |
| (excluding the stop | GUAUAAGUACGACAGCAGACCUCCUGAGGACGUGUUCCA | |
| codon) | GAACGGCUUUACCGCCUGGGGCAACAACGACAACGUGCU | |
| | GGAUCACCUGACCGGCAGAUCCUGUCAAGUGGGCAGCAG | |
| | CAAUAGCGCCUUCGUGUCCACCUCUAGCAGCAGACGGUAC | |
| | ACAGAGGUGUACCUGGAACACCGGAUGCAAGAGGCCGUG | |
| | GAAGCCGAAAGAGCCGGCAGAGGAACAGGCCACUUCAUC | |
| | GGCUACAUCUACGAAGUGCGGGCCGACAACAACUUCUAC | |
| | GGCGCUGCCAGCAGCUACUUCGAGUACGUGGACACCUACG | |
| | GCGACAACGCCGGAAGAAUACUGGCUGGCGCUCUGGCCAC | |
| | AUACCAGUCUGGAUAUCUGGCCCACAGACGGAUCCCUCCA | |
| | GAGAACAUUCGGAGAGUGACCCGGGUGUACCACAACGGC | |
| | AUUACCGGCGAGACAACCACCACCGAGUACAGCAACGCCA | |
| | GAUACGUGUCCCAGCAGACCCGGGCCAAUCCUAAUCCUUA | |
| | CACCAGCCGGGCCAAGAGAAGCGGAAGCGGCGCUCCCGUG | |
| | AAGCAGACCCUGAACUUCGACCUGCUGAAGCUGGCCGGCG | |
| | ACGUGGAGAGCAACCCCGGCCCCAUGGAGACACCCGCCCA | |
| | GCUGCUCUUCCUCCUGCUGCUCUGGCUGCCCGACACCACC | |
| | GGCAAGAACCUGGACUGCUGGGUGGACAACGAGGAGGAC | |
| | AUCGACGUGAUCCUGAAGAAGAGCACCAUCCUGAACCUG | |
| | GACAUCAACAACGACAUCAUCAGCGACAUCAGCGGCUUCA | |
| | ACAGCGCCGUGAUCACCUACCCCGACGCCCAACUGGUGCC | |
| | CGGCAUCAACGGCAAGGCCAUCCACCUGGUGAACAACGAG | |
| | GCCAGCGAGGUGAUCGUGCACAAGGCCAUGGACAUCGAG | |
| | UACAACGACAUGUUCAACAACUUCGCCGUGAGCUUCUGG | |
| | CUGCGGGUGCCCAAGGUGAGCGCCAGCCACCUGGAGCAGU | |
| | ACGGCACCAACGAGUACAGCAUCAUCAGCAGCAUGAAGA | |
| | AGCACAGCCUGAGCAUCGGCAGCGGCUGGAGCGUGAGCC | |
| | UGAAGGGCAACAACCUGAUCUGGACCCUGAAGGAUAGCG | |
| | CCGGCGAGGUGAGACAGAUCACCUUCCGGGACCUGCCCGA | |
| | CAAGUUCAACGCCUACCUGGCCAACAAGUGGGUGUUCAU | |
| | UACCAUCACCAACGACCGGCUGAGCAGCGCCAACCUGUAC | |
| | AUCAACGGCGUGCUGAUGGGCAGCGCCGAGAUCACCGGCC | |
| | UGGGAGCCAUCCGGGAGGACAACAACAUCGCCCUGAAGC | |
| | UGGACCGGUGCAACAACAAUAACCAGUACGUGAGCAUCG | |
| | ACAAGUUCCGGAUCUUCUGCAAGGCCCUGAACCCCAAGGA | |
| | GAUCGAGAAGCUGUACACCAGCUACCUGAGCAUCACCUUC | |
| | CUGCGGGACUUCUGGGGCAAUCCCCUGCGGUACGACACCG | |
| | AGUACUACCUGAUCCCCGUGGCCUCAAGCAGCAAGGACGU | |
| | GCAGUUGAAGAACAUCGCCGACUACAUGUACCUGACCAA | |
| | CGCCCCGAGCUACACCAACGGCAAGCUGAACAUCUACUAC | |
| | CGGCGGCUGUACAACGGCCUGAAGUUCAUCAUCAAGCGG | |
| | UACACCCCUAACAACGAGAUCGACAGCUUCGUGAAGAGC | |
| | GGCGACUUCAUCAAGCUGUACGUGAGCUACAACAACAAC | |
| | GAGCACAUCGUGGGCUACCCCAAGGACGGCAACGCCUUCA | |
| | ACAACCUGGACCGGAUCCUGCGGGUGGGCUACAACGCCCC | |
| | AGGCAUUCCCCUGUACAAGAAGAUGGAGGCCGUGAAGCU | |
| | GCGGGACCUGAAGACCUACAGCGUGCAGCUGAAACUGUA | |
| | CGACGACAAGAACGCCAGCCUGGGCCUGGUGGGCACCCAC | |
| | AACGGCCAGAUCGGCAACGACCCCAACCGGGACAUCCUGA | |
| | UCGCCAGCAACUGGUACUUUAACCACCUGAAGGACAAGA | |
| | UCCUGGGCUGCGACUGGUACUUCGUGCCCACCGACGAGGG | |
| | CUGGACCAACGAC | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding amino | METPAQLLFLLLLWLPDTTGDDPPATVYKYDSRPPEDVFQNG | 65 |
| acid sequence | FTAWGNNDNVLDHLTGRSCQVGSSNSAFVSTSSSRRYTEVYL | |
| | EHRMQEAVEAERAGRGTGHFIGYIYEVRADNNFYGAASSYFE | |
| | YVDTYGDNAGRILAGALATYQSGYLAHRRIPPENIRRVTRVY | |
| | HNGITGETTTTEYSNARYVSQQTRANPNPYTSRAKRSGSGAPV | |
| | KQTLNFDLLKLAGDVESNPGPMETPAQLLFLLLLWLPDTTGK | |
| | NLDCWVDNEEDIDVILKKSTILNLDINNDIISDISGFNSAVITYP | |
| | DAQLVPGINGKAIHLVNNEASEVIVHKAMDIEYNDMFNNFAV | |
| | SFWLRVPKVSASHLEQYGTNEYSIISSMKKHSLSIGSGWSVSL | |
| | KGNNLIWTLKDSAGEVRQITFRDLPDKFNAYLANKWVFITITN | |
| | DRLSSANLYINGVLMGSAEITGLGAIREDNNIALKLDRCNNNN | |
| | QYVSIDKFRIFCKALNPKEIEKLYTSYLSITFLRDFWGNPLRYD | |
| | TEYYLIPVASSSKDVQLKNIADYMYLTNAPSYTNGKLNIYYRR | |
| | LYNGLKFIIKRYTPNNEIDSFVKSGDFIKLYVSYNNNEHIVGYP | |
| | KDGNAFNNLDRILRVGYNAPGIPLYKKMEAVKLRDLKTYSVQ | |
| | LKLYDDKNASLGLVGTHNGQIGNDPNRDILIASNWYFNHLKD | |
| | KILGCDWYFVPTDEGWTND | |
| PolyA tail | 100 nt | |

C180_20AALinker_Tetanus

| SEQ ID NO: 66 consists of from 5′ end to 3′ end: | | 66 |
|---|---|---|
| 5′ UTR SEQ ID NO: 2, mRNA ORF SEQ ID NO: 67, and | | |
| 3′ UTR SEQ ID NO: 4. | | |
| 5′ UTR | GGGAAAUAAGAGAGAAAAGAAGAGUAAGAAGAAAUAUAA | 2 |
| | GACCCCGGCGCCGCCACC | |
| ORF of mRNA | AUGGAAACCCCUGCUCAGCUGCUGUUCCUGCUGCUGCUGU | 67 |
| Construct | GGCUGCCUGAUACCACAGGGGACGAUCCUCCAGCCACCGU | |
| (excluding the stop | GUAUAAGUACGACAGCAGACCUCCUGAGGACGUGUUCCA | |
| codon) | GAACGGCUUUACCGCCUGGGGCAACAACGACAACGUGCU | |
| | GGAUCACCUGACCGGCAGAUCCUGUCAAGUGGGCAGCAG | |
| | CAAUAGCGCCUUCGUGUCCACCUCUAGCAGCAGACGGUAC | |
| | ACAGAGGUGUACCUGGAACACCGGAUGCAAGAGGCCGUG | |
| | GAAGCCGAAAGAGCCGGCAGAGGAACAGGCCACUUCAUC | |
| | GGCUACAUCUACGAAGUGCGGGCCGACAACAACUUCUAC | |
| | GGCGCUGCCAGCAGCUACUUCGAGUACGUGGACACCUACG | |
| | GCGACAACGCCGGAAGAAUACUGGCUGGCGCUCUGGCCAC | |
| | AUACCAGUCUGGAUAUCUGGCCCACAGACGGAUCCCUCCA | |
| | GAGAACAUUCGGAGAGUGACCCGGGUGUACCACAACGGC | |
| | AUUACCGGCGAGACAACCACCACCGAGUACAGCAACGCCA | |
| | GAUACGUGUCCCAGCAGACCCGGGCCAAUCCUAAUCCUUA | |
| | CACCAGCGGAGGAGGUGGCUCUGGCGGAGGUGGAAGCGG | |
| | UGGAGGCGGAAGUGGUGGCGGAGGCAGCAAGAACCUGGA | |
| | CUGCUGGGUGGACAACGAGGAGGACAUCGACGUGAUCCU | |
| | GAAGAAGAGCACCAUCCUGAACCUGGACAUCAACAACGA | |
| | CAUCAUCAGCGACAUCAGCGGCUUCAACAGCGCCGUGAUC | |
| | ACCUACCCCGACGCCCAACUGGUGCCCGGCAUCAACGGCA | |
| | AGGCCAUCCACCUGGUGAACAACGAGGCCAGCGAGGUGA | |
| | UCGUGCACAAGGCCAUGGACAUCGAGUACAACGACAUGU | |
| | UCAACAACUUCGCCGUGAGCUUCUGGCUGCGGGUGCCCAA | |
| | GGUGAGCGCCAGCCACCUGGAGCAGUACGGCACCAACGAG | |
| | UACAGCAUCAUCAGCAGCAUGAAGAAGCACAGCCUGAGC | |
| | AUCGGCAGCGGCUGGAGCGUGAGCCUGAAGGGCAACAAC | |
| | CUGAUCUGGACCCUGAAGGAUAGCGCCGGCGAGGUGAGA | |
| | CAGAUCACCUUCCGGGACCUGCCCGACAAGUUCAACGCCU | |
| | ACCUGGCCAACAAGUGGGUGUUCAUUACCAUCACCAACG | |
| | ACCGGCUGAGCAGCGCCAACCUGUACAUCAACGGCGUGCU | |
| | GAUGGGCAGCGCCGAGAUCACCGGCCUGGGAGCCAUCCGG | |
| | GAGGACAACAACAUCGCCCUGAAGCUGGACCGGUGCAAC | |
| | AACAAUAACCAGUACGUGAGCAUCGACAAGUUCCGGAUC | |
| | UUCUGCAAGGCCCUGAACCCCAAGGAGAUCGAGAAGCUG | |
| | UACACCAGCUACCUGAGCAUCACCUUCCUGCGGGACUUCU | |
| | GGGGCAAUCCCCUGCGGUACGACACCGAGUACUACCUGAU | |
| | CCCCGUGGCCUCAAGCAGCAAGGACGUGCAGUUGAAGAA | |
| | CAUCGCCGACUACAUGUACCUGACCAACGCCCCGAGCUAC | |
| | ACCAACGGCAAGCUGAACAUCUACUACCGGCGGCUGUACA | |
| | ACGGCCUGAAGUUCAUCAUCAAGCGGUACACCCCUAACAA | |
| | CGAGAUCGACAGCUUCGUGAAGAGCGGCGACUUCAUCAA | |
| | GCUGUACGUGAGCUACAACAACAACGAGCACAUCGUGGG | |
| | CUACCCCAAGGACGGCAACGCCUUCAACAACCUGGACCGG | |
| | AUCCUGCGGGUGGGCUACAACGCCCCAGGCAUUCCCCUGU | |
| | ACAAGAAGAUGGAGGCCGUGAAGCUGCGGGACCUGAAGA | |
| | CCUACAGCGUGCAGCUGAAACUGUACGACGACAAGAACG | |
| | CCAGCCUGGGCCUGGUGGGCACCCACAACGGCCAGAUCGG | |
| | CAACGACCCCAACCGGGACAUCCUGAUCGCCAGCAACUGG | |
| | UACUUUAACCACCUGAAGGACAAGAUCCUGGGCUGCGAC | |
| | UGGUACUUCGUGCCCACCGACGAGGGCUGGACCAACGAC | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding amino | METPAQLLFLLLLWLPDTTGDDPPATVYKYDSRPPEDVFQNG | 68 |
| acid sequence | FTAWGNNDNVLDHLTGRSCQVGSSNSAFVSTSSSRRYTEVYL | |
| | EHRMQEAVEAERAGRGTGHFIGYIYEVRADNNFYGAASSYFE | |
| | YVDTYGDNAGRILAGALATYQSGYLAHRRIPPENIRRVTRVY | |
| | HNGITGETTTTEYSNARYVSQQTRANPNPYTSGGGGSGGGGS | |
| | GGGGSGGGGSKNLDCWVDNEEDIDVILKKSTILNLDINNDIISD | |
| | ISGFNSAVITYPDAQLVPGINGKAIHLVNNEASEVIVHKAMDIE | |
| | YNDMFNNFAVSFWLRVPKVSASHLEQYGTNEYSIISSMKKHS | |
| | LSIGSGWSVSLKGNNLIWTLKDSAGEVRQITFRDLPDKFNAYL | |
| | ANKWVFITITNDRLSSANLYINGVLMGSAEITGLGAIREDNNIA | |
| | LKLDRCNNNNQYVSIDKFRIFCKALNPKEIEKLYTSYLSITFLR | |
| | DFWGNPLRYDTEYYLIPVASSSKDVQLKNIADYMYLTNAPSY | |
| | TNGKLNIYYRRLYNGLKFIIKRYTPNNEIDSFVKSGDFIKLYVS | |
| | YNNNEHIVGYPKDGNAFNNLDRILRVGYNAPGIPLYKKMEAV | |
| | KLRDLKTYSVQLKLYDDKNASLGLVGTHNGQIGNDPNRDILI | |
| | ASNWYFNHLKDKILGCDWYFVPTDEGWTND | |
| PolyA tail | 100 nt | |

Tetanus_20AALinker_C180

| SEQ ID NO: 69 consists of from 5′ end to 3′ end: | | 69 |
|---|---|---|
| 5′ UTR SEQ ID NO: 2, mRNA ORF SEQ ID NO: 70, and | | |
| 3′ UTR SEQ ID NO: 4. | | |
| 5′ UTR | GGGAAAUAAGAGAGAAAAGAAGAGUAAGAAGAAAUAUAA | 2 |
| | GACCCCGGCGCCGCCACC | |
| ORF of mRNA | AUGGAAACCCCUGCUCAGCUGCUGUUCCUGCUGCUGCUGU | 70 |
| Construct | GGCUGCCUGAUACCACAGGGAAGAACCUGGACUGCUGGG | |
| (excluding the stop | UGGACAACGAGGAGGACAUCGACGUGAUCCUGAAGAAGA | |
| codon) | GCACCAUCCUGAACCUGGACAUCAACAACGACAUCAUCAG | |
| | CGACAUCAGCGGCUUCAACAGCGCCGUGAUCACCUACCCC | |
| | GACGCCCAACUGGUGCCCGGCAUCAACGGCAAGGCCAUCC | |
| | ACCUGGUGAACAACGAGGCCAGCGAGGUGAUCGUGCACA | |
| | AGGCCAUGGACAUCGAGUACAACGACAUGUUCAACAACU | |
| | UCGCCGUGAGCUUCUGGCUGCGGGUGCCCAAGGUGAGCG | |
| | CCAGCCACCUGGAGCAGUACGGCACCAACGAGUACAGCAU | |
| | CAUCAGCAGCAUGAAGAAGCACAGCCUGAGCAUCGGCAG | |
| | CGGCUGGAGCGUGAGCCUGAAGGGCAACAACCUGAUCUG | |
| | GACCCUGAAGGAUAGCGCCGGCGAGGUGAGACAGAUCAC | |
| | CUUCCGGGACCUGCCCGACAAGUUCAACGCCUACCUGGCC | |
| | AACAAGUGGGUGUUCAUUACCAUCACCAACGACCGGCUG | |
| | AGCAGCGCCAACCUGUACAUCAACGGCGUGCUGAUGGGC | |
| | AGCGCCGAGAUCACCGGCCUGGGAGCCAUCCGGGAGGACA | |
| | ACAACAUCGCCCUGAAGCUGGACCGGUGCAACAACAAUA | |
| | ACCAGUACGUGAGCAUCGACAAGUUCCGGAUCUUCUGCA | |
| | AGGCCCUGAACCCCAAGGAGAUCGAGAAGCUGUACACCA | |
| | GCUACCUGAGCAUCACCUUCCUGCGGGACUUCUGGGGCAA | |
| | UCCCCUGCGGUACGACACCGAGUACUACCUGAUCCCCGUG | |
| | GCCUCAAGCAGCAAGGACGUGCAGUUGAAGAACAUCGCC | |
| | GACUACAUGUACCUGACCAACGCCCCGAGCUACACCAACG | |
| | GCAAGCUGAACAUCUACUACCGGCGGCUGUACAACGGCCU | |
| | GAAGUUCAUCAUCAAGCGGUACACCCCUAACAACGAGAU | |
| | CGACAGCUUCGUGAAGAGCGGCGACUUCAUCAAGCUGUA | |
| | CGUGAGCUACAACAACAACGAGCACAUCGUGGGCUACCCC | |
| | AAGGACGGCAACGCCUUCAACAACCUGGACCGGAUCCUGC | |
| | GGGUGGGCUACAACGCCCCAGGCAUUCCCCUGUACAAGAA | |
| | GAUGGAGGCCGUGAAGCUGCGGGACCUGAAGACCUACAG | |
| | CGUGCAGCUGAAACUGUACGACGACAAGAACGCCAGCCU | |
| | GGGCCUGGUGGGCACCCACAACGGCCAGAUCGGCAACGAC | |
| | CCCAACCGGGACAUCCUGAUCGCCAGCAACUGGUACUUUA | |
| | ACCACCUGAAGGACAAGAUCCUGGGCUGCGACUGGUACU | |
| | UCGUGCCCACCGACGAGGGCUGGACCAACGACGGAGGAG | |
| | GUGGCUCUGGCGGAGGUGGAAGCGGUGGAGGCGGAAGUG | |
| | GUGGCGGAGGCAGCGACGAUCCUCCAGCCACCGUGUAUA | |
| | AGUACGACAGCAGACCUCCUGAGGACGUGUUCCAGAACG | |
| | GCUUUACCGCCUGGGGCAACAACGACAACGUGCUGGAUC | |
| | ACCUGACCGGCAGAUCCUGUCAAGUGGGCAGCAGCAAUA | |
| | GCGCCUUCGUGUCCACCUCUAGCAGCAGACGGUACACAGA | |
| | GGUGUACCUGGAACACCGGAUGCAAGAGGCCGUGGAAGC | |
| | CGAAAGAGCCGGCAGAGGAACAGGCCACUUCAUCGGCUA | |
| | CAUCUACGAAGUGCGGGCCGACAACAACUUCUACGGCGCU | |
| | GCCAGCAGCUACUUCGAGUACGUGGACACCUACGGCGACA | |
| | ACGCCGGAAGAAUACUGGCUGGCGCUCUGGCCACAUACCA | |
| | GUCUGGAUAUCUGGCCCACAGACGGAUCCCUCCAGAGAAC | |
| | AUUCGGAGAGUGACCCGGGUGUACCACAACGGCAUUACC | |
| | GGCGAGACAACCACCACCGAGUACAGCAACGCCAGAUACG | |
| | UGUCCCAGCAGACCCGGGCCAAUCCUAAUCCUUACACCAG | |
| | C | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding amino | METPAQLLFLLLLWLPDTTGKNLDCWVDNEEDIDVILKKSTIL | 71 |
| acid sequence | NLDINNDIISDISGFNSAVITYPDAQLVPGINGKAIHLVNNEASE | |
| | VIVHKAMDIEYNDMFNNFAVSFWLRVPKVSASHLEQYGTNE | |
| | YSIISSMKKHSLSIGSGWSVSLKGNNLIWTLKDSAGEVRQITFR | |
| | DLPDKFNAYLANKWVFITITNDRLSSANLYINGVLMGSAEITG | |
| | LGAIREDNNIALKLDRCNNNNQYVSIDKFRIFCKALNPKEIEKL | |
| | YTSYLSITFLRDFWGNPLRYDTEYYLIPVASSSKDVQLKNIAD | |
| | YMYLTNAPSYTNGKLNIYYRRLYNGLKFIIKRYTPNNEIDSFV | |
| | KSGDFIKLYVSYNNNEHIVGYPKDGNAFNNLDRILRVGYNAP | |
| | GIPLYKKMEAVKLRDLKTYSVQLKLYDDKNASLGLVGTHNG | |
| | QIGNDPNRDILIASNWYFNHLKDKILGCDWYFVPTDEGWTND | |
| | GGGGSGGGGSGGGGSGGGGSDDPPATVYKYDSRPPEDVFQN | |
| | GFTAWGNNDNVLDHLTGRSCQVGSSNSAFVSTSSSRRYTEVY | |
| | LEHRMQEAVEAERAGRGTGHFIGYIYEVRADNNFYGAASSYF | |
| | EYVDTYGDNAGRILAGALATYQSGYLAHRRIPPENIRRVTRVY | |
| | HNGITGETTTTEYSNARYVSQQTRANPNPYTS | |
| PolyA tail | 100 nt | |

TcfA_Furin2A_SphB1

| SEQ ID NO: 72 consists of from 5′ end to 3′ end: | | 72 |
|---|---|---|
| 5′ UTR SEQ ID NO: 2, mRNA ORF SEQ ID NO: 73, and | | |
| 3′ UTR SEQ ID NO: 4. | | |
| 5′ UTR | GGGAAAUAAGAGAGAAAAGAAGAGUAAGAAGAAAUAUAA | 2 |
| | GACCCCGGCGCCGCCACC | |
| ORF of mRNA | AUGGAGACACCAGCCCAGUUAUUAUUCCUCCUUCUCCUAU | 73 |
| Construct | GGCUUCCAGACACCACCGGCCUUAAGCUCCCUAGCCUCCU | |
| (excluding the stop | AACCGACGACGAGCUUAAGCUCGUGCUUCCUACCGGCAUG | |
| codon) | AGCCUCGAGGACUUCAAGAGAAGCCUCCAGGAGAGCGCCC | |
| | CUAGCGCCCUGGCCACCCCUCCUAGCAGCAGCCCUCCUGU | |
| | GGCCAAGCCUGGCCCUGGCAGCGUGGCCGAGGCCCCAUCG | |
| | GGCAGCGGCCACAAGGAUAACCCUAGCCCUCCAGUUGUGG | |
| | GCGUGGGUCCUGGAAUGGCCGAGAGCAGCGGCGGUCAUA | |
| | ACCCUGGCGUGGGCGGCGGCACCCACGAGAACGGCCUGCC | |
| | UGGCAUCGGCAAGGUGGGAGGCAGUGCCCCUGGUCCUGA | |
| | CACUAGCACCGGCUCUGGCCCAGAUGCCGGAAUGGCAAGC | |
| | GGCGCCGGCAGCACCAGCCCUGGCGCUUCAGGAAUGCCUC | |
| | CAAGUGAGGGCGAACGGCCUGAUUCCGGAAUGUCCGAUA | |
| | GCGGCAGAGGCGGAGAAUCAAGCGCCGGCGGACUGAACC | |
| | CUGAUGGCGCAGGCAAGCCACCUAGAGAGGAGGGUGAGC | |
| | CAGGUUCUAAGAGCCCUGCCGAUGGCGGCCAGGACGGCCC | |
| | UCCACCACCUCGAGACGGCGGAGACGCCGACCCUCAACCA | |
| | CCACGGGACGACGGCAACGGCGAACAGCAGCCUCCAAAGG | |
| | GCGGAGGUGAUGAGGGCCAAAGGCCUCCUCCUGCCGCGG | |
| | GCAAUGGUGGUAACGGCGGUAAUGGAAACGCGCAGCUAC | |
| | CUGAGAGAGGCGACGACGCCGGUCCUAAGCCGCCAGAGG | |
| | GUGAAGGUGGAGACGAGGGACCACAGCCACCUCAAGGAG | |
| | GAGGCGAGCAAGACGCCCCUGAGGUGCCACCUGUGGCUCC | |
| | AGCACCACCAGCCGGUAACGGUGUGUACGACCCUGGUACC | |
| | CACACCCUGACCACUCCUGCAUCGGCCGCCGUCUCACUUG | |
| | CGUCUAGCAGCCAUGGUGUUUGGCAGGCCGAGAUGAACC | |
| | GGGCCAAGAGAAGCGGAAGCGGCGCUCCCGUGAAGCAGA | |
| | CCCUGAACUUCGACCUGCUGAAGCUGGCCGGCGACGUGGA | |
| | GAGCAACCCCGGCCCCAUGGAGACGCCUGCCCAGCUGCUG | |
| | UUCCUGCUGCUUCUGUGGCUGCCCGACACCACCGGCGGCA | |
| | GCCCUGGUGGCAGAGCCCCAUCUGCGCCUCAGCCUGCUCC | |
| | AAGUCCCAGACCCGAGCCUGCCCCAGAACCAGCCCCAAAC | |
| | CCAGCUCCUAGGCCAGCUCCACAGCCACCCGCACCCGCUC | |
| | CCGGUGCACCCCGCCCACCAGCACCUCCGCCUGAGGCGCC | |
| | ACCUCCGGUGAUGCCACCGCCAGCUGUGCCUCCUCAGCUU | |
| | CCCGAGGUGCCCGCUGCCGAUCUGCCCAGAGUGCGAGCCC | |
| | CGCUGAGCACCUACAGAAGACCCCAGAGAACCGACUUCGU | |
| | GACCCCUACAGGAGGUCCCUUCUUCGCCAAGCAGGACAAG | |
| | GCCCUGAACACCAUCGACCUGAAGAUGGCCCACGAUCUGA | |
| | AGCUGAGAGGCUACAGAGUGAAGGUGGCCGUGGUGGACG | |
| | AGGGCGUGAGAAGCGACCACCCGCUGCUGAACGUGGAGA | |
| | AGAAGUACGGCGGCGACUACAUGGCCGACGGCACCAGAA | |
| | CCUACCCCGACCCCAAGAGACAGGGCAGACACGGCACUAG | |
| | CGUGGCACUUGUGCUGGCCGGCCAGGACACCGAUACCUAC | |
| | CGCGGCGGAGUGGCUCCGAACGCGGAUCUGUACAGCGCCA | |
| | ACAUCGGAACACGUGCCGGCCACGUGAGCGACGAGGCCGC | |
| | CUUCCACGCCUGGAACGACCUGCUGGGCCACGGCAUCAAG | |
| | AUCUUCAACAACAGCUUCGCCACCGAGGGCCCCGAGGGCG | |
| | AGCAGAGGGUUAAGGAGGACAGAAACGAGUACCACAGCG | |
| | CCGCCAACAAGCAGAACACAUACAUUGGCAGACUGGACA | |
| | GACUGGUGAGAGACGGCGCUUUGCUCAUCUUCGCCGCCG | |
| | GCAACGGCAGACCCUCCGGCAGAGCUUACAGCGAGGUGG | |
| | GCAGCGUGGGCAGAACCCCUAGAGUCGAGCCCCACCUGCA | |
| | GAGAGGCCUGAUCGUGGUUACCGCCGUGGACGAGAACGG | |
| | CCGACUCGAAACUUGGGCCAACAGGUGCGGCCAGGCCCAG | |
| | CAGUGGUGCCUGGCUGCACCCUCCACCGCCUACCUGCCCG | |
| | GCCUGGACAAGGACAACCCCGACAGCAUCCACGUCGAGCA | |
| | GGGCACCGCGCUCAGCGCCCCUCUUGUUACAGGCGCUGCC | |
| | GUCCUGGUGCAGGACAGAUUCCGCUGGAUGGACAACGAC | |
| | AACCUGAGAACCACUCUGCUAACCACCGCCCAGGAUAAGG | |
| | GCCCCUACGGCGUGGACCCUCAGUACGGCUGGGGCGUGCU | |
| | GGACGUUGGUCGGGCAGUGCAGGGCCCUGCACAGUUCGC | |
| | CUUCGGAGAUUUCGUGGCACGCGUGACUGAUACCUCAAC | |
| | UUUCGGCAACGACAUCUCCGGCGCUGGUGGGCUGGUAGU | |
| | GGACGGCCCAGGCGCCUUAGUUCUCGCAGGCAGCAACACC | |
| | UACGCCGGAAGGACUACAAUCAAGAGAGGCACCCUGGAC | |
| | GUAUUCGGAUCGGUGACCAGCGCCGUGACCGUGGAACCA | |
| | GGUGGCACUCUGACCGGCAUUGGCACUGUGGGCACCGUC | |
| | ACCAACCAGGGUACCGUUGUGAACAAGGAGGCCGGCCUG | |
| | CACGUGAAGGGCGACUAUAGCCAGACAGCACAGGGCCUG | |
| | CUCGUGACCGACAUAGGCUCUCUUCUGGACGUGAGUGGA | |
| | CGCGCCUCCCUGGCCGGACGCCUUCACGUCGACGACAUAA | |
| | GACCCGGCUACGUGGGCGGUGACGGGAAGUCCGUGCCCG | |
| | UGAUCAAGGCCGGCGCAGUUAGCGGCGUGUUCGCCACAC | |
| | UCACUAGAAGUCCAGGACUGCUCCUUAACGCUAGGCUCG | |
| | AUUACCGGCCACAGGCCGUGUACCUGACCAUGCGUCGGGC | |
| | CGAGCGUGUGCACGCGGCCGCCCAGCGCGGAGCCGACGAC | |
| | GGUCGGCGGGCCUCUGUCCUUGCCGUCGCCGAGAGGUUA | |
| | GACGCCGCCAUGAGAGAGCUGGACGCCCUGCCAGAGUCUC | |
| | AGCGUGACGCUGCCGCACCAGCCGCGGCCAUCGGAAGGAU | |
| | CCAGAGAGUACAGAGCAGAAAGGUGCUGCAAGAUAACCU | |
| | CUACAGCCUUGCUGGCGCUACAUACGCUAACGCCGCCGCU | |
| | GUCAAUACACUGGAACAGAACCGGUGGAUGGAUAGACUC | |
| | GAGAAU | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding amino | METPAQLLFLLLLWLPDTTGLKLPSLLTDDELKLVLPTGMSLE | 74 |
| acid sequence | DFKRSLQESAPSALATPPSSSPPVAKPGPGSVAEAPSGSGHKD | |
| | NPSPPVVGVGPGMAESSGGHNPGVGGGTHENGLPGIGKVGGS | |
| | APGPDTSTGSGPDAGMASGAGSTSPGASGMPPSEGERPDSGM | |
| | SDSGRGGESSAGGLNPDGAGKPPREEGEPGSKSPADGGQDGP | |
| | PPPRDGGDADPQPPRDDGNGEQQPPKGGGDEGQRPPPAAGNG | |
| | GNGGNGNAQLPERGDDAGPKPPEGEGGDEGPQPPQGGGEQD | |
| | APEVPPVAPAPPAGNGVYDPGTHTLTTPASAAVSLASSSHGV | |
| | WQAEMNRAKRSGSGAPVKQTLNFDLLKLAGDVESNPGPMET | |
| | PAQLLFLLLLWLPDTTGGSPGGRAPSAPQPAPSPRPEPAPEPAP | |
| | NPAPRPAPQPPAPAPGAPRPPAPPPEAPPPVMPPPAVPPQLPEV | |
| | PAADLPRVRAPLSTYRRPQRTDFVTPTGGPFFAKQDKALNTID | |
| | LKMAHDLKLRGYRVKVAVVDEGVRSDHPLLNVEKKYGGDY | |
| | MADGTRTYPDPKRQGRHGTSVALVLAGQDTDTYRGGVAPNA | |
| | DLYSANIGTRAGHVSDEAAFHAWNDLLGHGIKIFNNSFATEGP | |
| | EGEQRVKEDRNEYHSAANKONTYIGRLDRLVRDGALLIFAAG | |
| | NGRPSGRAYSEVGSVGRTPRVEPHLQRGLIVVTAVDENGRLE | |
| | TWANRCGQAQQWCLAAPSTAYLPGLDKDNPDSIHVEQGTAL | |
| | SAPLVTGAAVLVQDRFRWMDNDNLRTTLLTTAQDKGPYGVD | |
| | PQYGWGVLDVGRAVQGPAQFAFGDFVARVTDTSTFGNDISG | |
| | AGGLVVDGPGALVLAGSNTYAGRTTIKRGTLDVFGSVTSAVT | |
| | VEPGGTLTGIGTVGTVTNQGTVVNKEAGLHVKGDYSQTAQG | |
| | LLVTDIGSLLDVSGRASLAGRLHVDDIRPGYVGGDGKSVPVIK | |
| | AGAVSGVFATLTRSPGLLLNARLDYRPQAVYLTMRRAERVH | |
| | AAAQRGADDGRRASVLAVAERLDAAMRELDALPESQRDAAA | |
| | PAAAIGRIQRVQSRKVLQDNLYSLAGATYANAAAVNTLEQNR | |
| | WMDRLEN | |
| PolyA tail | 100 nt | |

TcfA_20AALinker_SphB1

| SEQ ID NO: 75 consists of from 5′ end to 3′ end: | | 75 |
|---|---|---|
| 5′ UTR SEQ ID NO: 2, mRNA ORF SEQ ID NO: 76, and | | |
| 3′ UTR SEQ ID NO: 4. | | |
| 5′ UTR | GGGAAAUAAGAGAGAAAAGAAGAGUAAGAAGAAAUAUAA | 2 |
| | GACCCCGGCGCCGCCACC | |
| ORF of mRNA | AUGGAGACACCAGCCCAGUUAUUAUUCCUCCUUCUCCUAU | 76 |
| Construct | GGCUUCCAGACACCACCGGCCUUAAGCUCCCUAGCCUCCU | |
| (excluding the stop | AACCGACGACGAGCUUAAGCUCGUGCUUCCUACCGGCAUG | |
| codon) | AGCCUCGAGGACUUCAAGAGAAGCCUCCAGGAGAGCGCCC | |
| | CUAGCGCCCUGGCCACCCCUCCUAGCAGCAGCCCUCCUGU | |
| | GGCCAAGCCUGGCCCUGGCAGCGUGGCCGAGGCCCCAUCG | |
| | GGCAGCGGCCACAAGGAUAACCCUAGCCCUCCAGUUGUGG | |
| | GCGUGGGUCCUGGAAUGGCCGAGAGCAGCGGCGGUCAUA | |
| | ACCCUGGCGUGGGCGGCGGCACCCACGAGAACGGCCUGCC | |
| | UGGCAUCGGCAAGGUGGGAGGCAGUGCCCCUGGUCCUGA | |
| | CACUAGCACCGGCUCUGGCCCAGAUGCCGGAAUGGCAAGC | |
| | GGCGCCGGCAGCACCAGCCCUGGCGCUUCAGGAAUGCCUC | |
| | CAAGUGAGGGCGAACGGCCUGAUUCCGGAAUGUCCGAUA | |
| | GCGGCAGAGGCGGAGAAUCAAGCGCCGGCGGACUGAACC | |
| | CUGAUGGCGCAGGCAAGCCACCUAGAGAGGAGGGUGAGC | |
| | CAGGUUCUAAGAGCCCUGCCGAUGGCGGCCAGGACGGCCC | |
| | UCCACCACCUCGAGACGGCGGAGACGCCGACCCUCAACCA | |
| | CCACGGGACGACGGCAACGGCGAACAGCAGCCUCCAAAGG | |
| | GCGGAGGUGAUGAGGGCCAAAGGCCUCCUCCUGCCGCGG | |
| | GCAAUGGUGGUAACGGCGGUAAUGGAAACGCGCAGCUAC | |
| | CUGAGAGAGGCGACGACGCCGGUCCUAAGCCGCCAGAGG | |
| | GUGAAGGUGGAGACGAGGGACCACAGCCACCUCAAGGAG | |
| | GAGGCGAGCAAGACGCCCCUGAGGUGCCACCUGUGGCUCC | |
| | AGCACCACCAGCCGGUAACGGUGUGUACGACCCUGGUACC | |
| | CACACCCUGACCACUCCUGCAUCGGCCGCCGUCUCACUUG | |
| | CGUCUAGCAGCCAUGGUGUUUGGCAGGCCGAGAUGAACG | |
| | GAGGAGGUGGCUCUGGCGGAGGUGGAAGCGGUGGAGGCG | |
| | GAAGUGGUGGCGGAGGCAGCGGCAGCCCUGGUGGCAGAG | |
| | CCCCAUCUGCGCCUCAGCCUGCUCCAAGUCCCAGACCCGA | |
| | GCCUGCCCCAGAACCAGCCCCAAACCCAGCUCCUAGGCCA | |
| | GCUCCACAGCCACCCGCACCCGCUCCCGGUGCACCCCGCC | |
| | CACCAGCACCUCCGCCUGAGGCGCCACCUCCGGUGAUGCC | |
| | ACCGCCAGCUGUGCCUCCUCAGCUUCCCGAGGUGCCCGCU | |
| | GCCGAUCUGCCCAGAGUGCGAGCCCCGCUGAGCACCUACA | |
| | GAAGACCCCAGAGAACCGACUUCGUGACCCCUACAGGAGG | |
| | UCCCUUCUUCGCCAAGCAGGACAAGGCCCUGAACACCAUC | |
| | GACCUGAAGAUGGCCCACGAUCUGAAGCUGAGAGGCUAC | |
| | AGAGUGAAGGUGGCCGUGGUGGACGAGGGCGUGAGAAGC | |
| | GACCACCCGCUGCUGAACGUGGAGAAGAAGUACGGCGGC | |
| | GACUACAUGGCCGACGGCACCAGAACCUACCCCGACCCCA | |
| | AGAGACAGGGCAGACACGGCACUAGCGUGGCACUUGUGC | |
| | UGGCCGGCCAGGACACCGAUACCUACCGCGGCGGAGUGGC | |
| | UCCGAACGCGGAUCUGUACAGCGCCAACAUCGGAACACGU | |
| | GCCGGCCACGUGAGCGACGAGGCCGCCUUCCACGCCUGGA | |
| | ACGACCUGCUGGGCCACGGCAUCAAGAUCUUCAACAACAG | |
| | CUUCGCCACCGAGGGCCCCGAGGGCGAGCAGAGGGUUAA | |
| | GGAGGACAGAAACGAGUACCACAGCGCCGCCAACAAGCA | |
| | GAACACAUACAUUGGCAGACUGGACAGACUGGUGAGAGA | |
| | CGGCGCUUUGCUCAUCUUCGCCGCCGGCAACGGCAGACCC | |
| | UCCGGCAGAGCUUACAGCGAGGUGGGCAGCGUGGGCAGA | |
| | ACCCCUAGAGUCGAGCCCCACCUGCAGAGAGGCCUGAUCG | |
| | UGGUUACCGCCGUGGACGAGAACGGCCGACUCGAAACUU | |
| | GGGCCAACAGGUGCGGCCAGGCCCAGCAGUGGUGCCUGGC | |
| | UGCACCCUCCACCGCCUACCUGCCCGGCCUGGACAAGGAC | |
| | AACCCCGACAGCAUCCACGUCGAGCAGGGCACCGCGCUCA | |
| | GCGCCCCUCUUGUUACAGGCGCUGCCGUCCUGGUGCAGGA | |
| | CAGAUUCCGCUGGAUGGACAACGACAACCUGAGAACCAC | |
| | UCUGCUAACCACCGCCCAGGAUAAGGGCCCCUACGGCGUG | |
| | GACCCUCAGUACGGCUGGGGCGUGCUGGACGUUGGUCGG | |
| | GCAGUGCAGGGCCCUGCACAGUUCGCCUUCGGAGAUUUC | |
| | GUGGCACGCGUGACUGAUACCUCAACUUUCGGCAACGAC | |
| | AUCUCCGGCGCUGGUGGGCUGGUAGUGGACGGCCCAGGC | |
| | GCCUUAGUUCUCGCAGGCAGCAACACCUACGCCGGAAGGA | |
| | CUACAAUCAAGAGAGGCACCCUGGACGUAUUCGGAUCGG | |
| | UGACCAGCGCCGUGACCGUGGAACCAGGUGGCACUCUGAC | |
| | CGGCAUUGGCACUGUGGGCACCGUCACCAACCAGGGUACC | |
| | GUUGUGAACAAGGAGGCCGGCCUGCACGUGAAGGGCGAC | |
| | UAUAGCCAGACAGCACAGGGCCUGCUCGUGACCGACAUA | |
| | GGCUCUCUUCUGGACGUGAGUGGACGCGCCUCCCUGGCCG | |
| | GACGCCUUCACGUCGACGACAUAAGACCCGGCUACGUGGG | |
| | CGGUGACGGGAAGUCCGUGCCCGUGAUCAAGGCCGGCGC | |
| | AGUUAGCGGCGUGUUCGCCACACUCACUAGAAGUCCAGG | |
| | ACUGCUCCUUAACGCUAGGCUCGAUUACCGGCCACAGGCC | |
| | GUGUACCUGACCAUGCGUCGGGCCGAGCGUGUGCACGCG | |
| | GCCGCCCAGCGCGGAGCCGACGACGGUCGGCGGGCCUCUG | |
| | UCCUUGCCGUCGCCGAGAGGUUAGACGCCGCCAUGAGAG | |
| | AGCUGGACGCCCUGCCAGAGUCUCAGCGUGACGCUGCCGC | |
| | ACCAGCCGCGGCCAUCGGAAGGAUCCAGAGAGUACAGAG | |
| | CAGAAAGGUGCUGCAAGAUAACCUCUACAGCCUUGCUGG | |
| | CGCUACAUACGCUAACGCCGCCGCUGUCAAUACACUGGAA | |
| | CAGAACCGGUGGAUGGAUAGACUCGAGAAU | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding amino | METPAQLLFLLLLWLPDTTGLKLPSLLTDDELKLVLPTGMSLE | 77 |
| acid sequence | DFKRSLQESAPSALATPPSSSPPVAKPGPGSVAEAPSGSGHKD | |
| | NPSPPVVGVGPGMAESSGGHNPGVGGGTHENGLPGIGKVGGS | |
| | APGPDTSTGSGPDAGMASGAGSTSPGASGMPPSEGERPDSGM | |
| | SDSGRGGESSAGGLNPDGAGKPPREEGEPGSKSPADGGQDGP | |
| | PPPRDGGDADPQPPRDDGNGEQQPPKGGGDEGQRPPPAAGNG | |
| | GNGGNGNAQLPERGDDAGPKPPEGEGGDEGPQPPQGGGEQD | |
| | APEVPPVAPAPPAGNGVYDPGTHTLTTPASAAVSLASSSHGV | |
| | WQAEMNGGGGSGGGGSGGGGSGGGGSGSPGGRAPSAPQPAP | |
| | SPRPEPAPEPAPNPAPRPAPQPPAPAPGAPRPPAPPPEAPPPVMP | |
| | PPAVPPQLPEVPAADLPRVRAPLSTYRRPQRTDFVTPTGGPFFA | |
| | KQDKALNTIDLKMAHDLKLRGYRVKVAVVDEGVRSDHPLLN | |
| | VEKKYGGDYMADGTRTYPDPKRQGRHGTSVALVLAGQDTD | |
| | TYRGGVAPNADLYSANIGTRAGHVSDEAAFHAWNDLLGHGI | |
| | KIFNNSFATEGPEGEQRVKEDRNEYHSAANKQNTYIGRLDRL | |
| | VRDGALLIFAAGNGRPSGRAYSEVGSVGRTPRVEPHLQRGLIV | |
| | VTAVDENGRLETWANRCGQAQQWCLAAPSTAYLPGLDKDNP | |
| | DSIHVEQGTALSAPLVTGAAVLVQDRFRWMDNDNLRTTLLTT | |
| | AQDKGPYGVDPQYGWGVLDVGRAVQGPAQFAFGDFVARVT | |
| | DTSTFGNDISGAGGLVVDGPGALVLAGSNTYAGRTTIKRGTL | |
| | DVFGSVTSAVTVEPGGTLTGIGTVGTVTNQGTVVNKEAGLHV | |
| | KGDYSQTAQGLLVTDIGSLLDVSGRASLAGRLHVDDIRPGYV | |
| | GGDGKSVPVIKAGAVSGVFATLTRSPGLLLNARLDYRPQAVY | |
| | LTMRRAERVHAAAQRGADDGRRASVLAVAERLDAAMRELD | |
| | ALPESQRDAAAPAAAIGRIQRVQSRKVLQDNLYSLAGATYAN | |
| | AAAVNTLEQNRWMDRLEN | |
| PolyA tail | 100 nt | |

SphB1_20AALinker_TcfA

| SEQ ID NO: 78 consists of from 5′ end to 3′ end: | | 78 |
|---|---|---|
| 5′ UTR SEQ ID NO: 2, mRNA ORF SEQ ID NO: 79, | | |
| and 3′ UTR SEQ ID NO: 4. | | |
| 5′ UTR | GGGAAAUAAGAGAGAAAAGAAGAGUAAGAAGAAAUAUAA | 2 |
| | GACCCCGGCGCCGCCACC | |
| ORF of mRNA | AUGGAGACACCAGCCCAGUUAUUAUUCCUCCUUCUCCUAU | 79 |
| Construct | GGCUUCCAGACACCACCGGCGGCAGCCCUGGUGGCAGAGC | |
| (excluding the stop | CCCAUCUGCGCCUCAGCCUGCUCCAAGUCCCAGACCCGAG | |
| codon) | CCUGCCCCAGAACCAGCCCCAAACCCAGCUCCUAGGCCAG | |
| | CUCCACAGCCACCCGCACCCGCUCCCGGUGCACCCCGCCC | |
| | ACCAGCACCUCCGCCUGAGGCGCCACCUCCGGUGAUGCCA | |
| | CCGCCAGCUGUGCCUCCUCAGCUUCCCGAGGUGCCCGCUG | |
| | CCGAUCUGCCCAGAGUGCGAGCCCCGCUGAGCACCUACAG | |
| | AAGACCCCAGAGAACCGACUUCGUGACCCCUACAGGAGGU | |
| | CCCUUCUUCGCCAAGCAGGACAAGGCCCUGAACACCAUCG | |
| | ACCUGAAGAUGGCCCACGAUCUGAAGCUGAGAGGCUACA | |
| | GAGUGAAGGUGGCCGUGGUGGACGAGGGCGUGAGAAGCG | |
| | ACCACCCGCUGCUGAACGUGGAGAAGAAGUACGGCGGCG | |
| | ACUACAUGGCCGACGGCACCAGAACCUACCCCGACCCCAA | |
| | GAGACAGGGCAGACACGGCACUAGCGUGGCACUUGUGCU | |
| | GGCCGGCCAGGACACCGAUACCUACCGCGGCGGAGUGGCU | |
| | CCGAACGCGGAUCUGUACAGCGCCAACAUCGGAACACGUG | |
| | CCGGCCACGUGAGCGACGAGGCCGCCUUCCACGCCUGGAA | |
| | CGACCUGCUGGGCCACGGCAUCAAGAUCUUCAACAACAGC | |
| | UUCGCCACCGAGGGCCCCGAGGGCGAGCAGAGGGUUAAG | |
| | GAGGACAGAAACGAGUACCACAGCGCCGCCAACAAGCAG | |
| | AACACAUACAUUGGCAGACUGGACAGACUGGUGAGAGAC | |
| | GGCGCUUUGCUCAUCUUCGCCGCCGGCAACGGCAGACCCU | |
| | CCGGCAGAGCUUACAGCGAGGUGGGCAGCGUGGGCAGAA | |
| | CCCCUAGAGUCGAGCCCCACCUGCAGAGAGGCCUGAUCGU | |
| | GGUUACCGCCGUGGACGAGAACGGCCGACUCGAAACUUG | |
| | GGCCAACAGGUGCGGCCAGGCCCAGCAGUGGUGCCUGGCU | |
| | GCACCCUCCACCGCCUACCUGCCCGGCCUGGACAAGGACA | |
| | ACCCCGACAGCAUCCACGUCGAGCAGGGCACCGCGCUCAG | |
| | CGCCCCUCUUGUUACAGGCGCUGCCGUCCUGGUGCAGGAC | |
| | AGAUUCCGCUGGAUGGACAACGACAACCUGAGAACCACU | |
| | CUGCUAACCACCGCCCAGGAUAAGGGCCCCUACGGCGUGG | |
| | ACCCUCAGUACGGCUGGGGCGUGCUGGACGUUGGUCGGG | |
| | CAGUGCAGGGCCCUGCACAGUUCGCCUUCGGAGAUUUCG | |
| | UGGCACGCGUGACUGAUACCUCAACUUUCGGCAACGACA | |
| | UCUCCGGCGCUGGUGGGCUGGUAGUGGACGGCCCAGGCG | |
| | CCUUAGUUCUCGCAGGCAGCAACACCUACGCCGGAAGGAC | |
| | UACAAUCAAGAGAGGCACCCUGGACGUAUUCGGAUCGGU | |
| | GACCAGCGCCGUGACCGUGGAACCAGGUGGCACUCUGACC | |
| | GGCAUUGGCACUGUGGGCACCGUCACCAACCAGGGUACCG | |
| | UUGUGAACAAGGAGGCCGGCCUGCACGUGAAGGGCGACU | |
| | AUAGCCAGACAGCACAGGGCCUGCUCGUGACCGACAUAG | |
| | GCUCUCUUCUGGACGUGAGUGGACGCGCCUCCCUGGCCGG | |
| | ACGCCUUCACGUCGACGACAUAAGACCCGGCUACGUGGGC | |
| | GGUGACGGGAAGUCCGUGCCCGUGAUCAAGGCCGGCGCA | |
| | GUUAGCGGCGUGUUCGCCACACUCACUAGAAGUCCAGGA | |
| | CUGCUCCUUAACGCUAGGCUCGAUUACCGGCCACAGGCCG | |
| | UGUACCUGACCAUGCGUCGGGCCGAGCGUGUGCACGCGGC | |
| | CGCCCAGCGCGGAGCCGACGACGGUCGGCGGGCCUCUGUC | |
| | CUUGCCGUCGCCGAGAGGUUAGACGCCGCCAUGAGAGAG | |
| | CUGGACGCCCUGCCAGAGUCUCAGCGUGACGCUGCCGCAC | |
| | CAGCCGCGGCCAUCGGAAGGAUCCAGAGAGUACAGAGCA | |
| | GAAAGGUGCUGCAAGAUAACCUCUACAGCCUUGCUGGCG | |
| | CUACAUACGCUAACGCCGCCGCUGUCAAUACACUGGAACA | |
| | GAACCGGUGGAUGGAUAGACUCGAGAAUGGAGGAGGUGG | |
| | CUCUGGCGGAGGUGGAAGCGGUGGAGGCGGAAGUGGUGG | |
| | CGGAGGCAGCCUUAAGCUCCCUAGCCUCCUAACCGACGAC | |
| | GAGCUUAAGCUCGUGCUUCCUACCGGCAUGAGCCUCGAG | |
| | GACUUCAAGAGAAGCCUCCAGGAGAGCGCCCCUAGCGCCC | |
| | UGGCCACCCCUCCUAGCAGCAGCCCUCCUGUGGCCAAGCC | |
| | UGGCCCUGGCAGCGUGGCCGAGGCCCCAUCGGGCAGCGGC | |
| | CACAAGGAUAACCCUAGCCCUCCAGUUGUGGGCGUGGGU | |
| | CCUGGAAUGGCCGAGAGCAGCGGCGGUCAUAACCCUGGC | |
| | GUGGGCGGCGGCACCCACGAGAACGGCCUGCCUGGCAUCG | |
| | GCAAGGUGGGAGGCAGUGCCCCUGGUCCUGACACUAGCA | |
| | CCGGCUCUGGCCCAGAUGCCGGAAUGGCAAGCGGCGCCGG | |
| | CAGCACCAGCCCUGGCGCUUCAGGAAUGCCUCCAAGUGAG | |
| | GGCGAACGGCCUGAUUCCGGAAUGUCCGAUAGCGGCAGA | |
| | GGCGGAGAAUCAAGCGCCGGCGGACUGAACCCUGAUGGC | |
| | GCAGGCAAGCCACCUAGAGAGGAGGGUGAGCCAGGUUCU | |
| | AAGAGCCCUGCCGAUGGCGGCCAGGACGGCCCUCCACCAC | |
| | CUCGAGACGGCGGAGACGCCGACCCUCAACCACCACGGGA | |
| | CGACGGCAACGGCGAACAGCAGCCUCCAAAGGGCGGAGG | |
| | UGAUGAGGGCCAAAGGCCUCCUCCUGCCGCGGGCAAUGG | |
| | UGGUAACGGCGGUAAUGGAAACGCGCAGCUACCUGAGAG | |
| | AGGCGACGACGCCGGUCCUAAGCCGCCAGAGGGUGAAGG | |
| | UGGAGACGAGGGACCACAGCCACCUCAAGGAGGAGGCGA | |
| | GCAAGACGCCCCUGAGGUGCCACCUGUGGCUCCAGCACCA | |
| | CCAGCCGGUAACGGUGUGUACGACCCUGGUACCCACACCC | |
| | UGACCACUCCUGCAUCGGCCGCCGUCUCACUUGCGUCUAG | |
| | CAGCCAUGGUGUUUGGCAGGCCGAGAUGAAC | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGGG | |
| | C | |
| Corresponding amino | METPAQLLFLLLLWLPDTTGGSPGGRAPSAPQPAPSPRPEPAPE | 80 |
| acid sequence | PAPNPAPRPAPQPPAPAPGAPRPPAPPPEAPPPVMPPPAVPPQL | |
| | PEVPAADLPRVRAPLSTYRRPQRTDFVTPTGGPFFAKQDKALN | |
| | TIDLKMAHDLKLRGYRVKVAVVDEGVRSDHPLLNVEKKYGG | |
| | DYMADGTRTYPDPKRQGRHGTSVALVLAGQDTDTYRGGVAP | |
| | NADLYSANIGTRAGHVSDEAAFHAWNDLLGHGIKIFNNSFAT | |
| | EGPEGEQRVKEDRNEYHSAANKQNTYIGRLDRLVRDGALLIF | |
| | AAGNGRPSGRAYSEVGSVGRTPRVEPHLQRGLIVVTAVDENG | |
| | RLETWANRCGQAQQWCLAAPSTAYLPGLDKDNPDSIHVEQG | |
| | TALSAPLVTGAAVLVQDRFRWMDNDNLRTTLLTTAQDKGPY | |
| | GVDPQYGWGVLDVGRAVQGPAQFAFGDFVARVTDTSTFGND | |
| | ISGAGGLVVDGPGALVLAGSNTYAGRTTIKRGTLDVFGSVTSA | |
| | VTVEPGGTLTGIGTVGTVTNQGTVVNKEAGLHVKGDYSQTA | |
| | QGLLVTDIGSLLDVSGRASLAGRLHVDDIRPGYVGGDGKSVP | |
| | VIKAGAVSGVFATLTRSPGLLLNARLDYRPQAVYLTMRRAER | |
| | VHAAAQRGADDGRRASVLAVAERLDAAMRELDALPESQRDA | |
| | AAPAAAIGRIQRVQSRKVLQDNLYSLAGATYANAAAVNTLEQ | |
| | NRWMDRLENGGGGSGGGGSGGGGSGGGGSLKLPSLLTDDEL | |
| | KLVLPTGMSLEDFKRSLQESAPSALATPPSSSPPVAKPGPGSVA | |
| | EAPSGSGHKDNPSPPVVGVGPGMAESSGGHNPGVGGGTHEN | |
| | GLPGIGKVGGSAPGPDTSTGSGPDAGMASGAGSTSPGASGMP | |
| | PSEGERPDSGMSDSGRGGESSAGGLNPDGAGKPPREEGEPGSK | |
| | SPADGGQDGPPPPRDGGDADPQPPRDDGNGEQQPPKGGGDE | |
| | GQRPPPAAGNGGNGGNGNAQLPERGDDAGPKPPEGEGGDEG | |
| | PQPPQGGGEQDAPEVPPVAPAPPAGNGVYDPGTHTLTTPASA | |
| | AVSLASSSHGVWQAEMN | |
| PolyA tail | 100 nt | |

FHA_FurinF2A_Fim2-3

| SEQ ID NO: 81 consists of from 5′ end to 3′ end: | | 81 |
|---|---|---|
| 5′ UTR SEQ ID NO: 2, mRNA ORF SEQ ID NO: 82, and | | |
| 3′ UTR SEQ ID NO: 4. | | |
| 5′ UTR | GGGAAAUAAGAGAGAAAAGAAGAGUAAGAAGAAAUAUAA | 2 |
| | GACCCCGGCGCCGCCACC | |
| ORF of mRNA | AUGGAGACCCCUGCCCAGCUGCUGUUCCUGCUGCUGCUUU | 82 |
| Construct | GGCUGCCUGACACCACCGGAAGCCUGUACGCCGAACACGA | |
| (excluding the stop | CGCCACCCUGACCCUGGCCCAAGGCACCCAGAGGGAUCUG | |
| codon) | GUGGUGGACCAGGACCACAUCCUGCCCGUGGCCGAGGGA | |
| | ACCCUGAGAGUCAAAGCCAAGAGCCUCACCACCGAGAUCG | |
| | AGACAGGCAACCCUGGCAGCCUCAUCGCCGAGGUGCAGGA | |
| | GAACAUCGACAACAAGCAGGCCAUCGUCGUGGGCAAAGA | |
| | CCUGACUCUGUCCAGCGCCCACGGCAACGUAGCCAACGAG | |
| | GCCAACGCCCUGCUGUGGGCUGCCGGUGAGCUGACCGUGA | |
| | AGGCCCAGAACAUCACCAACAAGAGAGCCGCCCUGAUUGA | |
| | GGCCGGCGGGAACGCCAGACUGACCGCUGCCGUCGCCCUU | |
| | CUGAACAAGCUGGGCAGAAUCAGAGCCGGCGAGGACAUG | |
| | CACCUGGACGCCCCUAGAAUCGAGAACACCGCCAAGCUGA | |
| | GCGGCGAGGUGCAGAGAAAGGGCGUGCAGGACGUCGGCG | |
| | GAGGCGAGCACGGCAGGUGGAGCGGCAUCGGCUACGUGA | |
| | ACUACUGGCUUCGGGCCGGCAACGGCAAGAAAGCCGGCAC | |
| | CAUCGCCGCACCUUGGUACGGUGGCGAUCUGACCGCAGAG | |
| | CAGAGCCUGAUCGAGGUGGGCAAGGACCUGUACCUGAAC | |
| | GCCGGCGCCAGAAAGGACGAGCACAGACACCUGCUGAACG | |
| | AGGGCGUGAUCCAGGCCGGAGGUCACGGCCACAUCGGCG | |
| | GCGACGUGGACAACAGGGCCGUGGUCCGUACCGUGAGCG | |
| | CCAUGGAGUACUUCAAGACCCCUCUGCCUGUGAGCCUGAC | |
| | CGCCCUGGACAAUAGAGCCGGACUCAGCCCAGCCACCUGG | |
| | AACUUCCAGAGCACCUACGAGCUGCUGGACUACCUGCUGG | |
| | ACCAGAACAGAUACGAGUACAUCUGGGGCCUGUACCCUA | |
| | CCUACACCGAGUGGAGCGUGAACACCCUGAAGAACCUGG | |
| | ACCUGGGCUACCAGGCCAAGCCUGCCCCUACCGCCCCUCC | |
| | UAUGCCUAAGGCCCCUGAGCUGGACCUGAGAGGCCAUACC | |
| | CUGGAGAGCGCCGAGGGCAGAAAGAUCUUCGGCGAGUAC | |
| | AAGAAGCUGCAGGGCGAGUACGAGAAGGCCAAGAUGGCC | |
| | GUGCAAGCCGUGGAGGCCUACGGCGAGGCCACCAGAAGA | |
| | GUGCACGACCAGCUCGGGCAGCGAUACGGCAAGGCCCUGG | |
| | GCGGCAUGGACGCCGAAACCAAGGAGGUGGACGGCAUCA | |
| | UCCAGGAGUUUGCGGCCGAUCUGAGAACCGUGUACGCCA | |
| | AACAGGCCGACCAAGCCACUAUCGACGCCGAGACUGACAA | |
| | GGUGGCCCAGAGAUACAAGAGCCAGAUCGACGCCGUGAG | |
| | ACUCCAACGGGCCAAGAGAAGCGGAAGCGGCGCUCCCGUG | |
| | AAGCAGACCCUGAACUUCGACCUGCUGAAGCUGGCCGGCG | |
| | ACGUGGAGAGCAACCCCGGCCCCAUGGAGACUCCAGCACA | |
| | GUUGCUUUUCCUGCUUCUGCUGUGGCUGCCAGACACAAC | |
| | UGGAGUGGAUCCACCUGUGGACUGCGGACGUGCAUUAGG | |
| | ACUGCACUUCUGGUCAAGUGCGAGCCUUAUAUCGGAUCA | |
| | GACCCCUGACGGCACAUUGAUCGGCAAGCCGGUGGUCGG | |
| | ACGGUCACUAUUGAGCAAGUCUUGCAAGGUGCCUGACGA | |
| | CAUUAAGGAAGACCUGUCAGACAAUCACGACGGCGAGCC | |
| | AGUUGAUAUCGUACUGGAGCUUGGCAGCAACUAUAAGAU | |
| | UAGGCCACAGUCGUACGGUCAUCCUGGAAUAGUGGUCGA | |
| | CCUGCCUUCCGGUUCAACAGAGGAGACAGGUAUCGCUAU | |
| | CUACAUCGACUUCGGCUCUAGCCCAAUGCAGAAGGUCGGC | |
| | GAGAGACAGUGGCUGUACCCACAGAAGGGUGAGGUGCUU | |
| | UUCGACGUGCUGACUAUUAACGGAGACAACGCCGAGGUU | |
| | CGGUACCAGGCCAUCAAGGUCGGUCCACUGAAGCGCCCAA | |
| | GAAAGCUGGUCCUUAGUCAGUUCCCAAACCUCUUCACUU | |
| | ACAAGUGGGUCUUCAUGAGAGGCACAAGCCAGGAGCGGG | |
| | UCCUCGCACAGGGCACCAUAGAUACUGACGUGGCCACGAG | |
| | CACCAUCGACCUGAAGACGUGCAGAUACACCAGUCAGACC | |
| | GUGUCGCUCCCAAUCAUUCAGAGGAGUGCCUUGACCGGU | |
| | GUUGGCACCACACUUGGCAUGACUGACUUCCAAAUGCCA | |
| | UUCUGGUGUUACGGCUGGCCUAAGGUGUCAGUUUACAUG | |
| | UCUGCGACAAAGACUCAAACAGGCGUAGACGGCGUAGCA | |
| | CUCCCAGCAACCGGACAGGCCGCCGGUAUGGCCUCCGGCG | |
| | UGGGCGUUCAGUUGAUUAACGGUAAGACGCAGCAGCCUG | |
| | UCAAGCUGGGACUCCAGGGCAAGAUUGCCCUGCCUGAGG | |
| | CACAGCAGACCGAAAGUGCAACAUUCUCACUGCCAAUGA | |
| | AGGCCCAGUACUACCAGACCAGUACCUCUACAUCUGCCGG | |
| | CAAGCUGUCCGUUACCUACGCCGUCACUCUGAAUUACGAC | |
| | GGCGGAGGCUCCGACGACGGCACAAUCGUGAUAACCGGU | |
| | ACAAUUACUGACACAACCUGUGUAAUCGAGGAUCCUUCC | |
| | GGCCCAUCGCAUACUAAGGUUGUUCAGCUGCCAAAGAUU | |
| | UCCAAGAACGCCCUCAAGGCCAACGGAGAUCAAGCUGGU | |
| | AGGACACCAUUCAUAAUUAAGCUAAAGGACUGUCCAUCC | |
| | UCCCUCGGUAACGGCGUGAAGGCCUAUUUCGAGCCAGGA | |
| | CCUACGACUGACUACUCUACAGGUGAUCUCAGAGCAUAU | |
| | AAGAUGGUUUACGCAACAAAUCCUCAGACACAGCUCAGC | |
| | AAUAUCGUCGCCGCCACUGAGGCUCAGGGAGUCCAGGUCC | |
| | GGAUCUCCAACUUAGACGACUCAAAGAUCACCAUGGGCG | |
| | CCAACGAGGCAACUCAGCAGGCCGCUGGAUUCGACCCAGA | |
| | GGUACAGACUGGCGGUACGUCCCGCACAGUCACCAUGCGG | |
| | UACCUGGCAAGUUACGUUAAGAAGAACGGUGACGUGGAG | |
| | GCGUCCGCCAUAACCACAUACGUGGGAUUCUCAGUGGUG | |
| | UACCCUGGUGGCGGCUCUGGCGGUGGCAGUAACGACGGU | |
| | ACUAUUGUAAUUACUGGAAGUAUUUCAGACCAAACGUGC | |
| | GUUAUCGAAGAACCAAGCACAUUAAACCAUAUUAAGGUG | |
| | GUGCAGUUGCCUAAGAUAUCAAAGAACGCUUUGAGGAAC | |
| | GACGGCGAUACUGCAGGCGCAACACCUUUCGAUAUAAAG | |
| | CUUAAGGAGUGCCCUCAGGCCCUGGGCGCCCUGAAGCUCU | |
| | ACUUCGAACCAGGCAUAACAACUAACUACGACACCGGUG | |
| | AUCUUAUUGCUUACAAGCAGACAUAUAACGCAGCCGGCA | |
| | ACGGCCAGCUUUCCACUGUAUCAUCCGCCACAAAGGCUAA | |
| | GGGCGUCGAGUUCAGGCUGGCGAAUCUCAACGGACAACA | |
| | CAUUAGAAUGGGUACUGAUAAGACCACCCAAGCUGCACA | |
| | GACCUUCACAGGUAAGGUCACUAACGGCGGUAAGAGUUA | |
| | UACACUGCGCUACCUGGCUAGCUACGUGAAGAAGCCAAA | |
| | GGAGGACGUCGACGCAGCACAGAUAACAUCUUACGUAGG | |
| | AUUCUCUGUUGUUUAUCCGGGUGGCGGUAGCAACGACGG | |
| | AACCAUUGUGAUCACUGGCUCAAUUUCCGACCAGACAGU | |
| | CAUU | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding amino | METPAQLLFLLLLWLPDTTGSLYAEHDATLTLAQGTQRDLVV | 83 |
| acid sequence | DQDHILPVAEGTLRVKAKSLTTEIETGNPGSLIAEVQENIDNKQ | |
| | AIVVGKDLTLSSAHGNVANEANALLWAAGELTVKAQNITNK | |
| | RAALIEAGGNARLTAAVALLNKLGRIRAGEDMHLDAPRIENT | |
| | AKLSGEVQRKGVQDVGGGEHGRWSGIGYVNYWLRAGNGKK | |
| | AGTIAAPWYGGDLTAEQSLIEVGKDLYLNAGARKDEHRHLLN | |
| | EGVIQAGGHGHIGGDVDNRAVVRTVSAMEYFKTPLPVSLTAL | |
| | DNRAGLSPATWNFQSTYELLDYLLDQNRYEYIWGLYPTYTE | |
| | WSVNTLKNLDLGYQAKPAPTAPPMPKAPELDLRGHTLESAEG | |
| | RKIFGEYKKLQGEYEKAKMAVQAVEAYGEATRRVHDQLGQR | |
| | YGKALGGMDAETKEVDGIIQEFAADLRTVYAKQADQATIDAE | |
| | TDKVAQRYKSQIDAVRLQRAKRSGSGAPVKQTLNFDLLKLAG | |
| | DVESNPGPMETPAQLLFLLLLWLPDTTGVDPPVDCGRALGLH | |
| | FWSSASLISDQTPDGTLIGKPVVGRSLLSKSCKVPDDIKEDLSD | |
| | NHDGEPVDIVLELGSNYKIRPQSYGHPGIVVDLPSGSTEETGIAI | |
| | YIDFGSSPMQKVGERQWLYPQKGEVLFDVLTINGDNAEVRYQ | |
| | AIKVGPLKRPRKLVLSQFPNLFTYKWVFMRGTSQERVLAQGTI | |
| | DTDVATSTIDLKTCRYTSQTVSLPIIQRSALTGVGTTLGMTDFQ | |
| | MPFWCYGWPKVSVYMSATKTQTGVDGVALPATGQAAGMAS | |
| | GVGVQLINGKTQQPVKLGLQGKIALPEAQQTESATFSLPMKA | |
| | QYYQTSTSTSAGKLSVTYAVTLNYDGGGSDDGTIVITGTITDT | |
| | TCVIEDPSGPSHTKVVQLPKISKNALKANGDQAGRTPFIIKLKD | |
| | CPSSLGNGVKAYFEPGPTTDYSTGDLRAYKMVYATNPQTQLS | |
| | NIVAATEAQGVQVRISNLDDSKITMGANEATQQAAGFDPEVQ | |
| | TGGTSRTVTMRYLASYVKKNGDVEASAITTYVGFSVVYPGGG | |
| | SGGGSNDGTIVITGSISDQTCVIEEPSTLNHIKVVQLPKISKNAL | |
| | RNDGDTAGATPFDIKLKECPQALGALKLYFEPGITTNYDTGDL | |
| | IAYKQTYNAAGNGQLSTVSSATKAKGVEFRLANLNGQHIRMG | |
| | TDKTTQAAQTFTGKVTNGGKSYTLRYLASYVKKPKEDVDAA | |
| | QITSYVGFSVVYPGGGSNDGTIVITGSISDQTVI | |
| PolyA tail | 100 nt | |

FHA_20AALinker_Fim2-3

| SEQ ID NO: 84 consists of from 5′ end to 3′ end: | | 84 |
|---|---|---|
| 5′ UTR SEQ ID NO: 2, mRNA ORF SEQ ID NO: 85, and | | |
| 3′ UTR SEQ ID NO: 4. | | |
| 5′ UTR | GGGAAAUAAGAGAGAAAAGAAGAGUAAGAAGAAAUAUAA | 2 |
| | GACCCCGGCGCCGCCACC | |
| ORF of mRNA | AUGGAGACCCCUGCCCAGCUGCUGUUCCUGCUGCUGCUUU | 85 |
| Construct | GGCUGCCUGACACCACCGGAAGCCUGUACGCCGAACACGA | |
| (excluding the stop | CGCCACCCUGACCCUGGCCCAAGGCACCCAGAGGGAUCUG | |
| codon) | GUGGUGGACCAGGACCACAUCCUGCCCGUGGCCGAGGGA | |
| | ACCCUGAGAGUCAAAGCCAAGAGCCUCACCACCGAGAUCG | |
| | AGACAGGCAACCCUGGCAGCCUCAUCGCCGAGGUGCAGGA | |
| | GAACAUCGACAACAAGCAGGCCAUCGUCGUGGGCAAAGA | |
| | CCUGACUCUGUCCAGCGCCCACGGCAACGUAGCCAACGAG | |
| | GCCAACGCCCUGCUGUGGGCUGCCGGUGAGCUGACCGUGA | |
| | AGGCCCAGAACAUCACCAACAAGAGAGCCGCCCUGAUUGA | |
| | GGCCGGCGGGAACGCCAGACUGACCGCUGCCGUCGCCCUU | |
| | CUGAACAAGCUGGGCAGAAUCAGAGCCGGCGAGGACAUG | |
| | CACCUGGACGCCCCUAGAAUCGAGAACACCGCCAAGCUGA | |
| | GCGGCGAGGUGCAGAGAAAGGGCGUGCAGGACGUCGGCG | |
| | GAGGCGAGCACGGCAGGUGGAGCGGCAUCGGCUACGUGA | |
| | ACUACUGGCUUCGGGCCGGCAACGGCAAGAAAGCCGGCAC | |
| | CAUCGCCGCACCUUGGUACGGUGGCGAUCUGACCGCAGAG | |
| | CAGAGCCUGAUCGAGGUGGGCAAGGACCUGUACCUGAAC | |
| | GCCGGCGCCAGAAAGGACGAGCACAGACACCUGCUGAACG | |
| | AGGGCGUGAUCCAGGCCGGAGGUCACGGCCACAUCGGCG | |
| | GCGACGUGGACAACAGGGCCGUGGUCCGUACCGUGAGCG | |
| | CCAUGGAGUACUUCAAGACCCCUCUGCCUGUGAGCCUGAC | |
| | CGCCCUGGACAAUAGAGCCGGACUCAGCCCAGCCACCUGG | |
| | AACUUCCAGAGCACCUACGAGCUGCUGGACUACCUGCUGG | |
| | ACCAGAACAGAUACGAGUACAUCUGGGGCCUGUACCCUA | |
| | CCUACACCGAGUGGAGCGUGAACACCCUGAAGAACCUGG | |
| | ACCUGGGCUACCAGGCCAAGCCUGCCCCUACCGCCCCUCC | |
| | UAUGCCUAAGGCCCCUGAGCUGGACCUGAGAGGCCAUACC | |
| | CUGGAGAGCGCCGAGGGCAGAAAGAUCUUCGGCGAGUAC | |
| | AAGAAGCUGCAGGGCGAGUACGAGAAGGCCAAGAUGGCC | |
| | GUGCAAGCCGUGGAGGCCUACGGCGAGGCCACCAGAAGA | |
| | GUGCACGACCAGCUCGGGCAGCGAUACGGCAAGGCCCUGG | |
| | GCGGCAUGGACGCCGAAACCAAGGAGGUGGACGGCAUCA | |
| | UCCAGGAGUUUGCGGCCGAUCUGAGAACCGUGUACGCCA | |
| | AACAGGCCGACCAAGCCACUAUCGACGCCGAGACUGACAA | |
| | GGUGGCCCAGAGAUACAAGAGCCAGAUCGACGCCGUGAG | |
| | ACUCCAAGGAGGAGGUGGCUCUGGCGGAGGUGGAAGCGG | |
| | UGGAGGCGGAAGUGGUGGCGGAGGCAGCGUGGAUCCACC | |
| | UGUGGACUGCGGACGUGCAUUAGGACUGCACUUCUGGUC | |
| | AAGUGCGAGCCUUAUAUCGGAUCAGACCCCUGACGGCAC | |
| | AUUGAUCGGCAAGCCGGUGGUCGGACGGUCACUAUUGAG | |
| | CAAGUCUUGCAAGGUGCCUGACGACAUUAAGGAAGACCU | |
| | GUCAGACAAUCACGACGGCGAGCCAGUUGAUAUCGUACU | |
| | GGAGCUUGGCAGCAACUAUAAGAUUAGGCCACAGUCGUA | |
| | CGGUCAUCCUGGAAUAGUGGUCGACCUGCCUUCCGGUUC | |
| | AACAGAGGAGACAGGUAUCGCUAUCUACAUCGACUUCGG | |
| | CUCUAGCCCAAUGCAGAAGGUCGGCGAGAGACAGUGGCU | |
| | GUACCCACAGAAGGGUGAGGUGCUUUUCGACGUGCUGAC | |
| | UAUUAACGGAGACAACGCCGAGGUUCGGUACCAGGCCAU | |
| | CAAGGUCGGUCCACUGAAGCGCCCAAGAAAGCUGGUCCU | |
| | UAGUCAGUUCCCAAACCUCUUCACUUACAAGUGGGUCUU | |
| | CAUGAGAGGCACAAGCCAGGAGCGGGUCCUCGCACAGGG | |
| | CACCAUAGAUACUGACGUGGCCACGAGCACCAUCGACCUG | |
| | AAGACGUGCAGAUACACCAGUCAGACCGUGUCGCUCCCAA | |
| | UCAUUCAGAGGAGUGCCUUGACCGGUGUUGGCACCACAC | |
| | UUGGCAUGACUGACUUCCAAAUGCCAUUCUGGUGUUACG | |
| | GCUGGCCUAAGGUGUCAGUUUACAUGUCUGCGACAAAGA | |
| | CUCAAACAGGCGUAGACGGCGUAGCACUCCCAGCAACCGG | |
| | ACAGGCCGCCGGUAUGGCCUCCGGCGUGGGCGUUCAGUU | |
| | GAUUAACGGUAAGACGCAGCAGCCUGUCAAGCUGGGACU | |
| | CCAGGGCAAGAUUGCCCUGCCUGAGGCACAGCAGACCGAA | |
| | AGUGCAACAUUCUCACUGCCAAUGAAGGCCCAGUACUACC | |
| | AGACCAGUACCUCUACAUCUGCCGGCAAGCUGUCCGUUAC | |
| | CUACGCCGUCACUCUGAAUUACGACGGCGGAGGCUCCGAC | |
| | GACGGCACAAUCGUGAUAACCGGUACAAUUACUGACACA | |
| | ACCUGUGUAAUCGAGGAUCCUUCCGGCCCAUCGCAUACUA | |
| | AGGUUGUUCAGCUGCCAAAGAUUUCCAAGAACGCCCUCA | |
| | AGGCCAACGGAGAUCAAGCUGGUAGGACACCAUUCAUAA | |
| | UUAAGCUAAAGGACUGUCCAUCCUCCCUCGGUAACGGCG | |
| | UGAAGGCCUAUUUCGAGCCAGGACCUACGACUGACUACU | |
| | CUACAGGUGAUCUCAGAGCAUAUAAGAUGGUUUACGCAA | |
| | CAAAUCCUCAGACACAGCUCAGCAAUAUCGUCGCCGCCAC | |
| | UGAGGCUCAGGGAGUCCAGGUCCGGAUCUCCAACUUAGA | |
| | CGACUCAAAGAUCACCAUGGGCGCCAACGAGGCAACUCAG | |
| | CAGGCCGCUGGAUUCGACCCAGAGGUACAGACUGGCGGU | |
| | ACGUCCCGCACAGUCACCAUGCGGUACCUGGCAAGUUACG | |
| | UUAAGAAGAACGGUGACGUGGAGGCGUCCGCCAUAACCA | |
| | CAUACGUGGGAUUCUCAGUGGUGUACCCUGGUGGCGGCU | |
| | CUGGCGGUGGCAGUAACGACGGUACUAUUGUAAUUACUG | |
| | GAAGUAUUUCAGACCAAACGUGCGUUAUCGAAGAACCAA | |
| | GCACAUUAAACCAUAUUAAGGUGGUGCAGUUGCCUAAGA | |
| | UAUCAAAGAACGCUUUGAGGAACGACGGCGAUACUGCAG | |
| | GCGCAACACCUUUCGAUAUAAAGCUUAAGGAGUGCCCUC | |
| | AGGCCCUGGGCGCCCUGAAGCUCUACUUCGAACCAGGCAU | |
| | AACAACUAACUACGACACCGGUGAUCUUAUUGCUUACAA | |
| | GCAGACAUAUAACGCAGCCGGCAACGGCCAGCUUUCCACU | |
| | GUAUCAUCCGCCACAAAGGCUAAGGGCGUCGAGUUCAGG | |
| | CUGGCGAAUCUCAACGGACAACACAUUAGAAUGGGUACU | |
| | GAUAAGACCACCCAAGCUGCACAGACCUUCACAGGUAAG | |
| | GUCACUAACGGCGGUAAGAGUUAUACACUGCGCUACCUG | |
| | GCUAGCUACGUGAAGAAGCCAAAGGAGGACGUCGACGCA | |
| | GCACAGAUAACAUCUUACGUAGGAUUCUCUGUUGUUUAU | |
| | CCGGGUGGCGGUAGCAACGACGGAACCAUUGUGAUCACU | |
| | GGCUCAAUUUCCGACCAGACAGUCAUU | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding amino | METPAQLLFLLLLWLPDTTGSLYAEHDATLTLAQGTQRDLVV | 86 |
| acid sequence | DQDHILPVAEGTLRVKAKSLTTEIETGNPGSLIAEVQENIDNKQ | |
| | AIVVGKDLTLSSAHGNVANEANALLWAAGELTVKAQNITNK | |
| | RAALIEAGGNARLTAAVALLNKLGRIRAGEDMHLDAPRIENT | |
| | AKLSGEVQRKGVQDVGGGEHGRWSGIGYVNYWLRAGNGKK | |
| | AGTIAAPWYGGDLTAEQSLIEVGKDLYLNAGARKDEHRHLLN | |
| | EGVIQAGGHGHIGGDVDNRAVVRTVSAMEYFKTPLPVSLTAL | |
| | DNRAGLSPATWNFQSTYELLDYLLDONRYEYIWGLYPTYTE | |
| | WSVNTLKNLDLGYQAKPAPTAPPMPKAPELDLRGHTLESAEG | |
| | RKIFGEYKKLQGEYEKAKMAVQAVEAYGEATRRVHDQLGQR | |
| | YGKALGGMDAETKEVDGIIQEFAADLRTVYAKQADQATIDAE | |
| | TDKVAQRYKSQIDAVRLQGGGGSGGGGSGGGGSGGGGSVDP | |
| | PVDCGRALGLHFWSSASLISDQTPDGTLIGKPVVGRSLLSKSC | |
| | KVPDDIKEDLSDNHDGEPVDIVLELGSNYKIRPQSYGHPGIVV | |
| | DLPSGSTEETGIAIYIDFGSSPMQKVGERQWLYPQKGEVLFDV | |
| | LTINGDNAEVRYQAIKVGPLKRPRKLVLSQFPNLFTYKWVFM | |
| | RGTSQERVLAQGTIDTDVATSTIDLKTCRYTSQTVSLPIIQRSA | |
| | LTGVGTTLGMTDFQMPFWCYGWPKVSVYMSATKTQTGVDG | |
| | VALPATGQAAGMASGVGVQLINGKTQQPVKLGLQGKIALPEA | |
| | QQTESATFSLPMKAQYYQTSTSTSAGKLSVTYAVTLNYDGGG | |
| | SDDGTIVITGTITDTTCVIEDPSGPSHTKVVQLPKISKNALKAN | |
| | GDQAGRTPFIIKLKDCPSSLGNGVKAYFEPGPTTDYSTGDLRA | |
| | YKMVYATNPQTQLSNIVAATEAQGVQVRISNLDDSKITMGAN | |
| | EATQQAAGFDPEVQTGGTSRTVTMRYLASYVKKNGDVEASAI | |
| | TTYVGFSVVYPGGGSGGGSNDGTIVITGSISDQTCVIEEPSTLN | |
| | HIKVVQLPKISKNALRNDGDTAGATPFDIKLKECPQALGALKL | |
| | YFEPGITTNYDTGDLIAYKQTYNAAGNGQLSTVSSATKAKGV | |
| | EFRLANLNGQHIRMGTDKTTQAAQTFTGKVTNGGKSYTLRYL | |
| | ASYVKKPKEDVDAAQITSYVGFSVVYPGGGSNDGTIVITGSIS | |
| | DQTVI | |
| PolyA tail | 100 nt | |

Fim2-3_20AALinker_FHA

| SEQ ID NO: 87 consists of from 5′ end to 3′ end: | | 87 |
|---|---|---|
| 5′ UTR SEQ ID NO: 2, mRNA ORF SEQ ID NO: 88, and | | |
| 3′ UTR SEQ ID NO: 4. | | |
| 5′ UTR | GGGAAAUAAGAGAGAAAAGAAGAGUAAGAAGAAAUAUAA | 2 |
| | GACCCCGGCGCCGCCACC | |
| ORF of mRNA | AUGGAGACCCCUGCCCAGCUGCUGUUCCUGCUGCUGCUUU | 88 |
| Construct | GGCUGCCUGACACCACCGGAGUGGAUCCACCUGUGGACUG | |
| (excluding the stop | CGGACGUGCAUUAGGACUGCACUUCUGGUCAAGUGCGAG | |
| codon) | CCUUAUAUCGGAUCAGACCCCUGACGGCACAUUGAUCGGC | |
| | AAGCCGGUGGUCGGACGGUCACUAUUGAGCAAGUCUUGC | |
| | AAGGUGCCUGACGACAUUAAGGAAGACCUGUCAGACAAU | |
| | CACGACGGCGAGCCAGUUGAUAUCGUACUGGAGCUUGGC | |
| | AGCAACUAUAAGAUUAGGCCACAGUCGUACGGUCAUCCU | |
| | GGAAUAGUGGUCGACCUGCCUUCCGGUUCAACAGAGGAG | |
| | ACAGGUAUCGCUAUCUACAUCGACUUCGGCUCUAGCCCAA | |
| | UGCAGAAGGUCGGCGAGAGACAGUGGCUGUACCCACAGA | |
| | AGGGUGAGGUGCUUUUCGACGUGCUGACUAUUAACGGAG | |
| | ACAACGCCGAGGUUCGGUACCAGGCCAUCAAGGUCGGUCC | |
| | ACUGAAGCGCCCAAGAAAGCUGGUCCUUAGUCAGUUCCC | |
| | AAACCUCUUCACUUACAAGUGGGUCUUCAUGAGAGGCAC | |
| | AAGCCAGGAGCGGGUCCUCGCACAGGGCACCAUAGAUAC | |
| | UGACGUGGCCACGAGCACCAUCGACCUGAAGACGUGCAG | |
| | AUACACCAGUCAGACCGUGUCGCUCCCAAUCAUUCAGAGG | |
| | AGUGCCUUGACCGGUGUUGGCACCACACUUGGCAUGACU | |
| | GACUUCCAAAUGCCAUUCUGGUGUUACGGCUGGCCUAAG | |
| | GUGUCAGUUUACAUGUCUGCGACAAAGACUCAAACAGGC | |
| | GUAGACGGCGUAGCACUCCCAGCAACCGGACAGGCCGCCG | |
| | GUAUGGCCUCCGGCGUGGGCGUUCAGUUGAUUAACGGUA | |
| | AGACGCAGCAGCCUGUCAAGCUGGGACUCCAGGGCAAGA | |
| | UUGCCCUGCCUGAGGCACAGCAGACCGAAAGUGCAACAU | |
| | UCUCACUGCCAAUGAAGGCCCAGUACUACCAGACCAGUAC | |
| | CUCUACAUCUGCCGGCAAGCUGUCCGUUACCUACGCCGUC | |
| | ACUCUGAAUUACGACGGCGGAGGCUCCGACGACGGCACA | |
| | AUCGUGAUAACCGGUACAAUUACUGACACAACCUGUGUA | |
| | AUCGAGGAUCCUUCCGGCCCAUCGCAUACUAAGGUUGUU | |
| | CAGCUGCCAAAGAUUUCCAAGAACGCCCUCAAGGCCAACG | |
| | GAGAUCAAGCUGGUAGGACACCAUUCAUAAUUAAGCUAA | |
| | AGGACUGUCCAUCCUCCCUCGGUAACGGCGUGAAGGCCUA | |
| | UUUCGAGCCAGGACCUACGACUGACUACUCUACAGGUGA | |
| | UCUCAGAGCAUAUAAGAUGGUUUACGCAACAAAUCCUCA | |
| | GACACAGCUCAGCAAUAUCGUCGCCGCCACUGAGGCUCAG | |
| | GGAGUCCAGGUCCGGAUCUCCAACUUAGACGACUCAAAG | |
| | AUCACCAUGGGCGCCAACGAGGCAACUCAGCAGGCCGCUG | |
| | GAUUCGACCCAGAGGUACAGACUGGCGGUACGUCCCGCAC | |
| | AGUCACCAUGCGGUACCUGGCAAGUUACGUUAAGAAGAA | |
| | CGGUGACGUGGAGGCGUCCGCCAUAACCACAUACGUGGG | |
| | AUUCUCAGUGGUGUACCCUGGUGGCGGCUCUGGCGGUGG | |
| | CAGUAACGACGGUACUAUUGUAAUUACUGGAAGUAUUUC | |
| | AGACCAAACGUGCGUUAUCGAAGAACCAAGCACAUUAAA | |
| | CCAUAUUAAGGUGGUGCAGUUGCCUAAGAUAUCAAAGAA | |
| | CGCUUUGAGGAACGACGGCGAUACUGCAGGCGCAACACC | |
| | UUUCGAUAUAAAGCUUAAGGAGUGCCCUCAGGCCCUGGG | |
| | CGCCCUGAAGCUCUACUUCGAACCAGGCAUAACAACUAAC | |
| | UACGACACCGGUGAUCUUAUUGCUUACAAGCAGACAUAU | |
| | AACGCAGCCGGCAACGGCCAGCUUUCCACUGUAUCAUCCG | |
| | CCACAAAGGCUAAGGGCGUCGAGUUCAGGCUGGCGAAUC | |
| | UCAACGGACAACACAUUAGAAUGGGUACUGAUAAGACCA | |
| | CCCAAGCUGCACAGACCUUCACAGGUAAGGUCACUAACGG | |
| | CGGUAAGAGUUAUACACUGCGCUACCUGGCUAGCUACGU | |
| | GAAGAAGCCAAAGGAGGACGUCGACGCAGCACAGAUAAC | |
| | AUCUUACGUAGGAUUCUCUGUUGUUUAUCCGGGUGGCGG | |
| | UAGCAACGACGGAACCAUUGUGAUCACUGGCUCAAUUUC | |
| | CGACCAGACAGUCAUUGGAGGAGGUGGCUCUGGCGGAGG | |
| | UGGAAGCGGUGGAGGCGGAAGUGGUGGCGGAGGCAGCAG | |
| | CCUGUACGCCGAACACGACGCCACCCUGACCCUGGCCCAA | |
| | GGCACCCAGAGGGAUCUGGUGGUGGACCAGGACCACAUC | |
| | CUGCCCGUGGCCGAGGGAACCCUGAGAGUCAAAGCCAAG | |
| | AGCCUCACCACCGAGAUCGAGACAGGCAACCCUGGCAGCC | |
| | UCAUCGCCGAGGUGCAGGAGAACAUCGACAACAAGCAGG | |
| | CCAUCGUCGUGGGCAAAGACCUGACUCUGUCCAGCGCCCA | |
| | CGGCAACGUAGCCAACGAGGCCAACGCCCUGCUGUGGGCU | |
| | GCCGGUGAGCUGACCGUGAAGGCCCAGAACAUCACCAACA | |
| | AGAGAGCCGCCCUGAUUGAGGCCGGCGGGAACGCCAGAC | |
| | UGACCGCUGCCGUCGCCCUUCUGAACAAGCUGGGCAGAAU | |
| | CAGAGCCGGCGAGGACAUGCACCUGGACGCCCCUAGAAUC | |
| | GAGAACACCGCCAAGCUGAGCGGCGAGGUGCAGAGAAAG | |
| | GGCGUGCAGGACGUCGGCGGAGGCGAGCACGGCAGGUGG | |
| | AGCGGCAUCGGCUACGUGAACUACUGGCUUCGGGCCGGC | |
| | AACGGCAAGAAAGCCGGCACCAUCGCCGCACCUUGGUACG | |
| | GUGGCGAUCUGACCGCAGAGCAGAGCCUGAUCGAGGUGG | |
| | GCAAGGACCUGUACCUGAACGCCGGCGCCAGAAAGGACG | |
| | AGCACAGACACCUGCUGAACGAGGGCGUGAUCCAGGCCG | |
| | GAGGUCACGGCCACAUCGGCGGCGACGUGGACAACAGGG | |
| | CCGUGGUCCGUACCGUGAGCGCCAUGGAGUACUUCAAGA | |
| | CCCCUCUGCCUGUGAGCCUGACCGCCCUGGACAAUAGAGC | |
| | CGGACUCAGCCCAGCCACCUGGAACUUCCAGAGCACCUAC | |
| | GAGCUGCUGGACUACCUGCUGGACCAGAACAGAUACGAG | |
| | UACAUCUGGGGCCUGUACCCUACCUACACCGAGUGGAGCG | |
| | UGAACACCCUGAAGAACCUGGACCUGGGCUACCAGGCCAA | |
| | GCCUGCCCCUACCGCCCCUCCUAUGCCUAAGGCCCCUGAG | |
| | CUGGACCUGAGAGGCCAUACCCUGGAGAGCGCCGAGGGC | |
| | AGAAAGAUCUUCGGCGAGUACAAGAAGCUGCAGGGCGAG | |
| | UACGAGAAGGCCAAGAUGGCCGUGCAAGCCGUGGAGGCC | |
| | UACGGCGAGGCCACCAGAAGAGUGCACGACCAGCUCGGGC | |
| | AGCGAUACGGCAAGGCCCUGGGCGGCAUGGACGCCGAAA | |
| | CCAAGGAGGUGGACGGCAUCAUCCAGGAGUUUGCGGCCG | |
| | AUCUGAGAACCGUGUACGCCAAACAGGCCGACCAAGCCAC | |
| | UAUCGACGCCGAGACUGACAAGGUGGCCCAGAGAUACAA | |
| | GAGCCAGAUCGACGCCGUGAGACUCCAA | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding amino | METPAQLLFLLLLWLPDTTGVDPPVDCGRALGLHFWSSASLIS | 89 |
| acid sequence | DQTPDGTLIGKPVVGRSLLSKSCKVPDDIKEDLSDNHDGEPVD | |
| | IVLELGSNYKIRPQSYGHPGIVVDLPSGSTEETGIAIYIDFGSSP | |
| | MQKVGERQWLYPQKGEVLFDVLTINGDNAEVRYQAIKVGPL | |
| | KRPRKLVLSQFPNLFTYKWVFMRGTSQERVLAQGTIDTDVAT | |
| | STIDLKTCRYTSQTVSLPIIQRSALTGVGTTLGMTDFQMPFWC | |
| | YGWPKVSVYMSATKTQTGVDGVALPATGQAAGMASGVGVQ | |
| | LINGKTQQPVKLGLQGKIALPEAQQTESATFSLPMKAQYYQTS | |
| | TSTSAGKLSVTYAVTLNYDGGGSDDGTIVITGTITDTTCVIEDP | |
| | SGPSHTKVVQLPKISKNALKANGDQAGRTPFIIKLKDCPSSLGN | |
| | GVKAYFEPGPTTDYSTGDLRAYKMVYATNPQTQLSNIVAATE | |
| | AQGVQVRISNLDDSKITMGANEATQQAAGFDPEVQTGGTSRT | |
| | VTMRYLASYVKKNGDVEASAITTYVGFSVVYPGGGSGGGSN | |
| | DGTIVITGSISDQTCVIEEPSTLNHIKVVQLPKISKNALRNDGDT | |
| | AGATPFDIKLKECPQALGALKLYFEPGITTNYDTGDLIAYKQT | |
| | YNAAGNGQLSTVSSATKAKGVEFRLANLNGQHIRMGTDKTT | |
| | QAAQTFTGKVTNGGKSYTLRYLASYVKKPKEDVDAAQITSYV | |
| | GFSVVYPGGGSNDGTIVITGSISDQTVIGGGGSGGGGSGGGGS | |
| | GGGGSSLYAEHDATLTLAQGTQRDLVVDQDHILPVAEGTLRV | |
| | KAKSLTTEIETGNPGSLIAEVQENIDNKQAIVVGKDLTLSSAHG | |
| | NVANEANALLWAAGELTVKAQNITNKRAALIEAGGNARLTA | |
| | AVALLNKLGRIRAGEDMHLDAPRIENTAKLSGEVQRKGVQDV | |
| | GGGEHGRWSGIGYVNYWLRAGNGKKAGTIAAPWYGGDLTA | |
| | EQSLIEVGKDLYLNAGARKDEHRHLLNEGVIQAGGHGHIGGD | |
| | VDNRAVVRTVSAMEYFKTPLPVSLTALDNRAGLSPATWNFQS | |
| | TYELLDYLLDQNRYEYIWGLYPTYTEWSVNTLKNLDLGYQA | |
| | KPAPTAPPMPKAPELDLRGHTLESAEGRKIFGEYKKLQGEYEK | |
| | AKMAVQAVEAYGEATRRVHDQLGQRYGKALGGMDAETKEV | |
| | DGIIQEFAADLRTVYAKQADQATIDAETDKVAQRYKSQIDAV | |
| | RLQ | |
| PolyA tail | 100 nt | |

RTX_FurinF2A_Diptheria

| SEQ ID NO: 90 consists of from 5′ end to 3′ end: | | 90 |
|---|---|---|
| 5′ UTR SEQ ID NO: 2, mRNA ORF SEQ ID NO: 91, and | | |
| 3′ UTR SEQ ID NO: 4. | | |
| 5′ UTR | GGGAAAUAAGAGAGAAAAGAAGAGUAAGAAGAAAUAUAA | 2 |
| | GACCCCGGCGCCGCCACC | |
| ORF of mRNA | AUGGAGACCCCUGCCCAGCUGCUGUUCCUGCUGCUGCUUU | 91 |
| Construct | GGCUGCCUGACACCACCGGAGUGGAUCCACCUGUGGACUG | |
| (excluding the stop | CGGACGUGCAUUAGGACUGCACUUCUGGUCAAGUGCGAG | |
| codon) | CCUUAUAUCGGAUCAGACCCCUGACGGCACAUUGAUCGGC | |
| | AAGCCGGUGGUCGGACGGUCACUAUUGAGCAAGUCUUGC | |
| | AAGGUGCCUGACGACAUUAAGGAAGACCUGUCAGACAAU | |
| | CACGACGGCGAGCCAGUUGAUAUCGUACUGGAGCUUGGC | |
| | AGCAACUAUAAGAUUAGGCCACAGUCGUACGGUCAUCCU | |
| | GGAAUAGUGGUCGACCUGCCUUCCGGUUCAACAGAGGAG | |
| | ACAGGUAUCGCUAUCUACAUCGACUUCGGCUCUAGCCCAA | |
| | UGCAGAAGGUCGGCGAGAGACAGUGGCUGUACCCACAGA | |
| | AGGGUGAGGUGCUUUUCGACGUGCUGACUAUUAACGGAG | |
| | ACAACGCCGAGGUUCGGUACCAGGCCAUCAAGGUCGGUCC | |
| | ACUGAAGCGCCCAAGAAAGCUGGUCCUUAGUCAGUUCCC | |
| | AAACCUCUUCACUUACAAGUGGGUCUUCAUGAGAGGCAC | |
| | AAGCCAGGAGCGGGUCCUCGCACAGGGCACCAUAGAUAC | |
| | UGACGUGGCCACGAGCACCAUCGACCUGAAGACGUGCAG | |
| | AUACACCAGUCAGACCGUGUCGCUCCCAAUCAUUCAGAGG | |
| | AGUGCCUUGACCGGUGUUGGCACCACACUUGGCAUGACU | |
| | GACUUCCAAAUGCCAUUCUGGUGUUACGGCUGGCCUAAG | |
| | GUGUCAGUUUACAUGUCUGCGACAAAGACUCAAACAGGC | |
| | GUAGACGGCGUAGCACUCCCAGCAACCGGACAGGCCGCCG | |
| | GUAUGGCCUCCGGCGUGGGCGUUCAGUUGAUUAACGGUA | |
| | AGACGCAGCAGCCUGUCAAGCUGGGACUCCAGGGCAAGA | |
| | UUGCCCUGCCUGAGGCACAGCAGACCGAAAGUGCAACAU | |
| | UCUCACUGCCAAUGAAGGCCCAGUACUACCAGACCAGUAC | |
| | CUCUACAUCUGCCGGCAAGCUGUCCGUUACCUACGCCGUC | |
| | ACUCUGAAUUACGACGGCGGAGGCUCCGACGACGGCACA | |
| | AUCGUGAUAACCGGUACAAUUACUGACACAACCUGUGUA | |
| | AUCGAGGAUCCUUCCGGCCCAUCGCAUACUAAGGUUGUU | |
| | CAGCUGCCAAAGAUUUCCAAGAACGCCCUCAAGGCCAACG | |
| | GAGAUCAAGCUGGUAGGACACCAUUCAUAAUUAAGCUAA | |
| | AGGACUGUCCAUCCUCCCUCGGUAACGGCGUGAAGGCCUA | |
| | UUUCGAGCCAGGACCUACGACUGACUACUCUACAGGUGA | |
| | UCUCAGAGCAUAUAAGAUGGUUUACGCAACAAAUCCUCA | |
| | GACACAGCUCAGCAAUAUCGUCGCCGCCACUGAGGCUCAG | |
| | GGAGUCCAGGUCCGGAUCUCCAACUUAGACGACUCAAAG | |
| | AUCACCAUGGGCGCCAACGAGGCAACUCAGCAGGCCGCUG | |
| | GAUUCGACCCAGAGGUACAGACUGGCGGUACGUCCCGCAC | |
| | AGUCACCAUGCGGUACCUGGCAAGUUACGUUAAGAAGAA | |
| | CGGUGACGUGGAGGCGUCCGCCAUAACCACAUACGUGGG | |
| | AUUCUCAGUGGUGUACCCUGGUGGCGGCUCUGGCGGUGG | |
| | CAGUAACGACGGUACUAUUGUAAUUACUGGAAGUAUUUC | |
| | AGACCAAACGUGCGUUAUCGAAGAACCAAGCACAUUAAA | |
| | CCAUAUUAAGGUGGUGCAGUUGCCUAAGAUAUCAAAGAA | |
| | CGCUUUGAGGAACGACGGCGAUACUGCAGGCGCAACACC | |
| | UUUCGAUAUAAAGCUUAAGGAGUGCCCUCAGGCCCUGGG | |
| | CGCCCUGAAGCUCUACUUCGAACCAGGCAUAACAACUAAC | |
| | UACGACACCGGUGAUCUUAUUGCUUACAAGCAGACAUAU | |
| | AACGCAGCCGGCAACGGCCAGCUUUCCACUGUAUCAUCCG | |
| | CCACAAAGGCUAAGGGCGUCGAGUUCAGGCUGGCGAAUC | |
| | UCAACGGACAACACAUUAGAAUGGGUACUGAUAAGACCA | |
| | CCCAAGCUGCACAGACCUUCACAGGUAAGGUCACUAACGG | |
| | CGGUAAGAGUUAUACACUGCGCUACCUGGCUAGCUACGU | |
| | GAAGAAGCCAAAGGAGGACGUCGACGCAGCACAGAUAAC | |
| | AUCUUACGUAGGAUUCUCUGUUGUUUAUCCGGGUGGCGG | |
| | UAGCAACGACGGAACCAUUGUGAUCACUGGCUCAAUUUC | |
| | CGACCAGACAGUCAUUGGAGGAGGUGGCUCUGGCGGAGG | |
| | UGGAAGCGGUGGAGGCGGAAGUGGUGGCGGAGGCAGCAG | |
| | CCUGUACGCCGAACACGACGCCACCCUGACCCUGGCCCAA | |
| | GGCACCCAGAGGGAUCUGGUGGUGGACCAGGACCACAUC | |
| | CUGCCCGUGGCCGAGGGAACCCUGAGAGUCAAAGCCAAG | |
| | AGCCUCACCACCGAGAUCGAGACAGGCAACCCUGGCAGCC | |
| | UCAUCGCCGAGGUGCAGGAGAACAUCGACAACAAGCAGG | |
| | CCAUCGUCGUGGGCAAAGACCUGACUCUGUCCAGCGCCCA | |
| | CGGCAACGUAGCCAACGAGGCCAACGCCCUGCUGUGGGCU | |
| | GCCGGUGAGCUGACCGUGAAGGCCCAGAACAUCACCAACA | |
| | AGAGAGCCGCCCUGAUUGAGGCCGGCGGGAACGCCAGAC | |
| | UGACCGCUGCCGUCGCCCUUCUGAACAAGCUGGGCAGAAU | |
| | GAGAACACCGCCAAGCUGAGCGGCGAGGUGCAGAGAAAG | |
| | GGCGUGCAGGACGUCGGCGGAGGCGAGCACGGCAGGUGG | |
| | AGCGGCAUCGGCUACGUGAACUACUGGCUUCGGGCCGGC | |
| | AACGGCAAGAAAGCCGGCACCAUCGCCGCACCUUGGUACG | |
| | GUGGCGAUCUGACCGCAGAGCAGAGCCUGAUCGAGGUGG | |
| | GCAAGGACCUGUACCUGAACGCCGGCGCCAGAAAGGACG | |
| | AGCACAGACACCUGCUGAACGAGGGCGUGAUCCAGGCCG | |
| | GAGGUCACGGCCACAUCGGCGGCGACGUGGACAACAGGG | |
| | CCGUGGUCCGUACCGUGAGCGCCAUGGAGUACUUCAAGA | |
| | CCCCUCUGCCUGUGAGCCUGACCGCCCUGGACAAUAGAGC | |
| | CGGACUCAGCCCAGCCACCUGGAACUUCCAGAGCACCUAC | |
| | GAGCUGCUGGACUACCUGCUGGACCAGAACAGAUACGAG | |
| | UACAUCUGGGGCCUGUACCCUACCUACACCGAGUGGAGCG | |
| | UGAACACCCUGAAGAACCUGGACCUGGGCUACCAGGCCAA | |
| | GCCUGCCCCUACCGCCCCUCCUAUGCCUAAGGCCCCUGAG | |
| | CUGGACCUGAGAGGCCAUACCCUGGAGAGCGCCGAGGGC | |
| | AGAAAGAUCUUCGGCGAGUACAAGAAGCUGCAGGGCGAG | |
| | UACGAGAAGGCCAAGAUGGCCGUGCAAGCCGUGGAGGCC | |
| | UACGGCGAGGCCACCAGAAGAGUGCACGACCAGCUCGGGC | |
| | AGCGAUACGGCAAGGCCCUGGGCGGCAUGGACGCCGAAA | |
| | CCAAGGAGGUGGACGGCAUCAUCCAGGAGUUUGCGGCCG | |
| | AUCUGAGAACCGUGUACGCCAAACAGGCCGACCAAGCCAC | |
| | UAUCGACGCCGAGACUGACAAGGUGGCCCAGAGAUACAA | |
| | GAGCCAGAUCGACGCCGUGAGACUCCAA | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding amino | METPAQLLFLLLLWLPDTTGTLEHVQHIIGGAGNDAITGNAHD | 92 |
| acid sequence | NFLAGGSGDDRLDGGAGNDVLVGGEGQNTVIGGAGDDVFLQ | |
| | DLGVWSNQLDGGAGVDTVKYNVHQPSEERLERMGDTGIHAD | |
| | LQKGTVEKWPALNLFSVDHVKNIENLHGSRLNDRIAGDDQDN | |
| | ELWGHDGNDVIRGRGGDDILRGGLGLDTLYGEDGNDIFLQDD | |
| | ETVSDDIDGGAGLDTVDYSAMIHPGRIVAPHEYGFGIEADLSR | |
| | EWVRKASALGVDYYDNVRNVENVIGTSMKDVLIGDAQANTL | |
| | MGQGGDDTVRGGDGDDLLFGGDGNDMLYGDAGNDVLYGG | |
| | LGDDTLEGGAGNDWFGQTQAREHDVLRGGDGVDTVDYSQT | |
| | GAHAGIAAGRIGLGILADLGAGRVDKLGEAGSSAYDTVSGIEN | |
| | VVGTELADRITGDAQANVLRGAGGADVLAGGEGDDVLLGGD | |
| | GDDQLSGDAGRDRLYGEAGDDWFFQDAANAGNLLDGGDGR | |
| | DTVDFSGPGRGLDAGAKGVFLSLGKGFASLMDEPETSNVLRN | |
| | IENAVGSARDDVLIGDAGANVLNGLAGNDVLSGGAGDDVLL | |
| | GDEGSDLLSGDAGNDDLFGGQGDDTYLFGVGYGHDTRAKRS | |
| | GSGAPVKQTLNFDLLKLAGDVESNPGPMSRKLFASILIGALLGI | |
| | GAPPSAHAGADDVVDSSKSFVMENFASYHGTKPGYVDSIQKG | |
| | IQKPKSGTQGNYDDDWEGFYSTDNKYDAAGYSVDNENPLSG | |
| | KAGGVVKVTYPGLTKVLALKVDNAETIKKELGLSLTEPLMEQ | |
| | VGTEEFIKRFGDGASRVVLSLPFAEGSSSVKYINNWEQAKALS | |
| | VELEINFETRGKRGQDAMYEYMAQACAGNRVRRSVGSSLSCI | |
| | NLDWDVIRDKTKTKIESLKEHGPIKNKMSESPNKAVSEEKAK | |
| | QYLEEFHQTALEHPELSELKTVTGTNPVFAGANYAAWAVNV | |
| | AQVIDSETADNLEKTTAALSILPGIGSVMGIADGAVHHNTEEIV | |
| | AQSIALSSLMVAQAIPLVGELVDIGFAAYNFVESIINLFQVVHN | |
| | SYNRPAYSPGHKTQPFLHDGYAVSWNTVEDSIIRTGFQGESGH | |
| | DIKITAENTPLPIAGVLLPTIPGKLDVNKAKTHISVNGRKIRMR | |
| | CRAIDGDVTFCRPKSPVYVGNGVHANLHVAFHRSSSEKIHSNE | |
| | ISSDSIGVLGYQKTVDHTKVNSKLSLFFEIKS | |
| PolyA tail | 100 nt | |

RTX_20AALinker_Diptheria

| SEQ ID NO: 93 consists of from 5′ end to 3′ end: | | 93 |
|---|---|---|
| 5′ UTR SEQ ID NO: 2, mRNA ORF SEQ ID NO: 94, and | | |
| 3′ UTR SEQ ID NO: 4. | | |
| 5′ UTR | GGGAAAUAAGAGAGAAAAGAAGAGUAAGAAGAAAUAUAA | 2 |
| | GACCCCGGCGCCGCCACC | |
| ORF of mRNA | AUGGAGACCCCUGCCCAGCUGCUGUUCCUGCUGCUGCUUU | 94 |
| Construct | GGCUGCCAGACACCACCGGAACCCUGGAGCACGUGCAGCA | |
| (excluding the stop | CAUCAUCGGCGGCGCCGGCAACGACGCCAUCACUGGCAAC | |
| codon) | GCCCACGACAACUUCCUGGCCGGCGGCAGCGGCGACGACA | |
| | GACUGGACGGCGGAGCAGGUAACGACGUGCUCGUCGGAG | |
| | GCGAGGGGCAGAACACCGUGAUUGGCGGAGCCGGAGACG | |
| | ACGUGUUCCUGCAGGACCUGGGCGUGUGGAGCAACCAGU | |
| | UAGACGGCGGCGCAGGUGUGGAUACUGUUAAGUACAACG | |
| | UGCACCAGCCUAGCGAGGAGAGACUGGAGAGAAUGGGCG | |
| | ACACCGGCAUCCACGCCGACCUGCAGAAGGGAACCGUGGA | |
| | GAAGUGGCCUGCCCUGAACCUGUUCAGCGUGGACCACGU | |
| | GAAGAACAUCGAGAACCUGCACGGCAGUAGACUAAACGA | |
| | CAGAAUUGCAGGAGACGAUCAGGACAACGAACUGUGGGG | |
| | UCACGACGGAAACGACGUGAUACGUGGCAGAGGAGGCGA | |
| | CGACAUCCUGCGGGGCGGCCUGGGCCUGGACACCCUGUAC | |
| | GGCGAGGACGGAAACGACAUCUUCCUCCAAGACGACGAA | |
| | ACCGUGAGCGACGAUAUCGACGGCGGUGCCGGAUUAGAC | |
| | ACCGUCGACUACAGCGCCAUGAUCCACCCUGGCAGAAUCG | |
| | UGGCCCCUCACGAGUACGGCUUCGGCAUCGAGGCUGACCU | |
| | GAGCAGAGAGUGGGUGAGAAAGGCCAGCGCGUUGGGUGU | |
| | AGACUACUACGACAACGUGAGAAACGUCGAGAACGUCAU | |
| | UGGCACCAGCAUGAAAGACGUGCUCAUCGGUGACGCUCA | |
| | GGCCAACACCCUGAUGGGCCAGGGCGGUGACGACACAGU | |
| | GCGAGGCGGAGACGGAGACGACCUGCUGUUCGGUGGCGA | |
| | CGGCAACGAUAUGUUAUACGGCGACGCUGGAAACGACGU | |
| | GUUAUACGGUGGACUGGGUGACGAUACAUUGGAGGGUGG | |
| | CGCGGGCAACGAUUGGUUCGGCCAGACACAAGCCAGAGA | |
| | GCACGACGUUCUACGUGGCGGCGACGGAGUGGACACUGU | |
| | GGACUACUCACAAACCGGCGCCCACGCCGGAAUCGCGGCC | |
| | GGGAGAAUCGGCUUGGGAAUCCUUGCUGACUUGGGUGCA | |
| | GGCCGGGUGGACAAGCUGGGUGAGGCAGGCUCAAGUGCC | |
| | UACGAUACAGUGAGCGGCAUUGAGAACGUAGUCGGCACG | |
| | GAGCUUGCUGAUCGGAUUACCGGAGACGCCCAAGCAAAC | |
| | GUGUUACGGGGUGCUGGAGGCGCUGACGUCCUCGCUGGC | |
| | GGUGAGGGCGACGACGUGCUGUUGGGAGGAGACGGUGAC | |
| | GACCAACUGUCGGGCGACGCGGGAAGGGAUAGACUGUAC | |
| | GGAGAAGCAGGAGACGACUGGUUCUUCCAGGACGCCGCC | |
| | AACGCGGGAAACCUGCUUGACGGUGGUGACGGACGAGAU | |
| | ACCGUAGAUUUCUCUGGUCCAGGCAGAGGCCUCGACGCA | |
| | GGCGCCAAGGGCGUCUUCCUGUCCCUCGGUAAGGGCUUCG | |
| | CCAGCCUUAUGGACGAACCUGAGACUUCUAACGUGCUGA | |
| | GAAAUAUUGAGAACGCCGUCGGCAGCGCCAGGGACGACG | |
| | UCUUAAUCGGCGACGCCGGUGCGAACGUCCUAAACGGCU | |
| | UGGCCGGAAACGACGUUUUAAGCGGAGGAGCUGGCGACG | |
| | ACGUACUGCUUGGCGACGAGGGCAGCGACCUCCUAUCAG | |
| | GUGACGCCGGUAACGACGACCUCUUCGGCGGACAGGGUG | |
| | ACGAUACUUAUUUGUUCGGCGUGGGCUACGGACACGACA | |
| | CUGGAGGAGGUGGCUCUGGCGGAGGUGGAAGCGGUGGAG | |
| | GCGGAAGUGGUGGCGGAGGCAGCGGAGCUGACGACGUGG | |
| | UGGACAGCAGCAAGAGCUUCGUGAUGGAGAACUUCGCCA | |
| | GCUACCACGGCACCAAGCCCGGCUACGUGGACAGUAUCCA | |
| | GAAGGGCAUCCAGAAGCCCAAGAGCGGCACCCAGGGCAAC | |
| | UACGACGACGACUGGGAGGGCUUCUACAGCACCGACAAC | |
| | AAGUACGACGCCGCCGGCUACAGCGUCGACAACGAGAACC | |
| | CACUGUCCGGCAAAGCCGGAGGCGUGGUGAAGGUGACCU | |
| | ACCCCGGCCUGACCAAGGUGCUGGCCCUGAAGGUGGACAA | |
| | CGCCGAGACCAUUAAGAAGGAGCUGGGCCUGAGCCUGAC | |
| | CGAGCCCCUGAUGGAGCAGGUGGGCACUGAGGAGUUCAU | |
| | CAAGCGUUUCGGGGACGGGGCGAGUAGAGUGGUGCUGAG | |
| | CCUGCCCUUCGCCGAGGGCAGCAGCAGCGUGAAGUACAUC | |
| | AACAACUGGGAGCAGGCCAAGGCCCUGAGCGUGGAGCUG | |
| | GAGAUCAACUUCGAGACCCGGGGCAAACGAGGCCAGGAC | |
| | GCCAUGUACGAGUACAUGGCACAGGCUUGCGCCGGCAAU | |
| | CGGGUGCGGAGAAGCGUGGGCUCUAGCCUGAGCUGCAUC | |
| | AAUCUGGACUGGGACGUGAUCCGGGACAAGACGAAGACC | |
| | AAGAUCGAGAGCCUGAAGGAGCACGGCCCCAUCAAGAAC | |
| | AAGAUGAGCGAGAGCCCCAACAAGGCCGUGAGCGAGGAG | |
| | AAGGCCAAGCAGUACCUGGAGGAGUUCCACCAGACCGCCU | |
| | UGGAACACCCCGAGCUGAGCGAGCUGAAGACUGUGACCG | |
| | GCACCAACCCCGUUUUCGCCGGCGCCAACUACGCCGCCUG | |
| | GGCCGUGAACGUGGCUCAGGUGAUCGACAGCGAGACCGC | |
| | CGACAACCUGGAGAAGACCACCGCCGCCCUGAGCAUCCUG | |
| | CCCGGCAUCGGCAGCGUGAUGGGCAUCGCUGACGGCGCCG | |
| | UGCACCACAACACCGAGGAGAUCGUGGCCCAGAGCAUCGC | |
| | CCUCAGCAGCCUGAUGGUGGCCCAGGCCAUUCCCCUGGUG | |
| | GGCGAGCUGGUGGACAUCGGCUUCGCCGCCUACAACUUCG | |
| | UGGAGAGCAUCAUCAACCUGUUCCAGGUGGUGCACAACA | |
| | GCUACAACCGGCCCGCCUAUAGCCCCGGACACAAGACCCA | |
| | GCCCUUUCUGCACGACGGCUACGCCGUGAGCUGGAACACC | |
| | GUGGAGGACAGCAUCAUCCGGACCGGCUUCCAGGGCGAG | |
| | AGCGGCCACGACAUCAAGAUCACCGCCGAGAACACCCCUC | |
| | UGCCCAUCGCCGGCGUUCUGCUGCCCACCAUCCCCGGCAA | |
| | GCUGGACGUGAACAAGGCCAAGACCCACAUCAGCGUGAA | |
| | CGGCCGGAAGAUCCGAAUGCGGUGCCGGGCAAUCGACGG | |
| | CGACGUGACCUUCUGCCGGCCUAAGAGCCCCGUGUACGUG | |
| | GGCAACGGCGUUCACGCCAACCUGCACGUGGCCUUCCACC | |
| | GGAGCAGCUCUGAGAAGAUCCACAGCAACGAGAUCAGCA | |
| | GCGACAGCAUCGGCGUGCUGGGCUACCAGAAGACCGUGG | |
| | ACCACACCAAGGUGAACAGCAAACUGAGCCUGUUCUUCG | |
| | AGAUCAAGAGC | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGGG | |
| | C | |
| Corresponding amino | METPAQLLFLLLLWLPDTTGTLEHVQHIIGGAGNDAITGNAHD | 95 |
| acid sequence | NFLAGGSGDDRLDGGAGNDVLVGGEGQNTVIGGAGDDVFLQ | |
| | DLGVWSNQLDGGAGVDTVKYNVHQPSEERLERMGDTGIHAD | |
| | LQKGTVEKWPALNLFSVDHVKNIENLHGSRLNDRIAGDDQDN | |
| | ELWGHDGNDVIRGRGGDDILRGGLGLDTLYGEDGNDIFLQDD | |
| | ETVSDDIDGGAGLDTVDYSAMIHPGRIVAPHEYGFGIEADLSR | |
| | EWVRKASALGVDYYDNVRNVENVIGTSMKDVLIGDAQANTL | |
| | MGQGGDDTVRGGDGDDLLFGGDGNDMLYGDAGNDVLYGG | |
| | LGDDTLEGGAGNDWFGQTQAREHDVLRGGDGVDTVDYSQT | |
| | GAHAGIAAGRIGLGILADLGAGRVDKLGEAGSSAYDTVSGIEN | |
| | VVGTELADRITGDAQANVLRGAGGADVLAGGEGDDVLLGGD | |
| | GDDQLSGDAGRDRLYGEAGDDWFFQDAANAGNLLDGGDGR | |
| | DTVDFSGPGRGLDAGAKGVFLSLGKGFASLMDEPETSNVLRN | |
| | IENAVGSARDDVLIGDAGANVLNGLAGNDVLSGGAGDDVLL | |
| | GDEGSDLLSGDAGNDDLFGGQGDDTYLFGVGYGHDTGGGGS | |
| | GGGGSGGGGSGGGGSGADDVVDSSKSFVMENFASYHGTKPG | |
| | YVDSIQKGIQKPKSGTQGNYDDDWEGFYSTDNKYDAAGYSV | |
| | DNENPLSGKAGGVVKVTYPGLTKVLALKVDNAETIKKELGLS | |
| | LTEPLMEQVGTEEFIKRFGDGASRVVLSLPFAEGSSSVKYINN | |
| | WEQAKALSVELEINFETRGKRGQDAMYEYMAQACAGNRVR | |
| | RSVGSSLSCINLDWDVIRDKTKTKIESLKEHGPIKNKMSESPNK | |
| | AVSEEKAKQYLEEFHQTALEHPELSELKTVTGTNPVFAGANY | |
| | AAWAVNVAQVIDSETADNLEKTTAALSILPGIGSVMGIADGA | |
| | VHHNTEEIVAQSIALSSLMVAQAIPLVGELVDIGFAAYNFVESII | |
| | NLFQVVHNSYNRPAYSPGHKTQPFLHDGYAVSWNTVEDSIIR | |
| | TGFQGESGHDIKITAENTPLPIAGVLLPTIPGKLDVNKAKTHISV | |
| | NGRKIRMRCRAIDGDVTFCRPKSPVYVGNGVHANLHVAFHRS | |
| | SSEKIHSNEISSDSIGVLGYQKTVDHTKVNSKLSLFFEIKS | |
| PolyA tail | 100 nt | |

Diptheria_20AALinker_RTX

| SEQ ID NO: 96 consists of from 5′ end to 3′ end: | | 96 |
|---|---|---|
| 5′ UTR SEQ ID NO: 2, mRNA ORF SEQ ID NO: 97, and | | |
| 3′ UTR SEQ ID NO: 4. | | |
| 5′ UTR | GGGAAAUAAGAGAGAAAAGAAGAGUAAGAAGAAAUAUAA | 2 |
| | GACCCCGGCGCCGCCACC | |
| ORF of mRNA | AUGGAGACCCCUGCCCAGCUGCUGUUCCUGCUGCUGCUUU | 97 |
| Construct | GGCUGCCAGACACCACCGGAGGAGCUGACGACGUGGUGG | |
| (excluding the stop | ACAGCAGCAAGAGCUUCGUGAUGGAGAACUUCGCCAGCU | |
| codon) | ACCACGGCACCAAGCCCGGCUACGUGGACAGUAUCCAGAA | |
| | GGGCAUCCAGAAGCCCAAGAGCGGCACCCAGGGCAACUAC | |
| | GACGACGACUGGGAGGGCUUCUACAGCACCGACAACAAG | |
| | UACGACGCCGCCGGCUACAGCGUCGACAACGAGAACCCAC | |
| | UGUCCGGCAAAGCCGGAGGCGUGGUGAAGGUGACCUACC | |
| | CCGGCCUGACCAAGGUGCUGGCCCUGAAGGUGGACAACGC | |
| | CGAGACCAUUAAGAAGGAGCUGGGCCUGAGCCUGACCGA | |
| | GCCCCUGAUGGAGCAGGUGGGCACUGAGGAGUUCAUCAA | |
| | GCGUUUCGGGGACGGGGCGAGUAGAGUGGUGCUGAGCCU | |
| | GCCCUUCGCCGAGGGCAGCAGCAGCGUGAAGUACAUCAAC | |
| | AACUGGGAGCAGGCCAAGGCCCUGAGCGUGGAGCUGGAG | |
| | AUCAACUUCGAGACCCGGGGCAAACGAGGCCAGGACGCCA | |
| | UGUACGAGUACAUGGCACAGGCUUGCGCCGGCAAUCGGG | |
| | UGCGGAGAAGCGUGGGCUCUAGCCUGAGCUGCAUCAAUC | |
| | UGGACUGGGACGUGAUCCGGGACAAGACGAAGACCAAGA | |
| | UCGAGAGCCUGAAGGAGCACGGCCCCAUCAAGAACAAGA | |
| | UGAGCGAGAGCCCCAACAAGGCCGUGAGCGAGGAGAAGG | |
| | CCAAGCAGUACCUGGAGGAGUUCCACCAGACCGCCUUGGA | |
| | ACACCCCGAGCUGAGCGAGCUGAAGACUGUGACCGGCACC | |
| | AACCCCGUUUUCGCCGGCGCCAACUACGCCGCCUGGGCCG | |
| | UGAACGUGGCUCAGGUGAUCGACAGCGAGACCGCCGACA | |
| | ACCUGGAGAAGACCACCGCCGCCCUGAGCAUCCUGCCCGG | |
| | CAUCGGCAGCGUGAUGGGCAUCGCUGACGGCGCCGUGCAC | |
| | CACAACACCGAGGAGAUCGUGGCCCAGAGCAUCGCCCUCA | |
| | GCAGCCUGAUGGUGGCCCAGGCCAUUCCCCUGGUGGGCGA | |
| | GCUGGUGGACAUCGGCUUCGCCGCCUACAACUUCGUGGA | |
| | GAGCAUCAUCAACCUGUUCCAGGUGGUGCACAACAGCUA | |
| | CAACCGGCCCGCCUAUAGCCCCGGACACAAGACCCAGCCC | |
| | UUUCUGCACGACGGCUACGCCGUGAGCUGGAACACCGUG | |
| | GAGGACAGCAUCAUCCGGACCGGCUUCCAGGGCGAGAGC | |
| | GGCCACGACAUCAAGAUCACCGCCGAGAACACCCCUCUGC | |
| | CCAUCGCCGGCGUUCUGCUGCCCACCAUCCCCGGCAAGCU | |
| | GGACGUGAACAAGGCCAAGACCCACAUCAGCGUGAACGG | |
| | CCGGAAGAUCCGAAUGCGGUGCCGGGCAAUCGACGGCGA | |
| | CGUGACCUUCUGCCGGCCUAAGAGCCCCGUGUACGUGGGC | |
| | AACGGCGUUCACGCCAACCUGCACGUGGCCUUCCACCGGA | |
| | GCAGCUCUGAGAAGAUCCACAGCAACGAGAUCAGCAGCG | |
| | ACAGCAUCGGCGUGCUGGGCUACCAGAAGACCGUGGACC | |
| | ACACCAAGGUGAACAGCAAACUGAGCCUGUUCUUCGAGA | |
| | UCAAGAGCGGAGGAGGUGGCUCUGGCGGAGGUGGAAGCG | |
| | GUGGAGGCGGAAGUGGUGGCGGAGGCAGCACCCUGGAGC | |
| | ACGUGCAGCACAUCAUCGGCGGCGCCGGCAACGACGCCAU | |
| | CACUGGCAACGCCCACGACAACUUCCUGGCCGGCGGCAGC | |
| | GGCGACGACAGACUGGACGGCGGAGCAGGUAACGACGUG | |
| | CUCGUCGGAGGCGAGGGGCAGAACACCGUGAUUGGCGGA | |
| | GCCGGAGACGACGUGUUCCUGCAGGACCUGGGCGUGUGG | |
| | AGCAACCAGUUAGACGGCGGCGCAGGUGUGGAUACUGUU | |
| | AAGUACAACGUGCACCAGCCUAGCGAGGAGAGACUGGAG | |
| | AGAAUGGGCGACACCGGCAUCCACGCCGACCUGCAGAAGG | |
| | GAACCGUGGAGAAGUGGCCUGCCCUGAACCUGUUCAGCG | |
| | UGGACCACGUGAAGAACAUCGAGAACCUGCACGGCAGUA | |
| | GACUAAACGACAGAAUUGCAGGAGACGAUCAGGACAACG | |
| | AACUGUGGGGUCACGACGGAAACGACGUGAUACGUGGCA | |
| | GAGGAGGCGACGACAUCCUGCGGGGCGGCCUGGGCCUGG | |
| | ACACCCUGUACGGCGAGGACGGAAACGACAUCUUCCUCCA | |
| | AGACGACGAAACCGUGAGCGACGAUAUCGACGGCGGUGC | |
| | CGGAUUAGACACCGUCGACUACAGCGCCAUGAUCCACCCU | |
| | GGCAGAAUCGUGGCCCCUCACGAGUACGGCUUCGGCAUCG | |
| | AGGCUGACCUGAGCAGAGAGUGGGUGAGAAAGGCCAGCG | |
| | CGUUGGGUGUAGACUACUACGACAACGUGAGAAACGUCG | |
| | AGAACGUCAUUGGCACCAGCAUGAAAGACGUGCUCAUCG | |
| | GUGACGCUCAGGCCAACACCCUGAUGGGCCAGGGCGGUG | |
| | ACGACACAGUGCGAGGCGGAGACGGAGACGACCUGCUGU | |
| | UCGGUGGCGACGGCAACGAUAUGUUAUACGGCGACGCUG | |
| | GAAACGACGUGUUAUACGGUGGACUGGGUGACGAUACAU | |
| | UGGAGGGUGGCGCGGGCAACGAUUGGUUCGGCCAGACAC | |
| | AAGCCAGAGAGCACGACGUUCUACGUGGCGGCGACGGAG | |
| | UGGACACUGUGGACUACUCACAAACCGGCGCCCACGCCGG | |
| | AAUCGCGGCCGGGAGAAUCGGCUUGGGAAUCCUUGCUGA | |
| | CUUGGGUGCAGGCCGGGUGGACAAGCUGGGUGAGGCAGG | |
| | CUCAAGUGCCUACGAUACAGUGAGCGGCAUUGAGAACGU | |
| | AGUCGGCACGGAGCUUGCUGAUCGGAUUACCGGAGACGC | |
| | CCAAGCAAACGUGUUACGGGGUGCUGGAGGCGCUGACGU | |
| | CCUCGCUGGCGGUGAGGGCGACGACGUGCUGUUGGGAGG | |
| | AGACGGUGACGACCAACUGUCGGGCGACGCGGGAAGGGA | |
| | UAGACUGUACGGAGAAGCAGGAGACGACUGGUUCUUCCA | |
| | GGACGCCGCCAACGCGGGAAACCUGCUUGACGGUGGUGA | |
| | CGGACGAGAUACCGUAGAUUUCUCUGGUCCAGGCAGAGG | |
| | CCUCGACGCAGGCGCCAAGGGCGUCUUCCUGUCCCUCGGU | |
| | AAGGGCUUCGCCAGCCUUAUGGACGAACCUGAGACUUCU | |
| | AACGUGCUGAGAAAUAUUGAGAACGCCGUCGGCAGCGCC | |
| | AGGGACGACGUCUUAAUCGGCGACGCCGGUGCGAACGUC | |
| | CUAAACGGCUUGGCCGGAAACGACGUUUUAAGCGGAGGA | |
| | GCUGGCGACGACGUACUGCUUGGCGACGAGGGCAGCGAC | |
| | CUCCUAUCAGGUGACGCCGGUAACGACGACCUCUUCGGCG | |
| | GACAGGGUGACGAUACUUAUUUGUUCGGCGUGGGCUACG | |
| | GACACGACACU | |
| 3′ UTR | UGAUAAUAGGCUGGAGCCUCGGUGGCCUAGCUUCUUGCC | 4 |
| | CCUUGGGCCUCCCCCCAGCCCCUCCUCCCCUUCCUGCACC | |
| | CGUACCCCCGUGGUCUUUGAAUAAAGUCUGAGUGGGCGG | |
| | C | |
| Corresponding amino | METPAQLLFLLLLWLPDTTGGADDVVDSSKSFVMENFASYHG | 98 |
| acid sequence | TKPGYVDSIQKGIQKPKSGTQGNYDDDWEGFYSTDNKYDAA | |
| | GYSVDNENPLSGKAGGVVKVTYPGLTKVLALKVDNAETIKKE | |
| | LGLSLTEPLMEQVGTEEFIKRFGDGASRVVLSLPFAEGSSSVKY | |
| | INNWEQAKALSVELEINFETRGKRGQDAMYEYMAQACAGNR | |
| | VRRSVGSSLSCINLDWDVIRDKTKTKIESLKEHGPIKNKMSESP | |
| | NKAVSEEKAKQYLEEFHQTALEHPELSELKTVTGTNPVFAGA | |
| | NYAAWAVNVAQVIDSETADNLEKTTAALSILPGIGSVMGIAD | |
| | GAVHHNTEEIVAQSIALSSLMVAQAIPLVGELVDIGFAAYNFV | |
| | ESIINLFQVVHNSYNRPAYSPGHKTQPFLHDGYAVSWNTVEDS | |
| | IIRTGFQGESGHDIKITAENTPLPIAGVLLPTIPGKLDVNKAKTH | |
| | ISVNGRKIRMRCRAIDGDVTFCRPKSPVYVGNGVHANLHVAF | |
| | HRSSSEKIHSNEISSDSIGVLGYQKTVDHTKVNSKLSLFFEIKSG | |
| | GGGSGGGGSGGGGSGGGGSTLEHVQHIIGGAGNDAITGNAHD | |
| | NFLAGGSGDDRLDGGAGNDVLVGGEGQNTVIGGAGDDVFLQ | |
| | DLGVWSNQLDGGAGVDTVKYNVHQPSEERLERMGDTGIHAD | |
| | LQKGTVEKWPALNLFSVDHVKNIENLHGSRLNDRIAGDDQDN | |
| | ELWGHDGNDVIRGRGGDDILRGGLGLDTLYGEDGNDIFLQDD | |
| | ETVSDDIDGGAGLDTVDYSAMIHPGRIVAPHEYGFGIEADLSR | |
| | EWVRKASALGVDYYDNVRNVENVIGTSMKDVLIGDAQANTL | |
| | MGQGGDDTVRGGDGDDLLFGGDGNDMLYGDAGNDVLYGG | |
| | LGDDTLEGGAGNDWFGQTQAREHDVLRGGDGVDTVDYSQT | |
| | GAHAGIAAGRIGLGILADLGAGRVDKLGEAGSSAYDTVSGIEN | |
| | VVGTELADRITGDAQANVLRGAGGADVLAGGEGDDVLLGGD | |
| | GDDQLSGDAGRDRLYGEAGDDWFFQDAANAGNLLDGGDGR | |
| | DTVDFSGPGRGLDAGAKGVFLSLGKGFASLMDEPETSNVLRN | |
| | IENAVGSARDDVLIGDAGANVLNGLAGNDVLSGGAGDDVLL | |
| | GDEGSDLLSGDAGNDDLFGGQGDDTYLFGVGYGHDT | |
| PolyA tail | 100 nt | |

What is claimed is:

1. A composition comprising:
at least one messenger ribonucleic acid (mRNA) polynucleotide having at least one open reading frame (ORF) encoding a Bordetella pertussis antigenic polypeptide selected from the group consisting of pertussis toxin antigenic polypeptides, autotransporter subtilisin-like protease (SPHB1) antigenic polypeptides, tracheal colonization factor A (TCFA) antigenic polypeptides, filamentous hemagglutinin (FHA) antigenic polypeptides, pertactin (PRN) antigenic polypeptides, fimbriae (FIM) antigenic polypeptides, adenylate cyclase antigenic polypeptides, Bordetella resistance to killing (Brk) antigenic polypeptides, and virulence-associated gene 8 (Vag8) antigenic polypeptides.

2. The composition of claim 1, comprising:
at least two messenger ribonucleic acid (mRNA) polynucleotides, each having at least one ORF and each encoding a Bordetella pertussis antigenic polypeptide selected from the group consisting of pertussis toxin antigenic polypeptides, autotransporter subtilisin-like protease (SPHB1) antigenic polypeptides, tracheal colonization factor A (TCFA) antigenic polypeptides, filamentous hemagglutinin (FHA) antigenic polypeptides, pertactin (PRN) antigenic polypeptides, fimbriae (FIM) antigenic polypeptides, adenylate cyclase antigenic polypeptides, Bordetella resistance to killing (Brk) antigenic polypeptides, and virulence-associated gene 8 (Vag8) antigenic polypeptides.

3. The composition of claim 1, comprising:
at least three messenger ribonucleic acid (mRNA) polynucleotides, each having at least one ORF, and each encoding a Bordetella pertussis antigenic polypeptide selected from the group consisting of pertussis toxin antigenic polypeptides, autotransporter subtilisin-like protease (SPHB1) antigenic polypeptides, tracheal colonization factor A (TCFA) antigenic polypeptides, filamentous hemagglutinin (FHA) antigenic polypeptides, pertactin (PRN) antigenic polypeptides, fimbriae (FIM) antigenic polypeptides, adenylate cyclase antigenic polypeptides, Bordetella resistance to killing (Brk) antigenic polypeptides, and virulence-associated gene 8 (Vag8) antigenic polypeptides.

4. The composition of claim 1, comprising:
at least four messenger ribonucleic acid (mRNA) polynucleotides, each having at least one ORF, and each encoding a Bordetella pertussis antigenic polypeptide selected from the group consisting of pertussis toxin antigenic polypeptides, autotransporter subtilisin-like protease (SPHB1) antigenic polypeptides, tracheal colonization factor A (TCFA) antigenic polypeptides, filamentous hemagglutinin (FHA) antigenic polypeptides, pertactin (PRN) antigenic polypeptides, fimbriae (FIM) antigenic polypeptides, adenylate cyclase antigenic polypeptides, Bordetella resistance to killing (Brk) antigenic polypeptides, and virulence-associated gene 8 (Vag8) antigenic polypeptides.

5. The composition of claim 1, comprising:
at least five messenger ribonucleic acid (mRNA) polynucleotides, each having at least one ORF, and encoding a Bordetella pertussis antigenic polypeptide selected from the group consisting of pertussis toxin antigenic polypeptides, autotransporter subtilisin-like protease (SPHB1) antigenic polypeptides, tracheal colonization factor A (TCFA) antigenic polypeptides, filamentous hemagglutinin (FHA) antigenic polypeptides, pertactin (PRN) antigenic polypeptides, fimbriae (FIM) antigenic polypeptides, adenylate cyclase antigenic polypeptides, Bordetella resistance to killing (Brk) antigenic polypeptides, and virulence-associated gene 8 (Vag8) antigenic polypeptides.

6. The composition of claim 1, comprising:
at least six messenger ribonucleic acid (mRNA) polynucleotides, each having at least one ORF, and each encoding a Bordetella pertussis antigenic polypeptide selected from the group consisting of pertussis toxin antigenic polypeptides, autotransporter subtilisin-like protease (SPHB1) antigenic polypeptides, tracheal colonization factor A (TCFA) antigenic polypeptides, filamentous hemagglutinin (FHA) antigenic polypeptides, pertactin (PRN) antigenic polypeptides, fimbriae (FIM) antigenic polypeptides, adenylate cyclase antigenic polypeptides, Bordetella resistance to killing (Brk) antigenic polypeptides, and virulence-associated gene 8 (Vag8) antigenic polypeptides.

7. The composition of claim 1, comprising:
at least seven messenger ribonucleic acid (mRNA) polynucleotides, each having at least one ORF, and each encoding a Bordetella pertussis antigenic polypeptide selected from the group consisting of pertussis toxin antigenic polypeptides, autotransporter subtilisin-like protease (SPHB1) antigenic polypeptides, tracheal colonization factor A (TCFA) antigenic polypeptides, filamentous hemagglutinin (FHA) antigenic polypeptides, pertactin (PRN) antigenic polypeptides, fimbriae (FIM) antigenic polypeptides, adenylate cyclase antigenic polypeptides, Bordetella resistance to killing (Brk) antigenic polypeptides, and virulence-associated gene 8 (Vag8) antigenic polypeptides.

8. The composition of claim 1, comprising:
at least eight messenger ribonucleic acid (mRNA) polynucleotides, each having at least one ORF, and each encoding a Bordetella pertussis antigenic polypeptide selected from the group consisting pertussis toxin antigenic polypeptides, autotransporter subtilisin-like protease (SPHB1) antigenic polypeptides, tracheal colonization factor A (TCFA) antigenic polypeptides, filamentous hemagglutinin (FHA) antigenic polypeptides, pertactin (PRN) antigenic polypeptides, fimbriae (FIM) antigenic polypeptides, adenylate cyclase antigenic polypeptides, Bordetella resistance to killing (Brk) antigenic polypeptides, and virulence-associated gene 8 (Vag8) antigenic polypeptides.

9. A composition, comprising:
at least nine messenger ribonucleic acid (mRNA) polynucleotides, each having at least one ORF, and each encoding at least one of each Bordetella pertussis antigenic polypeptide selected from the group consisting of pertussis toxin antigenic polypeptides, autotransporter subtilisin-like protease (SPHB1) antigenic polypeptides, tracheal colonization factor A (TCFA) antigenic polypeptides, filamentous hemagglutinin (FHA) antigenic polypeptides, pertactin (PRN) antigenic polypeptides, fimbriae (FIM) antigenic polypeptides, adenylate cyclase antigenic polypeptides, Bordetella resistance to killing (Brk) antigenic polypeptides, and virulence-associated gene 8 (Vag8) antigenic polypeptides.

10. The composition of any one of claims 1-9, wherein the pertussis toxin antigen polypeptide is selected from the group consisting of: S1 subunit, S2 subunit, S3 subunit, S4 subunit, S5 subunit, or a variant thereof.

11. The composition of claim 10, wherein the S1 subunit comprises an amino acid sequence that is at least 80%, 85%, 90%, 95%, or 98% identical to the sequence identified by SEQ ID NO: 8.

12. The composition of claim 11, wherein the S1 subunit comprises an amino acid sequence that is identical to the sequence identified by SEQ ID NO: 8.

13. The composition of any one of claims 10-12, wherein the mRNA encoding the S1 subunit comprises a nucleotide sequence that is at least 95% or 98% identical to the sequence identified by SEQ ID NO: 7.

14. The composition of claim 13, wherein the mRNA encoding the S1 subunit comprises a nucleotide sequence that is identical to the sequence identified by SEQ ID NO: 7.

15. The composition of any one of claims 10-14, wherein the mRNA encoding the S1 subunit comprises a nucleotide sequence that is at least 95% or 98% identical to the sequence identified by SEQ ID NO: 6.

16. The composition of claim 15, wherein the mRNA encoding the S1 subunit comprises a nucleotide sequence that is identical to the sequence identified by SEQ ID NO: 6.

17. The composition of claim 10, wherein the S1 subunit variant comprises an amino acid sequence that is at least 80%, 85%, 90%, 95%, or 98% identical to the sequence identified by SEQ ID NO: 5 or 11.

18. The composition of claim 17, wherein the S1 subunit variant comprises an amino acid sequence that is identical to the sequence identified by SEQ ID NO: 5 or 11.

19. The composition of claim 17 or 18, wherein the mRNA encoding the S1 subunit variant comprises a nucleotide sequence that is at least 95% or 98% identical to the sequence identified by SEQ ID NO: 3 or 10.

20. The composition of claim 19, wherein the mRNA encoding the S1 subunit variant comprises a nucleotide sequence that is identical to the sequence identified by SEQ ID NO: 3 or 10.

21. The composition of any one of claims 17-20, wherein the mRNA encoding the S1 subunit variant comprises a nucleotide sequence that is at least 95% or 98% identical to the sequence identified by SEQ ID NO: 1 or 9.

22. The composition of claim 21, wherein the mRNA encoding the S1 subunit variant comprises a nucleotide sequence that is identical to the sequence identified by SEQ ID NO: 1 or 9.

23. The composition of claim 10, wherein the S2 subunit variant comprises an amino acid sequence that is at least 80%, 85%, 90%, 95%, or 98% identical to the sequence identified by SEQ ID NO: 14.

24. The composition of claim 23, wherein the S2 subunit variant comprises an amino acid sequence that is identical to the sequence identified by SEQ ID NO: 14.

25. The composition of claim 23 or 24, wherein the mRNA encoding the S2 subunit variant comprises a nucleotide sequence that is at least 95% or 98% identical to the sequence identified by SEQ ID NO: 13.

26. The composition of claim 25, wherein the mRNA encoding the S2 subunit variant comprises a nucleotide sequence that is identical to the sequence identified by SEQ ID NO: 13.

27. The composition of any one of claims 23-26, wherein the mRNA encoding the S2 subunit variant comprises a nucleotide sequence that is at least 95% or 98% identical to the sequence identified by SEQ ID NO: 12.

28. The composition of claim 27, wherein the mRNA encoding the S2 subunit variant comprises a nucleotide sequence that is identical to the sequence identified by SEQ ID NO: 12.

29. The composition of claim 10, wherein the S3 subunit variant comprises an amino acid sequence that is at least 80%, 85%, 90%, 95%, or 98% identical to the sequence identified by SEQ ID NO: 17.

30. The composition of claim 29, wherein the S3 subunit variant comprises an amino acid sequence that is identical to the sequence identified by SEQ ID NO: 17.

31. The composition of claim 29 or 30, wherein the mRNA encoding the S3 subunit variant comprises a nucleotide sequence that is at least 95% or 98% identical to the sequence identified by SEQ ID NO: 16.

32. The composition of claim 31, wherein the mRNA encoding the S3 subunit variant comprises a nucleotide sequence that is identical to the sequence identified by SEQ ID NO: 16.

33. The composition of any one of claims 29-32, wherein the mRNA encoding the S3 subunit variant comprises a nucleotide sequence that is at least 95% or 98% identical to the sequence identified by SEQ ID NO: 15.

34. The composition of claim 33, wherein the mRNA encoding the S3 subunit variant comprises a nucleotide sequence that is identical to the sequence identified by SEQ ID NO: 15.

35. The composition of claim 10, wherein the S4 subunit comprises an amino acid sequence that is at least 80%, 85%, 90%, 95%, or 98% identical to the sequence identified by SEQ ID NO: 20.

36. The composition of claim 35, wherein the S4 subunit variant comprises an amino acid sequence that is identical to the sequence identified by SEQ ID NO: 20.

37. The composition of claim 35 or 36, wherein the mRNA encoding the S4 subunit comprises a nucleotide sequence that is at least 95% or 98% identical to the sequence identified by SEQ ID NO: 19.

38. The composition of claim 37, wherein the mRNA encoding the S4 subunit comprises a nucleotide sequence that is identical to the sequence identified by SEQ ID NO: 19.

39. The composition of any one of claims 35-38, wherein the mRNA encoding the S4 subunit comprises a nucleotide sequence that is at least 95% or 98% identical to the sequence identified by SEQ ID NO: 18.

40. The composition of claim 39, wherein the mRNA encoding the S4 subunit comprises a nucleotide sequence that is identical to the sequence identified by SEQ ID NO: 18.

41. The composition of claim 10, wherein the S5 subunit variant comprises an amino acid sequence that is at least 80%, 85%, 90%, 95%, or 98% identical to the sequence identified by SEQ ID NO: 23.

42. The composition of claim 41, wherein the S5 subunit variant comprises an amino acid sequence that is identical to the sequence identified by SEQ ID NO: 23.

43. The composition of claim 41 or 42, wherein the mRNA encoding the S5 subunit variant comprises a nucleotide sequence that is at least 95% or 98% identical to the sequence identified by SEQ ID NO: 22.

44. The composition of claim 43, wherein the mRNA encoding the S5 subunit variant comprises a nucleotide sequence that is identical to the sequence identified by SEQ ID NO: 22.

45. The composition of any one of claims 41-44, wherein the mRNA encoding the S5 subunit variant comprises a nucleotide sequence that is at least 95% or 98% identical to the sequence identified by SEQ ID NO: 21.

46. The composition of claim 45, wherein the mRNA encoding the S5 subunit variant comprises a nucleotide sequence that is identical to the sequence identified by SEQ ID NO: 21.

47. The composition of any one of claims 1-46, wherein the SPHB1 antigenic polypeptide comprises an amino acid sequence that is at least 80%, 85%, 90%, 95%, or 98% identical to the sequence identified by SEQ ID NO: 26.

48. The composition of claim 47, wherein the SPHB1 antigenic polypeptide comprises an amino acid sequence that comprises the sequence identified by SEQ ID NO: 26.

49. The composition of claim 47 or 48, wherein the mRNA encoding the SPHB1 antigenic polypeptide comprises a nucleotide sequence that is at least 95% or 98% identical to the sequence identified by SEQ ID NO: 25.

50. The composition of claim 49, wherein the mRNA encoding the SPHB1 antigenic polypeptide comprises a nucleotide sequence that is identical to the sequence identified by SEQ ID NO: 25.

51. The composition of any one of claims 47-50, wherein the mRNA encoding the SPHB1 antigenic polypeptide comprises a nucleotide sequence that is at least 95% or 98% identical to the sequence identified by SEQ ID NO: 24.

52. The composition of claim 51, wherein the mRNA encoding the SPHB1 antigenic polypeptide comprises a nucleotide sequence that is identical to the sequence identified by SEQ ID NO: 24.

53. The composition of any one of claims 1-52, wherein the TCFA antigenic polypeptide comprises an amino acid sequence that is at least 80%, 85%, 90%, 95%, or 98% identical to the sequence identified by SEQ ID NO: 29.

54. The composition of claim 53, wherein the TCFA antigenic polypeptide comprises an amino acid sequence that comprises the sequence identified by SEQ ID NO: 29.

55. The composition of claim 53 or 54, wherein the mRNA encoding the TCFA antigenic polypeptide comprises a nucleotide sequence that is at least 95% or 98% identical to the sequence identified by SEQ ID NO: 28.

56. The composition of claim 55, wherein the mRNA encoding the TCFA antigenic polypeptide comprises a nucleotide sequence that is identical to the sequence identified by SEQ ID NO: 28.

57. The composition of any one of claims 53-56, wherein the mRNA encoding the TCFA antigenic polypeptide comprises a nucleotide sequence that is at least 95% or 98% identical to the sequence identified by SEQ ID NO: 27.

58. The composition of claim 57, wherein the mRNA encoding the TCFA antigenic polypeptide comprises a nucleotide sequence that is identical to the sequence identified by SEQ ID NO: 27.

59. The composition of any one of claims 1-58, wherein the filamentous hemagglutinin antigenic polypeptide comprises FHA1, FHA2, or FHA3.

60. The composition of claim 59, wherein the FHA3 antigenic polypeptide comprises an amino acid sequence that is at least 80%, 85%, 90%, 95%, or 98% identical to the sequence identified by SEQ ID NO: 35.

61. The composition of claim 60, wherein the FHA3 antigenic polypeptide comprises the sequence identified by SEQ ID NO: 35.

62. The composition of claim 60 or 61, wherein the mRNA encoding the FHA3 antigenic polypeptide comprises a nucleotide sequence that is at least 95% or 98% identical to the sequence identified by SEQ ID NO: 34.

63. The composition of claim 62, wherein the mRNA encoding the FHA3 antigenic polypeptide comprises a nucleotide sequence that is identical to the sequence identified by SEQ ID NO: 34.

64. The composition of any one of claims 60-63, wherein the mRNA encoding the FHA3 antigenic polypeptide comprises a nucleotide sequence that is at least 95% or 98% identical to the sequence identified by SEQ ID NO: 33.

65. The composition of claim 64, wherein the mRNA encoding the FHA3 antigenic polypeptide comprises a nucleotide sequence that is identical to the sequence identified by SEQ ID NO: 33.

66. The composition of any one of claims 1-65, wherein the PRN antigenic polypeptide comprises an amino acid sequence that is at least 80%, 85%, 90%, 95%, or 98% identical to the sequence identified by SEQ ID NO: 32.

67. The composition of claim 66, wherein the PRN antigenic polypeptide comprises an amino acid sequence that comprises the sequence identified by SEQ ID NO: 32.

68. The composition of claim 66 or 67, wherein the mRNA encoding the PRN antigenic polypeptide comprises a nucleotide sequence that is at least 95% or 98% identical to the sequence identified by SEQ ID NO: 31.

69. The composition of claim 68, wherein the mRNA encoding the PRN antigenic polypeptide comprises a nucleotide sequence that is identical to the sequence identified by SEQ ID NO: 31.

70. The composition of any one of claims 66-69, wherein the mRNA encoding the PRN antigenic polypeptide comprises a nucleotide sequence that is at least 95% or 98% identical to the sequence identified by SEQ ID NO: 30.

71. The composition of claim 70, wherein the mRNA encoding the PRN antigenic polypeptide comprises a nucleotide sequence that is identical to the sequence identified by SEQ ID NO: 30.

72. The composition of any one of claims 1-71, wherein the FIM antigenic polypeptides are selected from the group consisting of: FIM1, FIM2, FIM 3, and domain-swapped constructs thereof.

73. The composition of claim 72, wherein the FIM antigenic polypeptide comprises an amino acid sequence that is at least 80%, 85%, 90%, 95%, or 98% identical to the sequence identified by SEQ ID NO: 38.

74. The composition of claim 73, wherein the FIM antigenic polypeptide comprises an amino acid sequence that comprises the sequence identified by SEQ ID NO: 38.

75. The composition of claim 73 or 74, wherein the mRNA encoding the FIM antigenic polypeptide comprises a nucleotide sequence that is at least 95% or 98% identical to the sequence identified by SEQ ID NO: 37.

76. The composition of claim 75, wherein the mRNA encoding the FIM antigenic polypeptide comprises a nucleotide sequence that is identical to the sequence identified by SEQ ID NO: 37.

77. The composition of any one of claims 73-76, wherein the mRNA encoding the FIM antigenic polypeptide comprises a nucleotide sequence that is at least 95% or 98% identical to the sequence identified by SEQ ID NO: 36.

78. The composition of claim 77, wherein the mRNA encoding the FIM antigenic polypeptide comprises a nucleotide sequence that is identical to the sequence identified by SEQ ID NO: 36.

79. The composition of any one of claims 1-78, wherein the adenylate cyclase antigenic polypeptides are selected from the group consisting of: ACT_{188LQ}, ACT_{H63A_K65A_S66G}, and the repeats-in-toxin (RTX) domain.

80. The composition of claim 79, wherein the RTX antigenic polypeptide comprises an amino acid sequence that is at least 80%, 85%, 90%, 95%, or 98% identical to the sequence identified by SEQ ID NO: 41.

81. The composition of claim 80, wherein the RTX antigenic polypeptide comprises an amino acid sequence that comprises the sequence identified by SEQ ID NO: 41.

82. The composition of claim 80 or 81, wherein the mRNA encoding the RTX antigenic polypeptide comprises a nucleotide sequence that is at least 95% or 98% identical to the sequence identified by SEQ ID NO: 40.

83. The composition of claim 82, wherein the mRNA encoding the RTX antigenic polypeptide comprises a nucleotide sequence that is identical to the sequence identified by SEQ ID NO: 40.

84. The composition of any one of claims 80-83, wherein the mRNA encoding the RTX antigenic polypeptide comprises a nucleotide sequence that is at least 95% or 98% identical to the sequence identified by SEQ ID NO: 39.

85. The composition of claim 84, wherein the mRNA encoding the RTX antigenic polypeptide comprises a nucleotide sequence that is identical to the sequence identified by SEQ ID NO: 39.

86. The composition of any one of claims 1-85, wherein the Brk antigenic polypeptide comprises an amino acid sequence that is at least 80%, 85%, 90%, 95%, or 98% identical to the sequence identified by SEQ ID NO: 44.

87. The composition of claim 86, wherein the Brk antigenic polypeptide comprises an amino acid sequence that comprises the sequence identified by SEQ ID NO: 44.

88. The composition of claim 86 or 87, wherein the mRNA encoding the Brk antigenic polypeptide comprises a nucleotide sequence that is at least 95% or 98% identical to the sequence identified by SEQ ID NO: 43.

89. The composition of claim 88, wherein the mRNA encoding the Brk antigenic polypeptide comprises a nucleotide sequence that is identical to the sequence identified by SEQ ID NO: 43.

90. The composition of any one of claims 86-89, wherein the mRNA encoding the Brk antigenic polypeptide comprises a nucleotide sequence that is at least 95% or 98% identical to the sequence identified by SEQ ID NO: 42.

91. The composition of claim 90, wherein the mRNA encoding the Brk antigenic polypeptide comprises a nucleotide sequence that is identical to the sequence identified by SEQ ID NO: 42.

92. The composition of any one of claims 1-91, wherein the Vag8 antigenic polypeptide comprises an amino acid sequence that is at least 80%, 85%, 90%, 95%, or 98% identical to the sequence identified by SEQ ID NO: 47.

93. The composition of claim 92, wherein the Vag8 antigenic polypeptide comprises an amino acid sequence that comprises the sequence identified by SEQ ID NO: 47.

94. The composition of claim 92 or 93, wherein the mRNA encoding the Vag8 antigenic polypeptide comprises a nucleotide sequence that is at least 95% or 98% identical to the sequence identified by SEQ ID NO: 46.

95. The composition of claim 94, wherein the mRNA encoding the Vag8 antigenic polypeptide comprises a nucleotide sequence that is identical to the sequence identified by SEQ ID NO: 46.

96. The composition of any one of claims 92-95, wherein the mRNA encoding the Vag8 antigenic polypeptide comprises a nucleotide sequence that is at least 95% or 98% identical to the sequence identified by SEQ ID NO: 45.

97. The composition of claim 97, wherein the mRNA encoding the Vag8 antigenic polypeptide comprises a nucleotide sequence that is identical to the sequence identified by SEQ ID NO: 45.

98. The composition of any one of claims 1-97, further comprising at least one mRNA polynucleotide having at least one ORF encoding a diphtheria antigenic polypeptide.

99. The composition of claim 98, wherein the diphtheria antigenic polypeptide comprises an amino acid sequence that is at least 80%, 85%, 90%, 95%, or 98% identical to the sequence identified by SEQ ID NO: 50.

100. The composition of claim 99, wherein the diphtheria antigenic polypeptide comprises an amino acid sequence that comprises the sequence identified by SEQ ID NO: 50.

101. The composition of claim 99 or 100, wherein the mRNA encoding the diphtheria antigenic polypeptide comprises a nucleotide sequence that is at least 95% or 98% identical to the sequence identified by SEQ ID NO: 49.

102. The composition of claim 101, wherein the mRNA encoding the diphtheria antigenic polypeptide comprises a nucleotide sequence that is identical to the sequence identified by SEQ ID NO: 49.

103. The composition of any one of claims 99-102, wherein the mRNA encoding the diphtheria antigenic polypeptide comprises a nucleotide sequence that is at least 95% or 98% identical to the sequence identified by SEQ ID NO: 48.

104. The composition of claim 103, wherein the mRNA encoding the diphtheria antigenic polypeptide comprises a nucleotide sequence that is identical to the sequence identified by SEQ ID NO: 48.

105. The composition of any one of claims 1-104, further comprising at least one mRNA polynucleotide having at least one ORF encoding a tetanus antigenic polypeptide.

106. The composition of claim 105, wherein the tetanus antigenic polypeptide comprises an amino acid sequence that is at least 80%, 85%, 90%, 95%, or 98% identical to the sequence identified by SEQ ID NO: 53.

107. The composition of claim 106, wherein the tetanus antigenic polypeptide comprises an amino acid sequence that comprises the sequence identified by SEQ ID NO: 53.

108. The composition of claim 106 or 107, wherein the mRNA encoding the tetanus antigenic polypeptide comprises a nucleotide sequence that is at least 95% or 98% identical to the sequence identified by SEQ ID NO: 52.

109. The composition of claim 108, wherein the mRNA encoding the tetanus antigenic polypeptide comprises a nucleotide sequence that is identical to the sequence identified by SEQ ID NO: 52.

110. The composition of any one of claims 106-109, wherein the mRNA encoding the tetanus antigenic polypeptide comprises a nucleotide sequence that is at least 95% or 98% identical to the sequence identified by SEQ ID NO: 51.

111. The composition of claim 110, wherein the mRNA encoding the tetanus antigenic polypeptide comprises a nucleotide sequence that is identical to the sequence identified by SEQ ID NO: 51.

112. A composition comprising:
at least one messenger ribonucleic acid (mRNA) polynucleotide having at least one open reading frame (ORF) encoding a diphtheria antigenic polypeptide.

113. The composition of claim 112, wherein the diphtheria antigenic polypeptide comprises an amino acid sequence that is at least 80%, 85%, 90%, 95%, or 98% identical to the sequence identified by SEQ ID NO: 50.

114. The composition of claim 113, wherein the diphtheria antigenic polypeptide comprises an amino acid sequence that comprises the sequence identified by SEQ ID NO: 50.

115. The composition of claim 113 or 114, wherein the mRNA encoding the diphtheria antigenic polypeptide comprises a nucleotide sequence that is at least 95% or 98% identical to the sequence identified by SEQ ID NO: 49.

116. The composition of claim 115, wherein the mRNA encoding the diphtheria antigenic polypeptide comprises a nucleotide sequence that is identical to the sequence identified by SEQ ID NO: 49.

117. The composition of any one of claims 113-116, wherein the mRNA encoding the diphtheria antigenic polypeptide comprises a nucleotide sequence that is at least 95% or 98% identical to the sequence identified by SEQ ID NO: 48.

118. The composition of claim 117, wherein the mRNA encoding the diphtheria antigenic polypeptide comprises a nucleotide sequence that is identical to the sequence identified by SEQ ID NO: 48.

119. A composition comprising:
at least one messenger ribonucleic acid (mRNA) polynucleotide having at least one open reading frame (ORF) encoding a tetanus antigenic polypeptide.

120. The composition of claim 119, wherein the tetanus antigenic polypeptide comprises an amino acid sequence that is at least 80%, 85%, 90%, 95%, or 98% identical to the sequence identified by SEQ ID NO: 53.

121. The composition of claim 120, wherein the tetanus antigenic polypeptide comprises an amino acid sequence that comprises the sequence identified by SEQ ID NO: 53.

122. The composition of claim 120 or 121, wherein the mRNA encoding the tetanus antigenic polypeptide comprises a nucleotide sequence that is at least 95% or 98% identical to the sequence identified by SEQ ID NO: 52.

123. The composition of claim 122, wherein the mRNA encoding the tetanus antigenic polypeptide comprises a nucleotide sequence that is identical to the sequence identified by SEQ ID NO: 52.

124. The composition of any one of claims 120-123, wherein the mRNA encoding the tetanus antigenic polypeptide comprises a nucleotide sequence that is at least 95% or 98% identical to the sequence identified by SEQ ID NO: 51.

125. The composition of claim 124, wherein the mRNA encoding the tetanus antigenic polypeptide comprises a nucleotide sequence that is identical to the sequence identified by SEQ ID NO: 51.

126. The composition of any one of the preceding claims, further comprising an mRNA encoding an antigenic fusion polypeptide selected from the group consisting of: pertussis antigenic fusion polypeptides, tetanus antigenic fusion polypeptides, diphtheria antigenic fusion polypeptides, or a combination thereof.

127. The composition of claim 126, wherein the antigenic fusion polypeptide comprises an amino acid sequence that is at least 80%, 85%, 90%, 95%, or 98% identical to a sequence selected from SEQ ID NOs: 56, 59, 62, 65, 68, 71, 74, 77, 80, 83, 86, 89, 92, 95, and 98.

128. The composition of claim 127, wherein the antigenic fusion polypeptide comprises an amino acid sequence that comprises a sequence selected from SEQ ID NOs: 56, 59, 62, 65, 68, 71, 74, 77, 80, 83, 86, 89, 92, 95, and 98.

129. The composition of claim 127 or 128, wherein the mRNA encoding the antigenic fusion polypeptide comprises a nucleotide sequence that is at least 95% or 98% identical to a sequence selected from SEQ ID NOs: 55, 58, 61, 64, 67, 70, 73, 76, 79, 82, 85, 88, 91, 94, and 97.

130. The composition of claim 129, wherein the mRNA encoding the antigenic fusion polypeptide comprises a nucleotide sequence that is identical to a sequence selected from SEQ ID NOs: 55, 58, 61, 64, 67, 70, 73, 76, 79, 82, 85, 88, 91, 94, and 97.

131. The composition of any one of claims 127-130, wherein the mRNA encoding the antigenic fusion polypeptide comprises a nucleotide sequence that is at least 95% or 98% identical to a sequence selected from SEQ ID NOs: 54, 57, 60, 63, 66, 69, 72, 75, 78, 81, 84, 87, 90, 93, and 96.

132. The composition of claim 131, wherein the mRNA encoding the antigenic fusion polypeptide comprises a nucleotide sequence that is identical to a sequence selected from SEQ ID NOs: 54, 57, 60, 63, 66, 69, 72, 75, 78, 81, 84, 87, 90, 93, and 96.

133. The composition of any one of the preceding claims, wherein the mRNA comprises a 5′ untranslated region (UTR) comprising the nucleotide sequence of SEQ ID NO: 2.

134. The composition of any one of the preceding claims, wherein the mRNA comprises a 3′ UTR comprising the nucleotide sequence of SEQ ID NO: 4.

135. The composition of any one of the preceding claims, wherein the mRNA further comprises a chemical modification.

136. The composition of claim 125, wherein the chemical modification is 1-methylpseudouridine.

137. The composition of any one of the preceding claims, further comprising a lipid nanoparticle.

138. The composition of claim 137, wherein the lipid nanoparticle comprises a PEG-modified lipid, a non-cationic lipid, a sterol, an ionizable amino lipid, or any combination thereof.

139. The composition of claim 137 or 138, wherein the lipid nanoparticle comprises 0.5-15 mol % PEG-modified lipid; 5-25 mol % non-cationic lipid; 25-55 mol % sterol; and 20-60 mol % ionizable amino lipid.

140. The composition of claim 138 or 139, wherein the PEG-modified lipid is 1,2 dimyristoyl-sn-glycerol, methoxypolyethyleneglycol (PEG2000 DMG), the non-cationic lipid is 1,2 distearoyl-sn-glycero-3-phosphocholine (DSPC), the sterol is cholesterol, and the ionizable amino lipid has the structure of Compound 1:

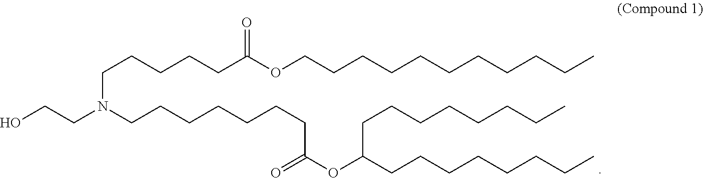 [SMILES: CCCCCCCCCCCOC(=O)CCCCCN(CCO)CCCCCCCC(=O)OC(CCCCCCCC)CCCCCCCC]

141. A method comprising administering to a subject the composition of any one of the preceding claims in an amount effective to induce a neutralizing antibody response against Bordetella pertussis in the subject.

142. The method of claim 141, wherein the composition is administered in an amount effective to induce a Th1 immune response, a Th17 immune response, a Th2 response, or a combination thereof in the subject.

143. The method of claim 141 or 142, wherein the composition is administered in an amount effective to reduce or eliminate symptoms of pertussis in the subject.

144. The method of any one of claims 141-143, wherein the composition is administered in an amount effective to reduce or eliminate colonization of the subject's respiratory tract.

145. The method of any one of claims 141-144, wherein the composition is administered in an amount effective to reduce or eliminate transmissibility of B. pertussis.

146. The method of any one of claims 141-145, wherein the composition is further administered a second time, as a boost.

147. The methods of claim 146, wherein the boost dose is administered 28 days after a first dose.


SEQUENCE LISTING (108 sequences)

SEQ ID NO: 1 - RNA, 776 nt, Artificial Sequence
  Synthetic
gggaaauaagagagaaaagaagaguaagaagaaauauaagaccccggcgccgccaccauggaaaccccugcucagcugcuguuccugcugcugcuguggcugccugauaccacaggggaugauccuccagccaccguguauaaguacgacagcagaccuccugaggacguguuccagaauggcuuuaccgccuggggcaacaacgacaacgugcuggaucaccugaccggcagauccugucaagugggcagcagcaauagcgccuucguguccaccucuagcagcagacgguacacagagguguaccuggaacaccggaugcaagaggccguggaagccgaaagagccggcagaggaacaggccacuucaucggcuacaucuacgaagugcgggccgacaacaacuucuacggcgcugccagcagcuacuucgaguacguggacaccuacggcgacaacgccggaagaauacuggcuggcgcucuggccacauaccagucuggauaucuggcccacagacggaucccuccagagaacauucggagagugacccggguguaccacaacggcauuaccggcgagacaaccaccaccgaguacagcaacgccagauacgugucccagcagacccgggccaauccuaauccuuacaccagcugauaauaggcuggagccucgguggccuagcuucuugccccuugggccuccccccagccccuccuccccuuccugcacccguacccccguggucuuugaauaaagucugagugggcggc

SEQ ID NO: 2 - RNA, 57 nt, Artificial sequence
  Synthetic
gggaaauaagagagaaaagaagaguaagaagaaauauaagaccccggcgccgccacc

SEQ ID NO: 3 - RNA, 600 nt, Artificial sequence
  Synthetic
auggaaaccccugcucagcugcuguuccugcugcugcuguggcugccugauaccacaggggaugauccuccagccaccguguauaaguacgacagcagaccuccugaggacguguuccagaauggcuuuaccgccuggggcaacaacgacaacgugcuggaucaccugaccggcagauccugucaagugggcagcagcaauagcgccuucguguccaccucuagcagcagacgguacacagagguguaccuggaacaccggaugcaagaggccguggaagccgaaagagccggcagaggaacaggccacuucaucggcuacaucuacgaagugcgggccgacaacaacuucuacggcgcugccagcagcuacuucgaguacguggacaccuacggcgacaacgccggaagaauacuggcuggcgcucuggccacauaccagucuggauaucuggcccacagacggaucccuccagagaacauucggagagugacccggguguaccacaacggcauuaccggcgagacaaccaccaccgaguacagcaacgccagauacgugucccagcagacccgggccaauccuaauccuuacaccagc

SEQ ID NO: 4 - RNA, 119 nt, Artificial sequence
  Synthetic
ugauaauaggcuggagccucgguggccuagcuucuugccccuugggccuccccccagccccuccuccccuuccugcacccguacccccguggucuuugaauaaagucugagugggcggc

SEQ ID NO: 5 - protein, 200 aa, Artificial sequence
  Synthetic
METPAQLLFLLLLWLPDTTGDDPPATVYKYDSRPPEDVFQNGFTAWGNNDNVLDHLTGRSCQVGSSNSAFVSTSSSRRYTEVYLEHRMQEAVEAERAGRGTGHFIGYIYEVRADNNFYGAASSYFEYVDTYGDNAGRILAGALATYQSGYLAHRRIPPENIRRVTRVYHNGITGETTTTEYSNARYVSQQTRANPNPYTS

SEQ ID NO: 6 - RNA, 3293 nt, Artificial sequence
  Synthetic
gggaaauaagagagaaaagaagaguaagaagaaauauaagaccccggcgccgccaccaugacugucauguugcagagggaaaugccggugagggcauaggucuggaauguggcguccucguagucguaauacugcucuuccuugcugacguggacgcguacggcaaggccggacauauagaucauguacagcaggcgccgcagcgcgucguacaugucucuguaccugccuucauacgggcuggcgcaggcuccgaugaccgauugcccgucccugacgaauaccgcgcacagccugcuguuggugcuggcgagcaggcgcguggccgugaccuugcuguaguagugauccgcagccgguugcccggucuuguauauggucgugaugcagaaaguuucgcgauaaaugaaccccgcgccuggcggcgcguccuugaugaugccgccguacgccuggcccagguacguaccgucauagagaccguauauggaccagccgggcgcuaucuggcgcaaauacgucugcaauucggcguugccgcgcaguucggccacggucaaggcgcggguuccguucgggcagcguccauaggcgccgcccuguugggugaacagugccuucggcgggaugacgaugccuggcgcaacggccugggccgugcgcaugcccagcagggcgagcaccaggaugggcagaaugugaugaagcagcuucuuguuguugaucagcauguugcgguguucccggaauggaaccagggcggaagaucgucucaagcggauugcgcggaaacgucgaaaccuccggaaggguucauucgcaauauccguugagcgggcagaucugcaguucgagcagaucgccgggagugcccggauacggcgagucuuccaccgucagcgcgauccggcucuugagcgcauacgcggauauggcaaagccaagcauggugucgaaccacgugucgugcucgugucccgcgucggacaggcacgcccggaccaggcuucugcccgacaugaaggcggucaggcagaacuccugauucuugcccuucaguuucaaggccagcuccuggacagugaaguucuuguacagaugggucggcaagccggcgacgucagccgggcuguauaugccgagcacggacaacaggauggaugcgaugguaugcaugggguucgccuucaggggcaauccugcuugccgcugcauucgaccauccggaucaguucgagcgcgggcuugccuucgaaagugaguugcuugggacccagaaacaugcgcagcggccguuguugcaugaagacggcgcgcaacaugacuuccaugggacugcugccgggacgcuugagauccuugccgaagcagaacggcacgugcgcguccgggcugcuggccgcggcgcccaguuuggcggcgaugccgcagaccagcaugcgcgucggggugacuucauacggcuucacggcuacgcuggugaccaccauauuggucuucaccagcacauaaggaacgucggccagggcgggggaaagaugcgucaucgcgccggaugccagcaaccacgccagggcgcgcacgcgcgaccgccgggcgccgcccugucccggggcguugguucgaguggggaagcgucucagcauaaggaugauccaggauugcagauggagaugucgguaagggcguaagucucgaacguugcguccucauagucguaauacuguucuuccuugcugacauggacgcguacggagaugccggccacguagaucagguaaagcauucuccgcagccggcuguacaugcuccaguacuugccgucauacgggcuggugcaggcgccaaugaccgguugcccgcuucugacgaagaccgcgcauagccugcuguuggugcuggagagcaggcgaguggcggugacguugcuguaguagugauccguugcggguugacccguauugcgcguggucaugaugcagaacgucguuuucaggucgaaugcgccgccggguguuccguccuugaucacgccgccauauucgccgccgagauaggugccaucguagagcgcaaauauugaccagccgcgcgucacaugacgcagguacuccugcagaucgccgcugccgcgcaauuccgccacggucagggcacgggucuuguucgcgcagcguccauaggggccgccaugcuggguaaucuguuccugcggcggaaugacgaugccuggcguggaggcccgcgccacgugagauccgaggagggccaacggcagaacggacaggagauggcagagcgucuugcggucgaucggcaugcuguucaauuaccggaguugggcggggcugggccuggucuagaacgaauacgcgaugcuuucguaguacacgagaaccaucgccucgccggcgcguucggaccaggcugccauggccucggagcuuucggccugccgcgccaugcaagcgccuaucaccggcgccaugcgcaccaaugugccgacgaucgacgcuacggaccuucgcgauguguagggguugggauuggcgcgagucugcuggcugacguagcgagcguuggaauacuccguggucguggucucgccggugaugccguugugauagacccgcguuacccugcggauguuuucgggcggaaugcgccggugugccagauauucgcucugguagguggccagcgcgccggcgaggauacggccggcauugucgccauaagugucgacguauucgaaguacgagcuggcggcgccguagaaauugcugucggcgcggacuucguagauguagccgaugaaguggccggugccccugccggcgcguucggccucgaccgcuuccugcaugcgauguucgagauagaccucgguauagcgccggcugcugcugguggagacgaaagcgcuguugcugcugccgaccuggcaggaacguccggucagauggucgagcacauugucguuguuuccccacgccgugaauccguucuggaaaacguccuccggcgggcgggagucauagcgguauacgguggcgggaggaucgucggcccaugccggcgaagucacgggcgccgugacggcaagaaucgccagccacgucagccagccuguucuugcgguuuggcgaauugcccgagugcaacgcaucccguugauaauaggcuggagccucgguggccuagcuucuugccccuugggccuccccccagccccuccuccccuuccugcacccguacccccguggucuuugaauaaagucugagugggcggc

SEQ ID NO: 7 - RNA, 3117 nt, Artificial sequence
  Synthetic
augacugucauguugcagagggaaaugccggugagggcauaggucuggaauguggcguccucguagucguaauacugcucuuccuugcugacguggacgcguacggcaaggccggacauauagaucauguacagcaggcgccgcagcgcgucguacaugucucuguaccugccuucauacgggcuggcgcaggcuccgaugaccgauugcccgucccugacgaauaccgcgcacagccugcuguuggugcuggcgagcaggcgcguggccgugaccuugcuguaguagugauccgcagccgguugcccggucuuguauauggucgugaugcagaaaguuucgcgauaaaugaaccccgcgccuggcggcgcguccuugaugaugccgccguacgccuggcccagguacguaccgucauagagaccguauauggaccagccgggcgcuaucuggcgcaaauacgucugcaauucggcguugccgcgcaguucggccacggucaaggcgcggguuccguucgggcagcguccauaggcgccgcccuguugggugaacagugccuucggcgggaugacgaugccuggcgcaacggccugggccgugcgcaugcccagcagggcgagcaccaggaugggcagaaugugaugaagcagcuucuuguuguugaucagcauguugcgguguucccggaauggaaccagggcggaagaucgucucaagcggauugcgcggaaacgucgaaaccuccggaaggguucauucgcaauauccguugagcgggcagaucugcaguucgagcagaucgccgggagugcccggauacggcgagucuuccaccgucagcgcgauccggcucuugagcgcauacgcggauauggcaaagccaagcauggugucgaaccacgugucgugcucgugucccgcgucggacaggcacgcccggaccaggcuucugcccgacaugaaggcggucaggcagaacuccugauucuugcccuucaguuucaaggccagcuccuggacagugaaguucuuguacagaugggucggcaagccggcgacgucagccgggcuguauaugccgagcacggacaacaggauggaugcgaugguaugcaugggguucgccuucaggggcaauccugcuugccgcugcauucgaccauccggaucaguucgagcgcgggcuugccuucgaaagugaguugcuugggacccagaaacaugcgcagcggccguuguugcaugaagacggcgcgcaacaugacuuccaugggacugcugccgggacgcuugagauccuugccgaagcagaacggcacgugcgcguccgggcugcuggccgcggcgcccaguuuggcggcgaugccgcagaccagcaugcgcgucggggugacuucauacggcuucacggcuacgcuggugaccaccauauuggucuucaccagcacauaaggaacgucggccagggcgggggaaagaugcgucaucgcgccggaugccagcaaccacgccagggcgcgcacgcgcgaccgccgggcgccgcccugucccggggcguugguucgaguggggaagcgucucagcauaaggaugauccaggauugcagauggagaugucgguaagggcguaagucucgaacguugcguccucauagucguaauacuguucuuccuugcugacauggacgcguacggagaugccggccacguagaucagguaaagcauucuccgcagccggcuguacaugcuccaguacuugccgucauacgggcuggugcaggcgccaaugaccgguugcccgcuucugacgaagaccgcgcauagccugcuguuggugcuggagagcaggcgaguggcggugacguugcuguaguagugauccguugcggguugacccguauugcgcguggucaugaugcagaacgucguuuucaggucgaaugcgccgccggguguuccguccuugaucacgccgccauauucgccgccgagauaggugccaucguagagcgcaaauauugaccagccgcgcgucacaugacgcagguacuccugcagaucgccgcugccgcgcaauuccgccacggucagggcacgggucuuguucgcgcagcguccauaggggccgccaugcuggguaaucuguuccugcggcggaaugacgaugccuggcguggaggcccgcgccacgugagauccgaggagggccaacggcagaacggacaggagauggcagagcgucuugcggucgaucggcaugcuguucaauuaccggaguugggcggggcugggccuggucuagaacgaauacgcgaugcuuucguaguacacgagaaccaucgccucgccggcgcguucggaccaggcugccauggccucggagcuuucggccugccgcgccaugcaagcgccuaucaccggcgccaugcgcaccaaugugccgacgaucgacgcuacggaccuucgcgauguguagggguugggauuggcgcgagucugcuggcugacguagcgagcguuggaauacuccguggucguggucucgccggugaugccguugugauagacccgcguuacccugcggauguuuucgggcggaaugcgccggugugccagauauucgcucugguagguggccagcgcgccggcgaggauacggccggcauugucgccauaagugucgacguauucgaaguacgagcuggcggcgccguagaaauugcugucggcgcggacuucguagauguagccgaugaaguggccggugccccugccggcgcguucggccucgaccgcuuccugcaugcgauguucgagauagaccucgguauagcgccggcugcugcugguggagacgaaagcgcuguugcugcugccgaccuggcaggaacguccggucagauggucgagcacauugucguuguuuccccacgccgugaauccguucuggaaaacguccuccggcgggcgggagucauagcgguauacgguggcgggaggaucgucggcccaugccggcgaagucacgggcgccgugacggcaagaaucgccagccacgucagccagccuguucuugcgguuuggcgaauugcccgagugcaacgcaucccgu

SEQ ID NO: 8 - protein, 269 aa, Artificial sequence
  Synthetic
MRCTRAIRQTARTGWLTWLAILAVTAPVTSPAWADDPPATVYRYDSRPPEDVFQNGFTAWGNNDNVLDHLTGRSCQVGSSNSAFVSTSSSRRYTEVYLEHRMQEAVEAERAGRGTGHFIGYIYEVRADNNFYGAASSYFEYVDTYGDNAGRILAGALATYQSEYLAHRRIPPENIRRVTRVYHNGITGETTTTEYSNARYVSQQTRANPNPYTSRRSVASIVGTLVRMAPVIGACMARQAESSEAMAAWSERAGEAMVLVYYESIAYSF

SEQ ID NO: 9 - RNA, 941 nt, Artificial sequence
  Synthetic
gggaaauaagagagaaaagaagaguaagaagaaauauaagaccccggcgccgccaccauggaaaccccugcucagcugcuguuccugcugcugcuguggcugccugauaccacaggggaugauccuccagccaccguguauaaguacgacagcagaccuccugaggacguguuccagaauggcuuuaccgccuggggcaacaacgacaacgugcuggaucaccugaccggcagauccugucaagugggcagcagcaauagcgccuucguguccaccucuagcagcagacgguacacagagguguaccuggaacaccggaugcaagaggccguggaagccgaaagagccggcagaggaacaggccacuucaucggcuacaucuacgaagugcgggccgacaacaacuucuacggcgcugccagcagcuacuucgaguacguggacaccuacggcgacaacgccggaagaauacuggcuggcgcucuggccacauaccagucuggauaucuggcccacagacggaucccuccagagaacauucggagagugacccggguguaccacaacggcauuaccggcgagacaaccaccaccgaguacagcaacgccagauacgugucccagcagacccgggccaauccuaauccuuacaccagcagaagaagcguggccagcaucgugggcacccuggugagaauggccccugugaucggcgccugcauggccagacaggccgagagcagcgaggccauggccgccuggagcgagagagccggagaggccauggugcugguguacuacgagagcaucgccuacagcuucugauaauaggcuggagccucgguggccuagcuucuugccccuugggccuccccccagccccuccuccccuuccugcacccguacccccguggucuuugaauaaagucugagugggcggc

SEQ ID NO: 10 - RNA, 765 nt, Artificial sequence
  Synthetic
auggaaaccccugcucagcugcuguuccugcugcugcuguggcugccugauaccacaggggaugauccuccagccaccguguauaaguacgacagcagaccuccugaggacguguuccagaauggcuuuaccgccuggggcaacaacgacaacgugcuggaucaccugaccggcagauccugucaagugggcagcagcaauagcgccuucguguccaccucuagcagcagacgguacacagagguguaccuggaacaccggaugcaagaggccguggaagccgaaagagccggcagaggaacaggccacuucaucggcuacaucuacgaagugcgggccgacaacaacuucuacggcgcugccagcagcuacuucgaguacguggacaccuacggcgacaacgccggaagaauacuggcuggcgcucuggccacauaccagucuggauaucuggcccacagacggaucccuccagagaacauucggagagugacccggguguaccacaacggcauuaccggcgagacaaccaccaccgaguacagcaacgccagauacgugucccagcagacccgggccaauccuaauccuuacaccagcagaagaagcguggccagcaucgugggcacccuggugagaauggccccugugaucggcgccugcauggccagacaggccgagagcagcgaggccauggccgccuggagcgagagagccggagaggccauggugcugguguacuacgagagcaucgccuacagcuuc

SEQ ID NO: 11 - protein, 255 aa, Artificial sequence
  Synthetic
METPAQLLFLLLLWLPDTTGDDPPATVYKYDSRPPEDVFQNGFTAWGNNDNVLDHLTGRSCQVGSSNSAFVSTSSSRRYTEVYLEHRMQEAVEAERAGRGTGHFIGYIYEVRADNNFYGAASSYFEYVDTYGDNAGRILAGALATYQSGYLAHRRIPPENIRRVTRVYHNGITGETTTTEYSNARYVSQQTRANPNPYTSRRSVASIVGTLVRMAPVIGACMARQAESSEAMAAWSERAGEAMVLVYYESIAYSF

SEQ ID NO: 12 - RNA, 824 nt, Artificial sequence
  Synthetic
gggaaauaagagagaaaagaagaguaagaagaaauauaagaccccggcgccgccaccauggagaccccugcccagcugcuguuccugcugcugcuguggcugccugacaccaccggcggcaucgugaucccuccucaggagcagaucacccagcacggcggcccuuacggcagaugcgccaacaaggccagagcccugaccguggccgagcugagaggcagcggcgaccugcaggaguaccugagacacgugaccagaggcuggagcaucuucgcccuguacgacggcaccuaccugggcggcgaguacggcggcgugaucaaggacggcaccccuggcggcgccuucgaccugaagaccaccuucugcaucaugaccaccagaaacaccggccagccugccaccgaccacuacuacagcaaggugaccgccaccagacugcugagcagcaccaacagcagacugugcgccguguucgugagaagcggccagccugugaucggcgccugcaccagcccuuacgacggcaaguacuggagcauguacagcagacugagaaagaugcuguaccugaucuacguggccggcaucagcgugagagugcacgugagcaaggaggagcaguacuacgacuacgaggacgccaccuucgagaccuacgcccugaccggcaucagcaucugcaacccuggcagcagccugugcugauaauaggcuggagccucgguggccuagcuucuugccccuugggccuccccccagccccuccuccccuuccugcacccguacccccguggucuuugaauaaagucugagugggcggc

SEQ ID NO: 13 - RNA, 648 nt, Artificial sequence
  Synthetic
auggagaccccugcccagcugcuguuccugcugcugcuguggcugccugacaccaccggcggcaucgugaucccuccucaggagcagaucacccagcacggcggcccuuacggcagaugcgccaacaaggccagagcccugaccguggccgagcugagaggcagcggcgaccugcaggaguaccugagacacgugaccagaggcuggagcaucuucgcccuguacgacggcaccuaccugggcggcgaguacggcggcgugaucaaggacggcaccccuggcggcgccuucgaccugaagaccaccuucugcaucaugaccaccagaaacaccggccagccugccaccgaccacuacuacagcaaggugaccgccaccagacugcugagcagcaccaacagcagacugugcgccguguucgugagaagcggccagccugugaucggcgccugcaccagcccuuacgacggcaaguacuggagcauguacagcagacugagaaagaugcuguaccugaucuacguggccggcaucagcgugagagugcacgugagcaaggaggagcaguacuacgacuacgaggacgccaccuucgagaccuacgcccugaccggcaucagcaucugcaacccuggcagcagccugugc

SEQ ID NO: 14 - protein, 216 aa, Artificial sequence
  Synthetic
METPAQLLFLLLLWLPDTTGGIVIPPQEQITQHGGPYGRCANKARALTVAELRGSGDLQEYLRHVTRGWSIFALYDGTYLGGEYGGVIKDGTPGGAFDLKTTFCIMTTRNTGQPATDHYYSKVTATRLLSSTNSRLCAVFVRSGQPVIGACTSPYDGKYWSMYSRLRKMLYLIYVAGISVRVHVSKEEQYYDYEDATFETYALTGISICNPGSSLC

SEQ ID NO: 15 - RNA, 824 nt, Artificial sequence
  Synthetic
gggaaauaagagagaaaagaagaguaagaagaaauauaagaccccggcgccgccaccauggagaccccugcccagcugcuguuccugcugcugcuguggcugccugacaccaccggcggcaucgugaucccuccuaaggcccuguucacccagcagggcggcgccuacggcagaugcccuaacggcaccagagcccugaccguggccgagcugagaggcaacgccgagcugcagaccuaccugagacagaucaccccuggcuggagcaucuacggccuguacgacggcaccuaccugggccaggccuacggcggcaucaucaaggacgccccuccuggcgccggcuucaucuacagagagaccuucugcaucaccaccaucuacaagaccggccagccugccgccgaccacuacuacagcaaggugaccgccaccagacugcuggccagcaccaacagcagacugugcgccguguucgugagagacggccagagcgugaucggcgccugcgccagcccuuacgagggcagauacagagacauguacgacgcccugagaagacugcuguacaugaucuacaugagcggccuggccgugagagugcacgugagcaaggaggagcaguacuacgacuacgaggacgccaccuuccagaccuacgcccugaccggcaucagccugugcaacccugccgccagcaucugcugauaauaggcuggagccucgguggccuagcuucuugccccuugggccuccccccagccccuccuccccuuccugcacccguacccccguggucuuugaauaaagucugagugggcggc

SEQ ID NO: 16 - RNA, 648 nt, Artificial sequence
  Synthetic
auggagaccccugcccagcugcuguuccugcugcugcuguggcugccugacaccaccggcggcaucgugaucccuccuaaggcccuguucacccagcagggcggcgccuacggcagaugcccuaacggcaccagagcccugaccguggccgagcugagaggcaacgccgagcugcagaccuaccugagacagaucaccccuggcuggagcaucuacggccuguacgacggcaccuaccugggccaggccuacggcggcaucaucaaggacgccccuccuggcgccggcuucaucuacagagagaccuucugcaucaccaccaucuacaagaccggccagccugccgccgaccacuacuacagcaaggugaccgccaccagacugcuggccagcaccaacagcagacugugcgccguguucgugagagacggccagagcgugaucggcgccugcgccagcccuuacgagggcagauacagagacauguacgacgcccugagaagacugcuguacaugaucuacaugagcggccuggccgugagagugcacgugagcaaggaggagcaguacuacgacuacgaggacgccaccuuccagaccuacgcccugaccggcaucagccugugcaacccugccgccagcaucugc

SEQ ID NO: 17 - protein, 216 aa, Artificial sequence
  Synthetic
METPAQLLFLLLLWLPDTTGGIVIPPKALFTQQGGAYGRCPNGTRALTVAELRGNAELQTYLRQITPGWSIYGLYDGTYLGQAYGGIIKDAPPGAGFIYRETFCITTIYKTGQPAADHYYSKVTATRLLASTNSRLCAVFVRDGQSVIGACASPYEGRYRDMYDALRRLLYMIYMSGLAVRVHVSKEEQYYDYEDATFQTYALTGISLCNPAASIC

SEQ ID NO: 18 - RNA, 566 nt, Artificial sequence
  Synthetic
gggaaauaagagagaaaagaagaguaagaagaaauauaagaccccggcgccgccaccauggagaccccugcccagcugcuguuccugcugcugcuguggcugccugacaccaccggcgacgugccuuacgugcuggugaagaccaacaugguggugaccagcguggccaugaagccuuacgaggugaccccuaccagaaugcuggugugcggcaucgccgccaagcugggcgccgccgccagcagcccugacgcccacgugccuuucugcuucggcaaggaccugaagagaccuggcagcagcccuauggaggugaugcugagagccguguucaugcagcagagaccucugagaauguuccugggcccuaagcagcugaccuucgagggcaagccugcccuggagcugaucagaaugguggagugcagcggcaagcaggacugcccuugauaauaggcuggagccucgguggccuagcuucuugccccuugggccuccccccagccccuccuccccuuccugcacccguacccccguggucuuugaauaaagucugagugggcggc

SEQ ID NO: 19 - RNA, 390 nt, Artificial sequence
  Synthetic
auggagaccccugcccagcugcuguuccugcugcugcuguggcugccugacaccaccggcgacgugccuuacgugcuggugaagaccaacaugguggugaccagcguggccaugaagccuuacgaggugaccccuaccagaaugcuggugugcggcaucgccgccaagcugggcgccgccgccagcagcccugacgcccacgugccuuucugcuucggcaaggaccugaagagaccuggcagcagcccuauggaggugaugcugagagccguguucaugcagcagagaccucugagaauguuccugggcccuaagcagcugaccuucgagggcaagccugcccuggagcugaucagaaugguggagugcagcggcaagcaggacugcccu

SEQ ID NO: 20 - protein, 130 aa, Artificial sequence
  Synthetic
METPAQLLFLLLLWLPDTTGDVPYVLVKTNMVVTSVAMKPYEVTPTRMLVCGIAAKLGAAASSPDAHVPFCFGKDLKRPGSSPMEVMLRAVFMQQRPLRMFLGPKQLTFEGKPALELIRMVECSGKQDCP

SEQ ID NO: 21 - RNA, 530 nt, Artificial sequence
  Synthetic
gggaaauaagagagaaaagaagaguaagaagaaauauaagaccccggcgccgccaccauggagaccccugcccagcugcuguuccugcugcugcuguggcugccugacaccaccggccugccuacccaccuguacaagaacuucgccgugcaggagcuggcccugaagcugaagggcaagaaccaggaguucugccugaccgccuucaugagcggcagaagccuggugagagccugccugagcgacgccggccacgagcacgacaccugguucgacaccaugcugggcuucgccaucagcgccuacgcccugaagagcagaaucgcccugaccguggaggacagcccuuacccuggcaccccuggcgaccugcuggagcugcagaucugcccucugaacggcuacugcgagugauaauaggcuggagccucgguggccuagcuucuugccccuugggccuccccccagccccuccuccccuuccugcacccguacccccguggucuuugaauaaagucugagugggcggc

SEQ ID NO: 22 - RNA, 354 nt, Artificial sequence
  Synthetic
auggagaccccugcccagcugcuguuccugcugcugcuguggcugccugacaccaccggccugccuacccaccuguacaagaacuucgccgugcaggagcuggcccugaagcugaagggcaagaaccaggaguucugccugaccgccuucaugagcggcagaagccuggugagagccugccugagcgacgccggccacgagcacgacaccugguucgacaccaugcugggcuucgccaucagcgccuacgcccugaagagcagaaucgcccugaccguggaggacagcccuuacccuggcaccccuggcgaccugcuggagcugcagaucugcccucugaacggcuacugcgag

SEQ ID NO: 23 - protein, 118 aa, Artificial sequence
  Synthetic
METPAQLLFLLLLWLPDTTGLPTHLYKNFAVQELALKLKGKNQEFCLTAFMSGRSLVRACLSDAGHEHDTWFDTMLGFAISAYALKSRIALTVEDSPYPGTPGDLLELQICPLNGYCE

SEQ ID NO: 24 - RNA, 2342 nt, Artificial sequence
  Synthetic
gggaaauaagagagaaaagaagaguaagaagaaauauaagaccccggcgccgccaccauggaaaccccugcacagcugcuguuccugcugcugcuguggcugcccgacacgaccggcggcucuccuggcggacgagcuccuagcgcaccccaacccgcuccuucuccuagaccugagccugccccagaaccagccccuaauccggcuccacggccugccccucagccuccugcaccugcuccuggagcaccaagaccuccugcuccuccaccugaggcuccuccucccguuaugccuccuccugccguuccuccucaauugccggaggugccugcugcagaucuuccucggguccgcgcuccccugagcacuuauagacgcccucagcgcaccgauuucguuaccccuaccggcggcccguucuucgccaaacaggacaaagcgcucaacaccauugaucugaagauggcccacgaucugaagcugagaggauacagaguaaagguugcaguaguggacgagggaguaagauccgaccacccacugcugaacguggagaagaaguacggcggcgacuacauggccgacggcacccgcaccuacccugacccuaagcgacagggcagacacgggacgucgguggcccuggugcuugccggccaggacaccgacaccuauagaggcggaguggccccuaacgcggacuuguauagcgccaacauuggcacucgugccggccacguaagcgacgaagccgcauuucacgccuggaacgaccuacucggccacggcaucaagauuuucaauaacagcuucgccacugaaggccccgagggagagcagagaguuaaggaggacagaaacgaguaccauuccgccgccaauaagcagaauaccuacauaggcagacuggaucgccucguucgggacggagcucuccuuaucuuugcugcaggcaacgggcgaccuagcggacgggccuacucagaggugggcagcguaggaaggacaccucgcgucgagccacaucuucagcgcggccugaucguggucacugcagucgacgagaacggucggcuggaaaccugggccaaccggugcggccaggcccagcaguggugccuugcugcuccuucaacagcauaucugccagggcuggauaaggacaacccugacagcauccacguugaacaggguaccgcccugagcgcaccgcucgugaccggcgccgcagugcugguacaggacagauuucgcuggauggacaacgacaaccugcgcaccacccuucuaaccaccgcucaggauaaggggccuuacggggucgauccucaguacggcuggggcgugcuggacgugggccgcgccgugcaaggcccugcccaguuugcuuuuggagacuucguggccagagucaccgauacuuccaccuucgggaacgauauuagcggcgcuggcggccuugugguagacggcccuggcgccuuaguccuggcagggaguaauacauacgccggcaggaccacuaucaagagaggcacccuggacguguucggaagcgugaccuccgcagucaccguggaaccuggaggcacucucaccggcauagggacuguagguaccgugaccaaucagggcacggugguuaauaaggaagccggccuccacgugaaaggggauuauucccagaccgcacagggccuacuggugacagauauuggcucucugcuggacgucagcggcagagcuagccucgccgguagacugcacguugacgauaucagaccuggguacguaggcggcgacggaaaguccgucccugugauuaaggccggugcggucagcgggguguucgccacccugacacgcucaccaggccugcucuugaacgcgaggcuggacuacagaccucaggcuguauaucugacuaugagacgcgcugaaagaguccacgccgccgcccagagaggagccgacgacggcagacgggcuuccgugcuugccgucgccgagagacucgacgcagccaugcgagagcuggacgcuuugccggaaagucagagggacgccgcagccccugccgcagcgauuggacggauucagcgggugcagagcagaaagguuuugcaggacaaccuguacucuuuagcuggcgcaaccuacgccaacgcugcugccgugaacacccuagaacagaacagguggauggauagacuggagaacugauaauaggcuggagccucgguggccuagcuucuugccccuugggccuccccccagccccuccuccccuuccugcacccguacccccguggucuuugaauaaagucugagugggcggc

SEQ ID NO: 25 - RNA, 2166 nt, Artificial sequence
  Synthetic
auggaaaccccugcacagcugcuguuccugcugcugcuguggcugcccgacacgaccggcggcucuccuggcggacgagcuccuagcgcaccccaacccgcuccuucuccuagaccugagccugccccagaaccagccccuaauccggcuccacggccugccccucagccuccugcaccugcuccuggagcaccaagaccuccugcuccuccaccugaggcuccuccucccguuaugccuccuccugccguuccuccucaauugccggaggugccugcugcagaucuuccucggguccgcgcuccccugagcacuuauagacgcccucagcgcaccgauuucguuaccccuaccggcggcccguucuucgccaaacaggacaaagcgcucaacaccauugaucugaagauggcccacgaucugaagcugagaggauacagaguaaagguugcaguaguggacgagggaguaagauccgaccacccacugcugaacguggagaagaaguacggcggcgacuacauggccgacggcacccgcaccuacccugacccuaagcgacagggcagacacgggacgucgguggcccuggugcuugccggccaggacaccgacaccuauagaggcggaguggccccuaacgcggacuuguauagcgccaacauuggcacucgugccggccacguaagcgacgaagccgcauuucacgccuggaacgaccuacucggccacggcaucaagauuuucaauaacagcuucgccacugaaggccccgagggagagcagagaguuaaggaggacagaaacgaguaccauuccgccgccaauaagcagaauaccuacauaggcagacuggaucgccucguucgggacggagcucuccuuaucuuugcugcaggcaacgggcgaccuagcggacgggccuacucagaggugggcagcguaggaaggacaccucgcgucgagccacaucuucagcgcggccugaucguggucacugcagucgacgagaacggucggcuggaaaccugggccaaccggugcggccaggcccagcaguggugccuugcugcuccuucaacagcauaucugccagggcuggauaaggacaacccugacagcauccacguugaacaggguaccgcccugagcgcaccgcucgugaccggcgccgcagugcugguacaggacagauuucgcuggauggacaacgacaaccugcgcaccacccuucuaaccaccgcucaggauaaggggccuuacggggucgauccucaguacggcuggggcgugcuggacgugggccgcgccgugcaaggcccugcccaguuugcuuuuggagacuucguggccagagucaccgauacuuccaccuucgggaacgauauuagcggcgcuggcggccuugugguagacggcccuggcgccuuaguccuggcagggaguaauacauacgccggcaggaccacuaucaagagaggcacccuggacguguucggaagcgugaccuccgcagucaccguggaaccuggaggcacucucaccggcauagggacuguagguaccgugaccaaucagggcacggugguuaauaaggaagccggccuccacgugaaaggggauuauucccagaccgcacagggccuacuggugacagauauuggcucucugcuggacgucagcggcagagcuagccucgccgguagacugcacguugacgauaucagaccuggguacguaggcggcgacggaaaguccgucccugugauuaaggccggugcggucagcgggguguucgccacccugacacgcucaccaggccugcucuugaacgcgaggcuggacuacagaccucaggcuguauaucugacuaugagacgcgcugaaagaguccacgccgccgcccagagaggagccgacgacggcagacgggcuuccgugcuugccgucgccgagagacucgacgcagccaugcgagagcuggacgcuuugccggaaagucagagggacgccgcagccccugccgcagcgauuggacggauucagcgggugcagagcagaaagguuuugcaggacaaccuguacucuuuagcuggcgcaaccuacgccaacgcugcugccgugaacacccuagaacagaacagguggauggauagacuggagaac

SEQ ID NO: 26 - protein, 722 aa, Artificial sequence
  Synthetic
METPAQLLFLLLLWLPDTTGGSPGGRAPSAPQPAPSPRPEPAPEPAPNPAPRPAPQPPAPAPGAPRPPAPPPEAPPPVMPPPAVPPQLPEVPAADLPRVRAPLSTYRRPQRTDFVTPTGGPFFAKQDKALNTIDLKMAHDLKLRGYRVKVAVVDEGVRSDHPLLNVEKKYGGDYMADGTRTYPDPKRQGRHGTSVALVLAGQDTDTYRGGVAPNADLYSANIGTRAGHVSDEAAFHAWNDLLGHGIKIFNNSFATEGPEGEQRVKEDRNEYHSAANKQNTYIGRLDRLVRDGALLIFAAGNGRPSGRAYSEVGSVGRTPRVEPHLQRGLIVVTAVDENGRLETWANRCGQAQQWCLAAPSTAYLPGLDKDNPDSIHVEQGTALSAPLVTGAAVLVQDRFRWMDNDNLRTTLLTTAQDKGPYGVDPQYGWGVLDVGRAVQGPAQFAFGDFVARVTDTSTFGNDISGAGGLVVDGPGALVLAGSNTYAGRTTIKRGTLDVFGSVTSAVTVEPGGTLTGIGTVGTVTNQGTVVNKEAGLHVKGDYSQTAQGLLVTDIGSLLDVSGRASLAGRLHVDDIRPGYVGGDGKSVPVIKAGAVSGVFATLTRSPGLLLNARLDYRPQAVYLTMRRAERVHAAAQRGADDGRRASVLAVAERLDAAMRELDALPESQRDAAAPAAAIGRIQRVQSRKVLQDNLYSLAGATYANAAAVNTLEQNRWMDRLEN

SEQ ID NO: 27 - RNA, 1205 nt, Artificial sequence
  Synthetic
gggaaauaagagagaaaagaagaguaagaagaaauauaagaccccggcgccgccaccauggaaacuccugcccagcugcuguuccugcuccuucuguggcugccugacaccaccggccugaagcugccuagccugcugaccgacgacgagcuuaagcuggugcugccuaccggcaugagccuggaggacuucaagagaagccugcaggagagcgccccuagcgcccuggccaccccuccuagcagcagcccuccuguggccaagccuggcccuggcagcguggccgaggcuccgucaggcagcggccacaaggacaacccuucaccuccuguugugggcgugggaccaggcauggccgagagcagcggcggacauaacccuggcguaggaggcggcacccacgagaacggccugccuggcaucggcaaggucgguggaagugccccuggcccagauaccagcacuggaagcggcccugacgccggcauggcuagcggcgccggcagcaccagcccuggcgccucagguaugccuccuuccgagggcgagcgcccugauaguggaaugucugacucaggaagaggcggagagagcagugccggcggccuaaacccugacggagcuggcaagccuccuagagaggagggagagccgggcuccaagagcccugccgacggcggccaggacggcccuccuccgccuagggacgguggcgacgccgacccucagccuccucgagacgacggcaacggcgaacagcagccaccuaaggguggcggcgacgagggccagcgcccaccuccggcggccggaaacggcgguaacggaggcaacggcaacgcgcaguugccagagagaggcgacgacgccgguccuaagccaccagagggcgaaggcggcgacgaaggaccacagccuccacaaggcggaggugaacaggacgccccugaggugccuccuguagcuccggcuccaccugcgggaaacgguguguacgacccugguacacacacccugaccacuccagccagcgccgccguaucucuugcuucauccucacacggugucuggcaggccgagaugaacugauaauaggcuggagccucgguggccuagcuucuugccccuugggccuccccccagccccuccuccccuuccugcacccguacccccguggucuuugaauaaagucugagugggcggc

SEQ ID NO: 28 - RNA, 1029 nt, Artificial sequence
  Synthetic
auggaaacuccugcccagcugcuguuccugcuccuucuguggcugccugacaccaccggccugaagcugccuagccugcugaccgacgacgagcuuaagcuggugcugccuaccggcaugagccuggaggacuucaagagaagccugcaggagagcgccccuagcgcccuggccaccccuccuagcagcagcccuccuguggccaagccuggcccuggcagcguggccgaggcuccgucaggcagcggccacaaggacaacccuucaccuccuguugugggcgugggaccaggcauggccgagagcagcggcggacauaacccuggcguaggaggcggcacccacgagaacggccugccuggcaucggcaaggucgguggaagugccccuggcccagauaccagcacuggaagcggcccugacgccggcauggcuagcggcgccggcagcaccagcccuggcgccucagguaugccuccuuccgagggcgagcgcccugauaguggaaugucugacucaggaagaggcggagagagcagugccggcggccuaaacccugacggagcuggcaagccuccuagagaggagggagagccgggcuccaagagcccugccgacggcggccaggacggcccuccuccgccuagggacgguggcgacgccgacccucagccuccucgagacgacggcaacggcgaacagcagccaccuaaggguggcggcgacgagggccagcgcccaccuccggcggccggaaacggcgguaacggaggcaacggcaacgcgcaguugccagagagaggcgacgacgccgguccuaagccaccagagggcgaaggcggcgacgaaggaccacagccuccacaaggcggaggugaacaggacgccccugaggugccuccuguagcuccggcuccaccugcgggaaacgguguguacgacccugguacacacacccugaccacuccagccagcgccgccguaucucuugcuucauccucacacggugucuggcaggccgagaugaac

SEQ ID NO: 29 - protein, 343 aa, Artificial sequence
  Synthetic
METPAQLLFLLLLWLPDTTGLKLPSLLTDDELKLVLPTGMSLEDFKRSLQESAPSALATPPSSSPPVAKPGPGSVAEAPSGSGHKDNPSPPVVGVGPGMAESSGGHNPGVGGGTHENGLPGIGKVGGSAPGPDTSTGSGPDAGMASGAGSTSPGASGMPPSEGERPDSGMSDSGRGGESSAGGLNPDGAGKPPREEGEPGSKSPADGGQDGPPPPRDGGDADPQPPRDDGNGEQQPPKGGGDEGQRPPPAAGNGGNGGNGNAQLPERGDDAGPKPPEGEGGDEGPQPPQGGGEQDAPEVPPVAPAPPAGNGVYDPGTHTLTTPASAAVSLASSSHGVWQAEMN

SEQ ID NO: 30 - RNA, 2057 nt, Artificial sequence
  Synthetic
gggaaauaagagagaaaagaagaguaagaagaaauauaagaccccggcgccgccaccauggagaccccugcccagcugcuguuccugcugcugcuuuggcugccagacaccaccggagacuggaacaaccaggccaucgugaagaccggcgagagacagcacggcauccacauccaaggcagcgaccccggcggcgugagaaccgccuccggcaccacaaucaaggugagcggcagacaggcccagggcauccugcuggagaacccugccgccgagcugcaguucagaaacggcgccgugaccagcagcggccagcuguccgacgacggcaucagaagauuccugggcaccgugaccgugaaggccggcaagcugguggccgaccacgccacccuggccaacgugggugauaccugggacgacgacgguauugcccuguacguggccggcgagcaagcacaggccagcaucgccgacagcacccugcaaggcgcaggcggcgugcagaucgagagaggcgccaacgugguggugcagagaagcgccauuguugacggcggccugcacaucggcgcccugcagagccugcagccugaggaccugccuccuagcagaguggugcugagagacacaaacgucgucgccgugccagcguccggagcuccagcagccgugagcgugcugggcgccagcgagcugacccucgacggaggacacaucaccgguggcagggccgccgguguggccgccaugcagggagccguugugcaucuacaaagagcuaccauccgacggggcgacgcuccggcugguggagcggucccuggcggugccguccccggaggagcagugccaggcgguuucggcccggguggauucggcccugugcuagacggcugguacggcguggacgugaguggcuccagcguggagcuggcccagaguauuguugaggccccugagcucggcgcugccaucagagugggccguggugccagagugaccguuagcgguggcucacuuagcgccccucacggcaacgugaucgaaacuggaggagcgagacgauucgcaccucaggccgccccucugagcauaacgcugcaagcgggcgcccacgcgcaaggcaaggcccugcuguacagagugcugccugagccugugaaguuaacucugacgggaggugccgacgcacaaggugacaucguggccacggaacuuccuagcaucccuggcacaucuaucggcccucuggacguugcguuagcuucgcaggcccguuggaccggcgccaccagagccguggacucccuaagcaucgacaacgccgugugggucaugaccgacaacucaaacgugggcgcgcugagauuggccucagacggcagugucgauuuccaacaaccugccgaagcaggaagauucaaggugcugacugucaauacacuggccggaagcggccuguucagaaugaacguguucgccgaccugggacucucagauaagcuggucgugaugcaggacgcaaguggccagcacagacugugggucagaaacucgggcuccgagccagccagugccaauacuuugcuguuggugcaaaccccucugggcagugccgccaccuucacucucgccaacaaggacggcaagguggacaucggcaccuacagauacaggcuggcagcaaacggaaacggccaguggagccuagucggagcaaaggccccuccugccccuaagccugcuccacagccuggcccacagccuccgcaaccuccgcagccucagccagaggccccagccccacaaccuccugcaggcagagagcugagugccgcugcuaacgccgccguuaauaccggaggugucggccucgcgucuaccuugugguacgccgagagcaaccgggccaagagaagcggaagcggcgcucccugauaauaggcuggagccucgguggccuagcuucuugccccuugggccuccccccagccccuccuccccuuccugcacccguacccccguggucuuugaauaaagucugagugggcggc

SEQ ID NO: 31 - RNA, 1881 nt, Artificial sequence
  Synthetic
auggagaccccugcccagcugcuguuccugcugcugcuuuggcugccagacaccaccggagacuggaacaaccaggccaucgugaagaccggcgagagacagcacggcauccacauccaaggcagcgaccccggcggcgugagaaccgccuccggcaccacaaucaaggugagcggcagacaggcccagggcauccugcuggagaacccugccgccgagcugcaguucagaaacggcgccgugaccagcagcggccagcuguccgacgacggcaucagaagauuccugggcaccgugaccgugaaggccggcaagcugguggccgaccacgccacccuggccaacgugggugauaccugggacgacgacgguauugcccuguacguggccggcgagcaagcacaggccagcaucgccgacagcacccugcaaggcgcaggcggcgugcagaucgagagaggcgccaacgugguggugcagagaagcgccauuguugacggcggccugcacaucggcgcccugcagagccugcagccugaggaccugccuccuagcagaguggugcugagagacacaaacgucgucgccgugccagcguccggagcuccagcagccgugagcgugcugggcgccagcgagcugacccucgacggaggacacaucaccgguggcagggccgccgguguggccgccaugcagggagccguugugcaucuacaaagagcuaccauccgacggggcgacgcuccggcugguggagcggucccuggcggugccguccccggaggagcagugccaggcgguuucggcccggguggauucggcccugugcuagacggcugguacggcguggacgugaguggcuccagcguggagcuggcccagaguauuguugaggccccugagcucggcgcugccaucagagugggccguggugccagagugaccguuagcgguggcucacuuagcgccccucacggcaacgugaucgaaacuggaggagcgagacgauucgcaccucaggccgccccucugagcauaacgcugcaagcgggcgcccacgcgcaaggcaaggcccugcuguacagagugcugccugagccugugaaguuaacucugacgggaggugccgacgcacaaggugacaucguggccacggaacuuccuagcaucccuggcacaucuaucggcccucuggacguugcguuagcuucgcaggcccguuggaccggcgccaccagagccguggacucccuaagcaucgacaacgccgugugggucaugaccgacaacucaaacgugggcgcgcugagauuggccucagacggcagugucgauuuccaacaaccugccgaagcaggaagauucaaggugcugacugucaauacacuggccggaagcggccuguucagaaugaacguguucgccgaccugggacucucagauaagcuggucgugaugcaggacgcaaguggccagcacagacugugggucagaaacucgggcuccgagccagccagugccaauacuuugcuguuggugcaaaccccucugggcagugccgccaccuucacucucgccaacaaggacggcaagguggacaucggcaccuacagauacaggcuggcagcaaacggaaacggccaguggagccuagucggagcaaaggccccuccugccccuaagccugcuccacagccuggcccacagccuccgcaaccuccgcagccucagccagaggccccagccccacaaccuccugcaggcagagagcugagugccgcugcuaacgccgccguuaauaccggaggugucggccucgcgucuaccuugugguacgccgagagcaaccgggccaagagaagcggaagcggcgcuccc

SEQ ID NO: 32 - protein, 617 aa, Artificial sequence
  Synthetic
METPAQLLFLLLLWLPDTTGDWNNQAIVKTGERQHGIHIQGSDPGGVRTASGTTIKVSGRQAQGILLENPAAELQFRNGAVTSSGQLSDDGIRRFLGTVTVKAGKLVADHATLANVGDTWDDDGIALYVAGEQAQASIADSTLQGAGGVQIERGANVVVQRSAIVDGGLHIGALQSLQPEDLPPSRVVLRDTNVVAVPASGAPAAVSVLGASELTLDGGHITGGRAAGVAAMQGAVVHLQRATIRRGDAPAGGAVPGGAVPGGAVPGGFGPGGFGPVLDGWYGVDVSGSSVELAQSIVEAPELGAAIRVGRGARVTVSGGSLSAPHGNVIETGGARRFAPQAAPLSITLQAGAHAQGKALLYRVLPEPVKLTLTGGADAQGDIVATELPSIPGTSIGPLDVALASQARWTGATRAVDSLSIDNAVWVMTDNSNVGALRLASDGSVDFQQPAEAGRFKVLTVNTLAGSGLFRMNVFADLGLSDKLVVMQDASGQHRLWVRNSGSEPASANTLLLVQTPLGSAATFTLANKDGKVDIGTYRYRLAANGNGQWSLVGAKAPPAPKPAPQPGPQPPQPPQPQPEAPAPQPPAGRELSAAANAAVNTGGVGLASTLWYAESN

SEQ ID NO: 33 - RNA, 1637 nt, Artificial sequence
  Synthetic
gggaaauaagagagaaaagaagaguaagaagaaauauaagaccccggcgccgccaccauggagaccccugcccagcugcuguuccugcugcugcuuuggcugccugacaccaccggaagccuguacgccgaacacgacgccacccugacccuggcccaaggcacccagagggaucuggugguggaccaggaccacauccugcccguggccgagggaacccugagagucaaagccaagagccucaccaccgagaucgagacaggcaacccuggcagccucaucgccgaggugcaggagaacaucgacaacaagcaggccaucgucgugggcaaagaccugacucuguccagcgcccacggcaacguagccaacgaggccaacgcccugcugugggcugccggugagcugaccgugaaggcccagaacaucaccaacaagagagccgcccugauugaggccggcgggaacgccagacugaccgcugccgucgcccuucugaacaagcugggcagaaucagagccggcgaggacaugcaccuggacgccccuagaaucgagaacaccgccaagcugagcggcgaggugcagagaaagggcgugcaggacgucggcggaggcgagcacggcagguggagcggcaucggcuacgugaacuacuggcuucgggccggcaacggcaagaaagccggcaccaucgccgcaccuugguacgguggcgaucugaccgcagagcagagccugaucgaggugggcaaggaccuguaccugaacgccggcgccagaaaggacgagcacagacaccugcugaacgagggcgugauccaggccggaggucacggccacaucggcggcgacguggacaacagggccgugguccguaccgugagcgccauggaguacuucaagaccccucugccugugagccugaccgcccuggacaauagagccggacucagcccagccaccuggaacuuccagagcaccuacgagcugcuggacuaccugcuggaccagaacagauacgaguacaucuggggccuguacccuaccuacaccgaguggagcgugaacacccugaagaaccuggaccugggcuaccaggccaagccugccccuaccgccccuccuaugccuaaggccccugagcuggaccugagaggccauacccuggagagcgccgagggcagaaagaucuucggcgaguacaagaagcugcagggcgaguacgagaaggccaagauggccgugcaagccguggaggccuacggcgaggccaccagaagagugcacgaccagcucgggcagcgauacggcaaggcccugggcggcauggacgccgaaaccaaggagguggacggcaucauccaggaguuugcggccgaucugagaaccguguacgccaaacaggccgaccaagccacuaucgacgccgagacugacaagguggcccagagauacaagagccagaucgacgccgugagacuccaacgggccaagagaagcggaagcggcgcucccugauaauaggcuggagccucgguggccuagcuucuugccccuugggccuccccccagccccuccuccccuuccugcacccguacccccguggucuuugaauaaagucugagugggcggc

SEQ ID NO: 34 - RNA, 1461 nt, Artificial sequence
  Synthetic
auggagaccccugcccagcugcuguuccugcugcugcuuuggcugccugacaccaccggaagccuguacgccgaacacgacgccacccugacccuggcccaaggcacccagagggaucuggugguggaccaggaccacauccugcccguggccgagggaacccugagagucaaagccaagagccucaccaccgagaucgagacaggcaacccuggcagccucaucgccgaggugcaggagaacaucgacaacaagcaggccaucgucgugggcaaagaccugacucuguccagcgcccacggcaacguagccaacgaggccaacgcccugcugugggcugccggugagcugaccgugaaggcccagaacaucaccaacaagagagccgcccugauugaggccggcgggaacgccagacugaccgcugccgucgcccuucugaacaagcugggcagaaucagagccggcgaggacaugcaccuggacgccccuagaaucgagaacaccgccaagcugagcggcgaggugcagagaaagggcgugcaggacgucggcggaggcgagcacggcagguggagcggcaucggcuacgugaacuacuggcuucgggccggcaacggcaagaaagccggcaccaucgccgcaccuugguacgguggcgaucugaccgcagagcagagccugaucgaggugggcaaggaccuguaccugaacgccggcgccagaaaggacgagcacagacaccugcugaacgagggcgugauccaggccggaggucacggccacaucggcggcgacguggacaacagggccgugguccguaccgugagcgccauggaguacuucaagaccccucugccugugagccugaccgcccuggacaauagagccggacucagcccagccaccuggaacuuccagagcaccuacgagcugcuggacuaccugcuggaccagaacagauacgaguacaucuggggccuguacccuaccuacaccgaguggagcgugaacacccugaagaaccuggaccugggcuaccaggccaagccugccccuaccgccccuccuaugccuaaggccccugagcuggaccugagaggccauacccuggagagcgccgagggcagaaagaucuucggcgaguacaagaagcugcagggcgaguacgagaaggccaagauggccgugcaagccguggaggccuacggcgaggccaccagaagagugcacgaccagcucgggcagcgauacggcaaggcccugggcggcauggacgccgaaaccaaggagguggacggcaucauccaggaguuugcggccgaucugagaaccguguacgccaaacaggccgaccaagccacuaucgacgccgagacugacaagguggcccagagauacaagagccagaucgacgccgugagacuccaacgggccaagagaagcggaagcggcgcuccc

SEQ ID NO: 35 - protein, 477 aa, Artificial sequence
  Synthetic
METPAQLLFLLLLWLPDTTGSLYAEHDATLTLAQGTQRDLVVDQDHILPVAEGTLRVKAKSLTTEIETGNPGSLIAEVQENIDNKQAIVVGKDLTLSSAHGNVANEANALLWAAGELTVKAQNITNKRAALIEAGGNARLTAAVALLNKLGRIRAGEDMHLDAPRIENTAKLSGEVQRKGVQDVGGGEHGRWSGIGYVNYWLRAGNGKKAGTIAAPWYGGDLTAEQSLIEVGKDLYLNAGARKDEHRHLLNEGVIQAGGHGHIGGDVDNRAVVRTVSAMEYFKTPLPVSLTALDNRAGLSPATWNFQSTYELLDYLLDQNRYEYIWGLYPTYTEWSVNTLKNLDLGYQAKPAPTAPPMPKAPELDLRGHTLESAEGRKIFGEYKKLQGEYEKAKMAVQAVEAYGEATRRVHDQLGQRYGKALGGMDAETKEVDGIIQEFAADLRTVYAKQADQATIDAETDKVAQRYKSQIDAVRLQ

SEQ ID NO: 36 - RNA, 2432 nt, Artificial sequence
  Synthetic
gggaaauaagagagaaaagaagaguaagaagaaauauaagaccccggcgccgccaccauggagaccccugcccagcugcuguuccugcugcugcuuuggcugccugacaccaccggaguggauccaccuguggacugcggacgugcauuaggacugcacuucuggucaagugcgagccuuauaucggaucagaccccugacggcacauugaucggcaagccgguggucggacggucacuauugagcaagucuugcaaggugccugacgacauuaaggaagaccugucagacaaucacgacggcgagccaguugauaucguacuggagcuuggcagcaacuauaagauuaggccacagucguacggucauccuggaauaguggucgaccugccuuccgguucaacagaggagacagguaucgcuaucuacaucgacuucggcucuagcccaaugcagaaggucggcgagagacaguggcuguacccacagaagggugaggugcuuuucgacgugcugacuauuaacggagacaacgccgagguucgguaccaggccaucaaggucgguccacugaagcgcccaagaaagcugguccuuagucaguucccaaaccucuucacuuacaagugggucuucaugagaggcacaagccaggagcggguccucgcacagggcaccauagauacugacguggccacgagcaccaucgaccugaagacgugcagauacaccagucagaccgugucgcucccaaucauucagaggagugccuugaccgguguuggcaccacacuuggcaugacugacuuccaaaugccauucugguguuacggcuggccuaaggugucaguuuacaugucugcgacaaagacucaaacaggcguagacggcguagcacucccagcaaccggacaggccgccgguauggccuccggcgugggcguucaguugauuaacgguaagacgcagcagccugucaagcugggacuccagggcaagauugcccugccugaggcacagcagaccgaaagugcaacauucucacugccaaugaaggcccaguacuaccagaccaguaccucuacaucugccggcaagcuguccguuaccuacgccgucacucugaauuacgacggcggaggcuccgacgacggcacaaucgugauaaccgguacaauuacugacacaaccuguguaaucgaggauccuuccggcccaucgcauacuaagguuguucagcugccaaagauuuccaagaacgcccucaaggccaacggagaucaagcugguaggacaccauucauaauuaagcuaaaggacuguccauccucccucgguaacggcgugaaggccuauuucgagccaggaccuacgacugacuacucuacaggugaucucagagcauauaagaugguuuacgcaacaaauccucagacacagcucagcaauaucgucgccgccacugaggcucagggaguccagguccggaucuccaacuuagacgacucaaagaucaccaugggcgccaacgaggcaacucagcaggccgcuggauucgacccagagguacagacuggcgguacgucccgcacagucaccaugcgguaccuggcaaguuacguuaagaagaacggugacguggaggcguccgccauaaccacauacgugggauucucagugguguacccugguggcggcucuggcgguggcaguaacgacgguacuauuguaauuacuggaaguauuucagaccaaacgugcguuaucgaagaaccaagcacauuaaaccauauuaagguggugcaguugccuaagauaucaaagaacgcuuugaggaacgacggcgauacugcaggcgcaacaccuuucgauauaaagcuuaaggagugcccucaggcccugggcgcccugaagcucuacuucgaaccaggcauaacaacuaacuacgacaccggugaucuuauugcuuacaagcagacauauaacgcagccggcaacggccagcuuuccacuguaucauccgccacaaaggcuaagggcgucgaguucaggcuggcgaaucucaacggacaacacauuagaauggguacugauaagaccacccaagcugcacagaccuucacagguaaggucacuaacggcgguaagaguuauacacugcgcuaccuggcuagcuacgugaagaagccaaaggaggacgucgacgcagcacagauaacaucuuacguaggauucucuguuguuuauccggguggcgguagcaacgacggaaccauugugaucacuggcucaauuuccgaccagacagucauuugauaauaggcuggagccucgguggccuagcuucuugccccuugggccuccccccagccccuccuccccuuccugcacccguacccccguggucuuugaauaaagucugagugggcggc

SEQ ID NO: 37 - RNA, 2256 nt, Artificial sequence
  Synthetic
auggagaccccugcccagcugcuguuccugcugcugcuuuggcugccugacaccaccggaguggauccaccuguggacugcggacgugcauuaggacugcacuucuggucaagugcgagccuuauaucggaucagaccccugacggcacauugaucggcaagccgguggucggacggucacuauugagcaagucuugcaaggugccugacgacauuaaggaagaccugucagacaaucacgacggcgagccaguugauaucguacuggagcuuggcagcaacuauaagauuaggccacagucguacggucauccuggaauaguggucgaccugccuuccgguucaacagaggagacagguaucgcuaucuacaucgacuucggcucuagcccaaugcagaaggucggcgagagacaguggcuguacccacagaagggugaggugcuuuucgacgugcugacuauuaacggagacaacgccgagguucgguaccaggccaucaaggucgguccacugaagcgcccaagaaagcugguccuuagucaguucccaaaccucuucacuuacaagugggucuucaugagaggcacaagccaggagcggguccucgcacagggcaccauagauacugacguggccacgagcaccaucgaccugaagacgugcagauacaccagucagaccgugucgcucccaaucauucagaggagugccuugaccgguguuggcaccacacuuggcaugacugacuuccaaaugccauucugguguuacggcuggccuaaggugucaguuuacaugucugcgacaaagacucaaacaggcguagacggcguagcacucccagcaaccggacaggccgccgguauggccuccggcgugggcguucaguugauuaacgguaagacgcagcagccugucaagcugggacuccagggcaagauugcccugccugaggcacagcagaccgaaagugcaacauucucacugccaaugaaggcccaguacuaccagaccaguaccucuacaucugccggcaagcuguccguuaccuacgccgucacucugaauuacgacggcggaggcuccgacgacggcacaaucgugauaaccgguacaauuacugacacaaccuguguaaucgaggauccuuccggcccaucgcauacuaagguuguucagcugccaaagauuuccaagaacgcccucaaggccaacggagaucaagcugguaggacaccauucauaauuaagcuaaaggacuguccauccucccucgguaacggcgugaaggccuauuucgagccaggaccuacgacugacuacucuacaggugaucucagagcauauaagaugguuuacgcaacaaauccucagacacagcucagcaauaucgucgccgccacugaggcucagggaguccagguccggaucuccaacuuagacgacucaaagaucaccaugggcgccaacgaggcaacucagcaggccgcuggauucgacccagagguacagacuggcgguacgucccgcacagucaccaugcgguaccuggcaaguuacguuaagaagaacggugacguggaggcguccgccauaaccacauacgugggauucucagugguguacccugguggcggcucuggcgguggcaguaacgacgguacuauuguaauuacuggaaguauuucagaccaaacgugcguuaucgaagaaccaagcacauuaaaccauauuaagguggugcaguugccuaagauaucaaagaacgcuuugaggaacgacggcgauacugcaggcgcaacaccuuucgauauaaagcuuaaggagugcccucaggcccugggcgcccugaagcucuacuucgaaccaggcauaacaacuaacuacgacaccggugaucuuauugcuuacaagcagacauauaacgcagccggcaacggccagcuuuccacuguaucauccgccacaaaggcuaagggcgucgaguucaggcuggcgaaucucaacggacaacacauuagaauggguacugauaagaccacccaagcugcacagaccuucacagguaaggucacuaacggcgguaagaguuauacacugcgcuaccuggcuagcuacgugaagaagccaaaggaggacgucgacgcagcacagauaacaucuuacguaggauucucuguuguuuauccggguggcgguagcaacgacggaaccauugugaucacuggcucaauuuccgaccagacagucauu

SEQ ID NO: 38 - protein, 752 aa, Artificial sequence
  Synthetic
METPAQLLFLLLLWLPDTTGVDPPVDCGRALGLHFWSSASLISDQTPDGTLIGKPVVGRSLLSKSCKVPDDIKEDLSDNHDGEPVDIVLELGSNYKIRPQSYGHPGIVVDLPSGSTEETGIAIYIDFGSSPMQKVGERQWLYPQKGEVLFDVLTINGDNAEVRYQAIKVGPLKRPRKLVLSQFPNLFTYKWVFMRGTSQERVLAQGTIDTDVATSTIDLKTCRYTSQTVSLPIIQRSALTGVGTTLGMTDFQMPFWCYGWPKVSVYMSATKTQTGVDGVALPATGQAAGMASGVGVQLINGKTQQPVKLGLQGKIALPEAQQTESATFSLPMKAQYYQTSTSTSAGKLSVTYAVTLNYDGGGSDDGTIVITGTITDTTCVIEDPSGPSHTKVVQLPKISKNALKANGDQAGRTPFIIKLKDCPSSLGNGVKAYFEPGPTTDYSTGDLRAYKMVYATNPQTQLSNIVAATEAQGVQVRISNLDDSKITMGANEATQQAAGFDPEVQTGGTSRTVTMRYLASYVKKNGDVEASAITTYVGFSVVYPGGGSGGGSNDGTIVITGSISDQTCVIEEPSTLNHIKVVQLPKISKNALRNDGDTAGATPFDIKLKECPQALGALKLYFEPGITTNYDTGDLIAYKQTYNAAGNGQLSTVSSATKAKGVEFRLANLNGQHIRMGTDKTTQAAQTFTGKVTNGGKSYTLRYLASYVKKPKEDVDAAQITSYVGFSVVYPGGGSNDGTIVITGSISDQTVI

SEQ ID NO: 39 - RNA, 2051 nt, Artificial sequence
  Synthetic
gggaaauaagagagaaaagaagaguaagaagaaauauaagaccccggcgccgccaccauggagaccccugcccagcugcuguuccugcugcugcuuuggcugccagacaccaccggaacccuggagcacgugcagcacaucaucggcggcgccggcaacgacgccaucacuggcaacgcccacgacaacuuccuggccggcggcagcggcgacgacagacuggacggcggagcagguaacgacgugcucgucggaggcgaggggcagaacaccgugauuggcggagccggagacgacguguuccugcaggaccugggcguguggagcaaccaguuagacggcggcgcagguguggauacuguuaaguacaacgugcaccagccuagcgaggagagacuggagagaaugggcgacaccggcauccacgccgaccugcagaagggaaccguggagaaguggccugcccugaaccuguucagcguggaccacgugaagaacaucgagaaccugcacggcaguagacuaaacgacagaauugcaggagacgaucaggacaacgaacuguggggucacgacggaaacgacgugauacguggcagaggaggcgacgacauccugcggggcggccugggccuggacacccuguacggcgaggacggaaacgacaucuuccuccaagacgacgaaaccgugagcgacgauaucgacggcggugccggauuagacaccgucgacuacagcgccaugauccacccuggcagaaucguggccccucacgaguacggcuucggcaucgaggcugaccugagcagagagugggugagaaaggccagcgcguuggguguagacuacuacgacaacgugagaaacgucgagaacgucauuggcaccagcaugaaagacgugcucaucggugacgcucaggccaacacccugaugggccagggcggugacgacacagugcgaggcggagacggagacgaccugcuguucgguggcgacggcaacgauauguuauacggcgacgcuggaaacgacguguuauacgguggacugggugacgauacauuggaggguggcgcgggcaacgauugguucggccagacacaagccagagagcacgacguucuacguggcggcgacggaguggacacuguggacuacucacaaaccggcgcccacgccggaaucgcggccgggagaaucggcuugggaauccuugcugacuugggugcaggccggguggacaagcugggugaggcaggcucaagugccuacgauacagugagcggcauugagaacguagucggcacggagcuugcugaucggauuaccggagacgcccaagcaaacguguuacggggugcuggaggcgcugacguccucgcuggcggugagggcgacgacgugcuguugggaggagacggugacgaccaacugucgggcgacgcgggaagggauagacuguacggagaagcaggagacgacugguucuuccaggacgccgccaacgcgggaaaccugcuugacgguggugacggacgagauaccguagauuucucugguccaggcagaggccucgacgcaggcgccaagggcgucuuccugucccucgguaagggcuucgccagccuuauggacgaaccugagacuucuaacgugcugagaaauauugagaacgccgucggcagcgccagggacgacgucuuaaucggcgacgccggugcgaacguccuaaacggcuuggccggaaacgacguuuuaagcggaggagcuggcgacgacguacugcuuggcgacgagggcagcgaccuccuaucaggugacgccgguaacgacgaccucuucggcggacagggugacgauacuuauuuguucggcgugggcuacggacacgacacucgggccaagagaagcggaagcggcgcucccugauaauaggcuggagccucgguggccuagcuucuugccccuugggccuccccccagccccuccuccccuuccugcacccguacccccguggucuuugaauaaagucugagugggcggc

SEQ ID NO: 40 - RNA, 1875 nt, Artificial sequence
  Synthetic
auggagaccccugcccagcugcuguuccugcugcugcuuuggcugccagacaccaccggaacccuggagcacgugcagcacaucaucggcggcgccggcaacgacgccaucacuggcaacgcccacgacaacuuccuggccggcggcagcggcgacgacagacuggacggcggagcagguaacgacgugcucgucggaggcgaggggcagaacaccgugauuggcggagccggagacgacguguuccugcaggaccugggcguguggagcaaccaguuagacggcggcgcagguguggauacuguuaaguacaacgugcaccagccuagcgaggagagacuggagagaaugggcgacaccggcauccacgccgaccugcagaagggaaccguggagaaguggccugcccugaaccuguucagcguggaccacgugaagaacaucgagaaccugcacggcaguagacuaaacgacagaauugcaggagacgaucaggacaacgaacuguggggucacgacggaaacgacgugauacguggcagaggaggcgacgacauccugcggggcggccugggccuggacacccuguacggcgaggacggaaacgacaucuuccuccaagacgacgaaaccgugagcgacgauaucgacggcggugccggauuagacaccgucgacuacagcgccaugauccacccuggcagaaucguggccccucacgaguacggcuucggcaucgaggcugaccugagcagagagugggugagaaaggccagcgcguuggguguagacuacuacgacaacgugagaaacgucgagaacgucauuggcaccagcaugaaagacgugcucaucggugacgcucaggccaacacccugaugggccagggcggugacgacacagugcgaggcggagacggagacgaccugcuguucgguggcgacggcaacgauauguuauacggcgacgcuggaaacgacguguuauacgguggacugggugacgauacauuggaggguggcgcgggcaacgauugguucggccagacacaagccagagagcacgacguucuacguggcggcgacggaguggacacuguggacuacucacaaaccggcgcccacgccggaaucgcggccgggagaaucggcuugggaauccuugcugacuugggugcaggccggguggacaagcugggugaggcaggcucaagugccuacgauacagugagcggcauugagaacguagucggcacggagcuugcugaucggauuaccggagacgcccaagcaaacguguuacggggugcuggaggcgcugacguccucgcuggcggugagggcgacgacgugcuguugggaggagacggugacgaccaacugucgggcgacgcgggaagggauagacuguacggagaagcaggagacgacugguucuuccaggacgccgccaacgcgggaaaccugcuugacgguggugacggacgagauaccguagauuucucugguccaggcagaggccucgacgcaggcgccaagggcgucuuccugucccucgguaagggcuucgccagccuuauggacgaaccugagacuucuaacgugcugagaaauauugagaacgccgucggcagcgccagggacgacgucuuaaucggcgacgccggugcgaacguccuaaacggcuuggccggaaacgacguuuuaagcggaggagcuggcgacgacguacugcuuggcgacgagggcagcgaccuccuaucaggugacgccgguaacgacgaccucuucggcggacagggugacgauacuuauuuguucggcgugggcuacggacacgacacucgggccaagagaagcggaagcggcgcuccc

SEQ ID NO: 41 - protein, 615 aa, Artificial sequence
  Synthetic
METPAQLLFLLLLWLPDTTGTLEHVQHIIGGAGNDAITGNAHDNFLAGGSGDDRLDGGAGNDVLVGGEGQNTVIGGAGDDVFLQDLGVWSNQLDGGAGVDTVKYNVHQPSEERLERMGDTGIHADLQKGTVEKWPALNLFSVDHVKNIENLHGSRLNDRIAGDDQDNELWGHDGNDVIRGRGGDDILRGGLGLDTLYGEDGNDIFLQDDETVSDDIDGGAGLDTVDYSAMIHPGRIVAPHEYGFGIEADLSREWVRKASALGVDYYDNVRNVENVIGTSMKDVLIGDAQANTLMGQGGDDTVRGGDGDDLLFGGDGNDMLYGDAGNDVLYGGLGDDTLEGGAGNDWFGQTQAREHDVLRGGDGVDTVDYSQTGAHAGIAAGRIGLGILADLGAGRVDKLGEAGSSAYDTVSGIENVVGTELADRITGDAQANVLRGAGGADVLAGGEGDDVLLGGDGDDQLSGDAGRDRLYGEAGDDWFFQDAANAGNLLDGGDGRDTVDFSGPGRGLDAGAKGVFLSLGKGFASLMDEPETSNVLRNIENAVGSARDDVLIGDAGANVLNGLAGNDVLSGGAGDDVLLGDEGSDLLSGDAGNDDLFGGQGDDTYLFGVGYGHDT

SEQ ID NO: 42 - RNA, 2354 nt, Artificial sequence
  Synthetic
gggaaauaagagagaaaagaagaguaagaagaaauauaagaccccggcgccgccaccauggacagcaagggcagcagccagaagggcucaagacugcucuuguugcucguggugagcaaccugcugcugccucaagguguggugggacaggccccucagccuccgguggccggcgccccacacgcccaagacgcagggcaggagggcgaguucgaccacagagacaacacccugaucgccguguucgacgacggcgugggcaucaaccuggacgacgacccugacgagcugggcgaaacagccccuccuacccugaaggacauccacaucagcguggagcacaagaacccuaugagcaagccugccaucggcgugagagugagcggggccgguagggcccugacccucgcuggcagcaccaucgacgcuaccgagggcggcaucccugccguugucagaagaggcggcacccuggaacuggacggggugaccgucgccggcggagagggcauggagccuaugaccgugagcgacgcaggcagccgacucagugugcggggaggugugcugggaggagaggccccgggcgugggguuagugcgagccgcucagggcggacaggcuagcauaaucgacgcaacccugcagagcauccugggacccgcccugauagcggacgguggcucaaucucaguggcuggaggcuccauugacauggacaugggcccaggcuucccuccuccaccaccuccucugccuggagcaccucuggccgcccacccuccgcucgacagaguggccgccgugcacgcuggacaggacggcaaggugacccugagagagguggcgcuccgggcgcacggaccccaggccacaggaguguacgccuacaugccuggcagcgagaucacacugcagggagguacuguguccguccagggagacgacggugccggagucgucgcaggagccggccugcuggacgcccugccuccuggaggaaccgugagacucgacgguaccaccguguccaccgacggcgcuaacaccgacgccgugcuggugagaggggacgccgccagagccgagguggugaacaccgugcugagaaccgccaagagcuuggccgcaggaguuuccgcgcagcacggaggccggguaacccugcggcagaccagaauugaaacggcuggugccggugccgaagguauuucgguacuaggauucgagccucagagcggcagcgguccagcuucgguagacaugcagggugguagcauuaccacgaccggcaacagggccgccgggaucgcgcugacccacggcuccgcgagacuggagggagucgccgugcgagcagaaggauccggauccagcgccgcccagcuggcaaacggaguucuugugguauccgcaggcucacucgccucugcgcagucuggcgcuauaucugugacugacaccccucugaagcugaugccaggagcucucgcuuccagcacuguaagcgucagacuuacugacggagccacugcacaaggagguaacggcguauuccugcagcagcauucaacuauaccuguggcuguggcucuugagagcggagcgcuggcacgcggcgacaucguggccgacgggaacaagccccucgacgccgguaucagcuugucaguggccucuggagccgccuggcacggggcuacccaagugcugcagucugccacgcugggcaaggguggcaccuggguuguuaacgccgacagcagagugcaggacaugagcaugcggggcggaaggguugaguuccaggcuccggccccugaggccagcuacaagaccuuaacacuccagaccuuagacggaaacggcguguucgugcugaacaccaacgucgcagcgggacagaacgaccagcugagaguuaccggcagggcugacggucagcacagaguccuggucaggaacgccggaggcgaggccgauagccggggugcgcgguuaggacugguucacacccagggccagggcaacgcaguauucagauuagcaaacguggguaaggccguggaccugggcacguggagauacaguuuggccgaggacccuaagacccacguguggagucugcaaagagccggucaggcucugucaggcgcugcuaacgccgccguuaacgcugcggaccugaguaguaucgcccuagcagaaagcaacgcccaccaucaccaucaucacugauaauaggcuggagccucgguggccuagcuucuugccccuugggccuccccccagccccuccuccccuuccugcacccguacccccguggucuuugaauaaagucugagugggcggc

SEQ ID NO: 43 - RNA, 2178 nt, Artificial sequence
  Synthetic
auggacagcaagggcagcagccagaagggcucaagacugcucuuguugcucguggugagcaaccugcugcugccucaagguguggugggacaggccccucagccuccgguggccggcgccccacacgcccaagacgcagggcaggagggcgaguucgaccacagagacaacacccugaucgccguguucgacgacggcgugggcaucaaccuggacgacgacccugacgagcugggcgaaacagccccuccuacccugaaggacauccacaucagcguggagcacaagaacccuaugagcaagccugccaucggcgugagagugagcggggccgguagggcccugacccucgcuggcagcaccaucgacgcuaccgagggcggcaucccugccguugucagaagaggcggcacccuggaacuggacggggugaccgucgccggcggagagggcauggagccuaugaccgugagcgacgcaggcagccgacucagugugcggggaggugugcugggaggagaggccccgggcgugggguuagugcgagccgcucagggcggacaggcuagcauaaucgacgcaacccugcagagcauccugggacccgcccugauagcggacgguggcucaaucucaguggcuggaggcuccauugacauggacaugggcccaggcuucccuccuccaccaccuccucugccuggagcaccucuggccgcccacccuccgcucgacagaguggccgccgugcacgcuggacaggacggcaaggugacccugagagagguggcgcuccgggcgcacggaccccaggccacaggaguguacgccuacaugccuggcagcgagaucacacugcagggagguacuguguccguccagggagacgacggugccggagucgucgcaggagccggccugcuggacgcccugccuccuggaggaaccgugagacucgacgguaccaccguguccaccgacggcgcuaacaccgacgccgugcuggugagaggggacgccgccagagccgagguggugaacaccgugcugagaaccgccaagagcuuggccgcaggaguuuccgcgcagcacggaggccggguaacccugcggcagaccagaauugaaacggcuggugccggugccgaagguauuucgguacuaggauucgagccucagagcggcagcgguccagcuucgguagacaugcagggugguagcauuaccacgaccggcaacagggccgccgggaucgcgcugacccacggcuccgcgagacuggagggagucgccgugcgagcagaaggauccggauccagcgccgcccagcuggcaaacggaguucuugugguauccgcaggcucacucgccucugcgcagucuggcgcuauaucugugacugacaccccucugaagcugaugccaggagcucucgcuuccagcacuguaagcgucagacuuacugacggagccacugcacaaggagguaacggcguauuccugcagcagcauucaacuauaccuguggcuguggcucuugagagcggagcgcuggcacgcggcgacaucguggccgacgggaacaagccccucgacgccgguaucagcuugucaguggccucuggagccgccuggcacggggcuacccaagugcugcagucugccacgcugggcaaggguggcaccuggguuguuaacgccgacagcagagugcaggacaugagcaugcggggcggaaggguugaguuccaggcuccggccccugaggccagcuacaagaccuuaacacuccagaccuuagacggaaacggcguguucgugcugaacaccaacgucgcagcgggacagaacgaccagcugagaguuaccggcagggcugacggucagcacagaguccuggucaggaacgccggaggcgaggccgauagccggggugcgcgguuaggacugguucacacccagggccagggcaacgcaguauucagauuagcaaacguggguaaggccguggaccugggcacguggagauacaguuuggccgaggacccuaagacccacguguggagucugcaaagagccggucaggcucugucaggcgcugcuaacgccgccguuaacgcugcggaccugaguaguaucgcccuagcagaaagcaacgcccaccaucaccaucaucac

SEQ ID NO: 44 - protein, 720 aa, Artificial sequence
  Synthetic
MDSKGSSQKGSRLLLLLVVSNLLLPQGVVGQAPQPPVAGAPHAQDAGQEGEFDHRDNTLIAVFDDGVGINLDDDPDELGETAPPTLKDIHISVEHKNPMSKPAIGVRVSGAGRALTLAGSTIDATEGGIPAVVRRGGTLELDGVTVAGGEGMEPMTVSDAGSRLSVRGGVLGGEAPGVGLVRAAQGGQASIIDATLQSILGPALIADGGSISVAGGSIDMDMGPGFPPPPPPLPGAPLAAHPPLDRVAAVHAGQDGKVTLREVALRAHGPQATGVYAYMPGSEITLQGGTVSVQGDDGAGVVAGAGLLDALPPGGTVRLDGTTVSTDGANTDAVLVRGDAARAEVVNTVLRTAKSLAAGVSAQHGGRVTLRQTRIETAGAGAEGISVLGFEPQSGSGPASVDMQGGSITTTGNRAAGIALTHGSARLEGVAVRAEGSGSSAAQLANGVLVVSAGSLASAQSGAISVTDTPLKLMPGALASSTVSVRLTDGATAQGGNGVFLQQHSTIPVAVALESGALARGDIVADGNKPLDAGISLSVASGAAWHGATQVLQSATLGKGGTWVVNADSRVQDMSMRGGRVEFQAPAPEASYKTLTLQTLDGNGVFVLNTNVAAGQNDQLRVTGRADGQHRVLVRNAGGEADSRGARLGLVHTQGQGNAVFRLANVGKAVDLGTWRYSLAEDPKTHVWSLQRAGQALSGAANAAVNAADLSSIALAESNA

SEQ ID NO: 45 - RNA, 2030 nt, Artificial sequence
  Synthetic
gggaaauaagagagaaaagaagaguaagaagaaauauaagaccccggcgccgccaccauggagacuccagcccaguuguuauuucuuuuguugcuuuggcucccagacaccaccgggcagagaaucgacggcggugccgccuucuugggcgacguggccaucgccaccaccaaggccagcgagcacgguaucaacgugaccggcagaaccgcugaggugagagucacgggaggcaccauccgcacuagcggaaaccaggcccagggccugagaguaggcaccgagaacgccccugacaacaccgcccugggcgcuagcguguuccugcagaacuugauuaucgagacgagcggcaccggugcucuaggcgugagcguucacgaaccacagggcggcggcgguacgagacugagcauguccggcaccacggugagaaccagaggggaugacaguuucgcccugcagcucagcggcccugccagcgccaccuugaacgacgucgcccuggagacugccggccagcaggccccugccgucgugcucuggcagggcgcacaguuaaacgcucagggacugguggugcaggugaacggagccggugucucagcgauccaugcgcaagacgccggcagcuucacacugagcggcagcgacaucaccgcgcgaggccuggagguggugggcaucuacgugcaggagggcaugcagggcacccucacuggcacacgggugacaacccagggcgacacugcaccugcacugcagguggaggaugcuggcacccacgugagcaugaauggcggcgcuuuguccaccucaggcgccaacagcccugccgccuggcugcuggcgggcggaucugcccaguucagagacaccguccugagaaccguaggcgaagcuagccacggcguggacguggcagcccacagugagguggagcucgcucaugcccagguaagggcagacggccagggagcccacgggcuggugguuaccagaagcucugcuauggugcgagcuggaucucugguugagaguacuggagauggcgcagccgcccugcuggagagcggccaucugacaguggauggaucagugguccacggccauggagcggcugguuuggagguugacggggagagcaacguuagucuccugaacggcgcucgauugagcagcgaccagccuaccgccaucagacugaucgacccuagaucagugcugaaccuggacaucaaggacagagcccaacugcugggcgacaucgcaccugaggcccagcagccugacggcuccccugagcaggcgagagugagaguggcccuggcugacgguggaacgugggccggccggacggacggcgccgugcacaccgugagacugcuggacagaggaguguggaccguaaccggcgacagccgaguggccgaggugaaacuggagggcggcacuuuggccuucgccccuccugcccagccuaagggcgccuucaagacccugguggccacccagggaauuuccggcacgggaaccauuguuaugaaugcccaccugccuaguggcacagccgacgugcuuguggccccucagggcuucggagauagacagguauugguggugaauaacaccgacgacggcacagaguccggcgcuaccaaggugccucugaucgaggacgagcagggccacaccgccuuuacccugggaaacaugggcggcagaguagaugccggagccagacaguacgaacugaccgccagugaggcacaggccgacaaagcccggaccuggcagcugaccccuaccaacgagcugagcaccaccgccaccgccgccguaaaugccauggcuauugcugcuagccaaagaauuuggcaggcggaaauggaucaucaccaucaucaucauugauaauaggcuggagccucgguggccuagcuucuugccccuugggccuccccccagccccuccuccccuuccugcacccguacccccguggucuuugaauaaagucugagugggcggc

SEQ ID NO: 46 - RNA, 1854 nt, Artificial sequence
  Synthetic
auggagacuccagcccaguuguuauuucuuuuguugcuuuggcucccagacaccaccgggcagagaaucgacggcggugccgccuucuugggcgacguggccaucgccaccaccaaggccagcgagcacgguaucaacgugaccggcagaaccgcugaggugagagucacgggaggcaccauccgcacuagcggaaaccaggcccagggccugagaguaggcaccgagaacgccccugacaacaccgcccugggcgcuagcguguuccugcagaacuugauuaucgagacgagcggcaccggugcucuaggcgugagcguucacgaaccacagggcggcggcgguacgagacugagcauguccggcaccacggugagaaccagaggggaugacaguuucgcccugcagcucagcggcccugccagcgccaccuugaacgacgucgcccuggagacugccggccagcaggccccugccgucgugcucuggcagggcgcacaguuaaacgcucagggacugguggugcaggugaacggagccggugucucagcgauccaugcgcaagacgccggcagcuucacacugagcggcagcgacaucaccgcgcgaggccuggagguggugggcaucuacgugcaggagggcaugcagggcacccucacuggcacacgggugacaacccagggcgacacugcaccugcacugcagguggaggaugcuggcacccacgugagcaugaauggcggcgcuuuguccaccucaggcgccaacagcccugccgccuggcugcuggcgggcggaucugcccaguucagagacaccguccugagaaccguaggcgaagcuagccacggcguggacguggcagcccacagugagguggagcucgcucaugcccagguaagggcagacggccagggagcccacgggcuggugguuaccagaagcucugcuauggugcgagcuggaucucugguugagaguacuggagauggcgcagccgcccugcuggagagcggccaucugacaguggauggaucagugguccacggccauggagcggcugguuuggagguugacggggagagcaacguuagucuccugaacggcgcucgauugagcagcgaccagccuaccgccaucagacugaucgacccuagaucagugcugaaccuggacaucaaggacagagcccaacugcugggcgacaucgcaccugaggcccagcagccugacggcuccccugagcaggcgagagugagaguggcccuggcugacgguggaacgugggccggccggacggacggcgccgugcacaccgugagacugcuggacagaggaguguggaccguaaccggcgacagccgaguggccgaggugaaacuggagggcggcacuuuggccuucgccccuccugcccagccuaagggcgccuucaagacccugguggccacccagggaauuuccggcacgggaaccauuguuaugaaugcccaccugccuaguggcacagccgacgugcuuguggccccucagggcuucggagauagacagguauugguggugaauaacaccgacgacggcacagaguccggcgcuaccaaggugccucugaucgaggacgagcagggccacaccgccuuuacccugggaaacaugggcggcagaguagaugccggagccagacaguacgaacugaccgccagugaggcacaggccgacaaagcccggaccuggcagcugaccccuaccaacgagcugagcaccaccgccaccgccgccguaaaugccauggcuauugcugcuagccaaagaauuuggcaggcggaaauggaucaucaccaucaucaucau

SEQ ID NO: 47 - protein, 618 aa, Artificial sequence
  Synthetic
METPAQLLFLLLLWLPDTTGQRIDGGAAFLGDVAIATTKASEHGINVTGRTAEVRVTGGTIRTSGNQAQGLRVGTENAPDNTALGASVFLQNLIIETSGTGALGVSVHEPQGGGGTRLSMSGTTVRTRGDDSFALQLSGPASATLNDVALETAGQQAPAVVLWQGAQLNAQGLVVQVNGAGVSAIHAQDAGSFTLSGSDITARGLEVVGIYVQEGMQGTLTGTRVTTQGDTAPALQVEDAGTHVSMNGGALSTSGANSPAAWLLAGGSAQFRDTVLRTVGEASHGVDVAAHSEVELAHAQVRADGQGAHGLVVTRSSAMVRAGSLVESTGDGAAALLESGHLTVDGSVVHGHGAAGLEVDGESNVSLLNGARLSSDQPTAIRLIDPRSVLNLDIKDRAQLLGDIAPEAQQPDGSPEQARVRVALADGGTWAGRTDGAVHTVRLLDRGVWTVTGDSRVAEVKLEGGTLAFAPPAQPKGAFKTLVATQGISGTGTIVMNAHLPSGTADVLVAPQGFGDRQVLVVNNTDDGTESGATKVPLIEDEQGHTAFTLGNMGGRVDAGARQYELTASEAQADKARTWQLTPTNELSTTATAAVNAMAIAASQRIWQAEMDHHHHHH

SEQ ID NO: 48 - RNA, 1856 nt, Artificial sequence
  Synthetic
gggaaauaagagagaaaagaagaguaagaagaaauauaagaccccggcgccgccaccaugagccggaagcuguucgccagcauccugaucggcgcccugcugggcauuggugccccaccaagcgcccacgcuggagcugacgacgugguggacagcagcaagagcuucgugauggagaacuucgccagcuaccacggcaccaagcccggcuacguggacaguauccagaagggcauccagaagcccaagagcggcacccagggcaacuacgacgacgacugggagggcuucuacagcaccgacaacaaguacgacgccgccggcuacagcgucgacaacgagaacccacuguccggcaaagccggaggcguggugaaggugaccuaccccggccugaccaaggugcuggcccugaagguggacaacgccgagaccauuaagaaggagcugggccugagccugaccgagccccugauggagcaggugggcacugaggaguucaucaagcguuucggggacggggcgaguagaguggugcugagccugcccuucgccgagggcagcagcagcgugaaguacaucaacaacugggagcaggccaaggcccugagcguggagcuggagaucaacuucgagacccggggcaaacgaggccaggacgccauguacgaguacauggcacaggcuugcgccggcaaucgggugcggagaagcgugggcucuagccugagcugcaucaaucuggacugggacgugauccgggacaagacgaagaccaagaucgagagccugaaggagcacggccccaucaagaacaagaugagcgagagccccaacaaggccgugagcgaggagaaggccaagcaguaccuggaggaguuccaccagaccgccuuggaacaccccgagcugagcgagcugaagacugugaccggcaccaaccccguuuucgccggcgccaacuacgccgccugggccgugaacguggcucaggugaucgacagcgagaccgccgacaaccuggagaagaccaccgccgcccugagcauccugcccggcaucggcagcgugaugggcaucgcugacggcgccgugcaccacaacaccgaggagaucguggcccagagcaucgcccucagcagccugaugguggcccaggccauuccccuggugggcgagcugguggacaucggcuucgccgccuacaacuucguggagagcaucaucaaccuguuccagguggugcacaacagcuacaaccggcccgccuauagccccggacacaagacccagcccuuucugcacgacggcuacgccgugagcuggaacaccguggaggacagcaucauccggaccggcuuccagggcgagagcggccacgacaucaagaucaccgccgagaacaccccucugcccaucgccggcguucugcugcccaccauccccggcaagcuggacgugaacaaggccaagacccacaucagcgugaacggccggaagauccgaaugcggugccgggcaaucgacggcgacgugaccuucugccggccuaagagccccguguacgugggcaacggcguucacgccaaccugcacguggccuuccaccggagcagcucugagaagauccacagcaacgagaucagcagcgacagcaucggcgugcugggcuaccagaagaccguggaccacaccaaggugaacagcaaacugagccuguucuucgagaucaagagcugauaauaggcuggagccucgguggccuagcuucuugccccuugggccuccccccagccccuccuccccuuccugcacccguacccccguggucuuugaauaaagucugagugggcggc

SEQ ID NO: 49 - RNA, 1680 nt, Artificial sequence
  Synthetic
augagccggaagcuguucgccagcauccugaucggcgcccugcugggcauuggugccccaccaagcgcccacgcuggagcugacgacgugguggacagcagcaagagcuucgugauggagaacuucgccagcuaccacggcaccaagcccggcuacguggacaguauccagaagggcauccagaagcccaagagcggcacccagggcaacuacgacgacgacugggagggcuucuacagcaccgacaacaaguacgacgccgccggcuacagcgucgacaacgagaacccacuguccggcaaagccggaggcguggugaaggugaccuaccccggccugaccaaggugcuggcccugaagguggacaacgccgagaccauuaagaaggagcugggccugagccugaccgagccccugauggagcaggugggcacugaggaguucaucaagcguuucggggacggggcgaguagaguggugcugagccugcccuucgccgagggcagcagcagcgugaaguacaucaacaacugggagcaggccaaggcccugagcguggagcuggagaucaacuucgagacccggggcaaacgaggccaggacgccauguacgaguacauggcacaggcuugcgccggcaaucgggugcggagaagcgugggcucuagccugagcugcaucaaucuggacugggacgugauccgggacaagacgaagaccaagaucgagagccugaaggagcacggccccaucaagaacaagaugagcgagagccccaacaaggccgugagcgaggagaaggccaagcaguaccuggaggaguuccaccagaccgccuuggaacaccccgagcugagcgagcugaagacugugaccggcaccaaccccguuuucgccggcgccaacuacgccgccugggccgugaacguggcucaggugaucgacagcgagaccgccgacaaccuggagaagaccaccgccgcccugagcauccugcccggcaucggcagcgugaugggcaucgcugacggcgccgugcaccacaacaccgaggagaucguggcccagagcaucgcccucagcagccugaugguggcccaggccauuccccuggugggcgagcugguggacaucggcuucgccgccuacaacuucguggagagcaucaucaaccuguuccagguggugcacaacagcuacaaccggcccgccuauagccccggacacaagacccagcccuuucugcacgacggcuacgccgugagcuggaacaccguggaggacagcaucauccggaccggcuuccagggcgagagcggccacgacaucaagaucaccgccgagaacaccccucugcccaucgccggcguucugcugcccaccauccccggcaagcuggacgugaacaaggccaagacccacaucagcgugaacggccggaagauccgaaugcggugccgggcaaucgacggcgacgugaccuucugccggccuaagagccccguguacgugggcaacggcguucacgccaaccugcacguggccuuccaccggagcagcucugagaagauccacagcaacgagaucagcagcgacagcaucggcgugcugggcuaccagaagaccguggaccacaccaaggugaacagcaaacugagccuguucuucgagaucaagagc

SEQ ID NO: 50 - protein, 560 aa, Unknown
  Diptheria toxin
MSRKLFASILIGALLGIGAPPSAHAGADDVVDSSKSFVMENFASYHGTKPGYVDSIQKGIQKPKSGTQGNYDDDWEGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVKYINNWEQAKALSVELEINFETRGKRGQDAMYEYMAQACAGNRVRRSVGSSLSCINLDWDVIRDKTKTKIESLKEHGPIKNKMSESPNKAVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKAKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 51 - RNA, 1589 nt, Artificial sequence
  Synthetic
gggaaauaagagagaaaagaagaguaagaagaaauauaagaccccggcgccgccaccauggagacacccgcccagcugcuguuccugcugcugcuguggcugcccgacaccaccggcaagaaccuggacugcuggguggacaacgaggaggacaucgacgugauccugaagaagagcaccauccugaaccuggacaucaacaacgacaucaucagcgacaucagcggcuucaacagcgccgugaucaccuaccccgacgcccaacuggugcccggcaucaacggcaaggccauccaccuggugaacaacgaggccagcgaggugaucgugcacaaggccauggacaucgaguacaacgacauguucaacaacuucgccgugagcuucuggcugcgggugcccaaggugagcgccagccaccuggagcaguacggcaccaacgaguacagcaucaucagcagcaugaagaagcacagccugagcaucggcagcggcuggagcgugagccugaagggcaacaaccugaucuggacccugaaggauagcgccggcgaggugagacagaucaccuuccgggaccugcccgacaaguucaacgccuaccuggccaacaaguggguguucauuaccaucaccaacgaccggcugagcagcgccaaccuguacaucaacggcgugcugaugggcagcgccgagaucaccggccugggagccauccgggaggacaacaacaucgcccugaagcuggaccggugcaacaacaauaaccaguacgugagcaucgacaaguuccggaucuucugcaaggcccugaaccccaaggagaucgagaagcuguacaccagcuaccugagcaucaccuuccugcgggacuucuggggcaauccccugcgguacgacaccgaguacuaccugauccccguggccucaagcagcaaggacgugcaguugaagaacaucgccgacuacauguaccugaccaacgccccgagcuacaccaacggcaagcugaacaucuacuaccggcggcuguacaacggccugaaguucaucaucaagcgguacaccccuaacaacgagaucgacagcuucgugaagagcggcgacuucaucaagcuguacgugagcuacaacaacaacgagcacaucgugggcuaccccaaggacggcaacgccuucaacaaccuggaccggauccugcgggugggcuacaacgccccaggcauuccccuguacaagaagauggaggccgugaagcugcgggaccugaagaccuacagcgugcagcugaaacuguacgacgacaagaacgccagccugggccuggugggcacccacaacggccagaucggcaacgaccccaaccgggacauccugaucgccagcaacugguacuuuaaccaccugaaggacaagauccugggcugcgacugguacuucgugcccaccgacgagggcuggaccaacgacugauaauaggcuggagccucgguggccuagcuucuugccccuugggccuccccccagccccuccuccccuuccugcacccguacccccguggucuuugaauaaagucugagugggcggc

SEQ ID NO: 52 - RNA, 1413 nt, Artificial sequence
  Synthetic
auggagacacccgcccagcugcuguuccugcugcugcuguggcugcccgacaccaccggcaagaaccuggacugcuggguggacaacgaggaggacaucgacgugauccugaagaagagcaccauccugaaccuggacaucaacaacgacaucaucagcgacaucagcggcuucaacagcgccgugaucaccuaccccgacgcccaacuggugcccggcaucaacggcaaggccauccaccuggugaacaacgaggccagcgaggugaucgugcacaaggccauggacaucgaguacaacgacauguucaacaacuucgccgugagcuucuggcugcgggugcccaaggugagcgccagccaccuggagcaguacggcaccaacgaguacagcaucaucagcagcaugaagaagcacagccugagcaucggcagcggcuggagcgugagccugaagggcaacaaccugaucuggacccugaaggauagcgccggcgaggugagacagaucaccuuccgggaccugcccgacaaguucaacgccuaccuggccaacaaguggguguucauuaccaucaccaacgaccggcugagcagcgccaaccuguacaucaacggcgugcugaugggcagcgccgagaucaccggccugggagccauccgggaggacaacaacaucgcccugaagcuggaccggugcaacaacaauaaccaguacgugagcaucgacaaguuccggaucuucugcaaggcccugaaccccaaggagaucgagaagcuguacaccagcuaccugagcaucaccuuccugcgggacuucuggggcaauccccugcgguacgacaccgaguacuaccugauccccguggccucaagcagcaaggacgugcaguugaagaacaucgccgacuacauguaccugaccaacgccccgagcuacaccaacggcaagcugaacaucuacuaccggcggcuguacaacggccugaaguucaucaucaagcgguacaccccuaacaacgagaucgacagcuucgugaagagcggcgacuucaucaagcuguacgugagcuacaacaacaacgagcacaucgugggcuaccccaaggacggcaacgccuucaacaaccuggaccggauccugcgggugggcuacaacgccccaggcauuccccuguacaagaagauggaggccgugaagcugcgggaccugaagaccuacagcgugcagcugaaacuguacgacgacaagaacgccagccugggccuggugggcacccacaacggccagaucggcaacgaccccaaccgggacauccugaucgccagcaacugguacuuuaaccaccugaaggacaagauccugggcugcgacugguacuucgugcccaccgacgagggcuggaccaacgac

SEQ ID NO: 53 - protein, 471 aa, Unknown
  Tetanus toxin
METPAQLLFLLLLWLPDTTGKNLDCWVDNEEDIDVILKKSTILNLDINNDIISDISGFNSAVITYPDAQLVPGINGKAIHLVNNEASEVIVHKAMDIEYNDMFNNFAVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNIALKLDRCNNNNQYVSIDKFRIFCKALNPKEIEKLYTSYLSITFLRDFWGNPLRYDTEYYLIPVASSSKDVQLKNIADYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 54 - RNA, 4253 nt, Artificial sequence
  Synthetic
gggaaauaagagagaaaagaagaguaagaagaaauauaagaccccggcgccgccaccauggagaccccugcccagcugcuguuccugcugcugcuuuggcugccagacaccaccggagacuggaacaaccaggccaucgugaagaccggcgagagacagcacggcauccacauccaaggcagcgaccccggcggcgugagaaccgccuccggcaccacaaucaaggugagcggcagacaggcccagggcauccugcuggagaacccugccgccgagcugcaguucagaaacggcgccgugaccagcagcggccagcuguccgacgacggcaucagaagauuccugggcaccgugaccgugaaggccggcaagcugguggccgaccacgccacccuggccaacgugggugauaccugggacgacgacgguauugcccuguacguggccggcgagcaagcacaggccagcaucgccgacagcacccugcaaggcgcaggcggcgugcagaucgagagaggcgccaacgugguggugcagagaagcgccauuguugacggcggccugcacaucggcgcccugcagagccugcagccugaggaccugccuccuagcagaguggugcugagagacacaaacgucgucgccgugccagcguccggagcuccagcagccgugagcgugcugggcgccagcgagcugacccucgacggaggacacaucaccgguggcagggccgccgguguggccgccaugcagggagccguugugcaucuacaaagagcuaccauccgacggggcgacgcuccggcugguggagcggucccuggcggugccguccccggaggagcagugccaggcgguuucggcccggguggauucggcccugugcuagacggcugguacggcguggacgugaguggcuccagcguggagcuggcccagaguauuguugaggccccugagcucggcgcugccaucagagugggccguggugccagagugaccguuagcgguggcucacuuagcgccccucacggcaacgugaucgaaacuggaggagcgagacgauucgcaccucaggccgccccucugagcauaacgcugcaagcgggcgcccacgcgcaaggcaaggcccugcuguacagagugcugccugagccugugaaguuaacucugacgggaggugccgacgcacaaggugacaucguggccacggaacuuccuagcaucccuggcacaucuaucggcccucuggacguugcguuagcuucgcaggcccguuggaccggcgccaccagagccguggacucccuaagcaucgacaacgccgugugggucaugaccgacaacucaaacgugggcgcgcugagauuggccucagacggcagugucgauuuccaacaaccugccgaagcaggaagauucaaggugcugacugucaauacacuggccggaagcggccuguucagaaugaacguguucgccgaccugggacucucagauaagcuggucgugaugcaggacgcaaguggccagcacagacugugggucagaaacucgggcuccgagccagccagugccaauacuuugcuguuggugcaaaccccucugggcagugccgccaccuucacucucgccaacaaggacggcaagguggacaucggcaccuacagauacaggcuggcagcaaacggaaacggccaguggagccuagucggagcaaaggccccuccugccccuaagccugcuccacagccuggcccacagccuccgcaaccuccgcagccucagccagaggccccagccccacaaccuccugcaggcagagagcugagugccgcugcuaacgccgccguuaauaccggaggugucggccucgcgucuaccuugugguacgccgagagcaaccgggccaagagaagcggaagcggcgcucccgugaagcagacccugaacuucgaccugcugaagcuggccggcgacguggagagcaaccccggccccauggagacucccgcccagcuccuguuucugcuguugcuguggcugcccgacacaaccggccaggccccucagccuccgguggccggcgccccacacgcccaagacgcagggcaggagggcgaguucgaccacagagacaacacccugaucgccguguucgacgacggcgugggcaucaaccuggacgacgacccugacgagcugggcgaaacagccccuccuacccugaaggacauccacaucagcguggagcacaagaacccuaugagcaagccugccaucggcgugagagugagcggggccgguagggcccugacccucgcuggcagcaccaucgacgcuaccgagggcggcaucccugccguugucagaagaggcggcacccuggaacuggacggggugaccgucgccggcggagagggcauggagccuaugaccgugagcgacgcaggcagccgacucagugugcggggaggugugcugggaggagaggccccgggcgugggguuagugcgagccgcucagggcggacaggcuagcauaaucgacgcaacccugcagagcauccugggacccgcccugauagcggacgguggcucaaucucaguggcuggaggcuccauugacauggacaugggcccaggcuucccuccuccaccaccuccucugccuggagcaccucuggccgcccacccuccgcucgacagaguggccgccgugcacgcuggacaggacgguaaggugacccugagagagguggcgcuccgggcgcacggaccccaggccacaggaguguacgccuacaugccuggcagcgagaucacacugcagggagguacuguguccguccagggagacgacggugccggagucgucgcaggagccggccugcuggacgcccugccuccuggaggaaccgugagacucgacgguaccaccguguccaccgacggcgcuaacaccgacgccgugcuggugagaggggacgccgccagagccgagguggugaacaccgugcugagaaccgccaagagcuuggccgcaggaguuuccgcgcagcacggaggccggguaacccugcggcagaccagaauugaaacggcuggugccggugccgaagguauuucgguacuaggauucgagccucagagcggcagcgguccagcuucgguagacaugcagggugguagcauuaccacgaccggcaacagggccgccgggaucgcgcugacccacggcuccgcgagacuggagggagucgccgugcgagcagaaggauccggauccagcgccgcccagcuggcaaacggaguucuugugguauccgcaggcucacucgccucugcgcagucuggcgcuauaucugugacugacaccccucugaagcugaugccaggagcucucgcuuccagcacuguaagcgucagacuuacugacggagccacugcacaaggagguaacggcguauuccugcagcagcauucaacuauaccuguggcuguggcucuugagagcggagcgcuggcacgcggcgacaucguggccgacgggaacaagccccucgacgccgguaucagcuugucaguggccucuggagccgccuggcacggggcuacccaagugcugcagucugccacgcugggcaaggguggcaccuggguuguuaacgccgacagcagagugcaggacaugagcaugcggggcggaaggguugaguuccaggcuccggccccugaggccagcuacaagaccuuaacacuccagaccuuagacggaaacggcguguucgugcugaacaccaacgucgcagcgggacagaacgaccagcugagaguuaccggcagggcugacggucagcacagaguccuggucaggaacgccggaggcgaggccgauagccggggugcgcgguuaggacugguucacacccagggccagggcaacgcaguauucagauuagcaaacguggguaaggccguggaccugggcacguggagauacaguuuggccgaggacccuaagacccacguguggagucugcaaagagccggucaggcucugucaggcgcugccaacgccgccgugaacgcugcggaccugaguaguaucgcccuagcagaaagcaacgccugauaauaggcuggagccucgguggccuagcuucuugccccuugggccuccccccagccccuccuccccuuccugcacccguacccccguggucuuugaauaaagucugagugggcggc

SEQ ID NO: 55 - RNA, 4077 nt, Artificial sequence
  Synthetic
auggagaccccugcccagcugcuguuccugcugcugcuuuggcugccagacaccaccggagacuggaacaaccaggccaucgugaagaccggcgagagacagcacggcauccacauccaaggcagcgaccccggcggcgugagaaccgccuccggcaccacaaucaaggugagcggcagacaggcccagggcauccugcuggagaacccugccgccgagcugcaguucagaaacggcgccgugaccagcagcggccagcuguccgacgacggcaucagaagauuccugggcaccgugaccgugaaggccggcaagcugguggccgaccacgccacccuggccaacgugggugauaccugggacgacgacgguauugcccuguacguggccggcgagcaagcacaggccagcaucgccgacagcacccugcaaggcgcaggcggcgugcagaucgagagaggcgccaacgugguggugcagagaagcgccauuguugacggcggccugcacaucggcgcccugcagagccugcagccugaggaccugccuccuagcagaguggugcugagagacacaaacgucgucgccgugccagcguccggagcuccagcagccgugagcgugcugggcgccagcgagcugacccucgacggaggacacaucaccgguggcagggccgccgguguggccgccaugcagggagccguugugcaucuacaaagagcuaccauccgacggggcgacgcuccggcugguggagcggucccuggcggugccguccccggaggagcagugccaggcgguuucggcccggguggauucggcccugugcuagacggcugguacggcguggacgugaguggcuccagcguggagcuggcccagaguauuguugaggccccugagcucggcgcugccaucagagugggccguggugccagagugaccguuagcgguggcucacuuagcgccccucacggcaacgugaucgaaacuggaggagcgagacgauucgcaccucaggccgccccucugagcauaacgcugcaagcgggcgcccacgcgcaaggcaaggcccugcuguacagagugcugccugagccugugaaguuaacucugacgggaggugccgacgcacaaggugacaucguggccacggaacuuccuagcaucccuggcacaucuaucggcccucuggacguugcguuagcuucgcaggcccguuggaccggcgccaccagagccguggacucccuaagcaucgacaacgccgugugggucaugaccgacaacucaaacgugggcgcgcugagauuggccucagacggcagugucgauuuccaacaaccugccgaagcaggaagauucaaggugcugacugucaauacacuggccggaagcggccuguucagaaugaacguguucgccgaccugggacucucagauaagcuggucgugaugcaggacgcaaguggccagcacagacugugggucagaaacucgggcuccgagccagccagugccaauacuuugcuguuggugcaaaccccucugggcagugccgccaccuucacucucgccaacaaggacggcaagguggacaucggcaccuacagauacaggcuggcagcaaacggaaacggccaguggagccuagucggagcaaaggccccuccugccccuaagccugcuccacagccuggcccacagccuccgcaaccuccgcagccucagccagaggccccagccccacaaccuccugcaggcagagagcugagugccgcugcuaacgccgccguuaauaccggaggugucggccucgcgucuaccuugugguacgccgagagcaaccgggccaagagaagcggaagcggcgcucccgugaagcagacccugaacuucgaccugcugaagcuggccggcgacguggagagcaaccccggccccauggagacucccgcccagcuccuguuucugcuguugcuguggcugcccgacacaaccggccaggccccucagccuccgguggccggcgccccacacgcccaagacgcagggcaggagggcgaguucgaccacagagacaacacccugaucgccguguucgacgacggcgugggcaucaaccuggacgacgacccugacgagcugggcgaaacagccccuccuacccugaaggacauccacaucagcguggagcacaagaacccuaugagcaagccugccaucggcgugagagugagcggggccgguagggcccugacccucgcuggcagcaccaucgacgcuaccgagggcggcaucccugccguugucagaagaggcggcacccuggaacuggacggggugaccgucgccggcggagagggcauggagccuaugaccgugagcgacgcaggcagccgacucagugugcggggaggugugcugggaggagaggccccgggcgugggguuagugcgagccgcucagggcggacaggcuagcauaaucgacgcaacccugcagagcauccugggacccgcccugauagcggacgguggcucaaucucaguggcuggaggcuccauugacauggacaugggcccaggcuucccuccuccaccaccuccucugccuggagcaccucuggccgcccacccuccgcucgacagaguggccgccgugcacgcuggacaggacgguaaggugacccugagagagguggcgcuccgggcgcacggaccccaggccacaggaguguacgccuacaugccuggcagcgagaucacacugcagggagguacuguguccguccagggagacgacggugccggagucgucgcaggagccggccugcuggacgcccugccuccuggaggaaccgugagacucgacgguaccaccguguccaccgacggcgcuaacaccgacgccgugcuggugagaggggacgccgccagagccgagguggugaacaccgugcugagaaccgccaagagcuuggccgcaggaguuuccgcgcagcacggaggccggguaacccugcggcagaccagaauugaaacggcuggugccggugccgaagguauuucgguacuaggauucgagccucagagcggcagcgguccagcuucgguagacaugcagggugguagcauuaccacgaccggcaacagggccgccgggaucgcgcugacccacggcuccgcgagacuggagggagucgccgugcgagcagaaggauccggauccagcgccgcccagcuggcaaacggaguucuugugguauccgcaggcucacucgccucugcgcagucuggcgcuauaucugugacugacaccccucugaagcugaugccaggagcucucgcuuccagcacuguaagcgucagacuuacugacggagccacugcacaaggagguaacggcguauuccugcagcagcauucaacuauaccuguggcuguggcucuugagagcggagcgcuggcacgcggcgacaucguggccgacgggaacaagccccucgacgccgguaucagcuugucaguggccucuggagccgccuggcacggggcuacccaagugcugcagucugccacgcugggcaaggguggcaccuggguuguuaacgccgacagcagagugcaggacaugagcaugcggggcggaaggguugaguuccaggcuccggccccugaggccagcuacaagaccuuaacacuccagaccuuagacggaaacggcguguucgugcugaacaccaacgucgcagcgggacagaacgaccagcugagaguuaccggcagggcugacggucagcacagaguccuggucaggaacgccggaggcgaggccgauagccggggugcgcgguuaggacugguucacacccagggccagggcaacgcaguauucagauuagcaaacguggguaaggccguggaccugggcacguggagauacaguuuggccgaggacccuaagacccacguguggagucugcaaagagccggucaggcucugucaggcgcugccaacgccgccgugaacgcugcggaccugaguaguaucgcccuagcagaaagcaacgcc

SEQ ID NO: 56 - protein, 1359 aa, Artificial sequence
  Synthetic
METPAQLLFLLLLWLPDTTGDWNNQAIVKTGERQHGIHIQGSDPGGVRTASGTTIKVSGRQAQGILLENPAAELQFRNGAVTSSGQLSDDGIRRFLGTVTVKAGKLVADHATLANVGDTWDDDGIALYVAGEQAQASIADSTLQGAGGVQIERGANVVVQRSAIVDGGLHIGALQSLQPEDLPPSRVVLRDTNVVAVPASGAPAAVSVLGASELTLDGGHITGGRAAGVAAMQGAVVHLQRATIRRGDAPAGGAVPGGAVPGGAVPGGFGPGGFGPVLDGWYGVDVSGSSVELAQSIVEAPELGAAIRVGRGARVTVSGGSLSAPHGNVIETGGARRFAPQAAPLSITLQAGAHAQGKALLYRVLPEPVKLTLTGGADAQGDIVATELPSIPGTSIGPLDVALASQARWTGATRAVDSLSIDNAVWVMTDNSNVGALRLASDGSVDFQQPAEAGRFKVLTVNTLAGSGLFRMNVFADLGLSDKLVVMQDASGQHRLWVRNSGSEPASANTLLLVQTPLGSAATFTLANKDGKVDIGTYRYRLAANGNGQWSLVGAKAPPAPKPAPQPGPQPPQPPQPQPEAPAPQPPAGRELSAAANAAVNTGGVGLASTLWYAESNRAKRSGSGAPVKQTLNFDLLKLAGDVESNPGPMETPAQLLFLLLLWLPDTTGQAPQPPVAGAPHAQDAGQEGEFDHRDNTLIAVFDDGVGINLDDDPDELGETAPPTLKDIHISVEHKNPMSKPAIGVRVSGAGRALTLAGSTIDATEGGIPAVVRRGGTLELDGVTVAGGEGMEPMTVSDAGSRLSVRGGVLGGEAPGVGLVRAAQGGQASIIDATLQSILGPALIADGGSISVAGGSIDMDMGPGFPPPPPPLPGAPLAAHPPLDRVAAVHAGQDGKVTLREVALRAHGPQATGVYAYMPGSEITLQGGTVSVQGDDGAGVVAGAGLLDALPPGGTVRLDGTTVSTDGANTDAVLVRGDAARAEVVNTVLRTAKSLAAGVSAQHGGRVTLRQTRIETAGAGAEGISVLGFEPQSGSGPASVDMQGGSITTTGNRAAGIALTHGSARLEGVAVRAEGSGSSAAQLANGVLVVSAGSLASAQSGAISVTDTPLKLMPGALASSTVSVRLTDGATAQGGNGVFLQQHSTIPVAVALESGALARGDIVADGNKPLDAGISLSVASGAAWHGATQVLQSATLGKGGTWVVNADSRVQDMSMRGGRVEFQAPAPEASYKTLTLQTLDGNGVFVLNTNVAAGQNDQLRVTGRADGQHRVLVRNAGGEADSRGARLGLVHTQGQGNAVFRLANVGKAVDLGTWRYSLAEDPKTHVWSLQRAGQALSGAANAAVNAADLSSIALAESNA

SEQ ID NO: 57 - RNA, 4157 nt, Artificial sequence
  Synthetic
gggaaauaagagagaaaagaagaguaagaagaaauauaagaccccggcgccgccaccauggagaccccugcccagcugcuguuccugcugcugcuuuggcugccagacaccaccggagacuggaacaaccaggccaucgugaagaccggcgagagacagcacggcauccacauccaaggcagcgaccccggcggcgugagaaccgccuccggcaccacaaucaaggugagcggcagacaggcccagggcauccugcuggagaacccugccgccgagcugcaguucagaaacggcgccgugaccagcagcggccagcuguccgacgacggcaucagaagauuccugggcaccgugaccgugaaggccggcaagcugguggccgaccacgccacccuggccaacgugggugauaccugggacgacgacgguauugcccuguacguggccggcgagcaagcacaggccagcaucgccgacagcacccugcaaggcgcaggcggcgugcagaucgagagaggcgccaacgugguggugcagagaagcgccauuguugacggcggccugcacaucggcgcccugcagagccugcagccugaggaccugccuccuagcagaguggugcugagagacacaaacgucgucgccgugccagcguccggagcuccagcagccgugagcgugcugggcgccagcgagcugacccucgacggaggacacaucaccgguggcagggccgccgguguggccgccaugcagggagccguugugcaucuacaaagagcuaccauccgacggggcgacgcuccggcugguggagcggucccuggcggugccguccccggaggagcagugccaggcgguuucggcccggguggauucggcccugugcuagacggcugguacggcguggacgugaguggcuccagcguggagcuggcccagaguauuguugaggccccugagcucggcgcugccaucagagugggccguggugccagagugaccguuagcgguggcucacuuagcgccccucacggcaacgugaucgaaacuggaggagcgagacgauucgcaccucaggccgccccucugagcauaacgcugcaagcgggcgcccacgcgcaaggcaaggcccugcuguacagagugcugccugagccugugaaguuaacucugacgggaggugccgacgcacaaggugacaucguggccacggaacuuccuagcaucccuggcacaucuaucggcccucuggacguugcguuagcuucgcaggcccguuggaccggcgccaccagagccguggacucccuaagcaucgacaacgccgugugggucaugaccgacaacucaaacgugggcgcgcugagauuggccucagacggcagugucgauuuccaacaaccugccgaagcaggaagauucaaggugcugacugucaauacacuggccggaagcggccuguucagaaugaacguguucgccgaccugggacucucagauaagcuggucgugaugcaggacgcaaguggccagcacagacugugggucagaaacucgggcuccgagccagccagugccaauacuuugcuguuggugcaaaccccucugggcagugccgccaccuucacucucgccaacaaggacggcaagguggacaucggcaccuacagauacaggcuggcagcaaacggaaacggccaguggagccuagucggagcaaaggccccuccugccccuaagccugcuccacagccuggcccacagccuccgcaaccuccgcagccucagccagaggccccagccccacaaccuccugcaggcagagagcugagugccgcugcuaacgccgccguuaauaccggaggugucggccucgcgucuaccuugugguacgccgagagcaacggaggagguggcucuggcggagguggaagcgguggaggcggaagugguggcggaggcagccaggccccucagccuccgguggccggcgccccacacgcccaagacgcagggcaggagggcgaguucgaccacagagacaacacccugaucgccguguucgacgacggcgugggcaucaaccuggacgacgacccugacgagcugggcgaaacagccccuccuacccugaaggacauccacaucagcguggagcacaagaacccuaugagcaagccugccaucggcgugagagugagcggggccgguagggcccugacccucgcuggcagcaccaucgacgcuaccgagggcggcaucccugccguugucagaagaggcggcacccuggaacuggacggggugaccgucgccggcggagagggcauggagccuaugaccgugagcgacgcaggcagccgacucagugugcggggaggugugcugggaggagaggccccgggcgugggguuagugcgagccgcucagggcggacaggcuagcauaaucgacgcaacccugcagagcauccugggacccgcccugauagcggacgguggcucaaucucaguggcuggaggcuccauugacauggacaugggcccaggcuucccuccuccaccaccuccucugccuggagcaccucuggccgcccacccuccgcucgacagaguggccgccgugcacgcuggacaggacgguaaggugacccugagagagguggcgcuccgggcgcacggaccccaggccacaggaguguacgccuacaugccuggcagcgagaucacacugcagggagguacuguguccguccagggagacgacggugccggagucgucgcaggagccggccugcuggacgcccugccuccuggaggaaccgugagacucgacgguaccaccguguccaccgacggcgcuaacaccgacgccgugcuggugagaggggacgccgccagagccgagguggugaacaccgugcugagaaccgccaagagcuuggccgcaggaguuuccgcgcagcacggaggccggguaacccugcggcagaccagaauugaaacggcuggugccggugccgaagguauuucgguacuaggauucgagccucagagcggcagcgguccagcuucgguagacaugcagggugguagcauuaccacgaccggcaacagggccgccgggaucgcgcugacccacggcuccgcgagacuggagggagucgccgugcgagcagaaggauccggauccagcgccgcccagcuggcaaacggaguucuugugguauccgcaggcucacucgccucugcgcagucuggcgcuauaucugugacugacaccccucugaagcugaugccaggagcucucgcuuccagcacuguaagcgucagacuuacugacggagccacugcacaaggagguaacggcguauuccugcagcagcauucaacuauaccuguggcuguggcucuugagagcggagcgcuggcacgcggcgacaucguggccgacgggaacaagccccucgacgccgguaucagcuugucaguggccucuggagccgccuggcacggggcuacccaagugcugcagucugccacgcugggcaaggguggcaccuggguuguuaacgccgacagcagagugcaggacaugagcaugcggggcggaaggguugaguuccaggcuccggccccugaggccagcuacaagaccuuaacacuccagaccuuagacggaaacggcguguucgugcugaacaccaacgucgcagcgggacagaacgaccagcugagaguuaccggcagggcugacggucagcacagaguccuggucaggaacgccggaggcgaggccgauagccggggugcgcgguuaggacugguucacacccagggccagggcaacgcaguauucagauuagcaaacguggguaaggccguggaccugggcacguggagauacaguuuggccgaggacccuaagacccacguguggagucugcaaagagccggucaggcucugucaggcgcugccaacgccgccgugaacgcugcggaccugaguaguaucgcccuagcagaaagcaacgccugauaauaggcuggagccucgguggccuagcuucuugccccuugggccuccccccagccccuccuccccuuccugcacccguacccccguggucuuugaauaaagucugagugggcggc

SEQ ID NO: 58 - RNA, 3981 nt, Artificial sequence
  Synthetic
auggagaccccugcccagcugcuguuccugcugcugcuuuggcugccagacaccaccggagacuggaacaaccaggccaucgugaagaccggcgagagacagcacggcauccacauccaaggcagcgaccccggcggcgugagaaccgccuccggcaccacaaucaaggugagcggcagacaggcccagggcauccugcuggagaacccugccgccgagcugcaguucagaaacggcgccgugaccagcagcggccagcuguccgacgacggcaucagaagauuccugggcaccgugaccgugaaggccggcaagcugguggccgaccacgccacccuggccaacgugggugauaccugggacgacgacgguauugcccuguacguggccggcgagcaagcacaggccagcaucgccgacagcacccugcaaggcgcaggcggcgugcagaucgagagaggcgccaacgugguggugcagagaagcgccauuguugacggcggccugcacaucggcgcccugcagagccugcagccugaggaccugccuccuagcagaguggugcugagagacacaaacgucgucgccgugccagcguccggagcuccagcagccgugagcgugcugggcgccagcgagcugacccucgacggaggacacaucaccgguggcagggccgccgguguggccgccaugcagggagccguugugcaucuacaaagagcuaccauccgacggggcgacgcuccggcugguggagcggucccuggcggugccguccccggaggagcagugccaggcgguuucggcccggguggauucggcccugugcuagacggcugguacggcguggacgugaguggcuccagcguggagcuggcccagaguauuguugaggccccugagcucggcgcugccaucagagugggccguggugccagagugaccguuagcgguggcucacuuagcgccccucacggcaacgugaucgaaacuggaggagcgagacgauucgcaccucaggccgccccucugagcauaacgcugcaagcgggcgcccacgcgcaaggcaaggcccugcuguacagagugcugccugagccugugaaguuaacucugacgggaggugccgacgcacaaggugacaucguggccacggaacuuccuagcaucccuggcacaucuaucggcccucuggacguugcguuagcuucgcaggcccguuggaccggcgccaccagagccguggacucccuaagcaucgacaacgccgugugggucaugaccgacaacucaaacgugggcgcgcugagauuggccucagacggcagugucgauuuccaacaaccugccgaagcaggaagauucaaggugcugacugucaauacacuggccggaagcggccuguucagaaugaacguguucgccgaccugggacucucagauaagcuggucgugaugcaggacgcaaguggccagcacagacugugggucagaaacucgggcuccgagccagccagugccaauacuuugcuguuggugcaaaccccucugggcagugccgccaccuucacucucgccaacaaggacggcaagguggacaucggcaccuacagauacaggcuggcagcaaacggaaacggccaguggagccuagucggagcaaaggccccuccugccccuaagccugcuccacagccuggcccacagccuccgcaaccuccgcagccucagccagaggccccagccccacaaccuccugcaggcagagagcugagugccgcugcuaacgccgccguuaauaccggaggugucggccucgcgucuaccuugugguacgccgagagcaacggaggagguggcucuggcggagguggaagcgguggaggcggaagugguggcggaggcagccaggccccucagccuccgguggccggcgccccacacgcccaagacgcagggcaggagggcgaguucgaccacagagacaacacccugaucgccguguucgacgacggcgugggcaucaaccuggacgacgacccugacgagcugggcgaaacagccccuccuacccugaaggacauccacaucagcguggagcacaagaacccuaugagcaagccugccaucggcgugagagugagcggggccgguagggcccugacccucgcuggcagcaccaucgacgcuaccgagggcggcaucccugccguugucagaagaggcggcacccuggaacuggacggggugaccgucgccggcggagagggcauggagccuaugaccgugagcgacgcaggcagccgacucagugugcggggaggugugcugggaggagaggccccgggcgugggguuagugcgagccgcucagggcggacaggcuagcauaaucgacgcaacccugcagagcauccugggacccgcccugauagcggacgguggcucaaucucaguggcuggaggcuccauugacauggacaugggcccaggcuucccuccuccaccaccuccucugccuggagcaccucuggccgcccacccuccgcucgacagaguggccgccgugcacgcuggacaggacgguaaggugacccugagagagguggcgcuccgggcgcacggaccccaggccacaggaguguacgccuacaugccuggcagcgagaucacacugcagggagguacuguguccguccagggagacgacggugccggagucgucgcaggagccggccugcuggacgcccugccuccuggaggaaccgugagacucgacgguaccaccguguccaccgacggcgcuaacaccgacgccgugcuggugagaggggacgccgccagagccgagguggugaacaccgugcugagaaccgccaagagcuuggccgcaggaguuuccgcgcagcacggaggccggguaacccugcggcagaccagaauugaaacggcuggugccggugccgaagguauuucgguacuaggauucgagccucagagcggcagcgguccagcuucgguagacaugcagggugguagcauuaccacgaccggcaacagggccgccgggaucgcgcugacccacggcuccgcgagacuggagggagucgccgugcgagcagaaggauccggauccagcgccgcccagcuggcaaacggaguucuugugguauccgcaggcucacucgccucugcgcagucuggcgcuauaucugugacugacaccccucugaagcugaugccaggagcucucgcuuccagcacuguaagcgucagacuuacugacggagccacugcacaaggagguaacggcguauuccugcagcagcauucaacuauaccuguggcuguggcucuugagagcggagcgcuggcacgcggcgacaucguggccgacgggaacaagccccucgacgccgguaucagcuugucaguggccucuggagccgccuggcacggggcuacccaagugcugcagucugccacgcugggcaaggguggcaccuggguuguuaacgccgacagcagagugcaggacaugagcaugcggggcggaaggguugaguuccaggcuccggccccugaggccagcuacaagaccuuaacacuccagaccuuagacggaaacggcguguucgugcugaacaccaacgucgcagcgggacagaacgaccagcugagaguuaccggcagggcugacggucagcacagaguccuggucaggaacgccggaggcgaggccgauagccggggugcgcgguuaggacugguucacacccagggccagggcaacgcaguauucagauuagcaaacguggguaaggccguggaccugggcacguggagauacaguuuggccgaggacccuaagacccacguguggagucugcaaagagccggucaggcucugucaggcgcugccaacgccgccgugaacgcugcggaccugaguaguaucgcccuagcagaaagcaacgcc

SEQ ID NO: 59 - protein, 1327 aa, Artificial sequence
  Synthetic
METPAQLLFLLLLWLPDTTGDWNNQAIVKTGERQHGIHIQGSDPGGVRTASGTTIKVSGRQAQGILLENPAAELQFRNGAVTSSGQLSDDGIRRFLGTVTVKAGKLVADHATLANVGDTWDDDGIALYVAGEQAQASIADSTLQGAGGVQIERGANVVVQRSAIVDGGLHIGALQSLQPEDLPPSRVVLRDTNVVAVPASGAPAAVSVLGASELTLDGGHITGGRAAGVAAMQGAVVHLQRATIRRGDAPAGGAVPGGAVPGGAVPGGFGPGGFGPVLDGWYGVDVSGSSVELAQSIVEAPELGAAIRVGRGARVTVSGGSLSAPHGNVIETGGARRFAPQAAPLSITLQAGAHAQGKALLYRVLPEPVKLTLTGGADAQGDIVATELPSIPGTSIGPLDVALASQARWTGATRAVDSLSIDNAVWVMTDNSNVGALRLASDGSVDFQQPAEAGRFKVLTVNTLAGSGLFRMNVFADLGLSDKLVVMQDASGQHRLWVRNSGSEPASANTLLLVQTPLGSAATFTLANKDGKVDIGTYRYRLAANGNGQWSLVGAKAPPAPKPAPQPGPQPPQPPQPQPEAPAPQPPAGRELSAAANAAVNTGGVGLASTLWYAESNGGGGSGGGGSGGGGSGGGGSQAPQPPVAGAPHAQDAGQEGEFDHRDNTLIAVFDDGVGINLDDDPDELGETAPPTLKDIHISVEHKNPMSKPAIGVRVSGAGRALTLAGSTIDATEGGIPAVVRRGGTLELDGVTVAGGEGMEPMTVSDAGSRLSVRGGVLGGEAPGVGLVRAAQGGQASIIDATLQSILGPALIADGGSISVAGGSIDMDMGPGFPPPPPPLPGAPLAAHPPLDRVAAVHAGQDGKVTLREVALRAHGPQATGVYAYMPGSEITLQGGTVSVQGDDGAGVVAGAGLLDALPPGGTVRLDGTTVSTDGANTDAVLVRGDAARAEVVNTVLRTAKSLAAGVSAQHGGRVTLRQTRIETAGAGAEGISVLGFEPQSGSGPASVDMQGGSITTTGNRAAGIALTHGSARLEGVAVRAEGSGSSAAQLANGVLVVSAGSLASAQSGAISVTDTPLKLMPGALASSTVSVRLTDGATAQGGNGVFLQQHSTIPVAVALESGALARGDIVADGNKPLDAGISLSVASGAAWHGATQVLQSATLGKGGTWVVNADSRVQDMSMRGGRVEFQAPAPEASYKTLTLQTLDGNGVFVLNTNVAAGQNDQLRVTGRADGQHRVLVRNAGGEADSRGARLGLVHTQGQGNAVFRLANVGKAVDLGTWRYSLAEDPKTHVWSLQRAGQALSGAANAAVNAADLSSIALAESNA

SEQ ID NO: 60 - RNA, 4157 nt, Artificial sequence
  Synthetic
gggaaauaagagagaaaagaagaguaagaagaaauauaagaccccggcgccgccaccauggagaccccugcccagcugcuguuccugcugcugcuuuggcugccagacaccaccggacaggccccucagccuccgguggccggcgccccacacgcccaagacgcagggcaggagggcgaguucgaccacagagacaacacccugaucgccguguucgacgacggcgugggcaucaaccuggacgacgacccugacgagcugggcgaaacagccccuccuacccugaaggacauccacaucagcguggagcacaagaacccuaugagcaagccugccaucggcgugagagugagcggggccgguagggcccugacccucgcuggcagcaccaucgacgcuaccgagggcggcaucccugccguugucagaagaggcggcacccuggaacuggacggggugaccgucgccggcggagagggcauggagccuaugaccgugagcgacgcaggcagccgacucagugugcggggaggugugcugggaggagaggccccgggcgugggguuagugcgagccgcucagggcggacaggcuagcauaaucgacgcaacccugcagagcauccugggacccgcccugauagcggacgguggcucaaucucaguggcuggaggcuccauugacauggacaugggcccaggcuucccuccuccaccaccuccucugccuggagcaccucuggccgcccacccuccgcucgacagaguggccgccgugcacgcuggacaggacgguaaggugacccugagagagguggcgcuccgggcgcacggaccccaggccacaggaguguacgccuacaugccuggcagcgagaucacacugcagggagguacuguguccguccagggagacgacggugccggagucgucgcaggagccggccugcuggacgcccugccuccuggaggaaccgugagacucgacgguaccaccguguccaccgacggcgcuaacaccgacgccgugcuggugagaggggacgccgccagagccgagguggugaacaccgugcugagaaccgccaagagcuuggccgcaggaguuuccgcgcagcacggaggccggguaacccugcggcagaccagaauugaaacggcuggugccggugccgaagguauuucgguacuaggauucgagccucagagcggcagcgguccagcuucgguagacaugcagggugguagcauuaccacgaccggcaacagggccgccgggaucgcgcugacccacggcuccgcgagacuggagggagucgccgugcgagcagaaggauccggauccagcgccgcccagcuggcaaacggaguucuugugguauccgcaggcucacucgccucugcgcagucuggcgcuauaucugugacugacaccccucugaagcugaugccaggagcucucgcuuccagcacuguaagcgucagacuuacugacggagccacugcacaaggagguaacggcguauuccugcagcagcauucaacuauaccuguggcuguggcucuugagagcggagcgcuggcacgcggcgacaucguggccgacgggaacaagccccucgacgccgguaucagcuugucaguggccucuggagccgccuggcacggggcuacccaagugcugcagucugccacgcugggcaaggguggcaccuggguuguuaacgccgacagcagagugcaggacaugagcaugcggggcggaaggguugaguuccaggcuccggccccugaggccagcuacaagaccuuaacacuccagaccuuagacggaaacggcguguucgugcugaacaccaacgucgcagcgggacagaacgaccagcugagaguuaccggcagggcugacggucagcacagaguccuggucaggaacgccggaggcgaggccgauagccggggugcgcgguuaggacugguucacacccagggccagggcaacgcaguauucagauuagcaaacguggguaaggccguggaccugggcacguggagauacaguuuggccgaggacccuaagacccacguguggagucugcaaagagccggucaggcucugucaggcgcugccaacgccgccgugaacgcugcggaccugaguaguaucgcccuagcagaaagcaacgccggaggagguggcucuggcggagguggaagcgguggaggcggaagugguggcggaggcagcgacuggaacaaccaggccaucgugaagaccggcgagagacagcacggcauccacauccaaggcagcgaccccggcggcgugagaaccgccuccggcaccacaaucaaggugagcggcagacaggcccagggcauccugcuggagaacccugccgccgagcugcaguucagaaacggcgccgugaccagcagcggccagcuguccgacgacggcaucagaagauuccugggcaccgugaccgugaaggccggcaagcugguggccgaccacgccacccuggccaacgugggugauaccugggacgacgacgguauugcccuguacguggccggcgagcaagcacaggccagcaucgccgacagcacccugcaaggcgcaggcggcgugcagaucgagagaggcgccaacgugguggugcagagaagcgccauuguugacggcggccugcacaucggcgcccugcagagccugcagccugaggaccugccuccuagcagaguggugcugagagacacaaacgucgucgccgugccagcguccggagcuccagcagccgugagcgugcugggcgccagcgagcugacccucgacggaggacacaucaccgguggcagggccgccgguguggccgccaugcagggagccguugugcaucuacaaagagcuaccauccgacggggcgacgcuccggcugguggagcggucccuggcggugccguccccggaggagcagugccaggcgguuucggcccggguggauucggcccugugcuagacggcugguacggcguggacgugaguggcuccagcguggagcuggcccagaguauuguugaggccccugagcucggcgcugccaucagagugggccguggugccagagugaccguuagcgguggcucacuuagcgccccucacggcaacgugaucgaaacuggaggagcgagacgauucgcaccucaggccgccccucugagcauaacgcugcaagcgggcgcccacgcgcaaggcaaggcccugcuguacagagugcugccugagccugugaaguuaacucugacgggaggugccgacgcacaaggugacaucguggccacggaacuuccuagcaucccuggcacaucuaucggcccucuggacguugcguuagcuucgcaggcccguuggaccggcgccaccagagccguggacucccuaagcaucgacaacgccgugugggucaugaccgacaacucaaacgugggcgcgcugagauuggccucagacggcagugucgauuuccaacaaccugccgaagcaggaagauucaaggugcugacugucaauacacuggccggaagcggccuguucagaaugaacguguucgccgaccugggacucucagauaagcuggucgugaugcaggacgcaaguggccagcacagacugugggucagaaacucgggcuccgagccagccagugccaauacuuugcuguuggugcaaaccccucugggcagugccgccaccuucacucucgccaacaaggacggcaagguggacaucggcaccuacagauacaggcuggcagcaaacggaaacggccaguggagccuagucggagcaaaggccccuccugccccuaagccugcuccacagccuggcccacagccuccgcaaccuccgcagccucagccagaggccccagccccacaaccuccugcaggcagagagcugagugccgcugcuaacgccgccguuaauaccggaggugucggccucgcgucuaccuugugguacgccgagagcaacugauaauaggcuggagccucgguggccuagcuucuugccccuugggccuccccccagccccuccuccccuuccugcacccguacccccguggucuuugaauaaagucugagugggcggc

SEQ ID NO: 61 - RNA, 3981 nt, Artificial sequence
  Synthetic
auggagaccccugcccagcugcuguuccugcugcugcuuuggcugccagacaccaccggacaggccccucagccuccgguggccggcgccccacacgcccaagacgcagggcaggagggcgaguucgaccacagagacaacacccugaucgccguguucgacgacggcgugggcaucaaccuggacgacgacccugacgagcugggcgaaacagccccuccuacccugaaggacauccacaucagcguggagcacaagaacccuaugagcaagccugccaucggcgugagagugagcggggccgguagggcccugacccucgcuggcagcaccaucgacgcuaccgagggcggcaucccugccguugucagaagaggcggcacccuggaacuggacggggugaccgucgccggcggagagggcauggagccuaugaccgugagcgacgcaggcagccgacucagugugcggggaggugugcugggaggagaggccccgggcgugggguuagugcgagccgcucagggcggacaggcuagcauaaucgacgcaacccugcagagcauccugggacccgcccugauagcggacgguggcucaaucucaguggcuggaggcuccauugacauggacaugggcccaggcuucccuccuccaccaccuccucugccuggagcaccucuggccgcccacccuccgcucgacagaguggccgccgugcacgcuggacaggacgguaaggugacccugagagagguggcgcuccgggcgcacggaccccaggccacaggaguguacgccuacaugccuggcagcgagaucacacugcagggagguacuguguccguccagggagacgacggugccggagucgucgcaggagccggccugcuggacgcccugccuccuggaggaaccgugagacucgacgguaccaccguguccaccgacggcgcuaacaccgacgccgugcuggugagaggggacgccgccagagccgagguggugaacaccgugcugagaaccgccaagagcuuggccgcaggaguuuccgcgcagcacggaggccggguaacccugcggcagaccagaauugaaacggcuggugccggugccgaagguauuucgguacuaggauucgagccucagagcggcagcgguccagcuucgguagacaugcagggugguagcauuaccacgaccggcaacagggccgccgggaucgcgcugacccacggcuccgcgagacuggagggagucgccgugcgagcagaaggauccggauccagcgccgcccagcuggcaaacggaguucuugugguauccgcaggcucacucgccucugcgcagucuggcgcuauaucugugacugacaccccucugaagcugaugccaggagcucucgcuuccagcacuguaagcgucagacuuacugacggagccacugcacaaggagguaacggcguauuccugcagcagcauucaacuauaccuguggcuguggcucuugagagcggagcgcuggcacgcggcgacaucguggccgacgggaacaagccccucgacgccgguaucagcuugucaguggccucuggagccgccuggcacggggcuacccaagugcugcagucugccacgcugggcaaggguggcaccuggguuguuaacgccgacagcagagugcaggacaugagcaugcggggcggaaggguugaguuccaggcuccggccccugaggccagcuacaagaccuuaacacuccagaccuuagacggaaacggcguguucgugcugaacaccaacgucgcagcgggacagaacgaccagcugagaguuaccggcagggcugacggucagcacagaguccuggucaggaacgccggaggcgaggccgauagccggggugcgcgguuaggacugguucacacccagggccagggcaacgcaguauucagauuagcaaacguggguaaggccguggaccugggcacguggagauacaguuuggccgaggacccuaagacccacguguggagucugcaaagagccggucaggcucugucaggcgcugccaacgccgccgugaacgcugcggaccugaguaguaucgcccuagcagaaagcaacgccggaggagguggcucuggcggagguggaagcgguggaggcggaagugguggcggaggcagcgacuggaacaaccaggccaucgugaagaccggcgagagacagcacggcauccacauccaaggcagcgaccccggcggcgugagaaccgccuccggcaccacaaucaaggugagcggcagacaggcccagggcauccugcuggagaacccugccgccgagcugcaguucagaaacggcgccgugaccagcagcggccagcuguccgacgacggcaucagaagauuccugggcaccgugaccgugaaggccggcaagcugguggccgaccacgccacccuggccaacgugggugauaccugggacgacgacgguauugcccuguacguggccggcgagcaagcacaggccagcaucgccgacagcacccugcaaggcgcaggcggcgugcagaucgagagaggcgccaacgugguggugcagagaagcgccauuguugacggcggccugcacaucggcgcccugcagagccugcagccugaggaccugccuccuagcagaguggugcugagagacacaaacgucgucgccgugccagcguccggagcuccagcagccgugagcgugcugggcgccagcgagcugacccucgacggaggacacaucaccgguggcagggccgccgguguggccgccaugcagggagccguugugcaucuacaaagagcuaccauccgacggggcgacgcuccggcugguggagcggucccuggcggugccguccccggaggagcagugccaggcgguuucggcccggguggauucggcccugugcuagacggcugguacggcguggacgugaguggcuccagcguggagcuggcccagaguauuguugaggccccugagcucggcgcugccaucagagugggccguggugccagagugaccguuagcgguggcucacuuagcgccccucacggcaacgugaucgaaacuggaggagcgagacgauucgcaccucaggccgccccucugagcauaacgcugcaagcgggcgcccacgcgcaaggcaaggcccugcuguacagagugcugccugagccugugaaguuaacucugacgggaggugccgacgcacaaggugacaucguggccacggaacuuccuagcaucccuggcacaucuaucggcccucuggacguugcguuagcuucgcaggcccguuggaccggcgccaccagagccguggacucccuaagcaucgacaacgccgugugggucaugaccgacaacucaaacgugggcgcgcugagauuggccucagacggcagugucgauuuccaacaaccugccgaagcaggaagauucaaggugcugacugucaauacacuggccggaagcggccuguucagaaugaacguguucgccgaccugggacucucagauaagcuggucgugaugcaggacgcaaguggccagcacagacugugggucagaaacucgggcuccgagccagccagugccaauacuuugcuguuggugcaaaccccucugggcagugccgccaccuucacucucgccaacaaggacggcaagguggacaucggcaccuacagauacaggcuggcagcaaacggaaacggccaguggagccuagucggagcaaaggccccuccugccccuaagccugcuccacagccuggcccacagccuccgcaaccuccgcagccucagccagaggccccagccccacaaccuccugcaggcagagagcugagugccgcugcuaacgccgccguuaauaccggaggugucggccucgcgucuaccuugugguacgccgagagcaac

SEQ ID NO: 62 - protein, 1327 aa, Artificial sequence
  Synthetic
METPAQLLFLLLLWLPDTTGQAPQPPVAGAPHAQDAGQEGEFDHRDNTLIAVFDDGVGINLDDDPDELGETAPPTLKDIHISVEHKNPMSKPAIGVRVSGAGRALTLAGSTIDATEGGIPAVVRRGGTLELDGVTVAGGEGMEPMTVSDAGSRLSVRGGVLGGEAPGVGLVRAAQGGQASIIDATLQSILGPALIADGGSISVAGGSIDMDMGPGFPPPPPPLPGAPLAAHPPLDRVAAVHAGQDGKVTLREVALRAHGPQATGVYAYMPGSEITLQGGTVSVQGDDGAGVVAGAGLLDALPPGGTVRLDGTTVSTDGANTDAVLVRGDAARAEVVNTVLRTAKSLAAGVSAQHGGRVTLRQTRIETAGAGAEGISVLGFEPQSGSGPASVDMQGGSITTTGNRAAGIALTHGSARLEGVAVRAEGSGSSAAQLANGVLVVSAGSLASAQSGAISVTDTPLKLMPGALASSTVSVRLTDGATAQGGNGVFLQQHSTIPVAVALESGALARGDIVADGNKPLDAGISLSVASGAAWHGATQVLQSATLGKGGTWVVNADSRVQDMSMRGGRVEFQAPAPEASYKTLTLQTLDGNGVFVLNTNVAAGQNDQLRVTGRADGQHRVLVRNAGGEADSRGARLGLVHTQGQGNAVFRLANVGKAVDLGTWRYSLAEDPKTHVWSLQRAGQALSGAANAAVNAADLSSIALAESNAGGGGSGGGGSGGGGSGGGGSDWNNQAIVKTGERQHGIHIQGSDPGGVRTASGTTIKVSGRQAQGILLENPAAELQFRNGAVTSSGQLSDDGIRRFLGTVTVKAGKLVADHATLANVGDTWDDDGIALYVAGEQAQASIADSTLQGAGGVQIERGANVVVQRSAIVDGGLHIGALQSLQPEDLPPSRVVLRDTNVVAVPASGAPAAVSVLGASELTLDGGHITGGRAAGVAAMQGAVVHLQRATIRRGDAPAGGAVPGGAVPGGAVPGGFGPGGFGPVLDGWYGVDVSGSSVELAQSIVEAPELGAAIRVGRGARVTVSGGSLSAPHGNVIETGGARRFAPQAAPLSITLQAGAHAQGKALLYRVLPEPVKLTLTGGADAQGDIVATELPSIPGTSIGPLDVALASQARWTGATRAVDSLSIDNAVWVMTDNSNVGALRLASDGSVDFQQPAEAGRFKVLTVNTLAGSGLFRMNVFADLGLSDKLVVMQDASGQHRLWVRNSGSEPASANTLLLVQTPLGSAATFTLANKDGKVDIGTYRYRLAANGNGQWSLVGAKAPPAPKPAPQPGPQPPQPPQPQPEAPAPQPPAGRELSAAANAAVNTGGVGLASTLWYAESN

SEQ ID NO: 63 - RNA, 2285 nt, Artificial sequence
  Synthetic
gggaaauaagagagaaaagaagaguaagaagaaauauaagaccccggcgccgccaccauggaaaccccugcucagcugcuguuccugcugcugcuguggcugccugauaccacaggggacgauccuccagccaccguguauaaguacgacagcagaccuccugaggacguguuccagaacggcuuuaccgccuggggcaacaacgacaacgugcuggaucaccugaccggcagauccugucaagugggcagcagcaauagcgccuucguguccaccucuagcagcagacgguacacagagguguaccuggaacaccggaugcaagaggccguggaagccgaaagagccggcagaggaacaggccacuucaucggcuacaucuacgaagugcgggccgacaacaacuucuacggcgcugccagcagcuacuucgaguacguggacaccuacggcgacaacgccggaagaauacuggcuggcgcucuggccacauaccagucuggauaucuggcccacagacggaucccuccagagaacauucggagagugacccggguguaccacaacggcauuaccggcgagacaaccaccaccgaguacagcaacgccagauacgugucccagcagacccgggccaauccuaauccuuacaccagccgggccaagagaagcggaagcggcgcucccgugaagcagacccugaacuucgaccugcugaagcuggccggcgacguggagagcaaccccggccccauggagacacccgcccagcugcucuuccuccugcugcucuggcugcccgacaccaccggcaagaaccuggacugcuggguggacaacgaggaggacaucgacgugauccugaagaagagcaccauccugaaccuggacaucaacaacgacaucaucagcgacaucagcggcuucaacagcgccgugaucaccuaccccgacgcccaacuggugcccggcaucaacggcaaggccauccaccuggugaacaacgaggccagcgaggugaucgugcacaaggccauggacaucgaguacaacgacauguucaacaacuucgccgugagcuucuggcugcgggugcccaaggugagcgccagccaccuggagcaguacggcaccaacgaguacagcaucaucagcagcaugaagaagcacagccugagcaucggcagcggcuggagcgugagccugaagggcaacaaccugaucuggacccugaaggauagcgccggcgaggugagacagaucaccuuccgggaccugcccgacaaguucaacgccuaccuggccaacaaguggguguucauuaccaucaccaacgaccggcugagcagcgccaaccuguacaucaacggcgugcugaugggcagcgccgagaucaccggccugggagccauccgggaggacaacaacaucgcccugaagcuggaccggugcaacaacaauaaccaguacgugagcaucgacaaguuccggaucuucugcaaggcccugaaccccaaggagaucgagaagcuguacaccagcuaccugagcaucaccuuccugcgggacuucuggggcaauccccugcgguacgacaccgaguacuaccugauccccguggccucaagcagcaaggacgugcaguugaagaacaucgccgacuacauguaccugaccaacgccccgagcuacaccaacggcaagcugaacaucuacuaccggcggcuguacaacggccugaaguucaucaucaagcgguacaccccuaacaacgagaucgacagcuucgugaagagcggcgacuucaucaagcuguacgugagcuacaacaacaacgagcacaucgugggcuaccccaaggacggcaacgccuucaacaaccuggaccggauccugcgggugggcuacaacgccccaggcauuccccuguacaagaagauggaggccgugaagcugcgggaccugaagaccuacagcgugcagcugaaacuguacgacgacaagaacgccagccugggccuggugggcacccacaacggccagaucggcaacgaccccaaccgggacauccugaucgccagcaacugguacuuuaaccaccugaaggacaagauccugggcugcgacugguacuucgugcccaccgacgagggcuggaccaacgacugauaauaggcuggagccucgguggccuagcuucuugccccuugggccuccccccagccccuccuccccuuccugcacccguacccccguggucuuugaauaaagucugagugggcggc

SEQ ID NO: 64 - RNA, 2109 nt, Artificial sequence
  Synthetic
auggaaaccccugcucagcugcuguuccugcugcugcuguggcugccugauaccacaggggacgauccuccagccaccguguauaaguacgacagcagaccuccugaggacguguuccagaacggcuuuaccgccuggggcaacaacgacaacgugcuggaucaccugaccggcagauccugucaagugggcagcagcaauagcgccuucguguccaccucuagcagcagacgguacacagagguguaccuggaacaccggaugcaagaggccguggaagccgaaagagccggcagaggaacaggccacuucaucggcuacaucuacgaagugcgggccgacaacaacuucuacggcgcugccagcagcuacuucgaguacguggacaccuacggcgacaacgccggaagaauacuggcuggcgcucuggccacauaccagucuggauaucuggcccacagacggaucccuccagagaacauucggagagugacccggguguaccacaacggcauuaccggcgagacaaccaccaccgaguacagcaacgccagauacgugucccagcagacccgggccaauccuaauccuuacaccagccgggccaagagaagcggaagcggcgcucccgugaagcagacccugaacuucgaccugcugaagcuggccggcgacguggagagcaaccccggccccauggagacacccgcccagcugcucuuccuccugcugcucuggcugcccgacaccaccggcaagaaccuggacugcuggguggacaacgaggaggacaucgacgugauccugaagaagagcaccauccugaaccuggacaucaacaacgacaucaucagcgacaucagcggcuucaacagcgccgugaucaccuaccccgacgcccaacuggugcccggcaucaacggcaaggccauccaccuggugaacaacgaggccagcgaggugaucgugcacaaggccauggacaucgaguacaacgacauguucaacaacuucgccgugagcuucuggcugcgggugcccaaggugagcgccagccaccuggagcaguacggcaccaacgaguacagcaucaucagcagcaugaagaagcacagccugagcaucggcagcggcuggagcgugagccugaagggcaacaaccugaucuggacccugaaggauagcgccggcgaggugagacagaucaccuuccgggaccugcccgacaaguucaacgccuaccuggccaacaaguggguguucauuaccaucaccaacgaccggcugagcagcgccaaccuguacaucaacggcgugcugaugggcagcgccgagaucaccggccugggagccauccgggaggacaacaacaucgcccugaagcuggaccggugcaacaacaauaaccaguacgugagcaucgacaaguuccggaucuucugcaaggcccugaaccccaaggagaucgagaagcuguacaccagcuaccugagcaucaccuuccugcgggacuucuggggcaauccccugcgguacgacaccgaguacuaccugauccccguggccucaagcagcaaggacgugcaguugaagaacaucgccgacuacauguaccugaccaacgccccgagcuacaccaacggcaagcugaacaucuacuaccggcggcuguacaacggccugaaguucaucaucaagcgguacaccccuaacaacgagaucgacagcuucgugaagagcggcgacuucaucaagcuguacgugagcuacaacaacaacgagcacaucgugggcuaccccaaggacggcaacgccuucaacaaccuggaccggauccugcgggugggcuacaacgccccaggcauuccccuguacaagaagauggaggccgugaagcugcgggaccugaagaccuacagcgugcagcugaaacuguacgacgacaagaacgccagccugggccuggugggcacccacaacggccagaucggcaacgaccccaaccgggacauccugaucgccagcaacugguacuuuaaccaccugaaggacaagauccugggcugcgacugguacuucgugcccaccgacgagggcuggaccaacgac

SEQ ID NO: 65 - protein, 703 aa, Artificial sequence
  Synthetic
METPAQLLFLLLLWLPDTTGDDPPATVYKYDSRPPEDVFQNGFTAWGNNDNVLDHLTGRSCQVGSSNSAFVSTSSSRRYTEVYLEHRMQEAVEAERAGRGTGHFIGYIYEVRADNNFYGAASSYFEYVDTYGDNAGRILAGALATYQSGYLAHRRIPPENIRRVTRVYHNGITGETTTTEYSNARYVSQQTRANPNPYTSRAKRSGSGAPVKQTLNFDLLKLAGDVESNPGPMETPAQLLFLLLLWLPDTTGKNLDCWVDNEEDIDVILKKSTILNLDINNDIISDISGFNSAVITYPDAQLVPGINGKAIHLVNNEASEVIVHKAMDIEYNDMFNNFAVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNIALKLDRCNNNNQYVSIDKFRIFCKALNPKEIEKLYTSYLSITFLRDFWGNPLRYDTEYYLIPVASSSKDVQLKNIADYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 66 - RNA, 2189 nt, Artificial sequence
  Synthetic
gggaaauaagagagaaaagaagaguaagaagaaauauaagaccccggcgccgccaccauggaaaccccugcucagcugcuguuccugcugcugcuguggcugccugauaccacaggggacgauccuccagccaccguguauaaguacgacagcagaccuccugaggacguguuccagaacggcuuuaccgccuggggcaacaacgacaacgugcuggaucaccugaccggcagauccugucaagugggcagcagcaauagcgccuucguguccaccucuagcagcagacgguacacagagguguaccuggaacaccggaugcaagaggccguggaagccgaaagagccggcagaggaacaggccacuucaucggcuacaucuacgaagugcgggccgacaacaacuucuacggcgcugccagcagcuacuucgaguacguggacaccuacggcgacaacgccggaagaauacuggcuggcgcucuggccacauaccagucuggauaucuggcccacagacggaucccuccagagaacauucggagagugacccggguguaccacaacggcauuaccggcgagacaaccaccaccgaguacagcaacgccagauacgugucccagcagacccgggccaauccuaauccuuacaccagcggaggagguggcucuggcggagguggaagcgguggaggcggaagugguggcggaggcagcaagaaccuggacugcuggguggacaacgaggaggacaucgacgugauccugaagaagagcaccauccugaaccuggacaucaacaacgacaucaucagcgacaucagcggcuucaacagcgccgugaucaccuaccccgacgcccaacuggugcccggcaucaacggcaaggccauccaccuggugaacaacgaggccagcgaggugaucgugcacaaggccauggacaucgaguacaacgacauguucaacaacuucgccgugagcuucuggcugcgggugcccaaggugagcgccagccaccuggagcaguacggcaccaacgaguacagcaucaucagcagcaugaagaagcacagccugagcaucggcagcggcuggagcgugagccugaagggcaacaaccugaucuggacccugaaggauagcgccggcgaggugagacagaucaccuuccgggaccugcccgacaaguucaacgccuaccuggccaacaaguggguguucauuaccaucaccaacgaccggcugagcagcgccaaccuguacaucaacggcgugcugaugggcagcgccgagaucaccggccugggagccauccgggaggacaacaacaucgcccugaagcuggaccggugcaacaacaauaaccaguacgugagcaucgacaaguuccggaucuucugcaaggcccugaaccccaaggagaucgagaagcuguacaccagcuaccugagcaucaccuuccugcgggacuucuggggcaauccccugcgguacgacaccgaguacuaccugauccccguggccucaagcagcaaggacgugcaguugaagaacaucgccgacuacauguaccugaccaacgccccgagcuacaccaacggcaagcugaacaucuacuaccggcggcuguacaacggccugaaguucaucaucaagcgguacaccccuaacaacgagaucgacagcuucgugaagagcggcgacuucaucaagcuguacgugagcuacaacaacaacgagcacaucgugggcuaccccaaggacggcaacgccuucaacaaccuggaccggauccugcgggugggcuacaacgccccaggcauuccccuguacaagaagauggaggccgugaagcugcgggaccugaagaccuacagcgugcagcugaaacuguacgacgacaagaacgccagccugggccuggugggcacccacaacggccagaucggcaacgaccccaaccgggacauccugaucgccagcaacugguacuuuaaccaccugaaggacaagauccugggcugcgacugguacuucgugcccaccgacgagggcuggaccaacgacugauaauaggcuggagccucgguggccuagcuucuugccccuugggccuccccccagccccuccuccccuuccugcacccguacccccguggucuuugaauaaagucugagugggcggc

SEQ ID NO: 67 - RNA, 2013 nt, Artificial sequence
  Synthetic
auggaaaccccugcucagcugcuguuccugcugcugcuguggcugccugauaccacaggggacgauccuccagccaccguguauaaguacgacagcagaccuccugaggacguguuccagaacggcuuuaccgccuggggcaacaacgacaacgugcuggaucaccugaccggcagauccugucaagugggcagcagcaauagcgccuucguguccaccucuagcagcagacgguacacagagguguaccuggaacaccggaugcaagaggccguggaagccgaaagagccggcagaggaacaggccacuucaucggcuacaucuacgaagugcgggccgacaacaacuucuacggcgcugccagcagcuacuucgaguacguggacaccuacggcgacaacgccggaagaauacuggcuggcgcucuggccacauaccagucuggauaucuggcccacagacggaucccuccagagaacauucggagagugacccggguguaccacaacggcauuaccggcgagacaaccaccaccgaguacagcaacgccagauacgugucccagcagacccgggccaauccuaauccuuacaccagcggaggagguggcucuggcggagguggaagcgguggaggcggaagugguggcggaggcagcaagaaccuggacugcuggguggacaacgaggaggacaucgacgugauccugaagaagagcaccauccugaaccuggacaucaacaacgacaucaucagcgacaucagcggcuucaacagcgccgugaucaccuaccccgacgcccaacuggugcccggcaucaacggcaaggccauccaccuggugaacaacgaggccagcgaggugaucgugcacaaggccauggacaucgaguacaacgacauguucaacaacuucgccgugagcuucuggcugcgggugcccaaggugagcgccagccaccuggagcaguacggcaccaacgaguacagcaucaucagcagcaugaagaagcacagccugagcaucggcagcggcuggagcgugagccugaagggcaacaaccugaucuggacccugaaggauagcgccggcgaggugagacagaucaccuuccgggaccugcccgacaaguucaacgccuaccuggccaacaaguggguguucauuaccaucaccaacgaccggcugagcagcgccaaccuguacaucaacggcgugcugaugggcagcgccgagaucaccggccugggagccauccgggaggacaacaacaucgcccugaagcuggaccggugcaacaacaauaaccaguacgugagcaucgacaaguuccggaucuucugcaaggcccugaaccccaaggagaucgagaagcuguacaccagcuaccugagcaucaccuuccugcgggacuucuggggcaauccccugcgguacgacaccgaguacuaccugauccccguggccucaagcagcaaggacgugcaguugaagaacaucgccgacuacauguaccugaccaacgccccgagcuacaccaacggcaagcugaacaucuacuaccggcggcuguacaacggccugaaguucaucaucaagcgguacaccccuaacaacgagaucgacagcuucgugaagagcggcgacuucaucaagcuguacgugagcuacaacaacaacgagcacaucgugggcuaccccaaggacggcaacgccuucaacaaccuggaccggauccugcgggugggcuacaacgccccaggcauuccccuguacaagaagauggaggccgugaagcugcgggaccugaagaccuacagcgugcagcugaaacuguacgacgacaagaacgccagccugggccuggugggcacccacaacggccagaucggcaacgaccccaaccgggacauccugaucgccagcaacugguacuuuaaccaccugaaggacaagauccugggcugcgacugguacuucgugcccaccgacgagggcuggaccaacgac

SEQ ID NO: 68 - protein, 671 aa, Artificial sequence
  Synthetic
METPAQLLFLLLLWLPDTTGDDPPATVYKYDSRPPEDVFQNGFTAWGNNDNVLDHLTGRSCQVGSSNSAFVSTSSSRRYTEVYLEHRMQEAVEAERAGRGTGHFIGYIYEVRADNNFYGAASSYFEYVDTYGDNAGRILAGALATYQSGYLAHRRIPPENIRRVTRVYHNGITGETTTTEYSNARYVSQQTRANPNPYTSGGGGSGGGGSGGGGSGGGGSKNLDCWVDNEEDIDVILKKSTILNLDINNDIISDISGFNSAVITYPDAQLVPGINGKAIHLVNNEASEVIVHKAMDIEYNDMFNNFAVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNIALKLDRCNNNNQYVSIDKFRIFCKALNPKEIEKLYTSYLSITFLRDFWGNPLRYDTEYYLIPVASSSKDVQLKNIADYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 69 - RNA, 2189 nt, Artificial sequence
  Synthetic
gggaaauaagagagaaaagaagaguaagaagaaauauaagaccccggcgccgccaccauggaaaccccugcucagcugcuguuccugcugcugcuguggcugccugauaccacagggaagaaccuggacugcuggguggacaacgaggaggacaucgacgugauccugaagaagagcaccauccugaaccuggacaucaacaacgacaucaucagcgacaucagcggcuucaacagcgccgugaucaccuaccccgacgcccaacuggugcccggcaucaacggcaaggccauccaccuggugaacaacgaggccagcgaggugaucgugcacaaggccauggacaucgaguacaacgacauguucaacaacuucgccgugagcuucuggcugcgggugcccaaggugagcgccagccaccuggagcaguacggcaccaacgaguacagcaucaucagcagcaugaagaagcacagccugagcaucggcagcggcuggagcgugagccugaagggcaacaaccugaucuggacccugaaggauagcgccggcgaggugagacagaucaccuuccgggaccugcccgacaaguucaacgccuaccuggccaacaaguggguguucauuaccaucaccaacgaccggcugagcagcgccaaccuguacaucaacggcgugcugaugggcagcgccgagaucaccggccugggagccauccgggaggacaacaacaucgcccugaagcuggaccggugcaacaacaauaaccaguacgugagcaucgacaaguuccggaucuucugcaaggcccugaaccccaaggagaucgagaagcuguacaccagcuaccugagcaucaccuuccugcgggacuucuggggcaauccccugcgguacgacaccgaguacuaccugauccccguggccucaagcagcaaggacgugcaguugaagaacaucgccgacuacauguaccugaccaacgccccgagcuacaccaacggcaagcugaacaucuacuaccggcggcuguacaacggccugaaguucaucaucaagcgguacaccccuaacaacgagaucgacagcuucgugaagagcggcgacuucaucaagcuguacgugagcuacaacaacaacgagcacaucgugggcuaccccaaggacggcaacgccuucaacaaccuggaccggauccugcgggugggcuacaacgccccaggcauuccccuguacaagaagauggaggccgugaagcugcgggaccugaagaccuacagcgugcagcugaaacuguacgacgacaagaacgccagccugggccuggugggcacccacaacggccagaucggcaacgaccccaaccgggacauccugaucgccagcaacugguacuuuaaccaccugaaggacaagauccugggcugcgacugguacuucgugcccaccgacgagggcuggaccaacgacggaggagguggcucuggcggagguggaagcgguggaggcggaagugguggcggaggcagcgacgauccuccagccaccguguauaaguacgacagcagaccuccugaggacguguuccagaacggcuuuaccgccuggggcaacaacgacaacgugcuggaucaccugaccggcagauccugucaagugggcagcagcaauagcgccuucguguccaccucuagcagcagacgguacacagagguguaccuggaacaccggaugcaagaggccguggaagccgaaagagccggcagaggaacaggccacuucaucggcuacaucuacgaagugcgggccgacaacaacuucuacggcgcugccagcagcuacuucgaguacguggacaccuacggcgacaacgccggaagaauacuggcuggcgcucuggccacauaccagucuggauaucuggcccacagacggaucccuccagagaacauucggagagugacccggguguaccacaacggcauuaccggcgagacaaccaccaccgaguacagcaacgccagauacgugucccagcagacccgggccaauccuaauccuuacaccagcugauaauaggcuggagccucgguggccuagcuucuugccccuugggccuccccccagccccuccuccccuuccugcacccguacccccguggucuuugaauaaagucugagugggcggc

SEQ ID NO: 70 - RNA, 2013 nt, Artificial sequence
  Synthetic
auggaaaccccugcucagcugcuguuccugcugcugcuguggcugccugauaccacagggaagaaccuggacugcuggguggacaacgaggaggacaucgacgugauccugaagaagagcaccauccugaaccuggacaucaacaacgacaucaucagcgacaucagcggcuucaacagcgccgugaucaccuaccccgacgcccaacuggugcccggcaucaacggcaaggccauccaccuggugaacaacgaggccagcgaggugaucgugcacaaggccauggacaucgaguacaacgacauguucaacaacuucgccgugagcuucuggcugcgggugcccaaggugagcgccagccaccuggagcaguacggcaccaacgaguacagcaucaucagcagcaugaagaagcacagccugagcaucggcagcggcuggagcgugagccugaagggcaacaaccugaucuggacccugaaggauagcgccggcgaggugagacagaucaccuuccgggaccugcccgacaaguucaacgccuaccuggccaacaaguggguguucauuaccaucaccaacgaccggcugagcagcgccaaccuguacaucaacggcgugcugaugggcagcgccgagaucaccggccugggagccauccgggaggacaacaacaucgcccugaagcuggaccggugcaacaacaauaaccaguacgugagcaucgacaaguuccggaucuucugcaaggcccugaaccccaaggagaucgagaagcuguacaccagcuaccugagcaucaccuuccugcgggacuucuggggcaauccccugcgguacgacaccgaguacuaccugauccccguggccucaagcagcaaggacgugcaguugaagaacaucgccgacuacauguaccugaccaacgccccgagcuacaccaacggcaagcugaacaucuacuaccggcggcuguacaacggccugaaguucaucaucaagcgguacaccccuaacaacgagaucgacagcuucgugaagagcggcgacuucaucaagcuguacgugagcuacaacaacaacgagcacaucgugggcuaccccaaggacggcaacgccuucaacaaccuggaccggauccugcgggugggcuacaacgccccaggcauuccccuguacaagaagauggaggccgugaagcugcgggaccugaagaccuacagcgugcagcugaaacuguacgacgacaagaacgccagccugggccuggugggcacccacaacggccagaucggcaacgaccccaaccgggacauccugaucgccagcaacugguacuuuaaccaccugaaggacaagauccugggcugcgacugguacuucgugcccaccgacgagggcuggaccaacgacggaggagguggcucuggcggagguggaagcgguggaggcggaagugguggcggaggcagcgacgauccuccagccaccguguauaaguacgacagcagaccuccugaggacguguuccagaacggcuuuaccgccuggggcaacaacgacaacgugcuggaucaccugaccggcagauccugucaagugggcagcagcaauagcgccuucguguccaccucuagcagcagacgguacacagagguguaccuggaacaccggaugcaagaggccguggaagccgaaagagccggcagaggaacaggccacuucaucggcuacaucuacgaagugcgggccgacaacaacuucuacggcgcugccagcagcuacuucgaguacguggacaccuacggcgacaacgccggaagaauacuggcuggcgcucuggccacauaccagucuggauaucuggcccacagacggaucccuccagagaacauucggagagugacccggguguaccacaacggcauuaccggcgagacaaccaccaccgaguacagcaacgccagauacgugucccagcagacccgggccaauccuaauccuuacaccagc

SEQ ID NO: 71 - protein, 671 aa, Artificial sequence
  Synthetic
METPAQLLFLLLLWLPDTTGKNLDCWVDNEEDIDVILKKSTILNLDINNDIISDISGFNSAVITYPDAQLVPGINGKAIHLVNNEASEVIVHKAMDIEYNDMFNNFAVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNIALKLDRCNNNNQYVSIDKFRIFCKALNPKEIEKLYTSYLSITFLRDFWGNPLRYDTEYYLIPVASSSKDVQLKNIADYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTNDGGGGSGGGGSGGGGSGGGGSDDPPATVYKYDSRPPEDVFQNGFTAWGNNDNVLDHLTGRSCQVGSSNSAFVSTSSSRRYTEVYLEHRMQEAVEAERAGRGTGHFIGYIYEVRADNNFYGAASSYFEYVDTYGDNAGRILAGALATYQSGYLAHRRIPPENIRRVTRVYHNGITGETTTTEYSNARYVSQQTRANPNPYTS

SEQ ID NO: 72 - RNA, 3467 nt, Artificial sequence
  Synthetic
gggaaauaagagagaaaagaagaguaagaagaaauauaagaccccggcgccgccaccauggagacaccagcccaguuauuauuccuccuucuccuauggcuuccagacaccaccggccuuaagcucccuagccuccuaaccgacgacgagcuuaagcucgugcuuccuaccggcaugagccucgaggacuucaagagaagccuccaggagagcgccccuagcgcccuggccaccccuccuagcagcagcccuccuguggccaagccuggcccuggcagcguggccgaggccccaucgggcagcggccacaaggauaacccuagcccuccaguugugggcguggguccuggaauggccgagagcagcggcggucauaacccuggcgugggcggcggcacccacgagaacggccugccuggcaucggcaaggugggaggcagugccccugguccugacacuagcaccggcucuggcccagaugccggaauggcaagcggcgccggcagcaccagcccuggcgcuucaggaaugccuccaagugagggcgaacggccugauuccggaauguccgauagcggcagaggcggagaaucaagcgccggcggacugaacccugauggcgcaggcaagccaccuagagaggagggugagccagguucuaagagcccugccgauggcggccaggacggcccuccaccaccucgagacggcggagacgccgacccucaaccaccacgggacgacggcaacggcgaacagcagccuccaaagggcggaggugaugagggccaaaggccuccuccugccgcgggcaauggugguaacggcgguaauggaaacgcgcagcuaccugagagaggcgacgacgccgguccuaagccgccagagggugaagguggagacgagggaccacagccaccucaaggaggaggcgagcaagacgccccugaggugccaccuguggcuccagcaccaccagccgguaacgguguguacgacccugguacccacacccugaccacuccugcaucggccgccgucucacuugcgucuagcagccaugguguuuggcaggccgagaugaaccgggccaagagaagcggaagcggcgcucccgugaagcagacccugaacuucgaccugcugaagcuggccggcgacguggagagcaaccccggccccauggagacgccugcccagcugcuguuccugcugcuucuguggcugcccgacaccaccggcggcagcccugguggcagagccccaucugcgccucagccugcuccaagucccagacccgagccugccccagaaccagccccaaacccagcuccuaggccagcuccacagccacccgcacccgcucccggugcaccccgcccaccagcaccuccgccugaggcgccaccuccggugaugccaccgccagcugugccuccucagcuucccgaggugcccgcugccgaucugcccagagugcgagccccgcugagcaccuacagaagaccccagagaaccgacuucgugaccccuacaggaggucccuucuucgccaagcaggacaaggcccugaacaccaucgaccugaagauggcccacgaucugaagcugagaggcuacagagugaagguggccgugguggacgagggcgugagaagcgaccacccgcugcugaacguggagaagaaguacggcggcgacuacauggccgacggcaccagaaccuaccccgaccccaagagacagggcagacacggcacuagcguggcacuugugcuggccggccaggacaccgauaccuaccgcggcggaguggcuccgaacgcggaucuguacagcgccaacaucggaacacgugccggccacgugagcgacgaggccgccuuccacgccuggaacgaccugcugggccacggcaucaagaucuucaacaacagcuucgccaccgagggccccgagggcgagcagaggguuaaggaggacagaaacgaguaccacagcgccgccaacaagcagaacacauacauuggcagacuggacagacuggugagagacggcgcuuugcucaucuucgccgccggcaacggcagacccuccggcagagcuuacagcgaggugggcagcgugggcagaaccccuagagucgagccccaccugcagagaggccugaucgugguuaccgccguggacgagaacggccgacucgaaacuugggccaacaggugcggccaggcccagcaguggugccuggcugcacccuccaccgccuaccugcccggccuggacaaggacaaccccgacagcauccacgucgagcagggcaccgcgcucagcgccccucuuguuacaggcgcugccguccuggugcaggacagauuccgcuggauggacaacgacaaccugagaaccacucugcuaaccaccgcccaggauaagggccccuacggcguggacccucaguacggcuggggcgugcuggacguuggucgggcagugcagggcccugcacaguucgccuucggagauuucguggcacgcgugacugauaccucaacuuucggcaacgacaucuccggcgcuggugggcugguaguggacggcccaggcgccuuaguucucgcaggcagcaacaccuacgccggaaggacuacaaucaagagaggcacccuggacguauucggaucggugaccagcgccgugaccguggaaccagguggcacucugaccggcauuggcacugugggcaccgucaccaaccaggguaccguugugaacaaggaggccggccugcacgugaagggcgacuauagccagacagcacagggccugcucgugaccgacauaggcucucuucuggacgugaguggacgcgccucccuggccggacgccuucacgucgacgacauaagacccggcuacgugggcggugacgggaaguccgugcccgugaucaaggccggcgcaguuagcggcguguucgccacacucacuagaaguccaggacugcuccuuaacgcuaggcucgauuaccggccacaggccguguaccugaccaugcgucgggccgagcgugugcacgcggccgcccagcgcggagccgacgacggucggcgggccucuguccuugccgucgccgagagguuagacgccgccaugagagagcuggacgcccugccagagucucagcgugacgcugccgcaccagccgcggccaucggaaggauccagagaguacagagcagaaaggugcugcaagauaaccucuacagccuugcuggcgcuacauacgcuaacgccgccgcugucaauacacuggaacagaaccgguggauggauagacucgagaauugauaauaggcuggagccucgguggccuagcuucuugccccuugggccuccccccagccccuccuccccuuccugcacccguacccccguggucuuugaauaaagucugagugggcggc

SEQ ID NO: 73 - RNA, 3291 nt, Artificial sequence
  Synthetic
auggagacaccagcccaguuauuauuccuccuucuccuauggcuuccagacaccaccggccuuaagcucccuagccuccuaaccgacgacgagcuuaagcucgugcuuccuaccggcaugagccucgaggacuucaagagaagccuccaggagagcgccccuagcgcccuggccaccccuccuagcagcagcccuccuguggccaagccuggcccuggcagcguggccgaggccccaucgggcagcggccacaaggauaacccuagcccuccaguugugggcguggguccuggaauggccgagagcagcggcggucauaacccuggcgugggcggcggcacccacgagaacggccugccuggcaucggcaaggugggaggcagugccccugguccugacacuagcaccggcucuggcccagaugccggaauggcaagcggcgccggcagcaccagcccuggcgcuucaggaaugccuccaagugagggcgaacggccugauuccggaauguccgauagcggcagaggcggagaaucaagcgccggcggacugaacccugauggcgcaggcaagccaccuagagaggagggugagccagguucuaagagcccugccgauggcggccaggacggcccuccaccaccucgagacggcggagacgccgacccucaaccaccacgggacgacggcaacggcgaacagcagccuccaaagggcggaggugaugagggccaaaggccuccuccugccgcgggcaauggugguaacggcgguaauggaaacgcgcagcuaccugagagaggcgacgacgccgguccuaagccgccagagggugaagguggagacgagggaccacagccaccucaaggaggaggcgagcaagacgccccugaggugccaccuguggcuccagcaccaccagccgguaacgguguguacgacccugguacccacacccugaccacuccugcaucggccgccgucucacuugcgucuagcagccaugguguuuggcaggccgagaugaaccgggccaagagaagcggaagcggcgcucccgugaagcagacccugaacuucgaccugcugaagcuggccggcgacguggagagcaaccccggccccauggagacgccugcccagcugcuguuccugcugcuucuguggcugcccgacaccaccggcggcagcccugguggcagagccccaucugcgccucagccugcuccaagucccagacccgagccugccccagaaccagccccaaacccagcuccuaggccagcuccacagccacccgcacccgcucccggugcaccccgcccaccagcaccuccgccugaggcgccaccuccggugaugccaccgccagcugugccuccucagcuucccgaggugcccgcugccgaucugcccagagugcgagccccgcugagcaccuacagaagaccccagagaaccgacuucgugaccccuacaggaggucccuucuucgccaagcaggacaaggcccugaacaccaucgaccugaagauggcccacgaucugaagcugagaggcuacagagugaagguggccgugguggacgagggcgugagaagcgaccacccgcugcugaacguggagaagaaguacggcggcgacuacauggccgacggcaccagaaccuaccccgaccccaagagacagggcagacacggcacuagcguggcacuugugcuggccggccaggacaccgauaccuaccgcggcggaguggcuccgaacgcggaucuguacagcgccaacaucggaacacgugccggccacgugagcgacgaggccgccuuccacgccuggaacgaccugcugggccacggcaucaagaucuucaacaacagcuucgccaccgagggccccgagggcgagcagaggguuaaggaggacagaaacgaguaccacagcgccgccaacaagcagaacacauacauuggcagacuggacagacuggugagagacggcgcuuugcucaucuucgccgccggcaacggcagacccuccggcagagcuuacagcgaggugggcagcgugggcagaaccccuagagucgagccccaccugcagagaggccugaucgugguuaccgccguggacgagaacggccgacucgaaacuugggccaacaggugcggccaggcccagcaguggugccuggcugcacccuccaccgccuaccugcccggccuggacaaggacaaccccgacagcauccacgucgagcagggcaccgcgcucagcgccccucuuguuacaggcgcugccguccuggugcaggacagauuccgcuggauggacaacgacaaccugagaaccacucugcuaaccaccgcccaggauaagggccccuacggcguggacccucaguacggcuggggcgugcuggacguuggucgggcagugcagggcccugcacaguucgccuucggagauuucguggcacgcgugacugauaccucaacuuucggcaacgacaucuccggcgcuggugggcugguaguggacggcccaggcgccuuaguucucgcaggcagcaacaccuacgccggaaggacuacaaucaagagaggcacccuggacguauucggaucggugaccagcgccgugaccguggaaccagguggcacucugaccggcauuggcacugugggcaccgucaccaaccaggguaccguugugaacaaggaggccggccugcacgugaagggcgacuauagccagacagcacagggccugcucgugaccgacauaggcucucuucuggacgugaguggacgcgccucccuggccggacgccuucacgucgacgacauaagacccggcuacgugggcggugacgggaaguccgugcccgugaucaaggccggcgcaguuagcggcguguucgccacacucacuagaaguccaggacugcuccuuaacgcuaggcucgauuaccggccacaggccguguaccugaccaugcgucgggccgagcgugugcacgcggccgcccagcgcggagccgacgacggucggcgggccucuguccuugccgucgccgagagguuagacgccgccaugagagagcuggacgcccugccagagucucagcgugacgcugccgcaccagccgcggccaucggaaggauccagagaguacagagcagaaaggugcugcaagauaaccucuacagccuugcuggcgcuacauacgcuaacgccgccgcugucaauacacuggaacagaaccgguggauggauagacucgagaau

SEQ ID NO: 74 - protein, 1097 aa, Artificial sequence
  Synthetic
METPAQLLFLLLLWLPDTTGLKLPSLLTDDELKLVLPTGMSLEDFKRSLQESAPSALATPPSSSPPVAKPGPGSVAEAPSGSGHKDNPSPPVVGVGPGMAESSGGHNPGVGGGTHENGLPGIGKVGGSAPGPDTSTGSGPDAGMASGAGSTSPGASGMPPSEGERPDSGMSDSGRGGESSAGGLNPDGAGKPPREEGEPGSKSPADGGQDGPPPPRDGGDADPQPPRDDGNGEQQPPKGGGDEGQRPPPAAGNGGNGGNGNAQLPERGDDAGPKPPEGEGGDEGPQPPQGGGEQDAPEVPPVAPAPPAGNGVYDPGTHTLTTPASAAVSLASSSHGVWQAEMNRAKRSGSGAPVKQTLNFDLLKLAGDVESNPGPMETPAQLLFLLLLWLPDTTGGSPGGRAPSAPQPAPSPRPEPAPEPAPNPAPRPAPQPPAPAPGAPRPPAPPPEAPPPVMPPPAVPPQLPEVPAADLPRVRAPLSTYRRPQRTDFVTPTGGPFFAKQDKALNTIDLKMAHDLKLRGYRVKVAVVDEGVRSDHPLLNVEKKYGGDYMADGTRTYPDPKRQGRHGTSVALVLAGQDTDTYRGGVAPNADLYSANIGTRAGHVSDEAAFHAWNDLLGHGIKIFNNSFATEGPEGEQRVKEDRNEYHSAANKQNTYIGRLDRLVRDGALLIFAAGNGRPSGRAYSEVGSVGRTPRVEPHLQRGLIVVTAVDENGRLETWANRCGQAQQWCLAAPSTAYLPGLDKDNPDSIHVEQGTALSAPLVTGAAVLVQDRFRWMDNDNLRTTLLTTAQDKGPYGVDPQYGWGVLDVGRAVQGPAQFAFGDFVARVTDTSTFGNDISGAGGLVVDGPGALVLAGSNTYAGRTTIKRGTLDVFGSVTSAVTVEPGGTLTGIGTVGTVTNQGTVVNKEAGLHVKGDYSQTAQGLLVTDIGSLLDVSGRASLAGRLHVDDIRPGYVGGDGKSVPVIKAGAVSGVFATLTRSPGLLLNARLDYRPQAVYLTMRRAERVHAAAQRGADDGRRASVLAVAERLDAAMRELDALPESQRDAAAPAAAIGRIQRVQSRKVLQDNLYSLAGATYANAAAVNTLEQNRWMDRLEN

SEQ ID NO: 75 - RNA, 3371 nt, Artificial sequence
  Synthetic
gggaaauaagagagaaaagaagaguaagaagaaauauaagaccccggcgccgccaccauggagacaccagcccaguuauuauuccuccuucuccuauggcuuccagacaccaccggccuuaagcucccuagccuccuaaccgacgacgagcuuaagcucgugcuuccuaccggcaugagccucgaggacuucaagagaagccuccaggagagcgccccuagcgcccuggccaccccuccuagcagcagcccuccuguggccaagccuggcccuggcagcguggccgaggccccaucgggcagcggccacaaggauaacccuagcccuccaguugugggcguggguccuggaauggccgagagcagcggcggucauaacccuggcgugggcggcggcacccacgagaacggccugccuggcaucggcaaggugggaggcagugccccugguccugacacuagcaccggcucuggcccagaugccggaauggcaagcggcgccggcagcaccagcccuggcgcuucaggaaugccuccaagugagggcgaacggccugauuccggaauguccgauagcggcagaggcggagaaucaagcgccggcggacugaacccugauggcgcaggcaagccaccuagagaggagggugagccagguucuaagagcccugccgauggcggccaggacggcccuccaccaccucgagacggcggagacgccgacccucaaccaccacgggacgacggcaacggcgaacagcagccuccaaagggcggaggugaugagggccaaaggccuccuccugccgcgggcaauggugguaacggcgguaauggaaacgcgcagcuaccugagagaggcgacgacgccgguccuaagccgccagagggugaagguggagacgagggaccacagccaccucaaggaggaggcgagcaagacgccccugaggugccaccuguggcuccagcaccaccagccgguaacgguguguacgacccugguacccacacccugaccacuccugcaucggccgccgucucacuugcgucuagcagccaugguguuuggcaggccgagaugaacggaggagguggcucuggcggagguggaagcgguggaggcggaagugguggcggaggcagcggcagcccugguggcagagccccaucugcgccucagccugcuccaagucccagacccgagccugccccagaaccagccccaaacccagcuccuaggccagcuccacagccacccgcacccgcucccggugcaccccgcccaccagcaccuccgccugaggcgccaccuccggugaugccaccgccagcugugccuccucagcuucccgaggugcccgcugccgaucugcccagagugcgagccccgcugagcaccuacagaagaccccagagaaccgacuucgugaccccuacaggaggucccuucuucgccaagcaggacaaggcccugaacaccaucgaccugaagauggcccacgaucugaagcugagaggcuacagagugaagguggccgugguggacgagggcgugagaagcgaccacccgcugcugaacguggagaagaaguacggcggcgacuacauggccgacggcaccagaaccuaccccgaccccaagagacagggcagacacggcacuagcguggcacuugugcuggccggccaggacaccgauaccuaccgcggcggaguggcuccgaacgcggaucuguacagcgccaacaucggaacacgugccggccacgugagcgacgaggccgccuuccacgccuggaacgaccugcugggccacggcaucaagaucuucaacaacagcuucgccaccgagggccccgagggcgagcagaggguuaaggaggacagaaacgaguaccacagcgccgccaacaagcagaacacauacauuggcagacuggacagacuggugagagacggcgcuuugcucaucuucgccgccggcaacggcagacccuccggcagagcuuacagcgaggugggcagcgugggcagaaccccuagagucgagccccaccugcagagaggccugaucgugguuaccgccguggacgagaacggccgacucgaaacuugggccaacaggugcggccaggcccagcaguggugccuggcugcacccuccaccgccuaccugcccggccuggacaaggacaaccccgacagcauccacgucgagcagggcaccgcgcucagcgccccucuuguuacaggcgcugccguccuggugcaggacagauuccgcuggauggacaacgacaaccugagaaccacucugcuaaccaccgcccaggauaagggccccuacggcguggacccucaguacggcuggggcgugcuggacguuggucgggcagugcagggcccugcacaguucgccuucggagauuucguggcacgcgugacugauaccucaacuuucggcaacgacaucuccggcgcuggugggcugguaguggacggcccaggcgccuuaguucucgcaggcagcaacaccuacgccggaaggacuacaaucaagagaggcacccuggacguauucggaucggugaccagcgccgugaccguggaaccagguggcacucugaccggcauuggcacugugggcaccgucaccaaccaggguaccguugugaacaaggaggccggccugcacgugaagggcgacuauagccagacagcacagggccugcucgugaccgacauaggcucucuucuggacgugaguggacgcgccucccuggccggacgccuucacgucgacgacauaagacccggcuacgugggcggugacgggaaguccgugcccgugaucaaggccggcgcaguuagcggcguguucgccacacucacuagaaguccaggacugcuccuuaacgcuaggcucgauuaccggccacaggccguguaccugaccaugcgucgggccgagcgugugcacgcggccgcccagcgcggagccgacgacggucggcgggccucuguccuugccgucgccgagagguuagacgccgccaugagagagcuggacgcccugccagagucucagcgugacgcugccgcaccagccgcggccaucggaaggauccagagaguacagagcagaaaggugcugcaagauaaccucuacagccuugcuggcgcuacauacgcuaacgccgccgcugucaauacacuggaacagaaccgguggauggauagacucgagaauugauaauaggcuggagccucgguggccuagcuucuugccccuugggccuccccccagccccuccuccccuuccugcacccguacccccguggucuuugaauaaagucugagugggcggc

SEQ ID NO: 76 - RNA, 3195 nt, Artificial sequence
  Synthetic
auggagacaccagcccaguuauuauuccuccuucuccuauggcuuccagacaccaccggccuuaagcucccuagccuccuaaccgacgacgagcuuaagcucgugcuuccuaccggcaugagccucgaggacuucaagagaagccuccaggagagcgccccuagcgcccuggccaccccuccuagcagcagcccuccuguggccaagccuggcccuggcagcguggccgaggccccaucgggcagcggccacaaggauaacccuagcccuccaguugugggcguggguccuggaauggccgagagcagcggcggucauaacccuggcgugggcggcggcacccacgagaacggccugccuggcaucggcaaggugggaggcagugccccugguccugacacuagcaccggcucuggcccagaugccggaauggcaagcggcgccggcagcaccagcccuggcgcuucaggaaugccuccaagugagggcgaacggccugauuccggaauguccgauagcggcagaggcggagaaucaagcgccggcggacugaacccugauggcgcaggcaagccaccuagagaggagggugagccagguucuaagagcccugccgauggcggccaggacggcccuccaccaccucgagacggcggagacgccgacccucaaccaccacgggacgacggcaacggcgaacagcagccuccaaagggcggaggugaugagggccaaaggccuccuccugccgcgggcaauggugguaacggcgguaauggaaacgcgcagcuaccugagagaggcgacgacgccgguccuaagccgccagagggugaagguggagacgagggaccacagccaccucaaggaggaggcgagcaagacgccccugaggugccaccuguggcuccagcaccaccagccgguaacgguguguacgacccugguacccacacccugaccacuccugcaucggccgccgucucacuugcgucuagcagccaugguguuuggcaggccgagaugaacggaggagguggcucuggcggagguggaagcgguggaggcggaagugguggcggaggcagcggcagcccugguggcagagccccaucugcgccucagccugcuccaagucccagacccgagccugccccagaaccagccccaaacccagcuccuaggccagcuccacagccacccgcacccgcucccggugcaccccgcccaccagcaccuccgccugaggcgccaccuccggugaugccaccgccagcugugccuccucagcuucccgaggugcccgcugccgaucugcccagagugcgagccccgcugagcaccuacagaagaccccagagaaccgacuucgugaccccuacaggaggucccuucuucgccaagcaggacaaggcccugaacaccaucgaccugaagauggcccacgaucugaagcugagaggcuacagagugaagguggccgugguggacgagggcgugagaagcgaccacccgcugcugaacguggagaagaaguacggcggcgacuacauggccgacggcaccagaaccuaccccgaccccaagagacagggcagacacggcacuagcguggcacuugugcuggccggccaggacaccgauaccuaccgcggcggaguggcuccgaacgcggaucuguacagcgccaacaucggaacacgugccggccacgugagcgacgaggccgccuuccacgccuggaacgaccugcugggccacggcaucaagaucuucaacaacagcuucgccaccgagggccccgagggcgagcagaggguuaaggaggacagaaacgaguaccacagcgccgccaacaagcagaacacauacauuggcagacuggacagacuggugagagacggcgcuuugcucaucuucgccgccggcaacggcagacccuccggcagagcuuacagcgaggugggcagcgugggcagaaccccuagagucgagccccaccugcagagaggccugaucgugguuaccgccguggacgagaacggccgacucgaaacuugggccaacaggugcggccaggcccagcaguggugccuggcugcacccuccaccgccuaccugcccggccuggacaaggacaaccccgacagcauccacgucgagcagggcaccgcgcucagcgccccucuuguuacaggcgcugccguccuggugcaggacagauuccgcuggauggacaacgacaaccugagaaccacucugcuaaccaccgcccaggauaagggccccuacggcguggacccucaguacggcuggggcgugcuggacguuggucgggcagugcagggcccugcacaguucgccuucggagauuucguggcacgcgugacugauaccucaacuuucggcaacgacaucuccggcgcuggugggcugguaguggacggcccaggcgccuuaguucucgcaggcagcaacaccuacgccggaaggacuacaaucaagagaggcacccuggacguauucggaucggugaccagcgccgugaccguggaaccagguggcacucugaccggcauuggcacugugggcaccgucaccaaccaggguaccguugugaacaaggaggccggccugcacgugaagggcgacuauagccagacagcacagggccugcucgugaccgacauaggcucucuucuggacgugaguggacgcgccucccuggccggacgccuucacgucgacgacauaagacccggcuacgugggcggugacgggaaguccgugcccgugaucaaggccggcgcaguuagcggcguguucgccacacucacuagaaguccaggacugcuccuuaacgcuaggcucgauuaccggccacaggccguguaccugaccaugcgucgggccgagcgugugcacgcggccgcccagcgcggagccgacgacggucggcgggccucuguccuugccgucgccgagagguuagacgccgccaugagagagcuggacgcccugccagagucucagcgugacgcugccgcaccagccgcggccaucggaaggauccagagaguacagagcagaaaggugcugcaagauaaccucuacagccuugcuggcgcuacauacgcuaacgccgccgcugucaauacacuggaacagaaccgguggauggauagacucgagaau

SEQ ID NO: 77 - protein, 1065 aa, Artificial sequence
  Synthetic
METPAQLLFLLLLWLPDTTGLKLPSLLTDDELKLVLPTGMSLEDFKRSLQESAPSALATPPSSSPPVAKPGPGSVAEAPSGSGHKDNPSPPVVGVGPGMAESSGGHNPGVGGGTHENGLPGIGKVGGSAPGPDTSTGSGPDAGMASGAGSTSPGASGMPPSEGERPDSGMSDSGRGGESSAGGLNPDGAGKPPREEGEPGSKSPADGGQDGPPPPRDGGDADPQPPRDDGNGEQQPPKGGGDEGQRPPPAAGNGGNGGNGNAQLPERGDDAGPKPPEGEGGDEGPQPPQGGGEQDAPEVPPVAPAPPAGNGVYDPGTHTLTTPASAAVSLASSSHGVWQAEMNGGGGSGGGGSGGGGSGGGGSGSPGGRAPSAPQPAPSPRPEPAPEPAPNPAPRPAPQPPAPAPGAPRPPAPPPEAPPPVMPPPAVPPQLPEVPAADLPRVRAPLSTYRRPQRTDFVTPTGGPFFAKQDKALNTIDLKMAHDLKLRGYRVKVAVVDEGVRSDHPLLNVEKKYGGDYMADGTRTYPDPKRQGRHGTSVALVLAGQDTDTYRGGVAPNADLYSANIGTRAGHVSDEAAFHAWNDLLGHGIKIFNNSFATEGPEGEQRVKEDRNEYHSAANKQNTYIGRLDRLVRDGALLIFAAGNGRPSGRAYSEVGSVGRTPRVEPHLQRGLIVVTAVDENGRLETWANRCGQAQQWCLAAPSTAYLPGLDKDNPDSIHVEQGTALSAPLVTGAAVLVQDRFRWMDNDNLRTTLLTTAQDKGPYGVDPQYGWGVLDVGRAVQGPAQFAFGDFVARVTDTSTFGNDISGAGGLVVDGPGALVLAGSNTYAGRTTIKRGTLDVFGSVTSAVTVEPGGTLTGIGTVGTVTNQGTVVNKEAGLHVKGDYSQTAQGLLVTDIGSLLDVSGRASLAGRLHVDDIRPGYVGGDGKSVPVIKAGAVSGVFATLTRSPGLLLNARLDYRPQAVYLTMRRAERVHAAAQRGADDGRRASVLAVAERLDAAMRELDALPESQRDAAAPAAAIGRIQRVQSRKVLQDNLYSLAGATYANAAAVNTLEQNRWMDRLEN

SEQ ID NO: 78 - RNA, 3371 nt, Artificial sequence
  Synthetic
gggaaauaagagagaaaagaagaguaagaagaaauauaagaccccggcgccgccaccauggagacaccagcccaguuauuauuccuccuucuccuauggcuuccagacaccaccggcggcagcccugguggcagagccccaucugcgccucagccugcuccaagucccagacccgagccugccccagaaccagccccaaacccagcuccuaggccagcuccacagccacccgcacccgcucccggugcaccccgcccaccagcaccuccgccugaggcgccaccuccggugaugccaccgccagcugugccuccucagcuucccgaggugcccgcugccgaucugcccagagugcgagccccgcugagcaccuacagaagaccccagagaaccgacuucgugaccccuacaggaggucccuucuucgccaagcaggacaaggcccugaacaccaucgaccugaagauggcccacgaucugaagcugagaggcuacagagugaagguggccgugguggacgagggcgugagaagcgaccacccgcugcugaacguggagaagaaguacggcggcgacuacauggccgacggcaccagaaccuaccccgaccccaagagacagggcagacacggcacuagcguggcacuugugcuggccggccaggacaccgauaccuaccgcggcggaguggcuccgaacgcggaucuguacagcgccaacaucggaacacgugccggccacgugagcgacgaggccgccuuccacgccuggaacgaccugcugggccacggcaucaagaucuucaacaacagcuucgccaccgagggccccgagggcgagcagaggguuaaggaggacagaaacgaguaccacagcgccgccaacaagcagaacacauacauuggcagacuggacagacuggugagagacggcgcuuugcucaucuucgccgccggcaacggcagacccuccggcagagcuuacagcgaggugggcagcgugggcagaaccccuagagucgagccccaccugcagagaggccugaucgugguuaccgccguggacgagaacggccgacucgaaacuugggccaacaggugcggccaggcccagcaguggugccuggcugcacccuccaccgccuaccugcccggccuggacaaggacaaccccgacagcauccacgucgagcagggcaccgcgcucagcgccccucuuguuacaggcgcugccguccuggugcaggacagauuccgcuggauggacaacgacaaccugagaaccacucugcuaaccaccgcccaggauaagggccccuacggcguggacccucaguacggcuggggcgugcuggacguuggucgggcagugcagggcccugcacaguucgccuucggagauuucguggcacgcgugacugauaccucaacuuucggcaacgacaucuccggcgcuggugggcugguaguggacggcccaggcgccuuaguucucgcaggcagcaacaccuacgccggaaggacuacaaucaagagaggcacccuggacguauucggaucggugaccagcgccgugaccguggaaccagguggcacucugaccggcauuggcacugugggcaccgucaccaaccaggguaccguugugaacaaggaggccggccugcacgugaagggcgacuauagccagacagcacagggccugcucgugaccgacauaggcucucuucuggacgugaguggacgcgccucccuggccggacgccuucacgucgacgacauaagacccggcuacgugggcggugacgggaaguccgugcccgugaucaaggccggcgcaguuagcggcguguucgccacacucacuagaaguccaggacugcuccuuaacgcuaggcucgauuaccggccacaggccguguaccugaccaugcgucgggccgagcgugugcacgcggccgcccagcgcggagccgacgacggucggcgggccucuguccuugccgucgccgagagguuagacgccgccaugagagagcuggacgcccugccagagucucagcgugacgcugccgcaccagccgcggccaucggaaggauccagagaguacagagcagaaaggugcugcaagauaaccucuacagccuugcuggcgcuacauacgcuaacgccgccgcugucaauacacuggaacagaaccgguggauggauagacucgagaauggaggagguggcucuggcggagguggaagcgguggaggcggaagugguggcggaggcagccuuaagcucccuagccuccuaaccgacgacgagcuuaagcucgugcuuccuaccggcaugagccucgaggacuucaagagaagccuccaggagagcgccccuagcgcccuggccaccccuccuagcagcagcccuccuguggccaagccuggcccuggcagcguggccgaggccccaucgggcagcggccacaaggauaacccuagcccuccaguugugggcguggguccuggaauggccgagagcagcggcggucauaacccuggcgugggcggcggcacccacgagaacggccugccuggcaucggcaaggugggaggcagugccccugguccugacacuagcaccggcucuggcccagaugccggaauggcaagcggcgccggcagcaccagcccuggcgcuucaggaaugccuccaagugagggcgaacggccugauuccggaauguccgauagcggcagaggcggagaaucaagcgccggcggacugaacccugauggcgcaggcaagccaccuagagaggagggugagccagguucuaagagcccugccgauggcggccaggacggcccuccaccaccucgagacggcggagacgccgacccucaaccaccacgggacgacggcaacggcgaacagcagccuccaaagggcggaggugaugagggccaaaggccuccuccugccgcgggcaauggugguaacggcgguaauggaaacgcgcagcuaccugagagaggcgacgacgccgguccuaagccgccagagggugaagguggagacgagggaccacagccaccucaaggaggaggcgagcaagacgccccugaggugccaccuguggcuccagcaccaccagccgguaacgguguguacgacccugguacccacacccugaccacuccugcaucggccgccgucucacuugcgucuagcagccaugguguuuggcaggccgagaugaacugauaauaggcuggagccucgguggccuagcuucuugccccuugggccuccccccagccccuccuccccuuccugcacccguacccccguggucuuugaauaaagucugagugggcggc

SEQ ID NO: 79 - RNA, 3195 nt, Artificial sequence
  Synthetic
auggagacaccagcccaguuauuauuccuccuucuccuauggcuuccagacaccaccggcggcagcccugguggcagagccccaucugcgccucagccugcuccaagucccagacccgagccugccccagaaccagccccaaacccagcuccuaggccagcuccacagccacccgcacccgcucccggugcaccccgcccaccagcaccuccgccugaggcgccaccuccggugaugccaccgccagcugugccuccucagcuucccgaggugcccgcugccgaucugcccagagugcgagccccgcugagcaccuacagaagaccccagagaaccgacuucgugaccccuacaggaggucccuucuucgccaagcaggacaaggcccugaacaccaucgaccugaagauggcccacgaucugaagcugagaggcuacagagugaagguggccgugguggacgagggcgugagaagcgaccacccgcugcugaacguggagaagaaguacggcggcgacuacauggccgacggcaccagaaccuaccccgaccccaagagacagggcagacacggcacuagcguggcacuugugcuggccggccaggacaccgauaccuaccgcggcggaguggcuccgaacgcggaucuguacagcgccaacaucggaacacgugccggccacgugagcgacgaggccgccuuccacgccuggaacgaccugcugggccacggcaucaagaucuucaacaacagcuucgccaccgagggccccgagggcgagcagaggguuaaggaggacagaaacgaguaccacagcgccgccaacaagcagaacacauacauuggcagacuggacagacuggugagagacggcgcuuugcucaucuucgccgccggcaacggcagacccuccggcagagcuuacagcgaggugggcagcgugggcagaaccccuagagucgagccccaccugcagagaggccugaucgugguuaccgccguggacgagaacggccgacucgaaacuugggccaacaggugcggccaggcccagcaguggugccuggcugcacccuccaccgccuaccugcccggccuggacaaggacaaccccgacagcauccacgucgagcagggcaccgcgcucagcgccccucuuguuacaggcgcugccguccuggugcaggacagauuccgcuggauggacaacgacaaccugagaaccacucugcuaaccaccgcccaggauaagggccccuacggcguggacccucaguacggcuggggcgugcuggacguuggucgggcagugcagggcccugcacaguucgccuucggagauuucguggcacgcgugacugauaccucaacuuucggcaacgacaucuccggcgcuggugggcugguaguggacggcccaggcgccuuaguucucgcaggcagcaacaccuacgccggaaggacuacaaucaagagaggcacccuggacguauucggaucggugaccagcgccgugaccguggaaccagguggcacucugaccggcauuggcacugugggcaccgucaccaaccaggguaccguugugaacaaggaggccggccugcacgugaagggcgacuauagccagacagcacagggccugcucgugaccgacauaggcucucuucuggacgugaguggacgcgccucccuggccggacgccuucacgucgacgacauaagacccggcuacgugggcggugacgggaaguccgugcccgugaucaaggccggcgcaguuagcggcguguucgccacacucacuagaaguccaggacugcuccuuaacgcuaggcucgauuaccggccacaggccguguaccugaccaugcgucgggccgagcgugugcacgcggccgcccagcgcggagccgacgacggucggcgggccucuguccuugccgucgccgagagguuagacgccgccaugagagagcuggacgcccugccagagucucagcgugacgcugccgcaccagccgcggccaucggaaggauccagagaguacagagcagaaaggugcugcaagauaaccucuacagccuugcuggcgcuacauacgcuaacgccgccgcugucaauacacuggaacagaaccgguggauggauagacucgagaauggaggagguggcucuggcggagguggaagcgguggaggcggaagugguggcggaggcagccuuaagcucccuagccuccuaaccgacgacgagcuuaagcucgugcuuccuaccggcaugagccucgaggacuucaagagaagccuccaggagagcgccccuagcgcccuggccaccccuccuagcagcagcccuccuguggccaagccuggcccuggcagcguggccgaggccccaucgggcagcggccacaaggauaacccuagcccuccaguugugggcguggguccuggaauggccgagagcagcggcggucauaacccuggcgugggcggcggcacccacgagaacggccugccuggcaucggcaaggugggaggcagugccccugguccugacacuagcaccggcucuggcccagaugccggaauggcaagcggcgccggcagcaccagcccuggcgcuucaggaaugccuccaagugagggcgaacggccugauuccggaauguccgauagcggcagaggcggagaaucaagcgccggcggacugaacccugauggcgcaggcaagccaccuagagaggagggugagccagguucuaagagcccugccgauggcggccaggacggcccuccaccaccucgagacggcggagacgccgacccucaaccaccacgggacgacggcaacggcgaacagcagccuccaaagggcggaggugaugagggccaaaggccuccuccugccgcgggcaauggugguaacggcgguaauggaaacgcgcagcuaccugagagaggcgacgacgccgguccuaagccgccagagggugaagguggagacgagggaccacagccaccucaaggaggaggcgagcaagacgccccugaggugccaccuguggcuccagcaccaccagccgguaacgguguguacgacccugguacccacacccugaccacuccugcaucggccgccgucucacuugcgucuagcagccaugguguuuggcaggccgagaugaac

SEQ ID NO: 80 - protein, 1065 aa, Artificial sequence
  Synthetic
METPAQLLFLLLLWLPDTTGGSPGGRAPSAPQPAPSPRPEPAPEPAPNPAPRPAPQPPAPAPGAPRPPAPPPEAPPPVMPPPAVPPQLPEVPAADLPRVRAPLSTYRRPQRTDFVTPTGGPFFAKQDKALNTIDLKMAHDLKLRGYRVKVAVVDEGVRSDHPLLNVEKKYGGDYMADGTRTYPDPKRQGRHGTSVALVLAGQDTDTYRGGVAPNADLYSANIGTRAGHVSDEAAFHAWNDLLGHGIKIFNNSFATEGPEGEQRVKEDRNEYHSAANKQNTYIGRLDRLVRDGALLIFAAGNGRPSGRAYSEVGSVGRTPRVEPHLQRGLIVVTAVDENGRLETWANRCGQAQQWCLAAPSTAYLPGLDKDNPDSIHVEQGTALSAPLVTGAAVLVQDRFRWMDNDNLRTTLLTTAQDKGPYGVDPQYGWGVLDVGRAVQGPAQFAFGDFVARVTDTSTFGNDISGAGGLVVDGPGALVLAGSNTYAGRTTIKRGTLDVFGSVTSAVTVEPGGTLTGIGTVGTVTNQGTVVNKEAGLHVKGDYSQTAQGLLVTDIGSLLDVSGRASLAGRLHVDDIRPGYVGGDGKSVPVIKAGAVSGVFATLTRSPGLLLNARLDYRPQAVYLTMRRAERVHAAAQRGADDGRRASVLAVAERLDAAMRELDALPESQRDAAAPAAAIGRIQRVQSRKVLQDNLYSLAGATYANAAAVNTLEQNRWMDRLENGGGGSGGGGSGGGGSGGGGSLKLPSLLTDDELKLVLPTGMSLEDFKRSLQESAPSALATPPSSSPPVAKPGPGSVAEAPSGSGHKDNPSPPVVGVGPGMAESSGGHNPGVGGGTHENGLPGIGKVGGSAPGPDTSTGSGPDAGMASGAGSTSPGASGMPPSEGERPDSGMSDSGRGGESSAGGLNPDGAGKPPREEGEPGSKSPADGGQDGPPPPRDGGDADPQPPRDDGNGEQQPPKGGGDEGQRPPPAAGNGGNGGNGNAQLPERGDDAGPKPPEGEGGDEGPQPPQGGGEQDAPEVPPVAPAPPAGNGVYDPGTHTLTTPASAAVSLASSSHGVWQAEMN

SEQ ID NO: 81 - RNA, 3959 nt, Artificial sequence
  Synthetic
gggaaauaagagagaaaagaagaguaagaagaaauauaagaccccggcgccgccaccauggagaccccugcccagcugcuguuccugcugcugcuuuggcugccugacaccaccggaagccuguacgccgaacacgacgccacccugacccuggcccaaggcacccagagggaucuggugguggaccaggaccacauccugcccguggccgagggaacccugagagucaaagccaagagccucaccaccgagaucgagacaggcaacccuggcagccucaucgccgaggugcaggagaacaucgacaacaagcaggccaucgucgugggcaaagaccugacucuguccagcgcccacggcaacguagccaacgaggccaacgcccugcugugggcugccggugagcugaccgugaaggcccagaacaucaccaacaagagagccgcccugauugaggccggcgggaacgccagacugaccgcugccgucgcccuucugaacaagcugggcagaaucagagccggcgaggacaugcaccuggacgccccuagaaucgagaacaccgccaagcugagcggcgaggugcagagaaagggcgugcaggacgucggcggaggcgagcacggcagguggagcggcaucggcuacgugaacuacuggcuucgggccggcaacggcaagaaagccggcaccaucgccgcaccuugguacgguggcgaucugaccgcagagcagagccugaucgaggugggcaaggaccuguaccugaacgccggcgccagaaaggacgagcacagacaccugcugaacgagggcgugauccaggccggaggucacggccacaucggcggcgacguggacaacagggccgugguccguaccgugagcgccauggaguacuucaagaccccucugccugugagccugaccgcccuggacaauagagccggacucagcccagccaccuggaacuuccagagcaccuacgagcugcuggacuaccugcuggaccagaacagauacgaguacaucuggggccuguacccuaccuacaccgaguggagcgugaacacccugaagaaccuggaccugggcuaccaggccaagccugccccuaccgccccuccuaugccuaaggccccugagcuggaccugagaggccauacccuggagagcgccgagggcagaaagaucuucggcgaguacaagaagcugcagggcgaguacgagaaggccaagauggccgugcaagccguggaggccuacggcgaggccaccagaagagugcacgaccagcucgggcagcgauacggcaaggcccugggcggcauggacgccgaaaccaaggagguggacggcaucauccaggaguuugcggccgaucugagaaccguguacgccaaacaggccgaccaagccacuaucgacgccgagacugacaagguggcccagagauacaagagccagaucgacgccgugagacuccaacgggccaagagaagcggaagcggcgcucccgugaagcagacccugaacuucgaccugcugaagcuggccggcgacguggagagcaaccccggccccauggagacuccagcacaguugcuuuuccugcuucugcuguggcugccagacacaacuggaguggauccaccuguggacugcggacgugcauuaggacugcacuucuggucaagugcgagccuuauaucggaucagaccccugacggcacauugaucggcaagccgguggucggacggucacuauugagcaagucuugcaaggugccugacgacauuaaggaagaccugucagacaaucacgacggcgagccaguugauaucguacuggagcuuggcagcaacuauaagauuaggccacagucguacggucauccuggaauaguggucgaccugccuuccgguucaacagaggagacagguaucgcuaucuacaucgacuucggcucuagcccaaugcagaaggucggcgagagacaguggcuguacccacagaagggugaggugcuuuucgacgugcugacuauuaacggagacaacgccgagguucgguaccaggccaucaaggucgguccacugaagcgcccaagaaagcugguccuuagucaguucccaaaccucuucacuuacaagugggucuucaugagaggcacaagccaggagcggguccucgcacagggcaccauagauacugacguggccacgagcaccaucgaccugaagacgugcagauacaccagucagaccgugucgcucccaaucauucagaggagugccuugaccgguguuggcaccacacuuggcaugacugacuuccaaaugccauucugguguuacggcuggccuaaggugucaguuuacaugucugcgacaaagacucaaacaggcguagacggcguagcacucccagcaaccggacaggccgccgguauggccuccggcgugggcguucaguugauuaacgguaagacgcagcagccugucaagcugggacuccagggcaagauugcccugccugaggcacagcagaccgaaagugcaacauucucacugccaaugaaggcccaguacuaccagaccaguaccucuacaucugccggcaagcuguccguuaccuacgccgucacucugaauuacgacggcggaggcuccgacgacggcacaaucgugauaaccgguacaauuacugacacaaccuguguaaucgaggauccuuccggcccaucgcauacuaagguuguucagcugccaaagauuuccaagaacgcccucaaggccaacggagaucaagcugguaggacaccauucauaauuaagcuaaaggacuguccauccucccucgguaacggcgugaaggccuauuucgagccaggaccuacgacugacuacucuacaggugaucucagagcauauaagaugguuuacgcaacaaauccucagacacagcucagcaauaucgucgccgccacugaggcucagggaguccagguccggaucuccaacuuagacgacucaaagaucaccaugggcgccaacgaggcaacucagcaggccgcuggauucgacccagagguacagacuggcgguacgucccgcacagucaccaugcgguaccuggcaaguuacguuaagaagaacggugacguggaggcguccgccauaaccacauacgugggauucucagugguguacccugguggcggcucuggcgguggcaguaacgacgguacuauuguaauuacuggaaguauuucagaccaaacgugcguuaucgaagaaccaagcacauuaaaccauauuaagguggugcaguugccuaagauaucaaagaacgcuuugaggaacgacggcgauacugcaggcgcaacaccuuucgauauaaagcuuaaggagugcccucaggcccugggcgcccugaagcucuacuucgaaccaggcauaacaacuaacuacgacaccggugaucuuauugcuuacaagcagacauauaacgcagccggcaacggccagcuuuccacuguaucauccgccacaaaggcuaagggcgucgaguucaggcuggcgaaucucaacggacaacacauuagaauggguacugauaagaccacccaagcugcacagaccuucacagguaaggucacuaacggcgguaagaguuauacacugcgcuaccuggcuagcuacgugaagaagccaaaggaggacgucgacgcagcacagauaacaucuuacguaggauucucuguuguuuauccggguggcgguagcaacgacggaaccauugugaucacuggcucaauuuccgaccagacagucauuugauaauaggcuggagccucgguggccuagcuucuugccccuugggccuccccccagccccuccuccccuuccugcacccguacccccguggucuuugaauaaagucugagugggcggc

SEQ ID NO: 82 - RNA, 3783 nt, Artificial sequence
  Synthetic
auggagaccccugcccagcugcuguuccugcugcugcuuuggcugccugacaccaccggaagccuguacgccgaacacgacgccacccugacccuggcccaaggcacccagagggaucuggugguggaccaggaccacauccugcccguggccgagggaacccugagagucaaagccaagagccucaccaccgagaucgagacaggcaacccuggcagccucaucgccgaggugcaggagaacaucgacaacaagcaggccaucgucgugggcaaagaccugacucuguccagcgcccacggcaacguagccaacgaggccaacgcccugcugugggcugccggugagcugaccgugaaggcccagaacaucaccaacaagagagccgcccugauugaggccggcgggaacgccagacugaccgcugccgucgcccuucugaacaagcugggcagaaucagagccggcgaggacaugcaccuggacgccccuagaaucgagaacaccgccaagcugagcggcgaggugcagagaaagggcgugcaggacgucggcggaggcgagcacggcagguggagcggcaucggcuacgugaacuacuggcuucgggccggcaacggcaagaaagccggcaccaucgccgcaccuugguacgguggcgaucugaccgcagagcagagccugaucgaggugggcaaggaccuguaccugaacgccggcgccagaaaggacgagcacagacaccugcugaacgagggcgugauccaggccggaggucacggccacaucggcggcgacguggacaacagggccgugguccguaccgugagcgccauggaguacuucaagaccccucugccugugagccugaccgcccuggacaauagagccggacucagcccagccaccuggaacuuccagagcaccuacgagcugcuggacuaccugcuggaccagaacagauacgaguacaucuggggccuguacccuaccuacaccgaguggagcgugaacacccugaagaaccuggaccugggcuaccaggccaagccugccccuaccgccccuccuaugccuaaggccccugagcuggaccugagaggccauacccuggagagcgccgagggcagaaagaucuucggcgaguacaagaagcugcagggcgaguacgagaaggccaagauggccgugcaagccguggaggccuacggcgaggccaccagaagagugcacgaccagcucgggcagcgauacggcaaggcccugggcggcauggacgccgaaaccaaggagguggacggcaucauccaggaguuugcggccgaucugagaaccguguacgccaaacaggccgaccaagccacuaucgacgccgagacugacaagguggcccagagauacaagagccagaucgacgccgugagacuccaacgggccaagagaagcggaagcggcgcucccgugaagcagacccugaacuucgaccugcugaagcuggccggcgacguggagagcaaccccggccccauggagacuccagcacaguugcuuuuccugcuucugcuguggcugccagacacaacuggaguggauccaccuguggacugcggacgugcauuaggacugcacuucuggucaagugcgagccuuauaucggaucagaccccugacggcacauugaucggcaagccgguggucggacggucacuauugagcaagucuugcaaggugccugacgacauuaaggaagaccugucagacaaucacgacggcgagccaguugauaucguacuggagcuuggcagcaacuauaagauuaggccacagucguacggucauccuggaauaguggucgaccugccuuccgguucaacagaggagacagguaucgcuaucuacaucgacuucggcucuagcccaaugcagaaggucggcgagagacaguggcuguacccacagaagggugaggugcuuuucgacgugcugacuauuaacggagacaacgccgagguucgguaccaggccaucaaggucgguccacugaagcgcccaagaaagcugguccuuagucaguucccaaaccucuucacuuacaagugggucuucaugagaggcacaagccaggagcggguccucgcacagggcaccauagauacugacguggccacgagcaccaucgaccugaagacgugcagauacaccagucagaccgugucgcucccaaucauucagaggagugccuugaccgguguuggcaccacacuuggcaugacugacuuccaaaugccauucugguguuacggcuggccuaaggugucaguuuacaugucugcgacaaagacucaaacaggcguagacggcguagcacucccagcaaccggacaggccgccgguauggccuccggcgugggcguucaguugauuaacgguaagacgcagcagccugucaagcugggacuccagggcaagauugcccugccugaggcacagcagaccgaaagugcaacauucucacugccaaugaaggcccaguacuaccagaccaguaccucuacaucugccggcaagcuguccguuaccuacgccgucacucugaauuacgacggcggaggcuccgacgacggcacaaucgugauaaccgguacaauuacugacacaaccuguguaaucgaggauccuuccggcccaucgcauacuaagguuguucagcugccaaagauuuccaagaacgcccucaaggccaacggagaucaagcugguaggacaccauucauaauuaagcuaaaggacuguccauccucccucgguaacggcgugaaggccuauuucgagccaggaccuacgacugacuacucuacaggugaucucagagcauauaagaugguuuacgcaacaaauccucagacacagcucagcaauaucgucgccgccacugaggcucagggaguccagguccggaucuccaacuuagacgacucaaagaucaccaugggcgccaacgaggcaacucagcaggccgcuggauucgacccagagguacagacuggcgguacgucccgcacagucaccaugcgguaccuggcaaguuacguuaagaagaacggugacguggaggcguccgccauaaccacauacgugggauucucagugguguacccugguggcggcucuggcgguggcaguaacgacgguacuauuguaauuacuggaaguauuucagaccaaacgugcguuaucgaagaaccaagcacauuaaaccauauuaagguggugcaguugccuaagauaucaaagaacgcuuugaggaacgacggcgauacugcaggcgcaacaccuuucgauauaaagcuuaaggagugcccucaggcccugggcgcccugaagcucuacuucgaaccaggcauaacaacuaacuacgacaccggugaucuuauugcuuacaagcagacauauaacgcagccggcaacggccagcuuuccacuguaucauccgccacaaaggcuaagggcgucgaguucaggcuggcgaaucucaacggacaacacauuagaauggguacugauaagaccacccaagcugcacagaccuucacagguaaggucacuaacggcgguaagaguuauacacugcgcuaccuggcuagcuacgugaagaagccaaaggaggacgucgacgcagcacagauaacaucuuacguaggauucucuguuguuuauccggguggcgguagcaacgacggaaccauugugaucacuggcucaauuuccgaccagacagucauu

SEQ ID NO: 83 - protein, 1261 aa, Artificial sequence
  Synthetic
METPAQLLFLLLLWLPDTTGSLYAEHDATLTLAQGTQRDLVVDQDHILPVAEGTLRVKAKSLTTEIETGNPGSLIAEVQENIDNKQAIVVGKDLTLSSAHGNVANEANALLWAAGELTVKAQNITNKRAALIEAGGNARLTAAVALLNKLGRIRAGEDMHLDAPRIENTAKLSGEVQRKGVQDVGGGEHGRWSGIGYVNYWLRAGNGKKAGTIAAPWYGGDLTAEQSLIEVGKDLYLNAGARKDEHRHLLNEGVIQAGGHGHIGGDVDNRAVVRTVSAMEYFKTPLPVSLTALDNRAGLSPATWNFQSTYELLDYLLDQNRYEYIWGLYPTYTEWSVNTLKNLDLGYQAKPAPTAPPMPKAPELDLRGHTLESAEGRKIFGEYKKLQGEYEKAKMAVQAVEAYGEATRRVHDQLGQRYGKALGGMDAETKEVDGIIQEFAADLRTVYAKQADQATIDAETDKVAQRYKSQIDAVRLQRAKRSGSGAPVKQTLNFDLLKLAGDVESNPGPMETPAQLLFLLLLWLPDTTGVDPPVDCGRALGLHFWSSASLISDQTPDGTLIGKPVVGRSLLSKSCKVPDDIKEDLSDNHDGEPVDIVLELGSNYKIRPQSYGHPGIVVDLPSGSTEETGIAIYIDFGSSPMQKVGERQWLYPQKGEVLFDVLTINGDNAEVRYQAIKVGPLKRPRKLVLSQFPNLFTYKWVFMRGTSQERVLAQGTIDTDVATSTIDLKTCRYTSQTVSLPIIQRSALTGVGTTLGMTDFQMPFWCYGWPKVSVYMSATKTQTGVDGVALPATGQAAGMASGVGVQLINGKTQQPVKLGLQGKIALPEAQQTESATFSLPMKAQYYQTSTSTSAGKLSVTYAVTLNYDGGGSDDGTIVITGTITDTTCVIEDPSGPSHTKVVQLPKISKNALKANGDQAGRTPFIIKLKDCPSSLGNGVKAYFEPGPTTDYSTGDLRAYKMVYATNPQTQLSNIVAATEAQGVQVRISNLDDSKITMGANEATQQAAGFDPEVQTGGTSRTVTMRYLASYVKKNGDVEASAITTYVGFSVVYPGGGSGGGSNDGTIVITGSISDQTCVIEEPSTLNHIKVVQLPKISKNALRNDGDTAGATPFDIKLKECPQALGALKLYFEPGITTNYDTGDLIAYKQTYNAAGNGQLSTVSSATKAKGVEFRLANLNGQHIRMGTDKTTQAAQTFTGKVTNGGKSYTLRYLASYVKKPKEDVDAAQITSYVGFSVVYPGGGSNDGTIVITGSISDQTVI

SEQ ID NO: 84 - RNA, 3863 nt, Artificial sequence
  Synthetic
gggaaauaagagagaaaagaagaguaagaagaaauauaagaccccggcgccgccaccauggagaccccugcccagcugcuguuccugcugcugcuuuggcugccugacaccaccggaagccuguacgccgaacacgacgccacccugacccuggcccaaggcacccagagggaucuggugguggaccaggaccacauccugcccguggccgagggaacccugagagucaaagccaagagccucaccaccgagaucgagacaggcaacccuggcagccucaucgccgaggugcaggagaacaucgacaacaagcaggccaucgucgugggcaaagaccugacucuguccagcgcccacggcaacguagccaacgaggccaacgcccugcugugggcugccggugagcugaccgugaaggcccagaacaucaccaacaagagagccgcccugauugaggccggcgggaacgccagacugaccgcugccgucgcccuucugaacaagcugggcagaaucagagccggcgaggacaugcaccuggacgccccuagaaucgagaacaccgccaagcugagcggcgaggugcagagaaagggcgugcaggacgucggcggaggcgagcacggcagguggagcggcaucggcuacgugaacuacuggcuucgggccggcaacggcaagaaagccggcaccaucgccgcaccuugguacgguggcgaucugaccgcagagcagagccugaucgaggugggcaaggaccuguaccugaacgccggcgccagaaaggacgagcacagacaccugcugaacgagggcgugauccaggccggaggucacggccacaucggcggcgacguggacaacagggccgugguccguaccgugagcgccauggaguacuucaagaccccucugccugugagccugaccgcccuggacaauagagccggacucagcccagccaccuggaacuuccagagcaccuacgagcugcuggacuaccugcuggaccagaacagauacgaguacaucuggggccuguacccuaccuacaccgaguggagcgugaacacccugaagaaccuggaccugggcuaccaggccaagccugccccuaccgccccuccuaugccuaaggccccugagcuggaccugagaggccauacccuggagagcgccgagggcagaaagaucuucggcgaguacaagaagcugcagggcgaguacgagaaggccaagauggccgugcaagccguggaggccuacggcgaggccaccagaagagugcacgaccagcucgggcagcgauacggcaaggcccugggcggcauggacgccgaaaccaaggagguggacggcaucauccaggaguuugcggccgaucugagaaccguguacgccaaacaggccgaccaagccacuaucgacgccgagacugacaagguggcccagagauacaagagccagaucgacgccgugagacuccaaggaggagguggcucuggcggagguggaagcgguggaggcggaagugguggcggaggcagcguggauccaccuguggacugcggacgugcauuaggacugcacuucuggucaagugcgagccuuauaucggaucagaccccugacggcacauugaucggcaagccgguggucggacggucacuauugagcaagucuugcaaggugccugacgacauuaaggaagaccugucagacaaucacgacggcgagccaguugauaucguacuggagcuuggcagcaacuauaagauuaggccacagucguacggucauccuggaauaguggucgaccugccuuccgguucaacagaggagacagguaucgcuaucuacaucgacuucggcucuagcccaaugcagaaggucggcgagagacaguggcuguacccacagaagggugaggugcuuuucgacgugcugacuauuaacggagacaacgccgagguucgguaccaggccaucaaggucgguccacugaagcgcccaagaaagcugguccuuagucaguucccaaaccucuucacuuacaagugggucuucaugagaggcacaagccaggagcggguccucgcacagggcaccauagauacugacguggccacgagcaccaucgaccugaagacgugcagauacaccagucagaccgugucgcucccaaucauucagaggagugccuugaccgguguuggcaccacacuuggcaugacugacuuccaaaugccauucugguguuacggcuggccuaaggugucaguuuacaugucugcgacaaagacucaaacaggcguagacggcguagcacucccagcaaccggacaggccgccgguauggccuccggcgugggcguucaguugauuaacgguaagacgcagcagccugucaagcugggacuccagggcaagauugcccugccugaggcacagcagaccgaaagugcaacauucucacugccaaugaaggcccaguacuaccagaccaguaccucuacaucugccggcaagcuguccguuaccuacgccgucacucugaauuacgacggcggaggcuccgacgacggcacaaucgugauaaccgguacaauuacugacacaaccuguguaaucgaggauccuuccggcccaucgcauacuaagguuguucagcugccaaagauuuccaagaacgcccucaaggccaacggagaucaagcugguaggacaccauucauaauuaagcuaaaggacuguccauccucccucgguaacggcgugaaggccuauuucgagccaggaccuacgacugacuacucuacaggugaucucagagcauauaagaugguuuacgcaacaaauccucagacacagcucagcaauaucgucgccgccacugaggcucagggaguccagguccggaucuccaacuuagacgacucaaagaucaccaugggcgccaacgaggcaacucagcaggccgcuggauucgacccagagguacagacuggcgguacgucccgcacagucaccaugcgguaccuggcaaguuacguuaagaagaacggugacguggaggcguccgccauaaccacauacgugggauucucagugguguacccugguggcggcucuggcgguggcaguaacgacgguacuauuguaauuacuggaaguauuucagaccaaacgugcguuaucgaagaaccaagcacauuaaaccauauuaagguggugcaguugccuaagauaucaaagaacgcuuugaggaacgacggcgauacugcaggcgcaacaccuuucgauauaaagcuuaaggagugcccucaggcccugggcgcccugaagcucuacuucgaaccaggcauaacaacuaacuacgacaccggugaucuuauugcuuacaagcagacauauaacgcagccggcaacggccagcuuuccacuguaucauccgccacaaaggcuaagggcgucgaguucaggcuggcgaaucucaacggacaacacauuagaauggguacugauaagaccacccaagcugcacagaccuucacagguaaggucacuaacggcgguaagaguuauacacugcgcuaccuggcuagcuacgugaagaagccaaaggaggacgucgacgcagcacagauaacaucuuacguaggauucucuguuguuuauccggguggcgguagcaacgacggaaccauugugaucacuggcucaauuuccgaccagacagucauuugauaauaggcuggagccucgguggccuagcuucuugccccuugggccuccccccagccccuccuccccuuccugcacccguacccccguggucuuugaauaaagucugagugggcggc

SEQ ID NO: 85 - RNA, 3687 nt, Artificial sequence
  Synthetic
auggagaccccugcccagcugcuguuccugcugcugcuuuggcugccugacaccaccggaagccuguacgccgaacacgacgccacccugacccuggcccaaggcacccagagggaucuggugguggaccaggaccacauccugcccguggccgagggaacccugagagucaaagccaagagccucaccaccgagaucgagacaggcaacccuggcagccucaucgccgaggugcaggagaacaucgacaacaagcaggccaucgucgugggcaaagaccugacucuguccagcgcccacggcaacguagccaacgaggccaacgcccugcugugggcugccggugagcugaccgugaaggcccagaacaucaccaacaagagagccgcccugauugaggccggcgggaacgccagacugaccgcugccgucgcccuucugaacaagcugggcagaaucagagccggcgaggacaugcaccuggacgccccuagaaucgagaacaccgccaagcugagcggcgaggugcagagaaagggcgugcaggacgucggcggaggcgagcacggcagguggagcggcaucggcuacgugaacuacuggcuucgggccggcaacggcaagaaagccggcaccaucgccgcaccuugguacgguggcgaucugaccgcagagcagagccugaucgaggugggcaaggaccuguaccugaacgccggcgccagaaaggacgagcacagacaccugcugaacgagggcgugauccaggccggaggucacggccacaucggcggcgacguggacaacagggccgugguccguaccgugagcgccauggaguacuucaagaccccucugccugugagccugaccgcccuggacaauagagccggacucagcccagccaccuggaacuuccagagcaccuacgagcugcuggacuaccugcuggaccagaacagauacgaguacaucuggggccuguacccuaccuacaccgaguggagcgugaacacccugaagaaccuggaccugggcuaccaggccaagccugccccuaccgccccuccuaugccuaaggccccugagcuggaccugagaggccauacccuggagagcgccgagggcagaaagaucuucggcgaguacaagaagcugcagggcgaguacgagaaggccaagauggccgugcaagccguggaggccuacggcgaggccaccagaagagugcacgaccagcucgggcagcgauacggcaaggcccugggcggcauggacgccgaaaccaaggagguggacggcaucauccaggaguuugcggccgaucugagaaccguguacgccaaacaggccgaccaagccacuaucgacgccgagacugacaagguggcccagagauacaagagccagaucgacgccgugagacuccaaggaggagguggcucuggcggagguggaagcgguggaggcggaagugguggcggaggcagcguggauccaccuguggacugcggacgugcauuaggacugcacuucuggucaagugcgagccuuauaucggaucagaccccugacggcacauugaucggcaagccgguggucggacggucacuauugagcaagucuugcaaggugccugacgacauuaaggaagaccugucagacaaucacgacggcgagccaguugauaucguacuggagcuuggcagcaacuauaagauuaggccacagucguacggucauccuggaauaguggucgaccugccuuccgguucaacagaggagacagguaucgcuaucuacaucgacuucggcucuagcccaaugcagaaggucggcgagagacaguggcuguacccacagaagggugaggugcuuuucgacgugcugacuauuaacggagacaacgccgagguucgguaccaggccaucaaggucgguccacugaagcgcccaagaaagcugguccuuagucaguucccaaaccucuucacuuacaagugggucuucaugagaggcacaagccaggagcggguccucgcacagggcaccauagauacugacguggccacgagcaccaucgaccugaagacgugcagauacaccagucagaccgugucgcucccaaucauucagaggagugccuugaccgguguuggcaccacacuuggcaugacugacuuccaaaugccauucugguguuacggcuggccuaaggugucaguuuacaugucugcgacaaagacucaaacaggcguagacggcguagcacucccagcaaccggacaggccgccgguauggccuccggcgugggcguucaguugauuaacgguaagacgcagcagccugucaagcugggacuccagggcaagauugcccugccugaggcacagcagaccgaaagugcaacauucucacugccaaugaaggcccaguacuaccagaccaguaccucuacaucugccggcaagcuguccguuaccuacgccgucacucugaauuacgacggcggaggcuccgacgacggcacaaucgugauaaccgguacaauuacugacacaaccuguguaaucgaggauccuuccggcccaucgcauacuaagguuguucagcugccaaagauuuccaagaacgcccucaaggccaacggagaucaagcugguaggacaccauucauaauuaagcuaaaggacuguccauccucccucgguaacggcgugaaggccuauuucgagccaggaccuacgacugacuacucuacaggugaucucagagcauauaagaugguuuacgcaacaaauccucagacacagcucagcaauaucgucgccgccacugaggcucagggaguccagguccggaucuccaacuuagacgacucaaagaucaccaugggcgccaacgaggcaacucagcaggccgcuggauucgacccagagguacagacuggcgguacgucccgcacagucaccaugcgguaccuggcaaguuacguuaagaagaacggugacguggaggcguccgccauaaccacauacgugggauucucagugguguacccugguggcggcucuggcgguggcaguaacgacgguacuauuguaauuacuggaaguauuucagaccaaacgugcguuaucgaagaaccaagcacauuaaaccauauuaagguggugcaguugccuaagauaucaaagaacgcuuugaggaacgacggcgauacugcaggcgcaacaccuuucgauauaaagcuuaaggagugcccucaggcccugggcgcccugaagcucuacuucgaaccaggcauaacaacuaacuacgacaccggugaucuuauugcuuacaagcagacauauaacgcagccggcaacggccagcuuuccacuguaucauccgccacaaaggcuaagggcgucgaguucaggcuggcgaaucucaacggacaacacauuagaauggguacugauaagaccacccaagcugcacagaccuucacagguaaggucacuaacggcgguaagaguuauacacugcgcuaccuggcuagcuacgugaagaagccaaaggaggacgucgacgcagcacagauaacaucuuacguaggauucucuguuguuuauccggguggcgguagcaacgacggaaccauugugaucacuggcucaauuuccgaccagacagucauu

SEQ ID NO: 86 - protein, 1229 aa, Artificial sequence
  Synthetic
METPAQLLFLLLLWLPDTTGSLYAEHDATLTLAQGTQRDLVVDQDHILPVAEGTLRVKAKSLTTEIETGNPGSLIAEVQENIDNKQAIVVGKDLTLSSAHGNVANEANALLWAAGELTVKAQNITNKRAALIEAGGNARLTAAVALLNKLGRIRAGEDMHLDAPRIENTAKLSGEVQRKGVQDVGGGEHGRWSGIGYVNYWLRAGNGKKAGTIAAPWYGGDLTAEQSLIEVGKDLYLNAGARKDEHRHLLNEGVIQAGGHGHIGGDVDNRAVVRTVSAMEYFKTPLPVSLTALDNRAGLSPATWNFQSTYELLDYLLDQNRYEYIWGLYPTYTEWSVNTLKNLDLGYQAKPAPTAPPMPKAPELDLRGHTLESAEGRKIFGEYKKLQGEYEKAKMAVQAVEAYGEATRRVHDQLGQRYGKALGGMDAETKEVDGIIQEFAADLRTVYAKQADQATIDAETDKVAQRYKSQIDAVRLQGGGGSGGGGSGGGGSGGGGSVDPPVDCGRALGLHFWSSASLISDQTPDGTLIGKPVVGRSLLSKSCKVPDDIKEDLSDNHDGEPVDIVLELGSNYKIRPQSYGHPGIVVDLPSGSTEETGIAIYIDFGSSPMQKVGERQWLYPQKGEVLFDVLTINGDNAEVRYQAIKVGPLKRPRKLVLSQFPNLFTYKWVFMRGTSQERVLAQGTIDTDVATSTIDLKTCRYTSQTVSLPIIQRSALTGVGTTLGMTDFQMPFWCYGWPKVSVYMSATKTQTGVDGVALPATGQAAGMASGVGVQLINGKTQQPVKLGLQGKIALPEAQQTESATFSLPMKAQYYQTSTSTSAGKLSVTYAVTLNYDGGGSDDGTIVITGTITDTTCVIEDPSGPSHTKVVQLPKISKNALKANGDQAGRTPFIIKLKDCPSSLGNGVKAYFEPGPTTDYSTGDLRAYKMVYATNPQTQLSNIVAATEAQGVQVRISNLDDSKITMGANEATQQAAGFDPEVQTGGTSRTVTMRYLASYVKKNGDVEASAITTYVGFSVVYPGGGSGGGSNDGTIVITGSISDQTCVIEEPSTLNHIKVVQLPKISKNALRNDGDTAGATPFDIKLKECPQALGALKLYFEPGITTNYDTGDLIAYKQTYNAAGNGQLSTVSSATKAKGVEFRLANLNGQHIRMGTDKTTQAAQTFTGKVTNGGKSYTLRYLASYVKKPKEDVDAAQITSYVGFSVVYPGGGSNDGTIVITGSISDQTVI

SEQ ID NO: 87 - RNA, 3863 nt, Artificial sequence
  Synthetic
gggaaauaagagagaaaagaagaguaagaagaaauauaagaccccggcgccgccaccauggagaccccugcccagcugcuguuccugcugcugcuuuggcugccugacaccaccggaguggauccaccuguggacugcggacgugcauuaggacugcacuucuggucaagugcgagccuuauaucggaucagaccccugacggcacauugaucggcaagccgguggucggacggucacuauugagcaagucuugcaaggugccugacgacauuaaggaagaccugucagacaaucacgacggcgagccaguugauaucguacuggagcuuggcagcaacuauaagauuaggccacagucguacggucauccuggaauaguggucgaccugccuuccgguucaacagaggagacagguaucgcuaucuacaucgacuucggcucuagcccaaugcagaaggucggcgagagacaguggcuguacccacagaagggugaggugcuuuucgacgugcugacuauuaacggagacaacgccgagguucgguaccaggccaucaaggucgguccacugaagcgcccaagaaagcugguccuuagucaguucccaaaccucuucacuuacaagugggucuucaugagaggcacaagccaggagcggguccucgcacagggcaccauagauacugacguggccacgagcaccaucgaccugaagacgugcagauacaccagucagaccgugucgcucccaaucauucagaggagugccuugaccgguguuggcaccacacuuggcaugacugacuuccaaaugccauucugguguuacggcuggccuaaggugucaguuuacaugucugcgacaaagacucaaacaggcguagacggcguagcacucccagcaaccggacaggccgccgguauggccuccggcgugggcguucaguugauuaacgguaagacgcagcagccugucaagcugggacuccagggcaagauugcccugccugaggcacagcagaccgaaagugcaacauucucacugccaaugaaggcccaguacuaccagaccaguaccucuacaucugccggcaagcuguccguuaccuacgccgucacucugaauuacgacggcggaggcuccgacgacggcacaaucgugauaaccgguacaauuacugacacaaccuguguaaucgaggauccuuccggcccaucgcauacuaagguuguucagcugccaaagauuuccaagaacgcccucaaggccaacggagaucaagcugguaggacaccauucauaauuaagcuaaaggacuguccauccucccucgguaacggcgugaaggccuauuucgagccaggaccuacgacugacuacucuacaggugaucucagagcauauaagaugguuuacgcaacaaauccucagacacagcucagcaauaucgucgccgccacugaggcucagggaguccagguccggaucuccaacuuagacgacucaaagaucaccaugggcgccaacgaggcaacucagcaggccgcuggauucgacccagagguacagacuggcgguacgucccgcacagucaccaugcgguaccuggcaaguuacguuaagaagaacggugacguggaggcguccgccauaaccacauacgugggauucucagugguguacccugguggcggcucuggcgguggcaguaacgacgguacuauuguaauuacuggaaguauuucagaccaaacgugcguuaucgaagaaccaagcacauuaaaccauauuaagguggugcaguugccuaagauaucaaagaacgcuuugaggaacgacggcgauacugcaggcgcaacaccuuucgauauaaagcuuaaggagugcccucaggcccugggcgcccugaagcucuacuucgaaccaggcauaacaacuaacuacgacaccggugaucuuauugcuuacaagcagacauauaacgcagccggcaacggccagcuuuccacuguaucauccgccacaaaggcuaagggcgucgaguucaggcuggcgaaucucaacggacaacacauuagaauggguacugauaagaccacccaagcugcacagaccuucacagguaaggucacuaacggcgguaagaguuauacacugcgcuaccuggcuagcuacgugaagaagccaaaggaggacgucgacgcagcacagauaacaucuuacguaggauucucuguuguuuauccggguggcgguagcaacgacggaaccauugugaucacuggcucaauuuccgaccagacagucauuggaggagguggcucuggcggagguggaagcgguggaggcggaagugguggcggaggcagcagccuguacgccgaacacgacgccacccugacccuggcccaaggcacccagagggaucuggugguggaccaggaccacauccugcccguggccgagggaacccugagagucaaagccaagagccucaccaccgagaucgagacaggcaacccuggcagccucaucgccgaggugcaggagaacaucgacaacaagcaggccaucgucgugggcaaagaccugacucuguccagcgcccacggcaacguagccaacgaggccaacgcccugcugugggcugccggugagcugaccgugaaggcccagaacaucaccaacaagagagccgcccugauugaggccggcgggaacgccagacugaccgcugccgucgcccuucugaacaagcugggcagaaucagagccggcgaggacaugcaccuggacgccccuagaaucgagaacaccgccaagcugagcggcgaggugcagagaaagggcgugcaggacgucggcggaggcgagcacggcagguggagcggcaucggcuacgugaacuacuggcuucgggccggcaacggcaagaaagccggcaccaucgccgcaccuugguacgguggcgaucugaccgcagagcagagccugaucgaggugggcaaggaccuguaccugaacgccggcgccagaaaggacgagcacagacaccugcugaacgagggcgugauccaggccggaggucacggccacaucggcggcgacguggacaacagggccgugguccguaccgugagcgccauggaguacuucaagaccccucugccugugagccugaccgcccuggacaauagagccggacucagcccagccaccuggaacuuccagagcaccuacgagcugcuggacuaccugcuggaccagaacagauacgaguacaucuggggccuguacccuaccuacaccgaguggagcgugaacacccugaagaaccuggaccugggcuaccaggccaagccugccccuaccgccccuccuaugccuaaggccccugagcuggaccugagaggccauacccuggagagcgccgagggcagaaagaucuucggcgaguacaagaagcugcagggcgaguacgagaaggccaagauggccgugcaagccguggaggccuacggcgaggccaccagaagagugcacgaccagcucgggcagcgauacggcaaggcccugggcggcauggacgccgaaaccaaggagguggacggcaucauccaggaguuugcggccgaucugagaaccguguacgccaaacaggccgaccaagccacuaucgacgccgagacugacaagguggcccagagauacaagagccagaucgacgccgugagacuccaaugauaauaggcuggagccucgguggccuagcuucuugccccuugggccuccccccagccccuccuccccuuccugcacccguacccccguggucuuugaauaaagucugagugggcggc

SEQ ID NO: 88 - RNA, 3687 nt, Artificial sequence
  Synthetic
auggagaccccugcccagcugcuguuccugcugcugcuuuggcugccugacaccaccggaguggauccaccuguggacugcggacgugcauuaggacugcacuucuggucaagugcgagccuuauaucggaucagaccccugacggcacauugaucggcaagccgguggucggacggucacuauugagcaagucuugcaaggugccugacgacauuaaggaagaccugucagacaaucacgacggcgagccaguugauaucguacuggagcuuggcagcaacuauaagauuaggccacagucguacggucauccuggaauaguggucgaccugccuuccgguucaacagaggagacagguaucgcuaucuacaucgacuucggcucuagcccaaugcagaaggucggcgagagacaguggcuguacccacagaagggugaggugcuuuucgacgugcugacuauuaacggagacaacgccgagguucgguaccaggccaucaaggucgguccacugaagcgcccaagaaagcugguccuuagucaguucccaaaccucuucacuuacaagugggucuucaugagaggcacaagccaggagcggguccucgcacagggcaccauagauacugacguggccacgagcaccaucgaccugaagacgugcagauacaccagucagaccgugucgcucccaaucauucagaggagugccuugaccgguguuggcaccacacuuggcaugacugacuuccaaaugccauucugguguuacggcuggccuaaggugucaguuuacaugucugcgacaaagacucaaacaggcguagacggcguagcacucccagcaaccggacaggccgccgguauggccuccggcgugggcguucaguugauuaacgguaagacgcagcagccugucaagcugggacuccagggcaagauugcccugccugaggcacagcagaccgaaagugcaacauucucacugccaaugaaggcccaguacuaccagaccaguaccucuacaucugccggcaagcuguccguuaccuacgccgucacucugaauuacgacggcggaggcuccgacgacggcacaaucgugauaaccgguacaauuacugacacaaccuguguaaucgaggauccuuccggcccaucgcauacuaagguuguucagcugccaaagauuuccaagaacgcccucaaggccaacggagaucaagcugguaggacaccauucauaauuaagcuaaaggacuguccauccucccucgguaacggcgugaaggccuauuucgagccaggaccuacgacugacuacucuacaggugaucucagagcauauaagaugguuuacgcaacaaauccucagacacagcucagcaauaucgucgccgccacugaggcucagggaguccagguccggaucuccaacuuagacgacucaaagaucaccaugggcgccaacgaggcaacucagcaggccgcuggauucgacccagagguacagacuggcgguacgucccgcacagucaccaugcgguaccuggcaaguuacguuaagaagaacggugacguggaggcguccgccauaaccacauacgugggauucucagugguguacccugguggcggcucuggcgguggcaguaacgacgguacuauuguaauuacuggaaguauuucagaccaaacgugcguuaucgaagaaccaagcacauuaaaccauauuaagguggugcaguugccuaagauaucaaagaacgcuuugaggaacgacggcgauacugcaggcgcaacaccuuucgauauaaagcuuaaggagugcccucaggcccugggcgcccugaagcucuacuucgaaccaggcauaacaacuaacuacgacaccggugaucuuauugcuuacaagcagacauauaacgcagccggcaacggccagcuuuccacuguaucauccgccacaaaggcuaagggcgucgaguucaggcuggcgaaucucaacggacaacacauuagaauggguacugauaagaccacccaagcugcacagaccuucacagguaaggucacuaacggcgguaagaguuauacacugcgcuaccuggcuagcuacgugaagaagccaaaggaggacgucgacgcagcacagauaacaucuuacguaggauucucuguuguuuauccggguggcgguagcaacgacggaaccauugugaucacuggcucaauuuccgaccagacagucauuggaggagguggcucuggcggagguggaagcgguggaggcggaagugguggcggaggcagcagccuguacgccgaacacgacgccacccugacccuggcccaaggcacccagagggaucuggugguggaccaggaccacauccugcccguggccgagggaacccugagagucaaagccaagagccucaccaccgagaucgagacaggcaacccuggcagccucaucgccgaggugcaggagaacaucgacaacaagcaggccaucgucgugggcaaagaccugacucuguccagcgcccacggcaacguagccaacgaggccaacgcccugcugugggcugccggugagcugaccgugaaggcccagaacaucaccaacaagagagccgcccugauugaggccggcgggaacgccagacugaccgcugccgucgcccuucugaacaagcugggcagaaucagagccggcgaggacaugcaccuggacgccccuagaaucgagaacaccgccaagcugagcggcgaggugcagagaaagggcgugcaggacgucggcggaggcgagcacggcagguggagcggcaucggcuacgugaacuacuggcuucgggccggcaacggcaagaaagccggcaccaucgccgcaccuugguacgguggcgaucugaccgcagagcagagccugaucgaggugggcaaggaccuguaccugaacgccggcgccagaaaggacgagcacagacaccugcugaacgagggcgugauccaggccggaggucacggccacaucggcggcgacguggacaacagggccgugguccguaccgugagcgccauggaguacuucaagaccccucugccugugagccugaccgcccuggacaauagagccggacucagcccagccaccuggaacuuccagagcaccuacgagcugcuggacuaccugcuggaccagaacagauacgaguacaucuggggccuguacccuaccuacaccgaguggagcgugaacacccugaagaaccuggaccugggcuaccaggccaagccugccccuaccgccccuccuaugccuaaggccccugagcuggaccugagaggccauacccuggagagcgccgagggcagaaagaucuucggcgaguacaagaagcugcagggcgaguacgagaaggccaagauggccgugcaagccguggaggccuacggcgaggccaccagaagagugcacgaccagcucgggcagcgauacggcaaggcccugggcggcauggacgccgaaaccaaggagguggacggcaucauccaggaguuugcggccgaucugagaaccguguacgccaaacaggccgaccaagccacuaucgacgccgagacugacaagguggcccagagauacaagagccagaucgacgccgugagacuccaa

SEQ ID NO: 89 - protein, 1229 aa, Artificial sequence
  Synthetic
METPAQLLFLLLLWLPDTTGVDPPVDCGRALGLHFWSSASLISDQTPDGTLIGKPVVGRSLLSKSCKVPDDIKEDLSDNHDGEPVDIVLELGSNYKIRPQSYGHPGIVVDLPSGSTEETGIAIYIDFGSSPMQKVGERQWLYPQKGEVLFDVLTINGDNAEVRYQAIKVGPLKRPRKLVLSQFPNLFTYKWVFMRGTSQERVLAQGTIDTDVATSTIDLKTCRYTSQTVSLPIIQRSALTGVGTTLGMTDFQMPFWCYGWPKVSVYMSATKTQTGVDGVALPATGQAAGMASGVGVQLINGKTQQPVKLGLQGKIALPEAQQTESATFSLPMKAQYYQTSTSTSAGKLSVTYAVTLNYDGGGSDDGTIVITGTITDTTCVIEDPSGPSHTKVVQLPKISKNALKANGDQAGRTPFIIKLKDCPSSLGNGVKAYFEPGPTTDYSTGDLRAYKMVYATNPQTQLSNIVAATEAQGVQVRISNLDDSKITMGANEATQQAAGFDPEVQTGGTSRTVTMRYLASYVKKNGDVEASAITTYVGFSVVYPGGGSGGGSNDGTIVITGSISDQTCVIEEPSTLNHIKVVQLPKISKNALRNDGDTAGATPFDIKLKECPQALGALKLYFEPGITTNYDTGDLIAYKQTYNAAGNGQLSTVSSATKAKGVEFRLANLNGQHIRMGTDKTTQAAQTFTGKVTNGGKSYTLRYLASYVKKPKEDVDAAQITSYVGFSVVYPGGGSNDGTIVITGSISDQTVIGGGGSGGGGSGGGGSGGGGSSLYAEHDATLTLAQGTQRDLVVDQDHILPVAEGTLRVKAKSLTTEIETGNPGSLIAEVQENIDNKQAIVVGKDLTLSSAHGNVANEANALLWAAGELTVKAQNITNKRAALIEAGGNARLTAAVALLNKLGRIRAGEDMHLDAPRIENTAKLSGEVQRKGVQDVGGGEHGRWSGIGYVNYWLRAGNGKKAGTIAAPWYGGDLTAEQSLIEVGKDLYLNAGARKDEHRHLLNEGVIQAGGHGHIGGDVDNRAVVRTVSAMEYFKTPLPVSLTALDNRAGLSPATWNFQSTYELLDYLLDQNRYEYIWGLYPTYTEWSVNTLKNLDLGYQAKPAPTAPPMPKAPELDLRGHTLESAEGRKIFGEYKKLQGEYEKAKMAVQAVEAYGEATRRVHDQLGQRYGKALGGMDAETKEVDGIIQEFAADLRTVYAKQADQATIDAETDKVAQRYKSQIDAVRLQ

SEQ ID NO: 90 - RNA, 3863 nt, Artificial sequence; identical to SEQ ID NO: 87
  Synthetic

SEQ ID NO: 91 - RNA, 3687 nt, Artificial sequence; identical to SEQ ID NO: 88
  Synthetic

SEQ ID NO: 92 - protein, 1207 aa, Artificial sequence
  Synthetic
METPAQLLFLLLLWLPDTTGTLEHVQHIIGGAGNDAITGNAHDNFLAGGSGDDRLDGGAGNDVLVGGEGQNTVIGGAGDDVFLQDLGVWSNQLDGGAGVDTVKYNVHQPSEERLERMGDTGIHADLQKGTVEKWPALNLFSVDHVKNIENLHGSRLNDRIAGDDQDNELWGHDGNDVIRGRGGDDILRGGLGLDTLYGEDGNDIFLQDDETVSDDIDGGAGLDTVDYSAMIHPGRIVAPHEYGFGIEADLSREWVRKASALGVDYYDNVRNVENVIGTSMKDVLIGDAQANTLMGQGGDDTVRGGDGDDLLFGGDGNDMLYGDAGNDVLYGGLGDDTLEGGAGNDWFGQTQAREHDVLRGGDGVDTVDYSQTGAHAGIAAGRIGLGILADLGAGRVDKLGEAGSSAYDTVSGIENVVGTELADRITGDAQANVLRGAGGADVLAGGEGDDVLLGGDGDDQLSGDAGRDRLYGEAGDDWFFQDAANAGNLLDGGDGRDTVDFSGPGRGLDAGAKGVFLSLGKGFASLMDEPETSNVLRNIENAVGSARDDVLIGDAGANVLNGLAGNDVLSGGAGDDVLLGDEGSDLLSGDAGNDDLFGGQGDDTYLFGVGYGHDTRAKRSGSGAPVKQTLNFDLLKLAGDVESNPGPMSRKLFASILIGALLGIGAPPSAHAGADDVVDSSKSFVMENFASYHGTKPGYVDSIQKGIQKPKSGTQGNYDDDWEGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVKYINNWEQAKALSVELEINFETRGKRGQDAMYEYMAQACAGNRVRRSVGSSLSCINLDWDVIRDKTKTKIESLKEHGPIKNKMSESPNKAVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKAKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 93 - RNA, 3686 nt, Artificial sequence
  Synthetic
gggaaauaagagagaaaagaagaguaagaagaaauauaagaccccggcgccgccaccauggagaccccugcccagcugcuguuccugcugcugcuuuggcugccagacaccaccggaacccuggagcacgugcagcacaucaucggcggcgccggcaacgacgccaucacuggcaacgcccacgacaacuuccuggccggcggcagcggcgacgacagacuggacggcggagcagguaacgacgugcucgucggaggcgaggggcagaacaccgugauuggcggagccggagacgacguguuccugcaggaccugggcguguggagcaaccaguuagacggcggcgcagguguggauacuguuaaguacaacgugcaccagccuagcgaggagagacuggagagaaugggcgacaccggcauccacgccgaccugcagaagggaaccguggagaaguggccugcccugaaccuguucagcguggaccacgugaagaacaucgagaaccugcacggcaguagacuaaacgacagaauugcaggagacgaucaggacaacgaacuguggggucacgacggaaacgacgugauacguggcagaggaggcgacgacauccugcggggcggccugggccuggacacccuguacggcgaggacggaaacgacaucuuccuccaagacgacgaaaccgugagcgacgauaucgacggcggugccggauuagacaccgucgacuacagcgccaugauccacccuggcagaaucguggccccucacgaguacggcuucggcaucgaggcugaccugagcagagagugggugagaaaggccagcgcguuggguguagacuacuacgacaacgugagaaacgucgagaacgucauuggcaccagcaugaaagacgugcucaucggugacgcucaggccaacacccugaugggccagggcggugacgacacagugcgaggcggagacggagacgaccugcuguucgguggcgacggcaacgauauguuauacggcgacgcuggaaacgacguguuauacgguggacugggugacgauacauuggaggguggcgcgggcaacgauugguucggccagacacaagccagagagcacgacguucuacguggcggcgacggaguggacacuguggacuacucacaaaccggcgcccacgccggaaucgcggccgggagaaucggcuugggaauccuugcugacuugggugcaggccggguggacaagcugggugaggcaggcucaagugccuacgauacagugagcggcauugagaacguagucggcacggagcuugcugaucggauuaccggagacgcccaagcaaacguguuacggggugcuggaggcgcugacguccucgcuggcggugagggcgacgacgugcuguugggaggagacggugacgaccaacugucgggcgacgcgggaagggauagacuguacggagaagcaggagacgacugguucuuccaggacgccgccaacgcgggaaaccugcuugacgguggugacggacgagauaccguagauuucucugguccaggcagaggccucgacgcaggcgccaagggcgucuuccugucccucgguaagggcuucgccagccuuauggacgaaccugagacuucuaacgugcugagaaauauugagaacgccgucggcagcgccagggacgacgucuuaaucggcgacgccggugcgaacguccuaaacggcuuggccggaaacgacguuuuaagcggaggagcuggcgacgacguacugcuuggcgacgagggcagcgaccuccuaucaggugacgccgguaacgacgaccucuucggcggacagggugacgauacuuauuuguucggcgugggcuacggacacgacacuggaggagguggcucuggcggagguggaagcgguggaggcggaagugguggcggaggcagcggagcugacgacgugguggacagcagcaagagcuucgugauggagaacuucgccagcuaccacggcaccaagcccggcuacguggacaguauccagaagggcauccagaagcccaagagcggcacccagggcaacuacgacgacgacugggagggcuucuacagcaccgacaacaaguacgacgccgccggcuacagcgucgacaacgagaacccacuguccggcaaagccggaggcguggugaaggugaccuaccccggccugaccaaggugcuggcccugaagguggacaacgccgagaccauuaagaaggagcugggccugagccugaccgagccccugauggagcaggugggcacugaggaguucaucaagcguuucggggacggggcgaguagaguggugcugagccugcccuucgccgagggcagcagcagcgugaaguacaucaacaacugggagcaggccaaggcccugagcguggagcuggagaucaacuucgagacccggggcaaacgaggccaggacgccauguacgaguacauggcacaggcuugcgccggcaaucgggugcggagaagcgugggcucuagccugagcugcaucaaucuggacugggacgugauccgggacaagacgaagaccaagaucgagagccugaaggagcacggccccaucaagaacaagaugagcgagagccccaacaaggccgugagcgaggagaaggccaagcaguaccuggaggaguuccaccagaccgccuuggaacaccccgagcugagcgagcugaagacugugaccggcaccaaccccguuuucgccggcgccaacuacgccgccugggccgugaacguggcucaggugaucgacagcgagaccgccgacaaccuggagaagaccaccgccgcccugagcauccugcccggcaucggcagcgugaugggcaucgcugacggcgccgugcaccacaacaccgaggagaucguggcccagagcaucgcccucagcagccugaugguggcccaggccauuccccuggugggcgagcugguggacaucggcuucgccgccuacaacuucguggagagcaucaucaaccuguuccagguggugcacaacagcuacaaccggcccgccuauagccccggacacaagacccagcccuuucugcacgacggcuacgccgugagcuggaacaccguggaggacagcaucauccggaccggcuuccagggcgagagcggccacgacaucaagaucaccgccgagaacaccccucugcccaucgccggcguucugcugcccaccauccccggcaagcuggacgugaacaaggccaagacccacaucagcgugaacggccggaagauccgaaugcggugccgggcaaucgacggcgacgugaccuucugccggccuaagagccccguguacgugggcaacggcguucacgccaaccugcacguggccuuccaccggagcagcucugagaagauccacagcaacgagaucagcagcgacagcaucggcgugcugggcuaccagaagaccguggaccacaccaaggugaacagcaaacugagccuguucuucgagaucaagagcugauaauaggcuggagccucgguggccuagcuucuugccccuugggccuccccccagccccuccuccccuuccugcacccguacccccguggucuuugaauaaagucugagugggcggc

SEQ ID NO: 94 - RNA, 3510 nt, Artificial sequence
  Synthetic
auggagaccccugcccagcugcuguuccugcugcugcuuuggcugccagacaccaccggaacccuggagcacgugcagcacaucaucggcggcgccggcaacgacgccaucacuggcaacgcccacgacaacuuccuggccggcggcagcggcgacgacagacuggacggcggagcagguaacgacgugcucgucggaggcgaggggcagaacaccgugauuggcggagccggagacgacguguuccugcaggaccugggcguguggagcaaccaguuagacggcggcgcagguguggauacuguuaaguacaacgugcaccagccuagcgaggagagacuggagagaaugggcgacaccggcauccacgccgaccugcagaagggaaccguggagaaguggccugcccugaaccuguucagcguggaccacgugaagaacaucgagaaccugcacggcaguagacuaaacgacagaauugcaggagacgaucaggacaacgaacuguggggucacgacggaaacgacgugauacguggcagaggaggcgacgacauccugcggggcggccugggccuggacacccuguacggcgaggacggaaacgacaucuuccuccaagacgacgaaaccgugagcgacgauaucgacggcggugccggauuagacaccgucgacuacagcgccaugauccacccuggcagaaucguggccccucacgaguacggcuucggcaucgaggcugaccugagcagagagugggugagaaaggccagcgcguuggguguagacuacuacgacaacgugagaaacgucgagaacgucauuggcaccagcaugaaagacgugcucaucggugacgcucaggccaacacccugaugggccagggcggugacgacacagugcgaggcggagacggagacgaccugcuguucgguggcgacggcaacgauauguuauacggcgacgcuggaaacgacguguuauacgguggacugggugacgauacauuggaggguggcgcgggcaacgauugguucggccagacacaagccagagagcacgacguucuacguggcggcgacggaguggacacuguggacuacucacaaaccggcgcccacgccggaaucgcggccgggagaaucggcuugggaauccuugcugacuugggugcaggccggguggacaagcugggugaggcaggcucaagugccuacgauacagugagcggcauugagaacguagucggcacggagcuugcugaucggauuaccggagacgcccaagcaaacguguuacggggugcuggaggcgcugacguccucgcuggcggugagggcgacgacgugcuguugggaggagacggugacgaccaacugucgggcgacgcgggaagggauagacuguacggagaagcaggagacgacugguucuuccaggacgccgccaacgcgggaaaccugcuugacgguggugacggacgagauaccguagauuucucugguccaggcagaggccucgacgcaggcgccaagggcgucuuccugucccucgguaagggcuucgccagccuuauggacgaaccugagacuucuaacgugcugagaaauauugagaacgccgucggcagcgccagggacgacgucuuaaucggcgacgccggugcgaacguccuaaacggcuuggccggaaacgacguuuuaagcggaggagcuggcgacgacguacugcuuggcgacgagggcagcgaccuccuaucaggugacgccgguaacgacgaccucuucggcggacagggugacgauacuuauuuguucggcgugggcuacggacacgacacuggaggagguggcucuggcggagguggaagcgguggaggcggaagugguggcggaggcagcggagcugacgacgugguggacagcagcaagagcuucgugauggagaacuucgccagcuaccacggcaccaagcccggcuacguggacaguauccagaagggcauccagaagcccaagagcggcacccagggcaacuacgacgacgacugggagggcuucuacagcaccgacaacaaguacgacgccgccggcuacagcgucgacaacgagaacccacuguccggcaaagccggaggcguggugaaggugaccuaccccggccugaccaaggugcuggcccugaagguggacaacgccgagaccauuaagaaggagcugggccugagccugaccgagccccugauggagcaggugggcacugaggaguucaucaagcguuucggggacggggcgaguagaguggugcugagccugcccuucgccgagggcagcagcagcgugaaguacaucaacaacugggagcaggccaaggcccugagcguggagcuggagaucaacuucgagacccggggcaaacgaggccaggacgccauguacgaguacauggcacaggcuugcgccggcaaucgggugcggagaagcgugggcucuagccugagcugcaucaaucuggacugggacgugauccgggacaagacgaagaccaagaucgagagccugaaggagcacggccccaucaagaacaagaugagcgagagccccaacaaggccgugagcgaggagaaggccaagcaguaccuggaggaguuccaccagaccgccuuggaacaccccgagcugagcgagcugaagacugugaccggcaccaaccccguuuucgccggcgccaacuacgccgccugggccgugaacguggcucaggugaucgacagcgagaccgccgacaaccuggagaagaccaccgccgcccugagcauccugcccggcaucggcagcgugaugggcaucgcugacggcgccgugcaccacaacaccgaggagaucguggcccagagcaucgcccucagcagccugaugguggcccaggccauuccccuggugggcgagcugguggacaucggcuucgccgccuacaacuucguggagagcaucaucaaccuguuccagguggugcacaacagcuacaaccggcccgccuauagccccggacacaagacccagcccuuucugcacgacggcuacgccgugagcuggaacaccguggaggacagcaucauccggaccggcuuccagggcgagagcggccacgacaucaagaucaccgccgagaacaccccucugcccaucgccggcguucugcugcccaccauccccggcaagcuggacgugaacaaggccaagacccacaucagcgugaacggccggaagauccgaaugcggugccgggcaaucgacggcgacgugaccuucugccggccuaagagccccguguacgugggcaacggcguucacgccaaccugcacguggccuuccaccggagcagcucugagaagauccacagcaacgagaucagcagcgacagcaucggcgugcugggcuaccagaagaccguggaccacaccaaggugaacagcaaacugagccuguucuucgagaucaagagc

SEQ ID NO: 95 - protein, 1170 aa, Artificial sequence
  Synthetic
METPAQLLFLLLLWLPDTTGTLEHVQHIIGGAGNDAITGNAHDNFLAGGSGDDRLDGGAGNDVLVGGEGQNTVIGGAGDDVFLQDLGVWSNQLDGGAGVDTVKYNVHQPSEERLERMGDTGIHADLQKGTVEKWPALNLFSVDHVKNIENLHGSRLNDRIAGDDQDNELWGHDGNDVIRGRGGDDILRGGLGLDTLYGEDGNDIFLQDDETVSDDIDGGAGLDTVDYSAMIHPGRIVAPHEYGFGIEADLSREWVRKASALGVDYYDNVRNVENVIGTSMKDVLIGDAQANTLMGQGGDDTVRGGDGDDLLFGGDGNDMLYGDAGNDVLYGGLGDDTLEGGAGNDWFGQTQAREHDVLRGGDGVDTVDYSQTGAHAGIAAGRIGLGILADLGAGRVDKLGEAGSSAYDTVSGIENVVGTELADRITGDAQANVLRGAGGADVLAGGEGDDVLLGGDGDDQLSGDAGRDRLYGEAGDDWFFQDAANAGNLLDGGDGRDTVDFSGPGRGLDAGAKGVFLSLGKGFASLMDEPETSNVLRNIENAVGSARDDVLIGDAGANVLNGLAGNDVLSGGAGDDVLLGDEGSDLLSGDAGNDDLFGGQGDDTYLFGVGYGHDTGGGGSGGGGSGGGGSGGGGSGADDVVDSSKSFVMENFASYHGTKPGYVDSIQKGIQKPKSGTQGNYDDDWEGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVKYINNWEQAKALSVELEINFETRGKRGQDAMYEYMAQACAGNRVRRSVGSSLSCINLDWDVIRDKTKTKIESLKEHGPIKNKMSESPNKAVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKAKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 96 - RNA, 3686 nt, Artificial sequence
  Synthetic
gggaaauaagagagaaaagaagaguaagaagaaauauaagaccccggcgccgccaccauggagaccccugcccagcugcuguuccugcugcugcuuuggcugccagacaccaccggaggagcugacgacgugguggacagcagcaagagcuucgugauggagaacuucgccagcuaccacggcaccaagcccggcuacguggacaguauccagaagggcauccagaagcccaagagcggcacccagggcaacuacgacgacgacugggagggcuucuacagcaccgacaacaaguacgacgccgccggcuacagcgucgacaacgagaacccacuguccggcaaagccggaggcguggugaaggugaccuaccccggccugaccaaggugcuggcccugaagguggacaacgccgagaccauuaagaaggagcugggccugagccugaccgagccccugauggagcaggugggcacugaggaguucaucaagcguuucggggacggggcgaguagaguggugcugagccugcccuucgccgagggcagcagcagcgugaaguacaucaacaacugggagcaggccaaggcccugagcguggagcuggagaucaacuucgagacccggggcaaacgaggccaggacgccauguacgaguacauggcacaggcuugcgccggcaaucgggugcggagaagcgugggcucuagccugagcugcaucaaucuggacugggacgugauccgggacaagacgaagaccaagaucgagagccugaaggagcacggccccaucaagaacaagaugagcgagagccccaacaaggccgugagcgaggagaaggccaagcaguaccuggaggaguuccaccagaccgccuuggaacaccccgagcugagcgagcugaagacugugaccggcaccaaccccguuuucgccggcgccaacuacgccgccugggccgugaacguggcucaggugaucgacagcgagaccgccgacaaccuggagaagaccaccgccgcccugagcauccugcccggcaucggcagcgugaugggcaucgcugacggcgccgugcaccacaacaccgaggagaucguggcccagagcaucgcccucagcagccugaugguggcccaggccauuccccuggugggcgagcugguggacaucggcuucgccgccuacaacuucguggagagcaucaucaaccuguuccagguggugcacaacagcuacaaccggcccgccuauagccccggacacaagacccagcccuuucugcacgacggcuacgccgugagcuggaacaccguggaggacagcaucauccggaccggcuuccagggcgagagcggccacgacaucaagaucaccgccgagaacaccccucugcccaucgccggcguucugcugcccaccauccccggcaagcuggacgugaacaaggccaagacccacaucagcgugaacggccggaagauccgaaugcggugccgggcaaucgacggcgacgugaccuucugccggccuaagagccccguguacgugggcaacggcguucacgccaaccugcacguggccuuccaccggagcagcucugagaagauccacagcaacgagaucagcagcgacagcaucggcgugcugggcuaccagaagaccguggaccacaccaaggugaacagcaaacugagccuguucuucgagaucaagagcggaggagguggcucuggcggagguggaagcgguggaggcggaagugguggcggaggcagcacccuggagcacgugcagcacaucaucggcggcgccggcaacgacgccaucacuggcaacgcccacgacaacuuccuggccggcggcagcggcgacgacagacuggacggcggagcagguaacgacgugcucgucggaggcgaggggcagaacaccgugauuggcggagccggagacgacguguuccugcaggaccugggcguguggagcaaccaguuagacggcggcgcagguguggauacuguuaaguacaacgugcaccagccuagcgaggagagacuggagagaaugggcgacaccggcauccacgccgaccugcagaagggaaccguggagaaguggccugcccugaaccuguucagcguggaccacgugaagaacaucgagaaccugcacggcaguagacuaaacgacagaauugcaggagacgaucaggacaacgaacuguggggucacgacggaaacgacgugauacguggcagaggaggcgacgacauccugcggggcggccugggccuggacacccuguacggcgaggacggaaacgacaucuuccuccaagacgacgaaaccgugagcgacgauaucgacggcggugccggauuagacaccgucgacuacagcgccaugauccacccuggcagaaucguggccccucacgaguacggcuucggcaucgaggcugaccugagcagagagugggugagaaaggccagcgcguuggguguagacuacuacgacaacgugagaaacgucgagaacgucauuggcaccagcaugaaagacgugcucaucggugacgcucaggccaacacccugaugggccagggcggugacgacacagugcgaggcggagacggagacgaccugcuguucgguggcgacggcaacgauauguuauacggcgacgcuggaaacgacguguuauacgguggacugggugacgauacauuggaggguggcgcgggcaacgauugguucggccagacacaagccagagagcacgacguucuacguggcggcgacggaguggacacuguggacuacucacaaaccggcgcccacgccggaaucgcggccgggagaaucggcuugggaauccuugcugacuugggugcaggccggguggacaagcugggugaggcaggcucaagugccuacgauacagugagcggcauugagaacguagucggcacggagcuugcugaucggauuaccggagacgcccaagcaaacguguuacggggugcuggaggcgcugacguccucgcuggcggugagggcgacgacgugcuguugggaggagacggugacgaccaacugucgggcgacgcgggaagggauagacuguacggagaagcaggagacgacugguucuuccaggacgccgccaacgcgggaaaccugcuugacgguggugacggacgagauaccguagauuucucugguccaggcagaggccucgacgcaggcgccaagggcgucuuccugucccucgguaagggcuucgccagccuuauggacgaaccugagacuucuaacgugcugagaaauauugagaacgccgucggcagcgccagggacgacgucuuaaucggcgacgccggugcgaacguccuaaacggcuuggccggaaacgacguuuuaagcggaggagcuggcgacgacguacugcuuggcgacgagggcagcgaccuccuaucaggugacgccgguaacgacgaccucuucggcggacagggugacgauacuuauuuguucggcgugggcuacggacacgacacuugauaauaggcuggagccucgguggccuagcuucuugccccuugggccuccccccagccccuccuccccuuccugcacccguacccccguggucuuugaauaaagucugagugggcggc

SEQ ID NO: 97 - RNA, 3510 nt, Artificial sequence
  Synthetic
auggagaccccugcccagcugcuguuccugcugcugcuuuggcugccagacaccaccggaggagcugacgacgugguggacagcagcaagagcuucgugauggagaacuucgccagcuaccacggcaccaagcccggcuacguggacaguauccagaagggcauccagaagcccaagagcggcacccagggcaacuacgacgacgacugggagggcuucuacagcaccgacaacaaguacgacgccgccggcuacagcgucgacaacgagaacccacuguccggcaaagccggaggcguggugaaggugaccuaccccggccugaccaaggugcuggcccugaagguggacaacgccgagaccauuaagaaggagcugggccugagccugaccgagccccugauggagcaggugggcacugaggaguucaucaagcguuucggggacggggcgaguagaguggugcugagccugcccuucgccgagggcagcagcagcgugaaguacaucaacaacugggagcaggccaaggcccugagcguggagcuggagaucaacuucgagacccggggcaaacgaggccaggacgccauguacgaguacauggcacaggcuugcgccggcaaucgggugcggagaagcgugggcucuagccugagcugcaucaaucuggacugggacgugauccgggacaagacgaagaccaagaucgagagccugaaggagcacggccccaucaagaacaagaugagcgagagccccaacaaggccgugagcgaggagaaggccaagcaguaccuggaggaguuccaccagaccgccuuggaacaccccgagcugagcgagcugaagacugugaccggcaccaaccccguuuucgccggcgccaacuacgccgccugggccgugaacguggcucaggugaucgacagcgagaccgccgacaaccuggagaagaccaccgccgcccugagcauccugcccggcaucggcagcgugaugggcaucgcugacggcgccgugcaccacaacaccgaggagaucguggcccagagcaucgcccucagcagccugaugguggcccaggccauuccccuggugggcgagcugguggacaucggcuucgccgccuacaacuucguggagagcaucaucaaccuguuccagguggugcacaacagcuacaaccggcccgccuauagccccggacacaagacccagcccuuucugcacgacggcuacgccgugagcuggaacaccguggaggacagcaucauccggaccggcuuccagggcgagagcggccacgacaucaagaucaccgccgagaacaccccucugcccaucgccggcguucugcugcccaccauccccggcaagcuggacgugaacaaggccaagacccacaucagcgugaacggccggaagauccgaaugcggugccgggcaaucgacggcgacgugaccuucugccggccuaagagccccguguacgugggcaacggcguucacgccaaccugcacguggccuuccaccggagcagcucugagaagauccacagcaacgagaucagcagcgacagcaucggcgugcugggcuaccagaagaccguggaccacaccaaggugaacagcaaacugagccuguucuucgagaucaagagcggaggagguggcucuggcggagguggaagcgguggaggcggaagugguggcggaggcagcacccuggagcacgugcagcacaucaucggcggcgccggcaacgacgccaucacuggcaacgcccacgacaacuuccuggccggcggcagcggcgacgacagacuggacggcggagcagguaacgacgugcucgucggaggcgaggggcagaacaccgugauuggcggagccggagacgacguguuccugcaggaccugggcguguggagcaaccaguuagacggcggcgcagguguggauacuguuaaguacaacgugcaccagccuagcgaggagagacuggagagaaugggcgacaccggcauccacgccgaccugcagaagggaaccguggagaaguggccugcccugaaccuguucagcguggaccacgugaagaacaucgagaaccugcacggcaguagacuaaacgacagaauugcaggagacgaucaggacaacgaacuguggggucacgacggaaacgacgugauacguggcagaggaggcgacgacauccugcggggcggccugggccuggacacccuguacggcgaggacggaaacgacaucuuccuccaagacgacgaaaccgugagcgacgauaucgacggcggugccggauuagacaccgucgacuacagcgccaugauccacccuggcagaaucguggccccucacgaguacggcuucggcaucgaggcugaccugagcagagagugggugagaaaggccagcgcguuggguguagacuacuacgacaacgugagaaacgucgagaacgucauuggcaccagcaugaaagacgugcucaucggugacgcucaggccaacacccugaugggccagggcggugacgacacagugcgaggcggagacggagacgaccugcuguucgguggcgacggcaacgauauguuauacggcgacgcuggaaacgacguguuauacgguggacugggugacgauacauuggaggguggcgcgggcaacgauugguucggccagacacaagccagagagcacgacguucuacguggcggcgacggaguggacacuguggacuacucacaaaccggcgcccacgccggaaucgcggccgggagaaucggcuugggaauccuugcugacuugggugcaggccggguggacaagcugggugaggcaggcucaagugccuacgauacagugagcggcauugagaacguagucggcacggagcuugcugaucggauuaccggagacgcccaagcaaacguguuacggggugcuggaggcgcugacguccucgcuggcggugagggcgacgacgugcuguugggaggagacggugacgaccaacugucgggcgacgcgggaagggauagacuguacggagaagcaggagacgacugguucuuccaggacgccgccaacgcgggaaaccugcuugacgguggugacggacgagauaccguagauuucucugguccaggcagaggccucgacgcaggcgccaagggcgucuuccugucccucgguaagggcuucgccagccuuauggacgaaccugagacuucuaacgugcugagaaauauugagaacgccgucggcagcgccagggacgacgucuuaaucggcgacgccggugcgaacguccuaaacggcuuggccggaaacgacguuuuaagcggaggagcuggcgacgacguacugcuuggcgacgagggcagcgaccuccuaucaggugacgccgguaacgacgaccucuucggcggacagggugacgauacuuauuuguucggcgugggcuacggacacgacacu

SEQ ID NO: 98 - protein, 1170 aa, Artificial sequence
  Synthetic
METPAQLLFLLLLWLPDTTGGADDVVDSSKSFVMENFASYHGTKPGYVDSIQKGIQKPKSGTQGNYDDDWEGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVKYINNWEQAKALSVELEINFETRGKRGQDAMYEYMAQACAGNRVRRSVGSSLSCINLDWDVIRDKTKTKIESLKEHGPIKNKMSESPNKAVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKAKTHISVNGRKIRMRCRAIDGDVTFCRPKSPVYVGNGVHANLHVAFHRSSSEKIHSNEISSDSIGVLGYQKTVDHTKVNSKLSLFFEIKSGGGGSGGGGSGGGGSGGGGSTLEHVQHIIGGAGNDAITGNAHDNFLAGGSGDDRLDGGAGNDVLVGGEGQNTVIGGAGDDVFLQDLGVWSNQLDGGAGVDTVKYNVHQPSEERLERMGDTGIHADLQKGTVEKWPALNLFSVDHVKNIENLHGSRLNDRIAGDDQDNELWGHDGNDVIRGRGGDDILRGGLGLDTLYGEDGNDIFLQDDETVSDDIDGGAGLDTVDYSAMIHPGRIVAPHEYGFGIEADLSREWVRKASALGVDYYDNVRNVENVIGTSMKDVLIGDAQANTLMGQGGDDTVRGGDGDDLLFGGDGNDMLYGDAGNDVLYGGLGDDTLEGGAGNDWFGQTQAREHDVLRGGDGVDTVDYSQTGAHAGIAAGRIGLGILADLGAGRVDKLGEAGSSAYDTVSGIENVVGTELADRITGDAQANVLRGAGGADVLAGGEGDDVLLGGDGDDQLSGDAGRDRLYGEAGDDWFFQDAANAGNLLDGGDGRDTVDFSGPGRGLDAGAKGVFLSLGKGFASLMDEPETSNVLRNIENAVGSARDDVLIGDAGANVLNGLAGNDVLSGGAGDDVLLGDEGSDLLSGDAGNDDLFGGQGDDTYLFGVGYGHDT

SEQ ID NO: 99 - RNA, 47 nt, Artificial sequence
  Synthetic
gggaaauaagagagaaaagaagaguaagaagaaauauaagagccacc

SEQ ID NO: 100 - RNA, 119 nt, Artificial sequence
  Synthetic
ugauaauaggcuggagccucgguggccaugcuucuugccccuugggccuccccccagccccuccuccccuuccugcacccguacccccguggucuuugaauaaagucugagugggcggc

SEQ ID NO: 101 - protein, 15 aa, Artificial sequence
  Synthetic
GGSGGSGGSGGSGGG

SEQ ID NO: 102 - protein, 8 aa, Artificial sequence
  Synthetic
GGGSGGGS

SEQ ID NO: 103 - protein, 7 aa, Artificial sequence
  Synthetic
GGGSGGG

SEQ ID NO: 104 - protein, 4 aa, Artificial sequence
  Synthetic
GGGS

SEQ ID NO: 105 - protein, 4 aa, Artificial sequence
  Synthetic
  REPEAT (1)..(4): may be repeated 1 to 5 times
GGGS

SEQ ID NO: 106 - protein, 4 aa, Artificial sequence
  Synthetic
GSGG

SEQ ID NO: 107 - protein, 4 aa, Artificial sequence
  Synthetic
  REPEAT (1)..(4): may be repeated 1 to 5 times
GSGG

SEQ ID NO: 108 - RNA, 11 nt, Artificial sequence
  Synthetic
gggauccuacc